(19) United States
(12) Patent Application Publication
(10) Pub. No.: US 2025/0171419 A1
(43) Pub. Date: May 29, 2025

(54) NOVEL NICOTINAMIDE PHOSPHORIBOSYLTRANSFERASE INHIBITORS AND USES THEREOF

(71) Applicant: Heidelberg Pharma Research GmbH, Ladenburg (DE)

(72) Inventors: Pablo Ruedas-Batuecas, Ladenburg (DE); Hendrik Gruss, Ladenburg (DE); Anikó Pálfi, Ladenburg (DE); Sarah-Jane Neuberth, Ladenburg (DE); Torsten Hechler, Ladenburg (DE)

(21) Appl. No.: 18/957,044

(22) Filed: Nov. 22, 2024

(30) Foreign Application Priority Data
Nov. 24, 2023 (EP) 23212107.9

(51) Int. Cl. C07D 401/14; A61K 31/454; A61K 31/4545; A61K 47/68; A61P 35/00

(52) U.S. Cl. CPC C07D 401/14; A61K 31/454; A61K 31/4545; A61K 47/6851; A61P 35/00

(57) ABSTRACT

The present disclosure pertains to the provision of NAMPT inhibitors, a method of synthesizing the same as well as their use in antibody-drug-conjugates. The present disclosure further pertains to pharmaceutical compositions comprising the NAMPT inhibitors of the disclosure and their use in cancer treatment. In a further aspect, the present disclosure pertains to a method of treatment using the NAMPT inhibitors of the disclosure.

### CROSS-REFERENCE TO RELATED APPLICATIONS

This application claims priority to European Patent Application No. 23212107.9, filed 24 Nov. 2023, the entire contents of which are incorporated herein by reference.

### FIELD

The present disclosure relates to compounds and compositions for inhibition of nicotinamide phosphoryltransferase (“NAMPT”), their synthesis and antibody-drug conjugates comprising such NAMPT inhibitors and their application.

### BACKGROUND

Nicotine adenine dinucleotide (NAD+) is an essential cofactor in metabolic redox chemistry. which is composed chemically of two nucleotides (nicotinamide and adenosine) joined through their phosphate group which is conserved in every cell system throughout evolution.

From a functional perspective, NAD+ is considered a multifaceted molecule. First, it has a major role in bioenergetic redox pathways, where it is reduced to NADH and acts as an electron transfer molecule in cellular energetics without being catabolized. Additionally, it plays a vital role in biosynthetic pathways and helps maintain redox stability in cells by neutralizing reactive oxygen species (ROS) generated as a result of metabolic activity. When phosphorylated by NAD+ kinase, it generates NADP+.

NAMPT (nicotinamide phosphoribosyltransferase also known as pre-B-cell-colony-enhancing factor (PBEF), NMPRT, NMPETase or NAmPRTase, international nomenclature E.C.2.4.2.12) is a key enzyme that plays a crucial role in the most widely used salvage pathway for NAD⁺ regeneration in cancer cells. NAMPT is a dimeric class of type II phosphoribosyl transferases with a molecular weight of about 52 kDa. Each monomer consists of a catalytical pocket domain that binds to its substrate NAM with a Kₘ value of around 1 μM. However, the enzyme is typically auto-phosphorylated at His247, resulting in a 1×10³ fold increase in enzyme activity and an 1.6×10⁴ fold enhanced affinity for NAM, leading to an effective Kₘ value of around 5 nM. In mammals, two isoforms of NAMPT exist: an intracellular and extracellular variant (iNAMPT and eNAMPT, respectively). The intracellular form of NAMPT is responsible for the enzymatic reaction which transforms NAM into nicotine-adenine mononucleotide (NAMN) and is primarily found in the cytosol and nucleus, along with its possible presence in mitochondria. NAMPT is the rate-limiting enzyme in the production of NAD⁺ and its inhibition leads to a rapid depletion of NAD⁺.

Cancer cells proliferate continuously such that these cells have to adapt to a stressful and dynamic microenvironment. This results in an increased need for energy, macromolecules and the maintenance of the cellular redox status by cancer cells. NAD⁺ is used as an electron carrier in glycolysis, which is up-regulated in cancer cells due to the Warburg effect, as well as in mitochondrial oxidative phosphorylation. NAD⁺ also serves as a substrate for several enzymes, such as poly-ADP-ribose polymerases (PARPs) and sirtuins (SIRTs) which are involved in DNA repair and gene expression, processes which are often aberrantly regulated in cancer cells and which lead to a higher consumption of NAD⁺.

Phosphorylated forms of NAD⁺/NADH also exist and are often employed for biosynthetic and/or cell protection purposes in addition to energy generation. They are also involved in the cellular response to oxidative stress. For these reasons, many cancer cells have an increased need for NAD⁺ and its synthesis is constantly required, rendering cancer cells particularly sensitive to NAMPT inhibition.

NAMPT has been implicated in the regulation of cell viability during genotoxic or oxidative stress and that NAMPT inhibitors are potentially useful for the treatment of e.g. inflammation, metabolic disorders and cancer.

Daporinad also known as APO866, FK866, WK175 or WK22 ((E)-N-[4-(1-benzoylpiperidin-4-yl)butyl]-3-(pyrldine-3-yl)-acrylamide) is a potent and selective inhibitor of NAMPT which interferes with NAD biosynthesis, ATP generation and induces cell death. In vivo efficacy of daporinad was shown in murine renal cell carcinoma model RENCA. Clinical trials with daporinad have been completed for the treatment of chronic lymphocytic leukemia (CLL), cutaneous T cell lymphoma (CTL), and advanced melanoma.

CHS-828 is also known as GMIX1778 (N-[6-(4-chlorophenoxy)hexyl]-N′-cyano-N″-4-pyridinyl-guanidine), an inhibitor of NAMPT demonstrated highly cytotoxic effects in vitro and in vivo in human breast and lung cancer cell line-derived in vivo models. Observed responses in the clinical trials with the compound were stable disease which has led to the assumption that the lack of significant activity in clinical trials may result from the inability to dose NAMPT inhibitors to higher drug exposures due to dose-limiting toxicities of which thrombocytopenia has been the most significant dose-limiting toxicity in patients treated in clinical trials in phases I and II of solid tumors. The use of targeted NAMPT inhibitors through their use in antibody-drug conjugates (ADCs) has been tried. Corresponding ADCs show in vitro efficacy, however, in vivo NAMPT inhibitor-based ADCs have not proven as efficacious in animal models as well as in clinical trials as anticipated.

Published NAMPT inhibitors like CHS828 or Seattle genetics lead inhibitor 4 (Mol Cancer Ther. 2018 December; 17(12):2633-2642) present the chemical moiety cyanoguanidine-pyridine as the key interacting group with the NAMPT catalytic pocket. Due to the ADC endo lysosomal processing pathway this cyanoguanidine moiety can be counterproductive due to stability issues.

There is thus an unmet medical need for NAMPT inhibitor-based ADCs that show in vivo efficacy.

### SUMMARY

It has been surprisingly found that NAMPT inhibitors according to the current disclosure having the structure of

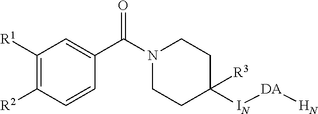

### (I)

wherein R¹, R², R³, R^{L}, IN, DA, HN are independently:
R¹ is OH, NH₂, N₃, SH, H, NHR^{L}; OR^{L}, or SR^{L};
R² is OH, NH₂, N₃, SH, H, NHR^{L}, OR^{L}, or SR^{L};
R³ is H-bond donor group, as OH, NH₂, SH, SO₃H, COOH, or CONH₂;
I_{N} is an interconnecting unit, selected from C₁₋₆ alkyl, 5 or 6-membered aromatic ring, a 5 or 6-membered heteroaromatic ring, or a combination thereof;
DA is a H-bond donor acceptor group selected from cyanoguanidine, acrylamide, urea, or thiourea;
H_{N}=heteroaromatic or heterocyclic ring or a combination thereof, preferably pyridyl, isoindolinyl, indolyl, isoquinolinyl, quinolinyl, or imidazopyridinyl;
R^{L} is a linker having the structure L-Z,
L is a linker selected from a cleavable or non-cleavable linker;
Z is a thiol-reactive, or amine-reactive chemical moiety; and
wherein if R¹ is NHR^{L}, OR^{L}, or SR^{L}, R² is not NHR^{L}, OR^{L}, SR^{L} and if R² is NHR^{L}, OR^{L}, SR^{L}, R¹ is not NHR^{L} OR^{L}, SR^{L}′;
   are highly potent NAMPT inhibitors and retain their activity when used as payloads in antibody-drug conjugates and are stable in relation to lyosomal hydration.

In some embodiments Z is selected from a group comprising azides, amines, alkynes, tetrazines, strained cyclooctynes, aldehydes, HIPS reagents (Hydrazino-Pictet-Spengler) and other reactive groups useful in bioconjugations and bio-orthogonal reactions.

In preferred embodiments Z is selected from a group comprising thiol-reactive or amine-reactive chemical moieties, azides, amines, alkynes, tetrazines, strained cyclooctynes, aldehydes and HIPS reagents (Hydrazino-Pictet-Spengler), in more preferred embodiments Z is selected from a group comprising thiol-reactive or amine-reactive chemical moieties, azides, amines, alkynes and tetrazines.

Thus, in a first aspect the present disclosure pertains to the provision of compounds according to structure (I) above.

In a second aspect, the present disclosure pertains to a method of synthesizing compounds according to formula (I), wherein the method comprises the reaction step of reacting intermediate (i1) with intermediate (i2) to yield intermediate (i3).

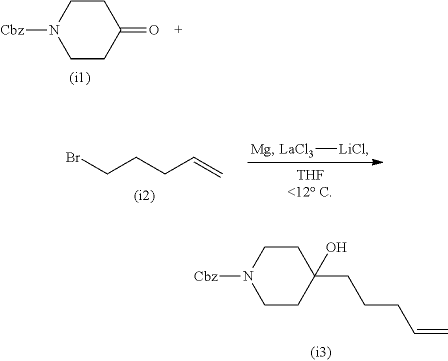

In a third aspect, the present disclosure pertains to antibody-drug conjugates (ADCs) comprising the NAMPT inhibitors of the disclosure as well as their use in cancer treatment.

### BRIEF DESCRIPTION OF THE DRAWINGS

FIG. 1: General synthesis scheme of the NAMPT inhibitors of the disclosure.

FIG. 2A: In vivo efficacy study of L540 subcutaneous xenograft model in CB17 SCID mice, 10 mice per group. Effect on Tumor volume. Control experiment using Brentuximab as exemplary antibody to test the NAMPT inhibitors of the disclosure. (97=c22b.8′).

FIG. 2B: In vivo efficacy study of L540 subcutaneous xenograft model in CB17 SCID mice, 10 mice per group. Effect on overall survival. Control experiment using Brentuximab as exemplary antibody to test the NAMPT inhibitors of the disclosure. (97=c22b.8′).

FIG. 3: Cytotoxic profile of free inhibitor 94 (c22a) compared with anti-CD30 mAb (Brentuximab)-97 (-c22b.8′). The ADC has an about 10⁴-fold higher toxicity in comparison to the free inhibitor.

FIG. 4A: In vivo efficacy study of L540 disseminated model in NXG mice, 10 mice per group. Effect on Body weight.

FIG. 4B: In vivo efficacy study of L540 disseminated model in NXG mice, 10 mice per group. Effect on overall survival.

FIG. 5: Cytotoxicity of free urea-isoindoline inhibitor (c42a) compared with alpha-amanitin in L540 cells (EC₅₀ (M)).

FIG. 6: Comparison of brentuximab (anti-CD30) high DAR (DAR≈10) ADC loaded with linker-payload c42b.10 with brentuximab high DAR ADC loaded with control linker-payload 37 (not according to disclosure), using free Brentuximab mAb as negative control on L540 CD30+ cell line.

FIG. 7: Comparison of trastuzumab ADC DAR≈10, linker payload C42b.10′ vs control payload 36 (not according to disclosure). in MDA-MB-453 HER2⁺ cell line using culture conditions as disclosed in WO 2021/013693.

FIG. 8: In vivo efficacy study of NCI-N87 subcutaneous tumors with single and multiple dose treatment with Trastuzumab based high DAR (DAR≈10) ADC loaded with linker-payload C42b.10 compared to multiple dose treatment with commercially available FK866 NAMPT inhibitor and multiple dose PBS group as untreated control in NMRI-nu mice.

FIG. 9: In vivo efficacy study of L540 subcutaneous tumors with single dose treatment with Brentuximab-based high DAR (DAR≈10) ADC loaded with linker-payload C22b.8 compared to single dose treatment with brentuximab-based high DAR ADC loaded with linker-payload 37 (not according to disclosure) and single dose PBS group as untreated control in CB-17 SCID mice.

FIG. 10: In vitro lysosomal stability assay: A) Comparison of cyanoguanidine 36 recovery vs. time with and without lysosomal extract, normalised to T0 value. B) Mass spec detection of hydrated compound 113 along the experiment time course. C) Cytotoxicity assay of compound 113 compared with 36, and α-amanitin as assay control in L540 cells EC50 (M).

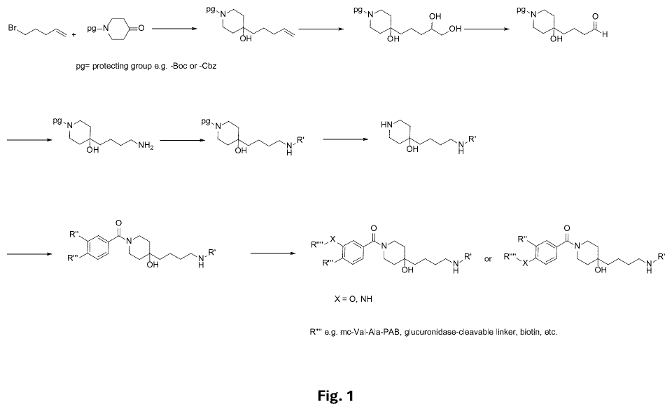

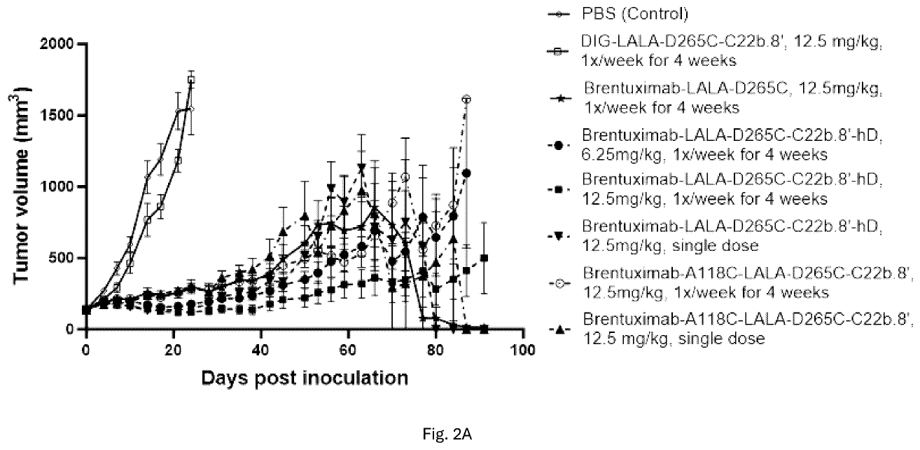

### DETAILED DESCRIPTION

Before the disclosure is described in detail, it is to be understood that this disclosure is not limited to the particular component parts of the devices described or process steps of the methods described as such devices and methods may vary. It is also to be understood that the terminology used herein is for purposes of describing particular embodiments only and is not intended to be limiting. It must be noted that, as used in the specification and the appended claims, the singular forms “a”, “an” and “the” include singular and/or plural referents unless the context clearly dictates otherwise. It is moreover to be understood that, in case parameter ranges are given which are delimited by numeric values, the ranges are deemed to include these limitation values. Recitation of ranges of values herein is merely intended to serve as a shorthand method of referring individually to each separate value falling within the range. Unless otherwise indicated herein, each individual value is incorporated into the specification as if it were individually recited herein.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers particularly for prior art documents that disclose standard or routine methods. In that case, the incorporation by reference has mainly the purpose to provide sufficient enabling disclosure and avoid lengthy repetitions. The definitions of the chemical groups as used herein shall have the meaning and be defined as provided in “Compendium of Chemical Terminology” (“Gold Book”) published by the International Union of Pure and Applied Chemistry (IUPAC) version 2.3.3, goldbook.iupac.org, ISBN: 0-9678550-9-8), the content of which is hereby incorporated by reference.

Throughout this application the term “about” is used which shall refer to +/−10% of the numerical value with which it is used.

According to a first aspect, the present disclosure pertains to a compound having the structure according to formula (I)

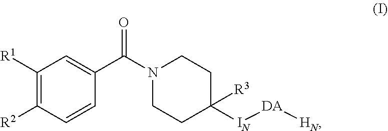

wherein R¹, R², R³, R^{L}, I_{N}, DA, H_{N} are independently:
R¹ is OH, NH₂, N₃, SH, H, NHR^{L}; OR^{L}, or SR^{L};
R² is OH, NH₂, N₃, SH, H, NHR^{L}, OR^{L}, or SR^{L};
R³ is H-bond donor group, as OH, NH₂, SH, SO₃H, COOH, or CONH₂;
I_{N} is an interconnecting unit, selected from C1-6 alkyl, 5 or 6-membered aromatic ring, a 5 or 6-membered heteroaromatic ring, or a combination thereof;
DA is a H-bond donor acceptor group selected from cyanoguanidine, acrylamide, urea, or thiourea;
H_{N} is heteroaromatic or heterocyclic ring or a combination thereof, preferably, pyridyl, isoindolinyl, indolyl, isoquinolinyl, quinolinyl, or imidazopyridinyl;
R^{L} is a linker having the structure L-Z;
L is a linker selected from a cleavable or non-cleavable linker;
Z is a thiol-reactive, or amine-reactive chemical moiety; and
wherein if R¹ is NHR^{L}, OR^{L}, or SR^{L}, R² is not NHR^{L}, OR^{L}, SR^{L} and if R² is NHR^{L}, OR^{L}, SR^{L}, R¹ is not NHR^{L} OR^{L}, SR^{L}.

In some embodiments Z is selected from a group comprising azides, amines, alkynes, tetrazines, strained cyclooctynes, aldehydes, HIPS reagents (Hydrazino-Pictet-Spengler) and other reactive groups useful in bioconjugations and bio-orthogonal chemistry.

In preferred embodiments Z is selected from a group comprising thiol-reactive or amine-reactive chemical moieties, azides, amines, alkynes, tetrazines, strained cyclooctynes, aldehydes and HIPS reagents (Hydrazino-Pictet-Spengler), in more preferred embodiments Z is selected from a group comprising thiol-reactive or amine-reactive chemical moieties, azides, amines, alkynes and tetrazines.

According to one embodiment, the heteroaromatic or heterocyclic ring H_{N} of the compound (I) is selected from

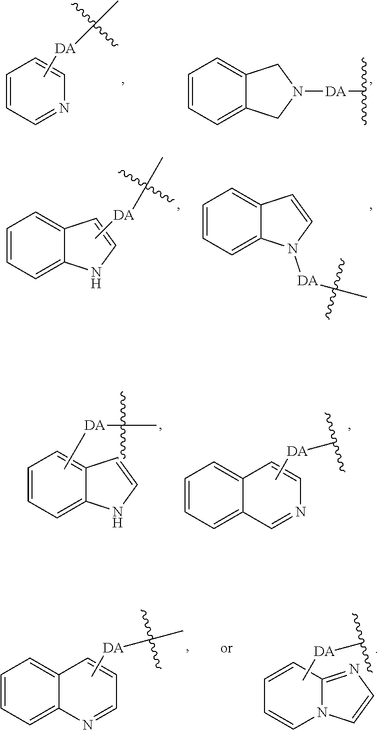

wherein R¹, R², R³, DA and I_{N} are as disclosed above.

According to one embodiment, the H-bond donor acceptor group DA of compound (I) of the disclosure is selected from cyanoguanidine, acrylamide, urea, or thiourea

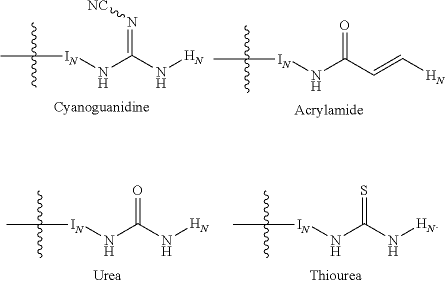

In case H_{N} is a heterocycle in which DA is bound to the nitrogen atom of the heterocycle then the nitrogen atom of the heterocycle is part of DA, e.g. in case H_{N} is isoindolyl or indolyl. For example, if H_{N} is an isoindolyl moiety and DA is one of cyanoguanidine, urea or thiourea, then DA-H_{N} is selected from:

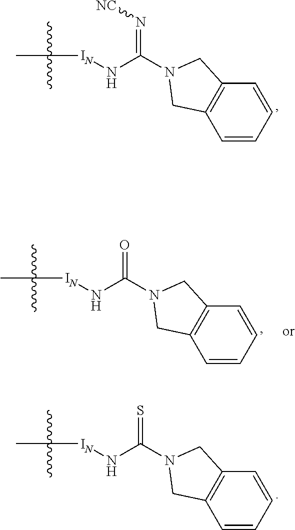

In case H_{N} is an indolyl moiety and DA is one of cyanoguanidine, urea or thiourea, then DA-H_{N} is selected from:

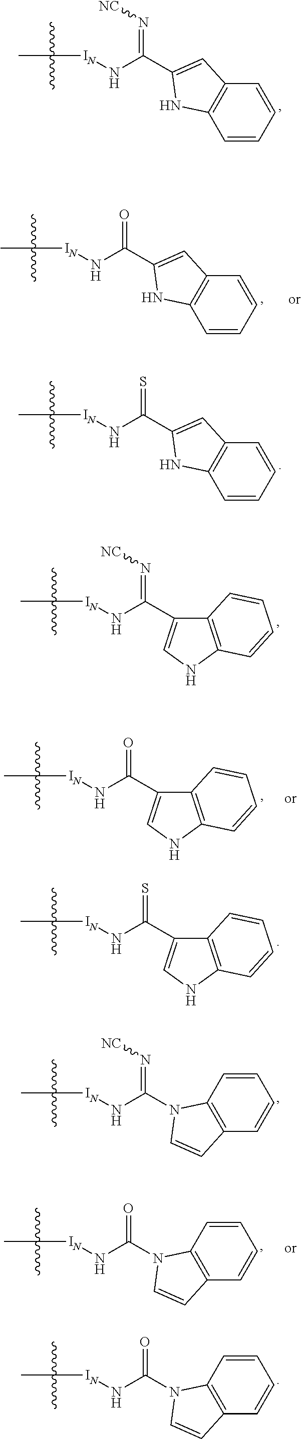

In one embodiment, the interconnecting unit I_{N} of compound (I) as disclosed herein is selected from C1-6 alkyl, 5- or 6-membered aromatic ring, a 5- or 6-membered heteroaromatic ring, or a combination thereof. Accordingly, compound (I) of the disclosure as disclosed herein is selected from one or more of the below groups:

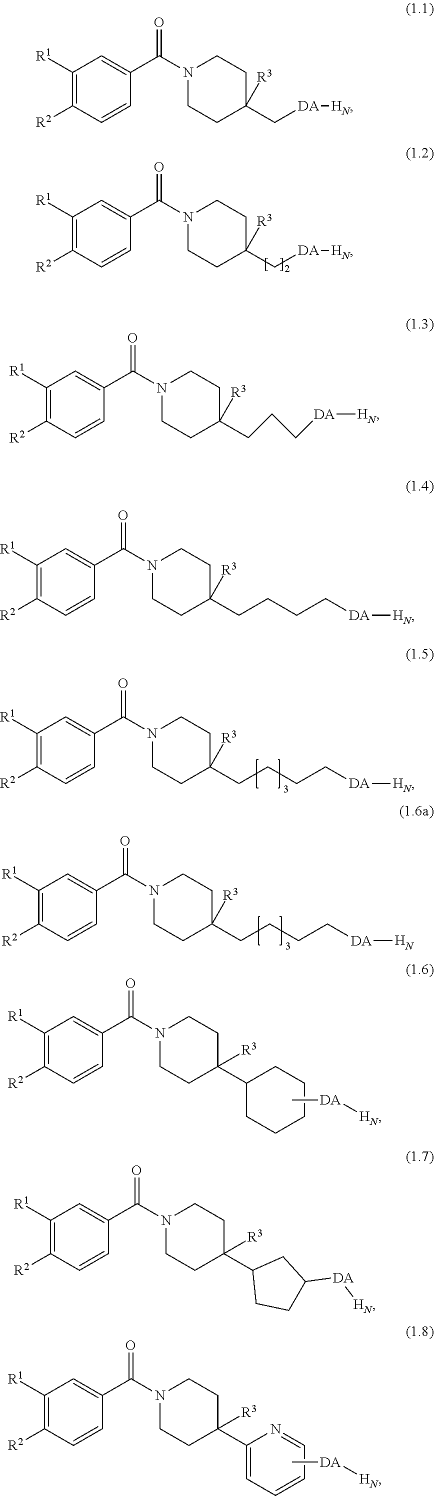

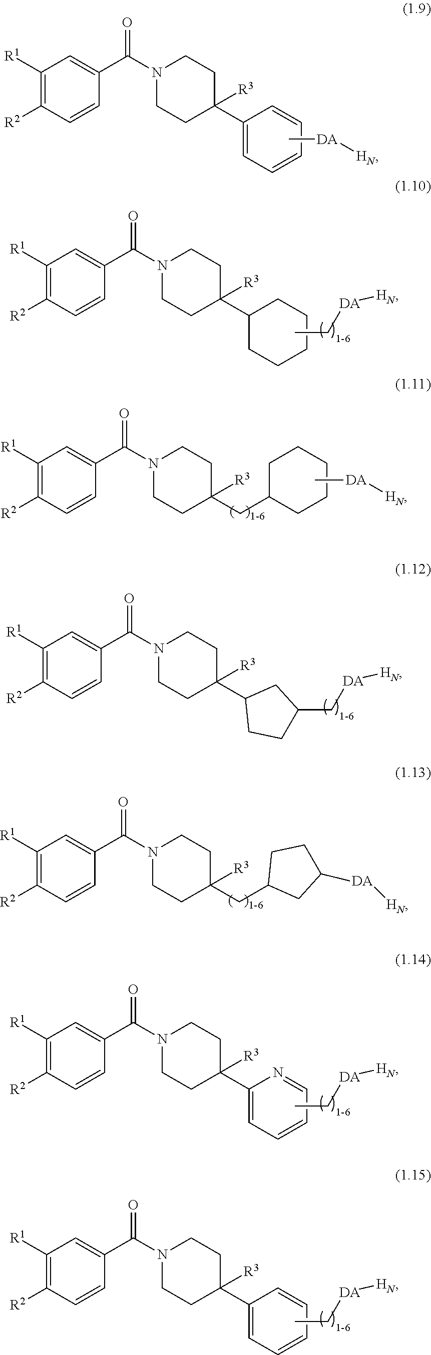

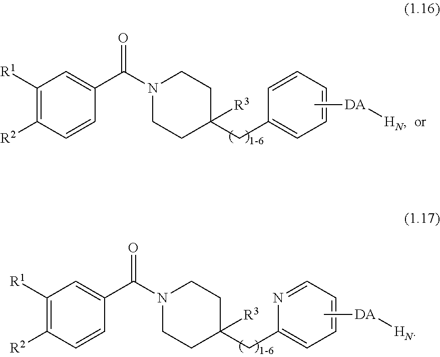

According to preferred embodiments, the interconnecting unit I_{N} of the compound (I) as disclosed herein is selected from C₁₋₆ alkyl.

In some embodiments, the compound of the present disclosure is selected from a compound having the structure

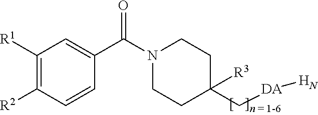

wherein D_{A}, H_{N}, R¹, R² and R³ are as defined herein.

According to some embodiments, R³ in the compound of the present disclosure as disclosed above is selected from the group consisting of OH, NH₂, SH, SO₃H, COOH, and CONH₂, DA is cyanoguanidine and H_{N} is selected from the group consisting of pyridyl, isoindolinyl, indolyl, isoquinolinyl, quinolinyl, and imidazopyridinyl.

According to some embodiments, R³ is selected from the group consisting of OH, NH₂, SH, SO₃H, COOH, and CONH₂, DA is acrylamide and H_{N} is selected from the group consisting of pyridyl, isoindolinyl, indolyl, isoquinolinyl, quinolinyl, and imidazopyridinyl.

According to some embodiments, R³ in the compound of the present disclosure as disclosed above is selected from the group consisting of OH, NH₂, SH, SO₃H, COOH, and CONH₂, DA is urea and H_{N} is selected from the group consisting of pyridyl, isoindolinyl, indolyl, isoquinolinyl, quinolinyl, and imidazopyridinyl.

According to some embodiments, in the compound of the present disclosure as disclosed above R³ is selected from the group consisting of OH, NH₂, SH, SO₃H, COOH, and CONH₂, DA is thiourea and H_{N} is selected from the group consisting of pyridyl, isoindolinyl, indolyl, isoquinolinyl, quinolinyl, and imidazopyridinyl, preferably, R³ is selected from OH, SH, or SO₃H, more preferably R³ is selected from OH or SH.

According to preferred embodiments, the compound of the present disclosure is selected from

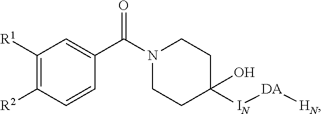

wherein R¹, R², I_{N}, DA and H_{N} are as disclosed above.

According to one embodiment, the compound of the present disclosure is selected from the group of compounds as shown below wherein R³ is OH, R¹, R² are as defined above, D_{A} is cyanoguanidine, H_{N} is selected from the group consisting of pyridyl, isoindolinyl, indolyl, isoquinolinyl, quinolinyl, and imidazopyridinyl, and I_{N} is selected from the group consisting of —(CH₂)ₙ—, cycloalkyl-(CH₂)ₙ—, aryl-(CH₂)ₙ—, heteroaryl-(CH₂)ₙ—, wherein n is 0 to 6 (e.g. 0, 1, 2, 3, 4, 5, or 6):

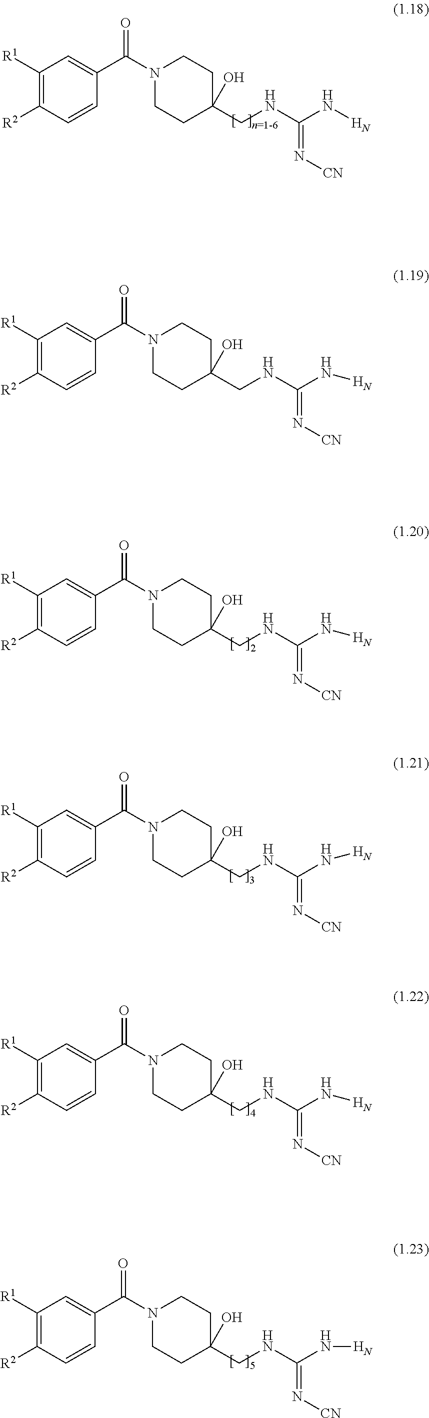

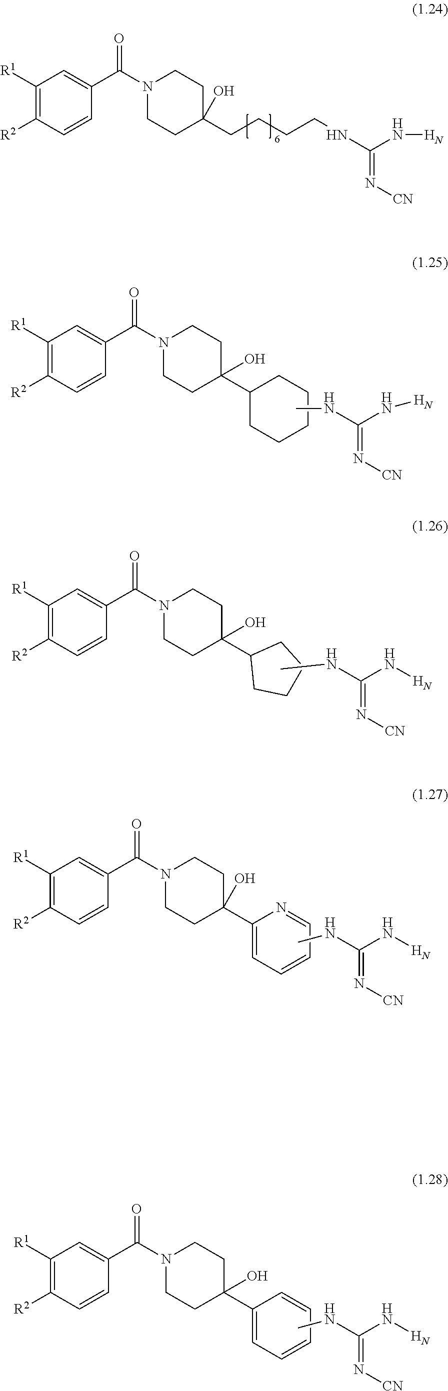

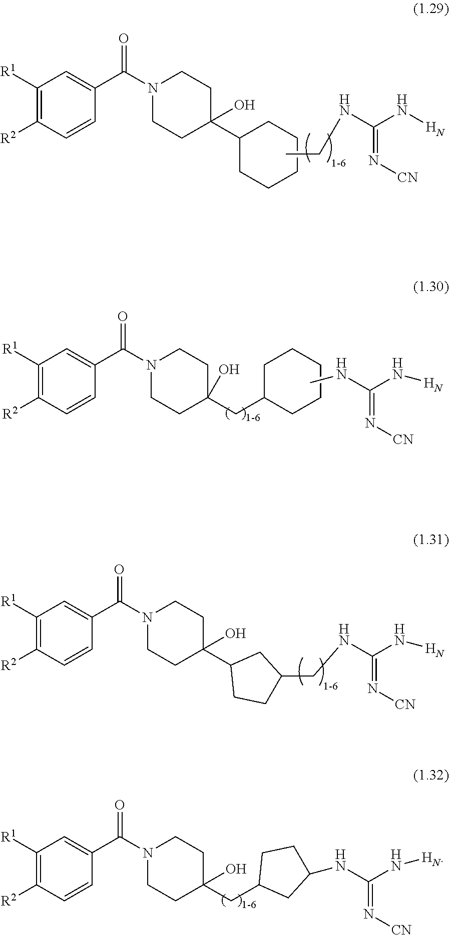

According to one embodiment, the inventive compound is selected from the group of compounds in which R³ is OH, R¹, R² are as defined above, DA is acrylamide, H_{N} is selected from the group consisting of pyridyl, isoindolinyl, indolyl, isoquinolinyl, quinolinyl, and imidazopyridinyl and I_{N} is selected from the group consisting of —(CH₂)ₙ—, cycloalkyl-(CH₂)ₙ—, aryl-(CH₂)ₙ—, heteroaryl-(CH₂)ₙ—, wherein n is 0 to 6 (e.g. 0, 1, 2, 3, 4, 5, or 6) or 1 to 6 (e.g. 1, 2, 3, 4, 5, or 6):

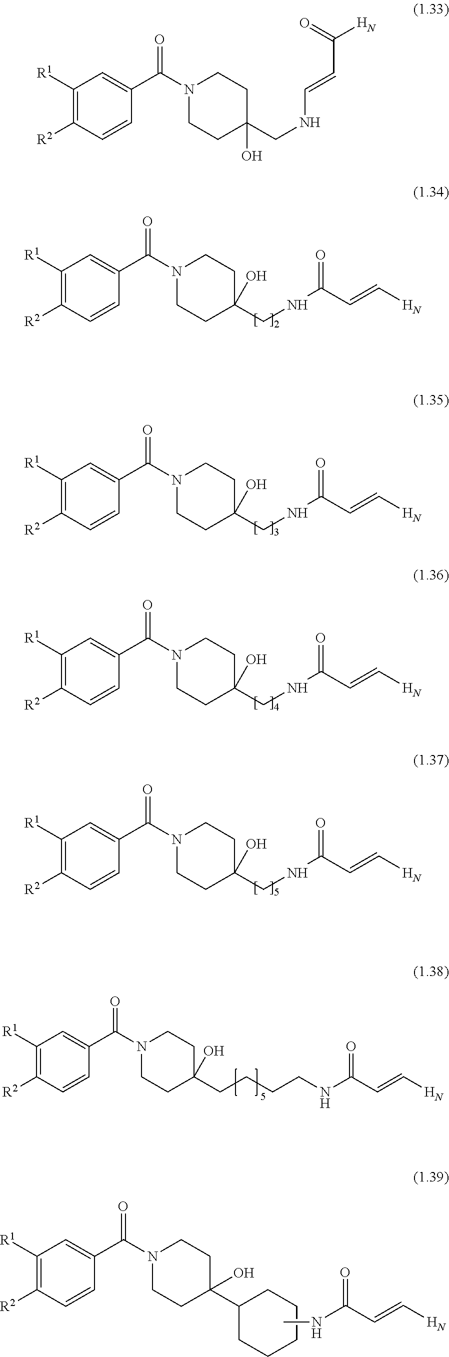

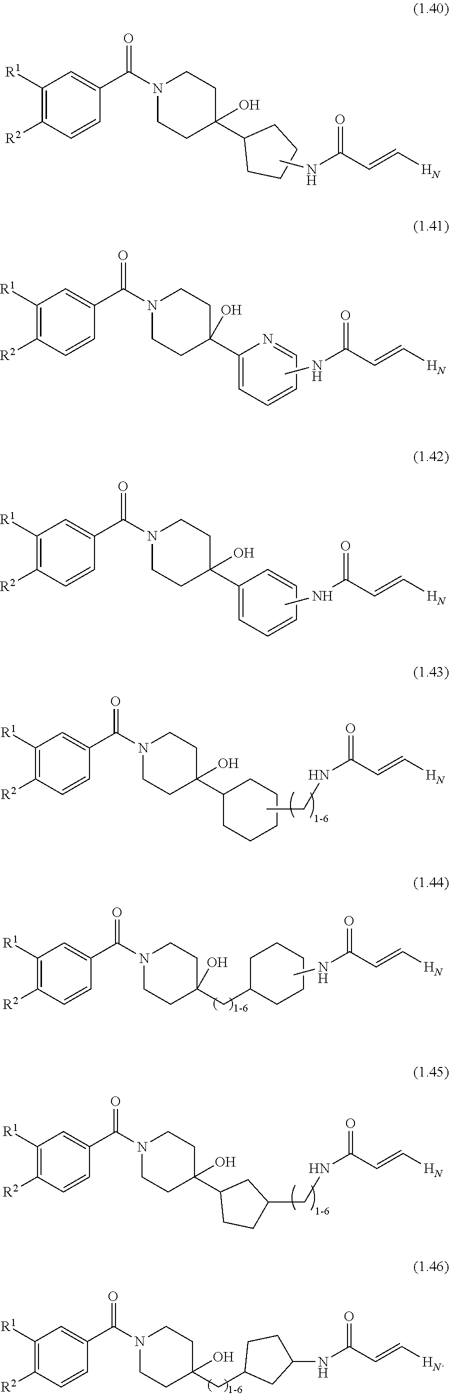

According to one embodiment, the compound of the present disclosure is selected from the group of compounds in which R³ is OH, R¹, R^{Z} are as defined above, D_{A} is urea, H_{N} is selected from the group consisting of pyridyl, isoindolinyl, indolyl, isoquinolinyl, quinolinyl, and imidazopyridinyl and I_{N} is selected from the group consisting of —(CH₂)ₙ—, cycloalkyl-(CH₂)ₙ—, aryl-(CH₂)ₙ—, heteroaryl-(CH₂)ₙ—, wherein n is 0 to 6 (e.g. 0, 1, 2, 3, 4, 5, or 6) or 1 to 6:

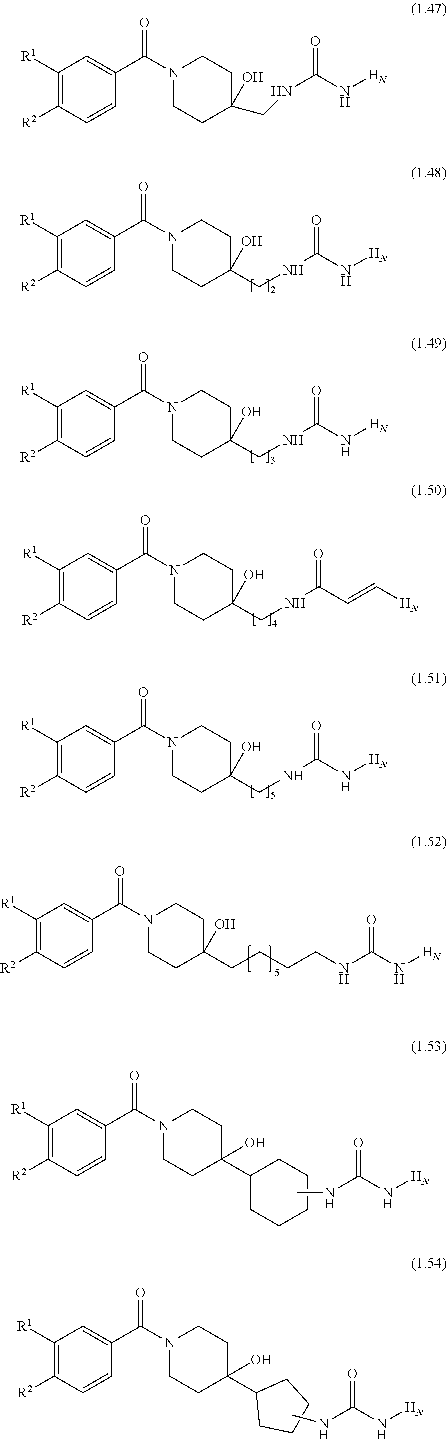

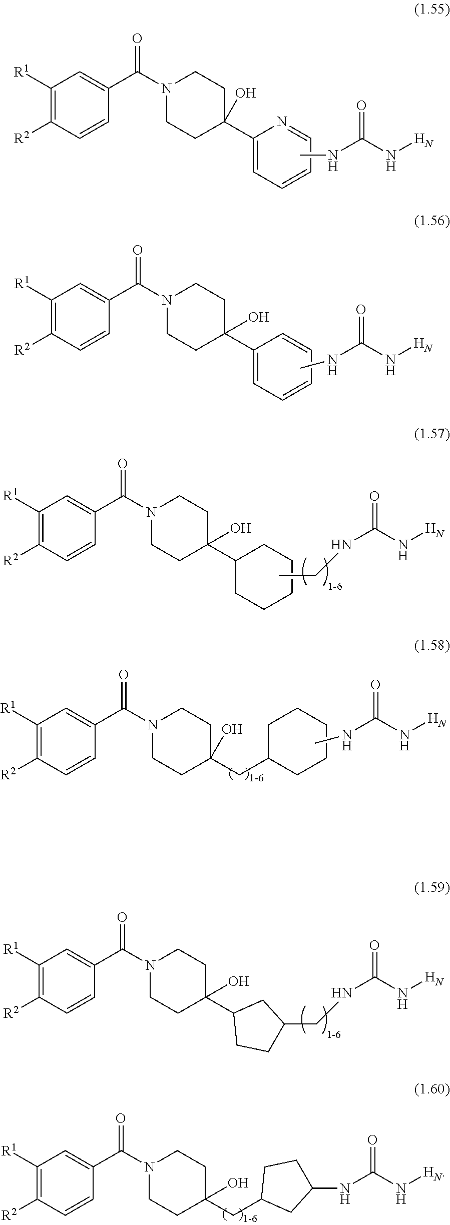

According to one embodiment, the inventive compound is selected from the group of compounds in which R³ is OH, R¹, R² are as defined above, D_{A} is thiourea, H_{N} is selected from the group consisting of pyridyl, isoindolinyl, indolyl, isoquinolinyl, quinolinyl, and imidazopyridinyl and I_{N} is selected from the group consisting of —(CH₂)ₙ—, cycloalkyl-(CH₂)ₙ—, aryl-(CH₂)ₙ—, heteroaryl-(CH₂)ₙ—, wherein n is 0 to 6 (e.g. 0, 1, 2, 3, 4, 5, or 6) or 1 to 6 (e.g. 1, 2, 3, 4, 5, or 6):

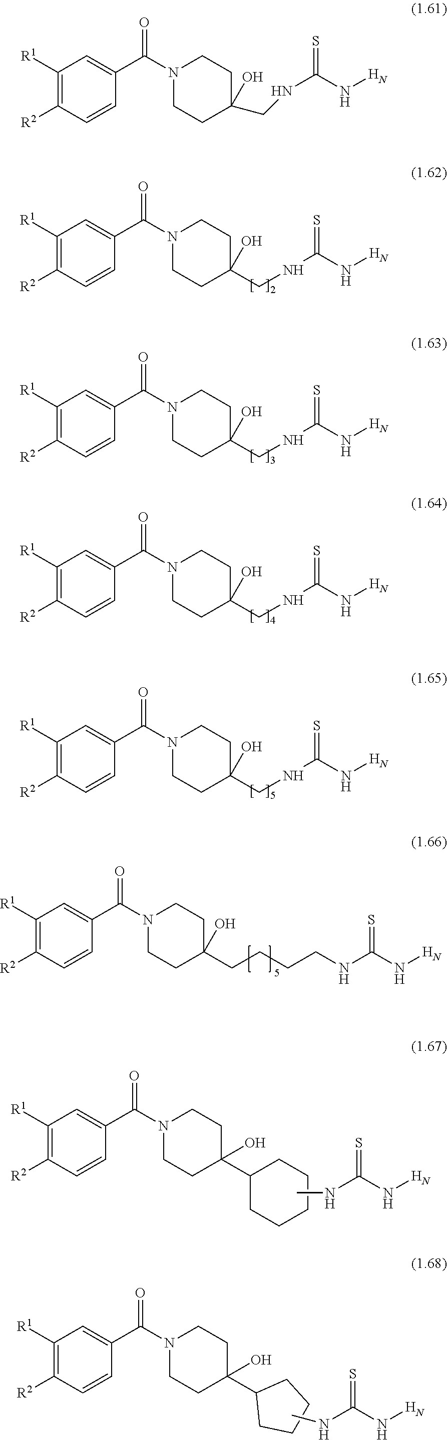

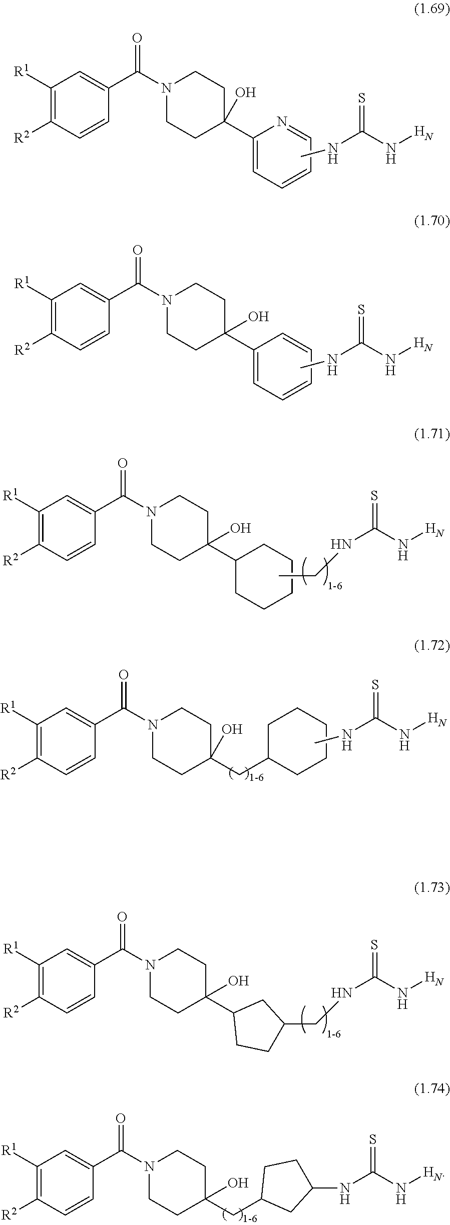

According to some embodiments, the compound of the present disclosure as disclosed above is selected from compounds in which R³═OH, e.g. in compound (I), or in any one of compounds (1.1), (1.2), (1.3), (1.4), (1.5), (1.6), (1.7), (1.8), (1.9), (1.10), (1.11), (1.12), (1.13), (1.14), (1.15), (1.16), or (1.17), and wherein DA is cyanoguanidine, and H_{N} is selected from the group consisting of pyridyl, isoindolinyl, indolyl, isoquinolinyl, quinolinyl, and imidazopyridinyl, preferably, the compound of the present disclosure is selected from R³═OH, D_{A}=cyanoguanidine and H_{N} is selected from the group consisting of pyridyl, isoindolinyl, indolyl, isoquinolinyl, quinolinyl, and imidazopyridinyl.

In some embodiments, the compound is selected from (1.1), (1.2), (1.3), (1.4), (1.5) with R¹, R² as disclosed herein above:

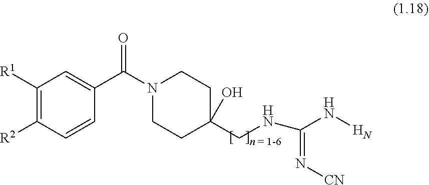

According to a preferred embodiment, the compound of the present disclosure is selected from the group consisting of (1.1), (1.2), (1.3), (1.4), and (1.5) wherein R³═OH, DA is acrylamide and H_{N} is selected from the group consisting of pyridyl, isoindolinyl, indolyl, isoquinolinyl, quinolinyl, and imidazopyridinyl, with R¹, R² as disclosed herein above:

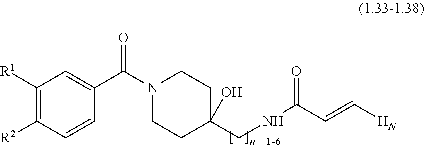

According to some embodiments, the inventive compound as disclosed above comprises R³═OH, e.g. in compound (I), or in any one of compounds (1.1), (1.2), (1.3), (1.4), (1.5), (1.6), (1.7), (1.8), (1.9), (1.10), (1.11), (1.12), (1.13), (1.14), (1.15), (1.16), or (1.17), wherein D_{A} is urea, and H_{N} is selected from the group consisting of pyridyl, isoindolinyl, indolyl, isoquinolinyl, quinolinyl, and imidazopyridinyl.

According to preferred embodiments, the compound of the present disclosure is selected from the group of compounds (1.1), (1.2), (1.3), (1.4), and (1.5) comprising R³═OH, DA being urea and H_{N} is selected from the group consisting of pyridyl, isoindolinyl, indolyl, isoquinolinyl, quinolinyl, and imidazopyridinyl, with R¹, R² as disclosed herein above:

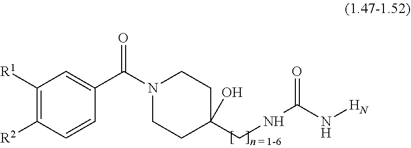

According to some embodiments, the compound of the present disclosure as disclosed above comprises R³═OH, e.g. in compound (I), or in any one of compounds (1.1), (1.2), (1.3), (1.4), (1.5), (1.6), (1.7), (1.8), (1.9), (1.10), (1.11), (1.12), (1.13), (1.14), (1.15), (1.16), or (1.17), wherein DA is thiourea, and H_{N} is selected from the group consisting of pyridyl, isoindolinyl, indolyl, isoquinolinyl, quinolinyl, and imidazopyridinyl,

According to preferred embodiments, the compound of the present disclosure is selected from the group consisting of (1.1), (1.2), (1.3), (1.4), and (1.5) wherein R³ is OH, DA is thiourea and H_{N} is selected from the group consisting of pyridyl, isoindolinyl, indolyl, isoquinolinyl, quinolinyl, and imidazopyridinyl, with R¹, R² as disclosed herein above:

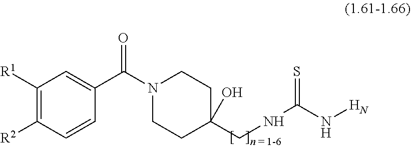

According to some embodiments, the compound as disclosed herein is selected from the group consisting of compounds (1.75)-(1.77) wherein D_{A} is cyanoguanidine, H_{N} is pyridyl, I_{N} is C₁-C₆ alkyl and R¹, R² are as defined above:

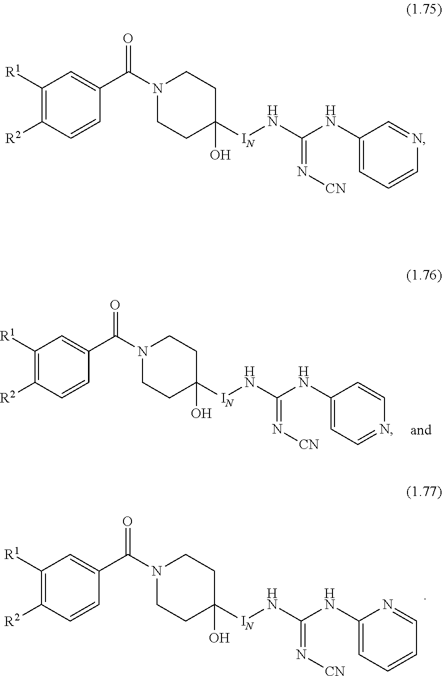

According to some embodiments, the compound as disclosed herein is compound (1.78) or (1.78a), wherein D_{A} is cyanoguanidine, H_{N} is isoindoline, I_{N} is C₁-C₆ alkyl and R¹, R² are as defined above:

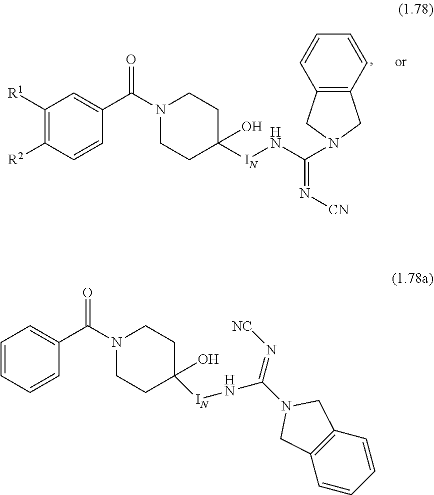

preferably, I_{N} is selected from C₃-C₆ alkyl, e.g. I_{N} is C₃, C₄, C₅, or C₆ alkyl.

According to some embodiments, the compound as disclosed herein is compound (1.79), or (1.80) wherein D_{A} is cyanoguanidine and H_{N} is isoquinoline

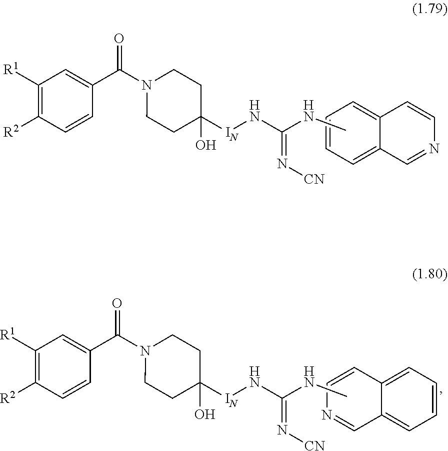

wherein I_{N} is C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.81), or (1.82) wherein D_{A} is cyanoguanidine and H_{N} is quinoline

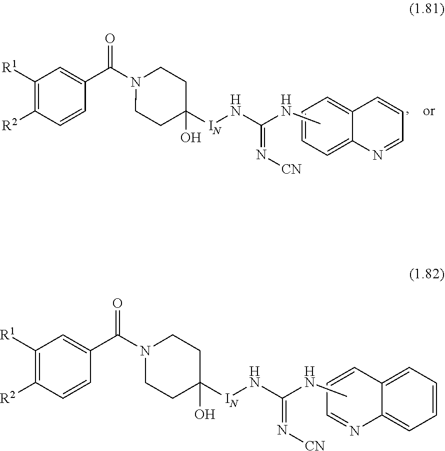

wherein I_{N} is C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound of the present disclosure as disclosed herein is selected from the group consisting of compounds (1.83), (1.84), (1.85), (1.86), (1.87), (1.88), and (1.89) wherein D_{A} is cyanoguanidine and H_{N} is indole:

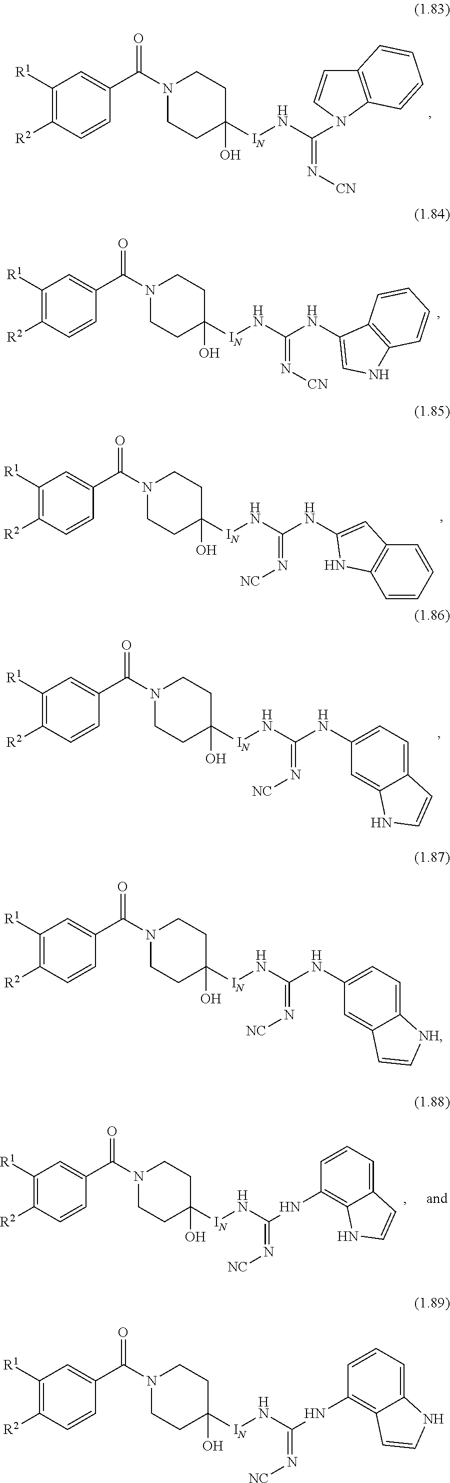

for which I_{N} selected from C₁-C₆ alkyl and R¹, R² are as disclosed above, preferably, I_{N} is selected from C₃-C₆ alkyl, e.g. I_{N} is C₃, C₄, C₅, or C₆ alkyl.

According to some embodiments, the compound as disclosed herein is compound (1.90), or (1.91), in which D_{A} is cyanoguanidine and H_{N} is imidazopyridine:

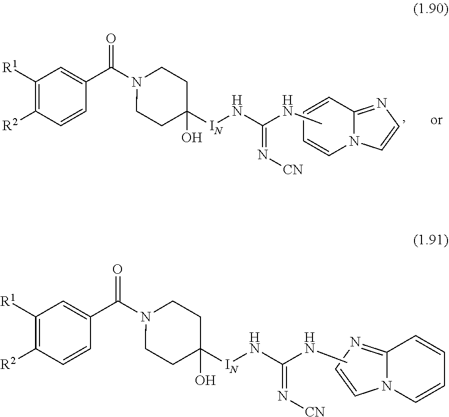

wherein I_{N} selected from C₁-C₆ alkyl and R¹, R² are as disclosed above, preferably, I_{N} is selected from C₃-C₆ alkyl, e.g. I_{N} is C₃, C₄, C₅, or C₆ alkyl.

According to some embodiments, the compound as disclosed herein is selected from the group consisting of compounds (1.92)-(1.94) wherein D_{A} is urea, H_{N} is pyridyl, I_{N} is C₁-C₆ alkyl and R¹, R² are as defined above:

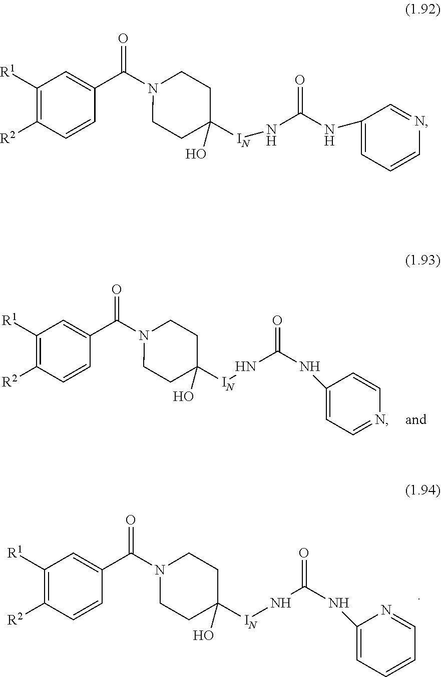

According to some embodiments, the compound as disclosed herein is compound (1.95) wherein D_{A} is urea, H_{N} is isoindoline, I_{N} is C₁-C₆ alkyl and R, R² are as defined above:

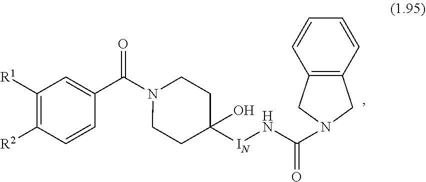

preferably, I_{N} is selected from 3-C₆ alkyl, e.g. I_{N} is C₃, C₄, C₅, or C₆ alkyl.

According to some embodiments, the compound as disclosed herein is compound (1.96), or (1.97) wherein D_{A} is cyanoguanidine and H_{N} is isoquinoline

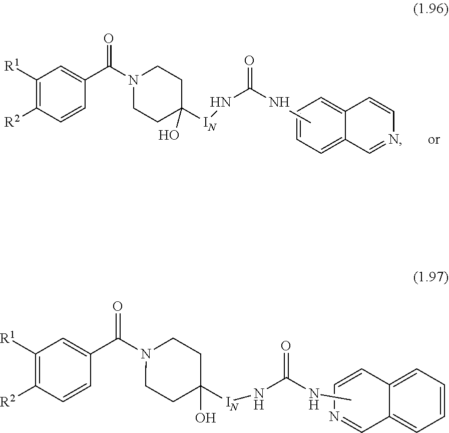

wherein I_{N} selected from C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.98), or (1.99) wherein D_{A} is cyanoguanidine and H_{N} is quinoline

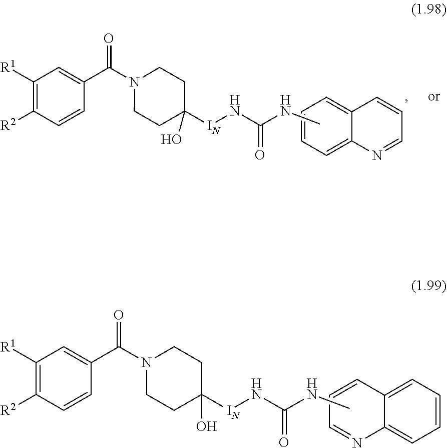

wherein I_{N} selected from C₁-C₆ alkyl and R, R² are as disclosed above, preferably, I_{N} is selected from C₃-C₆ alkyl, e.g. I_{N} is C₃, C₄, C₅, or C₆ alkyl.

According to some embodiments, the compound as disclosed herein is selected from the group consisting of compounds (1.100), (1.101), (1.102), (1.103), (1.104), (1.105), and (1.106) wherein D_{A} is urea and H_{N} is indole.

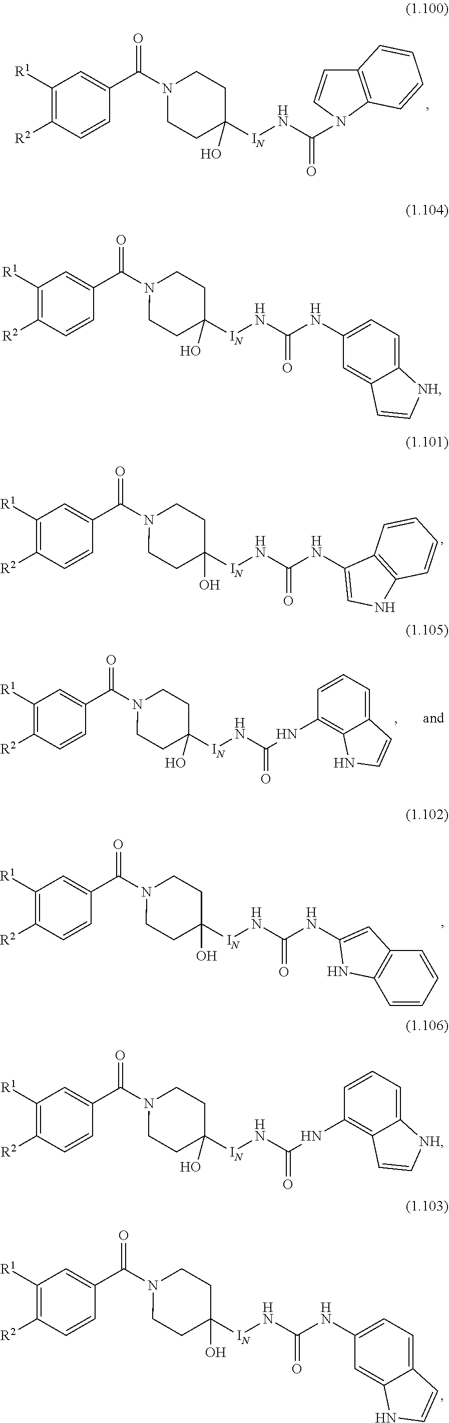

wherein I_{N} selected from C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.107), or (1.108), wherein D_{A} is urea and H_{N} is imidazopyridine:

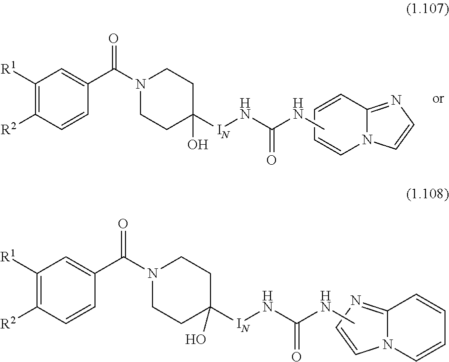

wherein I_{N} selected from C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is selected from compounds (1.109)-(1.111) wherein D_{A} is thiourea, H_{N} is pyridyl, I_{N} is C₁-C₆ alkyl and R¹, R² are as defined above:

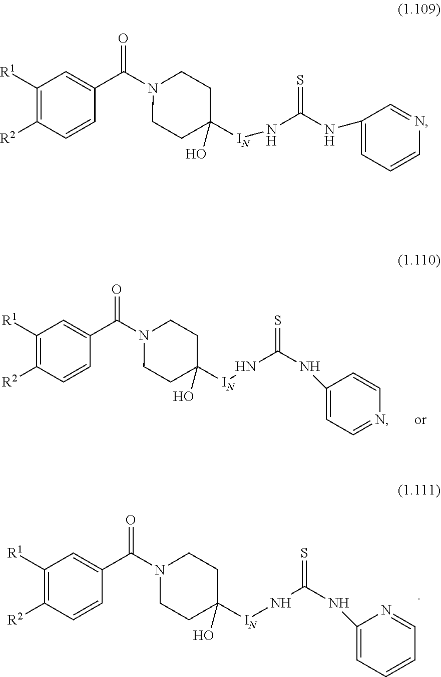

According to some embodiments, the compound as disclosed herein is compound (1.112) wherein D_{A} is thiourea, H_{N} is isoindoline, I_{N} is C₁-C₆ alkyl and R¹, R² are as defined above:

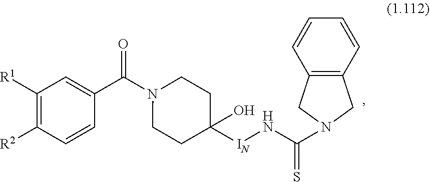

preferably, I_{N} is selected from C₃-C₆ alkyl, e.g. I_{N} is C₃, C₄, C₅, or C₆ alkyl.

According to some embodiments, the compound as disclosed herein is selected from compounds (1.113), or (1.114) wherein D_{A} is cyanoguanidine and H_{N} is isoquinoline

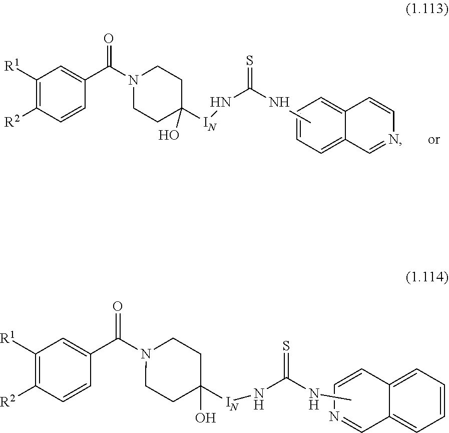

wherein I_{N} is C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.115), or (1.116) wherein D_{A} is cyanoguanidine and H_{N} is quinoline

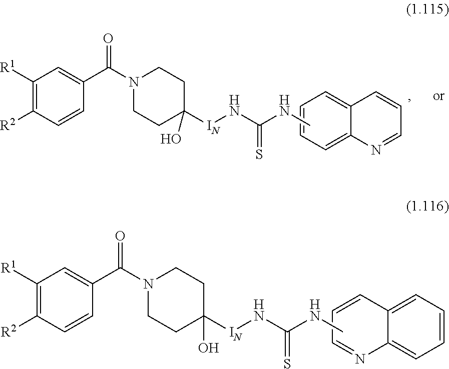

wherein I_{N} selected from C₁-C₆ alkyl and R¹, R² are as disclosed above, preferably, I_{N} is selected from C₃-C₆ alkyl, e.g. I_{N} is C₃, C₄, C₅, or C₆ alkyl.

According to some embodiments, the compound as disclosed herein is selected from the group consisting of (1.117), (1.118), (1.119), (1.120), (1.121), (1.122), and (1.123) wherein D_{A} is thiourea and H_{N} is indole:

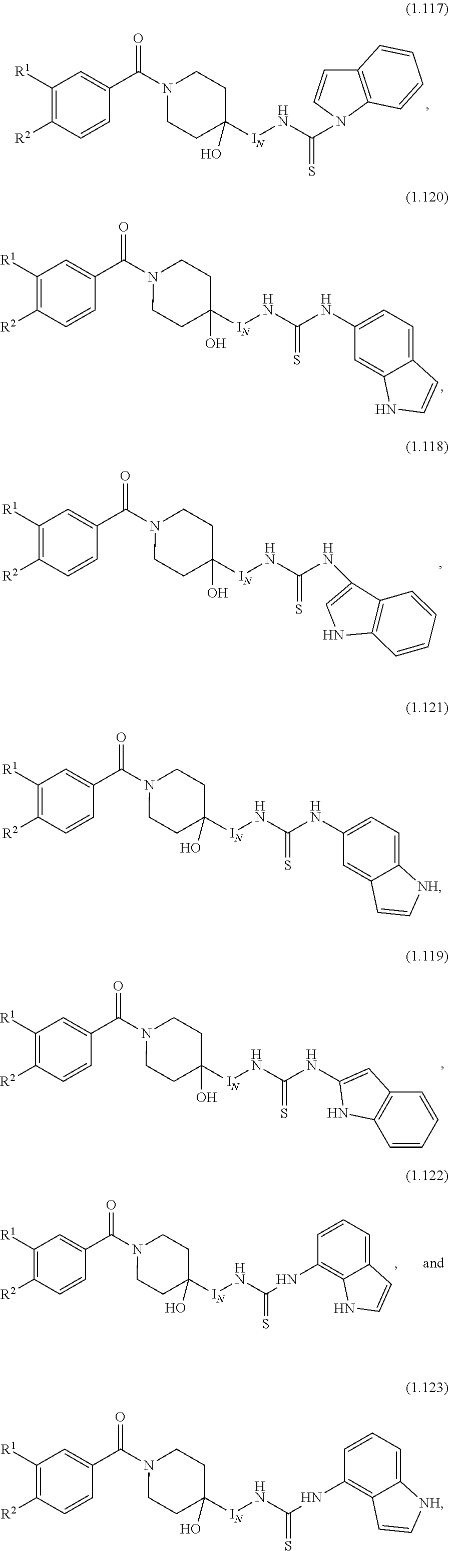

wherein I_{N} is C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.124), or (1.125), wherein D_{A} is urea and H_{N} is imidazopyridine:

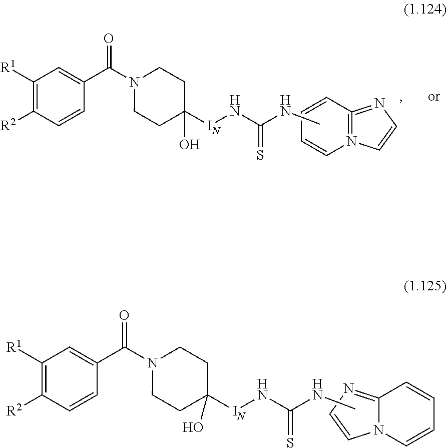

wherein I_{N} is C₁-C₆ alkyl and R, R² are as disclosed above, preferably, I_{N} is C₃-C₆ alkyl, e.g. I_{N} is C₃, C₄, C₅, or C₆ alkyl.

According to preferred embodiments, the inventive compound is selected from the group consisting of compounds (1.75)-(1.125), e.g. (1.75), (1.76), (1.77), (1.78), (1.79), (1.80), (1.81), (1.82), (1.83), (1.84), (1.85), (1.86), (1.87), (1.88), (1.89), (1.90), (1.91), (1.92), (1.93), (1.94), (1.95), (1.96), (1.97), (1.98), (1.99), (1.100), (1.101), (1.102), (1.103), (1.104), (1.105), (1.106), (1.107), (1.108), (1.109), (1.110), (1.111), (1.112), (1.113), (1.114), (1.115), (1.116), (1.117), (1.118), (1.119), (1.120), (1.121), (1.122), (1.123), (1.124), and (1.125) wherein I_{N} is C₄ alkyl for each compound and R¹, R² are as defined above.

According to more preferred embodiments, the compound of the present disclosure is selected from the group consisting of compounds (1.75), (1.83), (1.84), (1.85), (1.92), (1.93), (1.100), (1.101), and (1.102), wherein I_{N} is 4 alkyl for each compound and R¹, R² are as defined above:

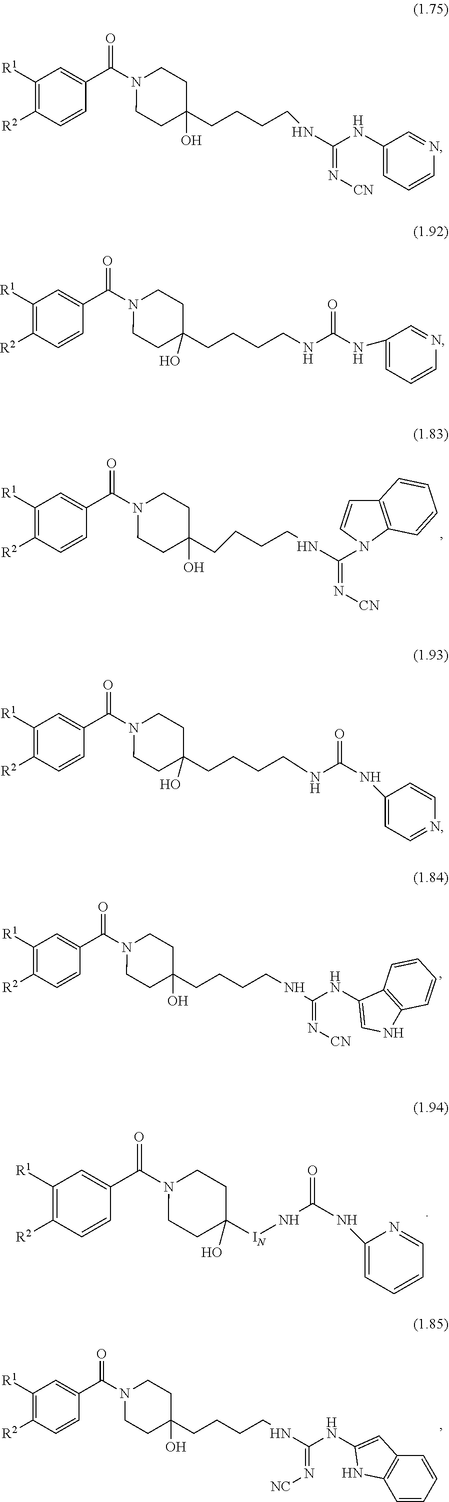

According to some embodiments, the compound as disclosed herein is compound (1.95) wherein D_{A} is urea, H_{N} is isoindoline, I_{N} is C₁-C₆ alkyl and R¹, R² are as defined above:

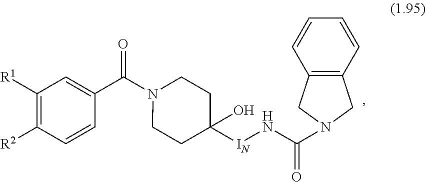

preferably, I_{N} is selected from C₃-C₆ alkyl, e.g. I_{N} is C₃, C₄, C₅, or C₆ alkyl.

According to some embodiments, the compound as disclosed herein is compound (1.96), or (1.97) wherein D_{A} is cyanoguanidine and H_{N} is isoquinoline

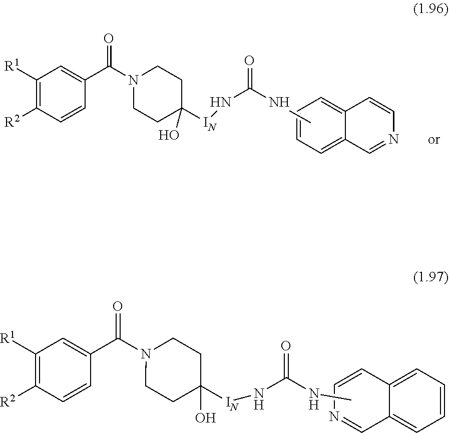

wherein I_{N} selected from C₁-C₆ alkyl and R¹, R² are as disclosed above, preferably, I_{N} is C₃-C₆ alkyl, e.g. I_{N} is C₃, C₄, C₅, or C₆ alkyl.

According to some embodiments, the compound as disclosed herein is compound (1.98), or (1.99) wherein D_{A} is cyanoguanidine and H_{N} is quinoline

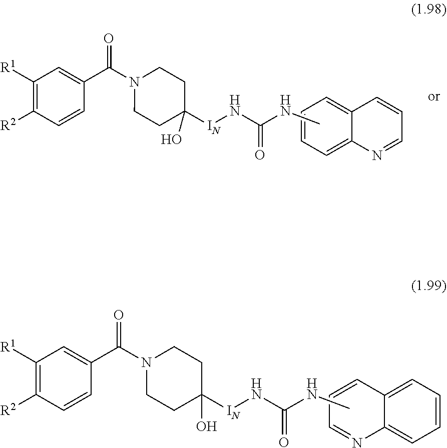

wherein I_{N} selected from C₁-C₆ alkyl and R¹, R² are as disclosed above, preferably, I_{N} is selected from C₃-C₆ alkyl, e.g. I_{N} is C₃, C₄, C₅, or C₆ alkyl.

According to some embodiments, the compound as disclosed herein is selected from the group consisting of compounds (1.100), (1.101), (1.102), (1.103), (1.104), (1.105), and (1.106) wherein D_{A} is urea and H_{N} is indole:

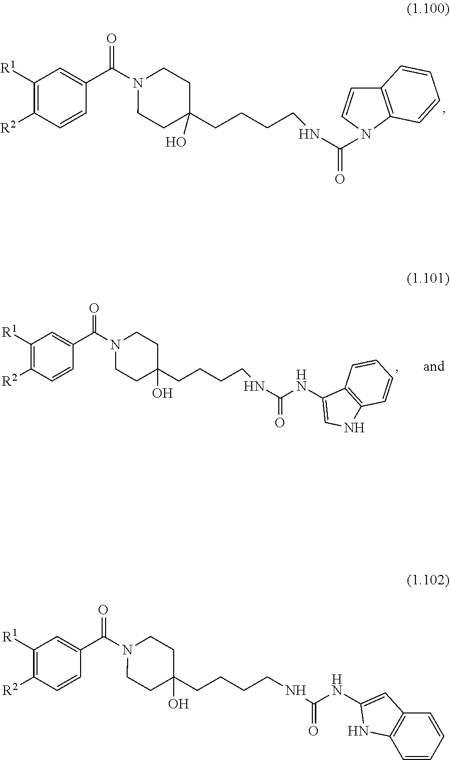

According to preferred embodiments, in the compound of the present disclosure wherein R¹ is NH₂ and R² is H, is

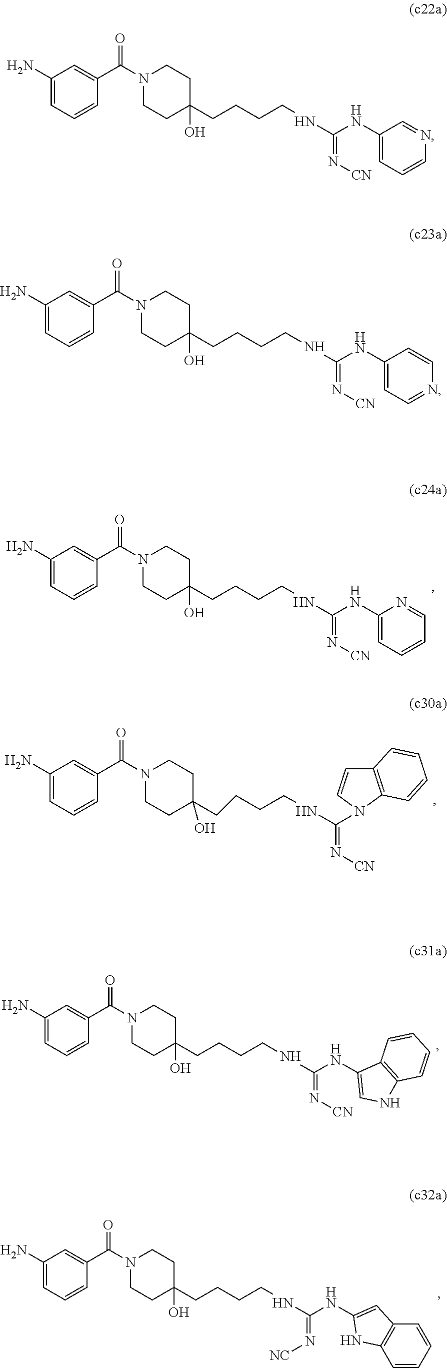 [SMILES: N#C/N=C(/NCCCCC1(O)CCN(C(=O)c2cccc(N)c2)CC1)Nc1cccnc1 ; N#C/N=C(/NCCCCC1(O)CCN(C(=O)c2cccc(N)c2)CC1)Nc1ccncc1 ; N#C/N=C(/NCCCCC1(O)CCN(C(=O)c2cccc(N)c2)CC1)Nc1ccccn1 ; N#C/N=C(/NCCCCC1(O)CCN(C(=O)c2cccc(N)c2)CC1)n1ccc2ccccc21 ; N#C/N=C(/NCCCCC1(O)CCN(C(=O)c2cccc(N)c2)CC1)Nc1c[nH]c2ccccc12 ; N#C/N=C(\NCCCCC1(O)CCN(C(=O)c2cccc(N)c2)CC1)Nc1cc2ccccc2[nH]1]

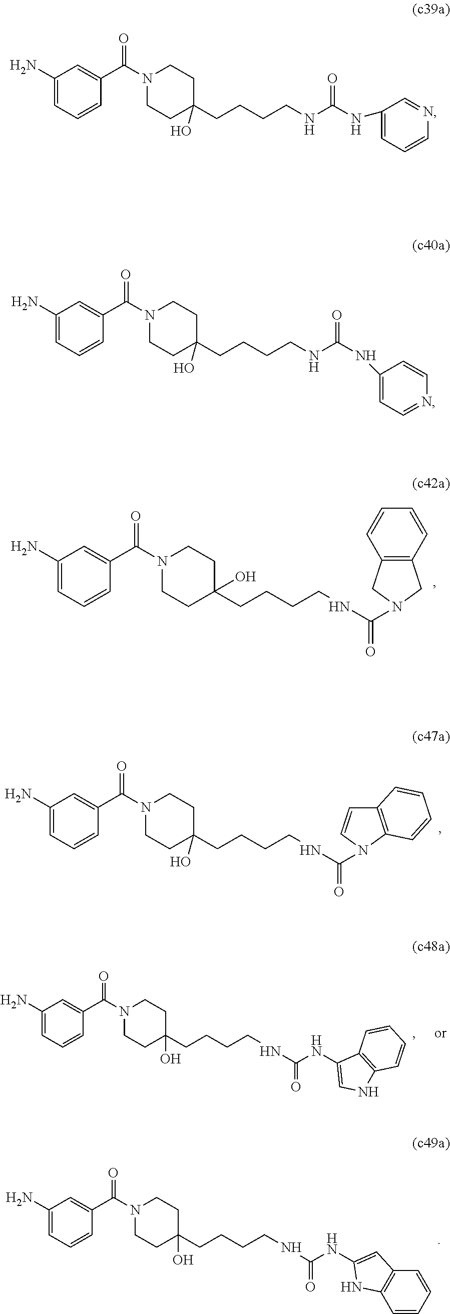 [SMILES: Nc1cccc(C(=O)N2CCC(O)(CCCCNC(=O)Nc3cccnc3)CC2)c1 ; Nc1cccc(C(=O)N2CCC(O)(CCCCNC(=O)Nc3ccncc3)CC2)c1 ; Nc1cccc(C(=O)N2CCC(O)(CCCCNC(=O)N3Cc4ccccc4C3)CC2)c1 ; Nc1cccc(C(=O)N2CCC(O)(CCCCNC(=O)n3ccc4ccccc43)CC2)c1 ; Nc1cccc(C(=O)N2CCC(O)(CCCCNC(=O)Nc3c[nH]c4ccccc34)CC2)c1 ; Nc1cccc(C(=O)N2CCC(O)(CCCCNC(=O)Nc3cc4ccccc4[nH]3)CC2)c1]

According to particularly preferred embodiments, the compound as disclosed herein is (c42a) or (c22a).

According to some embodiments, in the compound as disclosed above R¹ is NR^{L} and R² is selected from H, OH, NH₂, N₃, and SH.

According to some embodiments, in the compound as disclosed above R¹ is OR^{L} and R² is selected from H, OH, NH₂, N₃, and SH.

According to some embodiments, in the compound as disclosed above R¹ is SR^{L} and R² is selected from H, OH, NH₂, N₃, and SH.

According to preferred embodiments, in the compound as disclosed above R¹ is NR^{L}, R² is H and R^{L} is a cleavable linker. A “cleavable linker” is understood as comprising at least one cleavage site. As used herein, the term “cleavage site” shall refer to a moiety that is susceptible to specific cleavage at a defined position under defined conditions. Said conditions are, e.g., specific enzymes or a reductive environment in specific body or cell compartments.

According to some embodiments, the cleavage site can be cleavable by at least one protease selected from the group consisting of cysteine protease, metalloprotease, serine protease, threonine protease, and aspartic protease.

Cysteine proteases, also known as thiol proteases, are proteases that share a common catalytic mechanism that involves a nucleophilic cysteine thiol in a catalytic triad or dyad.

Metalloproteases are proteases whose catalytic mechanism involves a metal. Most metalloproteases require zinc, but some use cobalt. The metal ion is coordinated to the protein via three ligands. The ligands coordinating the metal ion can vary with histidine, glutamate, aspartate, lysine, and arginine. The fourth coordination position is taken up by a labile water molecule.

Serine proteases are enzymes that cleave peptide bonds in proteins; serine serves as the nucleophilic amino acid at the enzyme's active site. Serine proteases fall into two broad categories based on their structure: chymotrypsin-like (trypsin-like) or subtilisin-like.

Threonine proteases are a family of proteolytic enzymes harboring a threonine (Thr) residue within the active site. The prototype members of this class of enzymes are the catalytic subunits of the proteasome, however, the acyltransferases convergently evolved the same active site geometry and mechanism.

Aspartic proteases are a catalytic type of protease enzymes that use an activated water molecule bound to one or more aspartate residues for catalysis of their peptide substrates. In general, they have two highly conserved aspartates in the active site and are optimally active at acidic pH. Nearly all known aspartyl proteases are inhibited by pepstatin.

In some embodiments of the present disclosure, the cleavable site is cleavable by at least one agent selected from the group consisting of Cathepsin A or B, matrix metalloproteinases (MMPs), elastases, glutathione (GSH), ┘-glucuronidase and ┘-galactosidase, preferably Cathepsin B.

In some embodiments, the cleavable linker is a pH-sensitive linker and is sensitive to hydrolysis at certain pH values. Typically, the pH-sensitive linker is cleavable under acidic conditions. This cleavage strategy generally takes advantage of the lower pH in the endosomal (pH ˜5-6) and lysosomal (pH ˜4.8) intracellular compartments, as compared to the cytosol (pH ˜7.4), to trigger hydrolysis of an acid labile group in the linker, such as a hydrazone which have been described in e.g. Jain et al. (2015) Pharm Res 32:3526-40. In some embodiments, the linker is an acid labile and/or hydrolyzable linker. For example, an acid labile linker that is hydrolyzable in the lysosome, and contains an acid labile group (e.g., a hydrazone, a semicarbazone, a thiosemicarbazone, a cis-aconitic amide, an orthoester, an acetal, a ketal, or the like) can be used. Corresponding linkers have e.g. been disclosed in U.S. Pat. Nos. 5,122,368; 5,824,805; 5,622,929; Dubowchik and Walker (1999) Pharm. Therapeutics 83:67-123; Neville et al. (1989) Biol. Chem. 264: 14653-61 the content of which is hereby incorporated in entirety. Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, e.g., a thioether attached to the therapeutic agent via an acylhydrazone bond), as disclosed in e.g. U.S. Pat. No. 5,622,929.

According to some embodiments of the present disclosure, the cleavable linker of the disclosure is an enzymatically cleavable linker. Enzymatically cleavable linkers comprise a cleavage site that is an enzymatically cleavable moiety comprising two or more amino acids. Preferably, said enzymatically cleavable moiety comprises a phenylalanine-lysine (Phe-Lys), valine-lysine (Val-Lys), phenylalanine-alanine (Phe-Ala), valine-alanine (Val-Ala), phenylalanine-citrulline (Phe-Cit), or valine-citrulline (Val-Cit) dipeptide, cyclobutane-1,1-dicarboxamide (cBu)-Ala, cBu-Ala, cBu-Cit, Glu-Val-Ala, Glu-Val-Cit, Glu-cBu-Ala, Glu-cBu-Cit, or e.g. a valine-alanine-valine (Val-Ala-Val), leucine-alanine-leucine (Leu-Ala-Leu), glycine-phenylalanine-lysine (Gly-Phe-Lys), isoleucine-alanine-leucine (Ile-Ala-Leu) tripeptide, a phenylalanine-lysine-glycine-proline-leucin-glycine (Phe Lys Gly Pro Leu Gly) or alanine-alanine-proline-valine (Ala Ala Pro Val) peptide, or a ┘-glucuronide or ┘-galactoside.

In preferred embodiments, the cleavable linker L being part of NR^{L} according to the disclosure as disclosed above is a self-immolative linker. The term “self-immolative linker” or “elf-immolative spacer” refers to a bifunctional chemical moiety that is capable of covalently linking two chemical moieties into a normally stable tripartate molecule. The self-immolative spacer is capable of spontaneously separating from the second moiety if the bond to the first moiety is cleaved. Corresponding self-immolative linkers are e.g. disclosed in WO03026577 disclosing p-amidobenzylether-comprising linkers, or in WO2005/112919 and which may e.g. also be used in the linkers of the disclosure. Alternative self-immolative spacers that may e.g. be used in the linkers of the disclosure comprise a glycine-proline (gly-pro) dipeptide, which undergoes spontaneous cyclization upon cleavage of the linker L.

In particularly preferred embodiments, the enzymatically cleavable linker L according to the disclosure comprises a dipeptide selected from Phe-Lys, Val-Lys, Phe-Ala, Val-Ala, Phe-Cit and Val-Cit, particularly wherein the cleavable linker further comprises a p-aminobenzyl (PAB) spacer between the dipeptides and the compound of the disclosure.

The cleavable linker L according to the disclosure as disclosed above may e.g. further comprise a linear C₂-C₆ alkyl chain to which the amine or thiol-reactive moiety Z is attached, for example, a cleavable linker L according to the disclosure may comprise the structure:

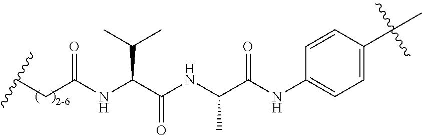

In some embodiments, the linker R^{L} is a non-cleavable linker. A “non-cleavable linker” is understood not to be subject to enzymatical cleavage by e.g. cathepsin B and is released from the conjugates of the present disclosure during degradation (e.g. during lysosomal degradation) from the antibody moiety of the conjugate of the present disclosure inside the target cell. Non-cleavable linkers suitable for use according to the present disclosure may e.g. include one or more groups selected from a bond, —(C═O)—, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted, and/or may include one or more heteroatoms (e.g., S, N, or O) in place of one or more carbon atoms. Non-limiting examples of such groups include (CH₂)ₚ, (C═O)(CH₂)ₚ, and polyethyleneglycol (PEG; (CH₂CH₂O)ₚ), units, wherein p is an integer from 1-6, independently selected for each occasion.

In some embodiments, the non-cleavable linker according to the present disclosure comprises one or more of a bond, —(C═O)—, a —C(O)NH— group, an —OC(O)NH— group, C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, a —(CH₂CH₂O)p- group where p is an integer from 1-6, wherein each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene, in some embodiments these groups are substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

For example, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene of the non-cleavable linker as disclosed herein may optionally be interrupted by one or more heteroatoms selected from O, S and N and may e.g. be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto and nitro.

According to preferred embodiments, the non-cleavable linker of the conjugate of the present disclosure comprises a —(CH₂)ₙ— unit, where n is an integer from 2-12, e.g. 4-6, 8, 10, or 2-6, e.g. n is 1, 2, 3, 4, 5, or 6.

In a preferred embodiment, the non-cleavable linker according to the present disclosure comprises —(CH₂)ₙ— wherein n is 6 and the linker is represented by the formula:

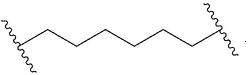 [SMILES: *CCCCCC*]

According to some embodiments the cleavable and non-cleavable linkers of the present disclosure as disclosed above comprise an amine or thiol reactive moiety Z which is useful for coupling the compound of the present disclosure comprising linkers having the structure L-Z to reactive thiols or amines of e.g. a binding moiety, such as an antibody. For example, Z may be a thiol-reactive group selected from bromo acetamide, iodo acetamide, methylsulfonylbenzothiazole, 4,6-dichloro-1,3,5-triazin-2-ylamino group methyl-sulfonyl phenyltetrazole or methylsulfonyl phenyloxadiazole, pyridine-2-thiol, 5-nitropyridine-2-thiol, methanethiosulfonate, or preferably maleimide.

Examples of a lysine-reactive Z moieties includes N-hydrxysuccinimide (see e.g. Haque et al. Chem Commun (Camb). 2021 Oct. 14; 57(82): 10689-10702). Thiol-reactive moieties are, however, preferred. Corresponding protocols for coupling the compound of the present disclosure comprising a thiol-reactive L-Z moiety are e.g. disclosed in WO 2016/142049 A1.

In some embodiments Z is selected from a group comprising azides, amines, alkynes, tetrazines, strained cyclooctynes, aldehydes, HIPS reagents (Hydrazino-Pictet-Spengler) and other reactive groups useful in bioconjugations and bio-orthogonal reactions.

In preferred embodiments Z is selected from a group comprising thiol-reactive or amine-reactive chemical moieties, azides, amines, alkynes, tetrazines, strained cyclooctynes, aldehydes and HIPS reagents (Hydrazino-Pictet-Spengler), in more preferred embodiments Z is selected from a group comprising thiol-reactive or amine-reactive chemical moieties, azides, amines, alkynes and tetrazines.

According to preferred embodiments, the linker-compound (Z-L-T) according to the present disclosure comprises a cleavable linker as defined above and a thiol-reactive moiety Z, wherein the compound is selected from one of

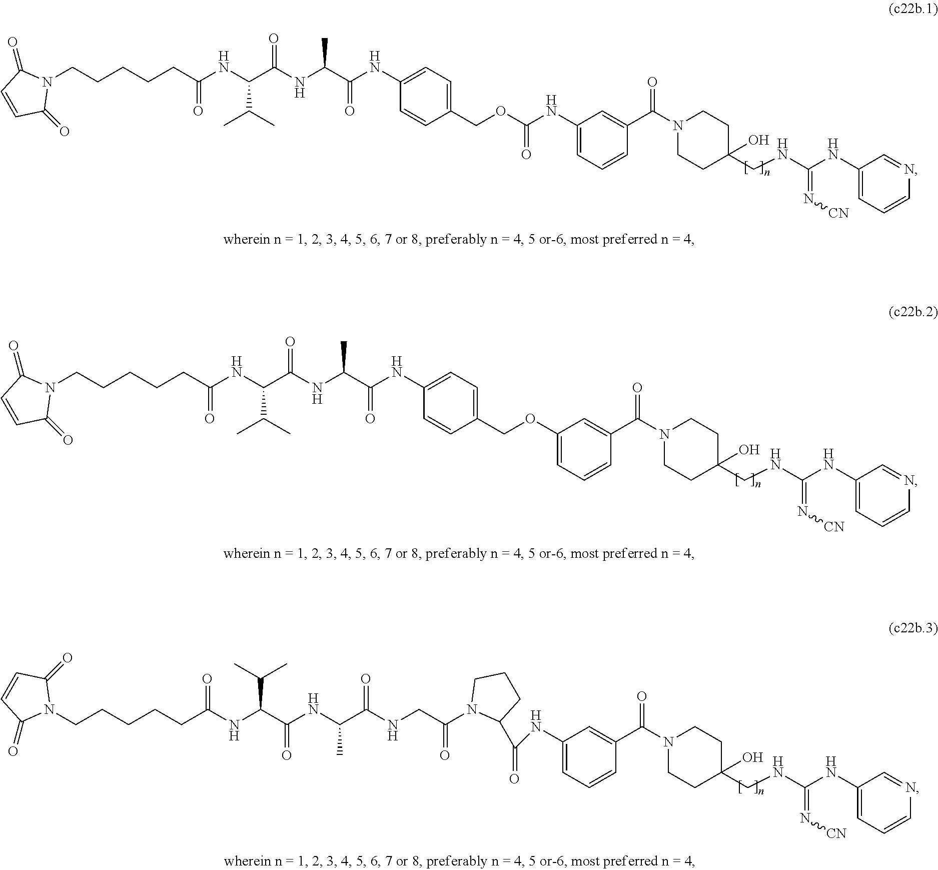

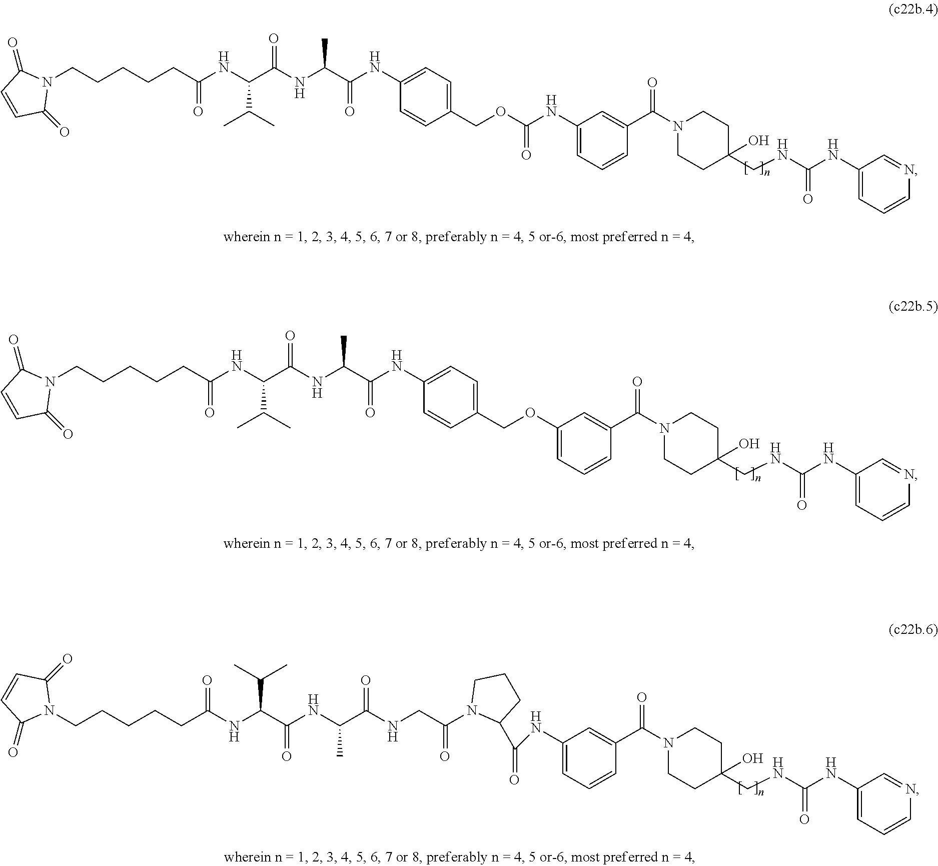

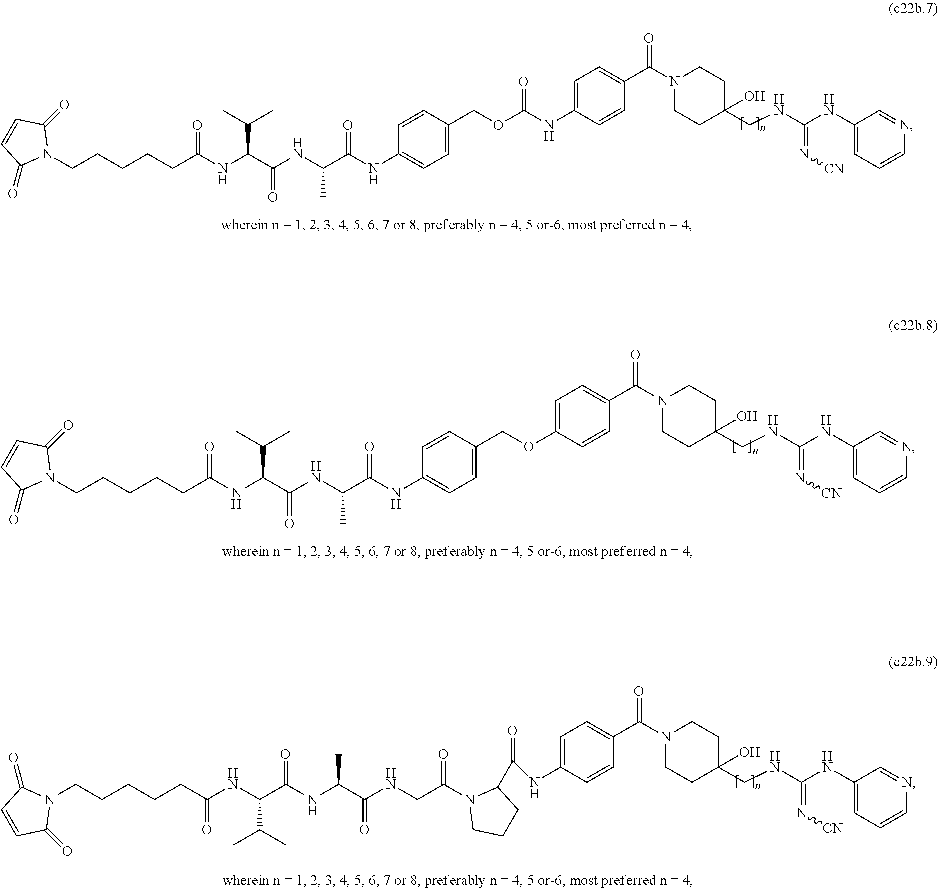

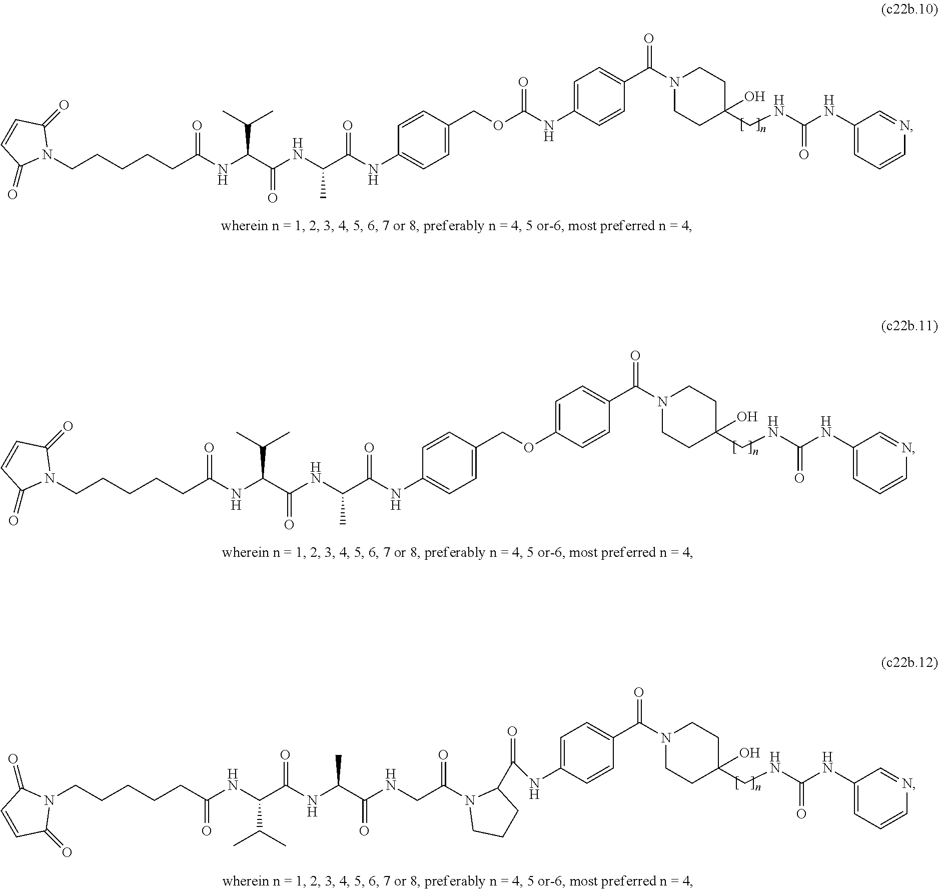

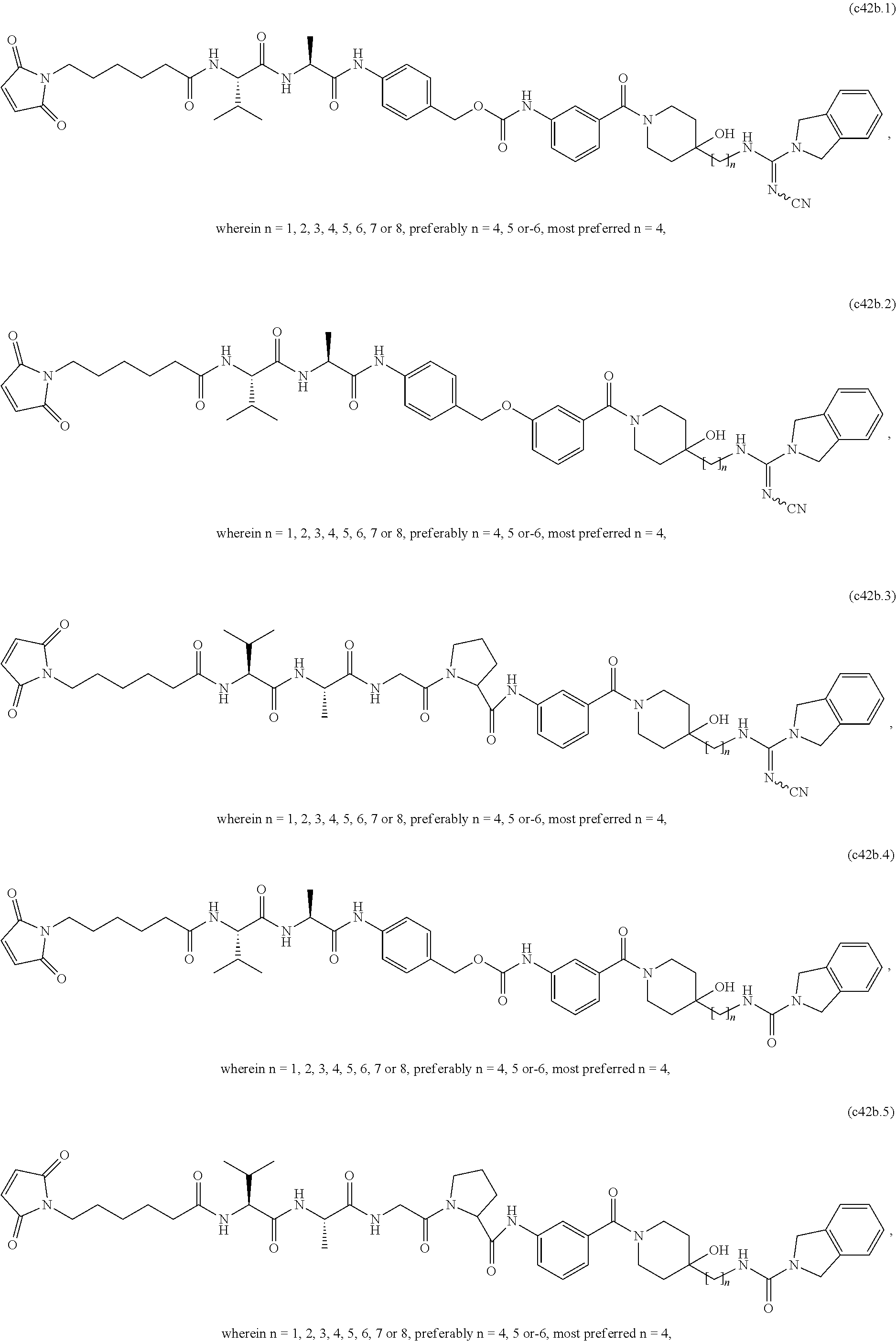

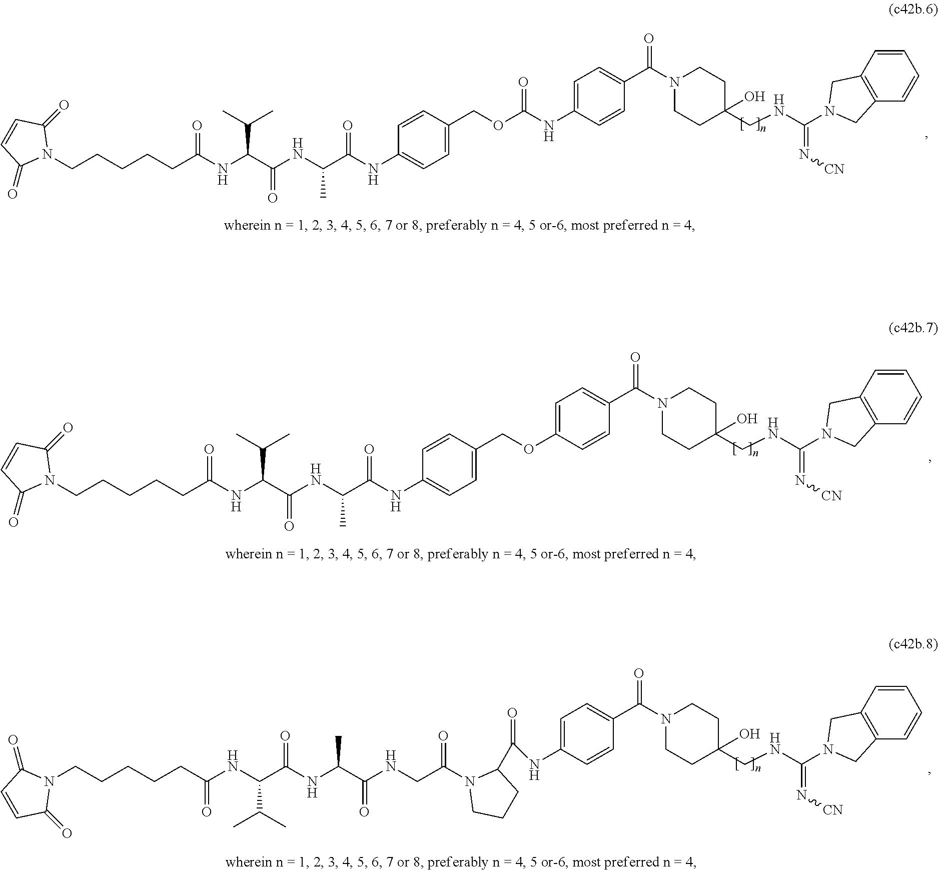

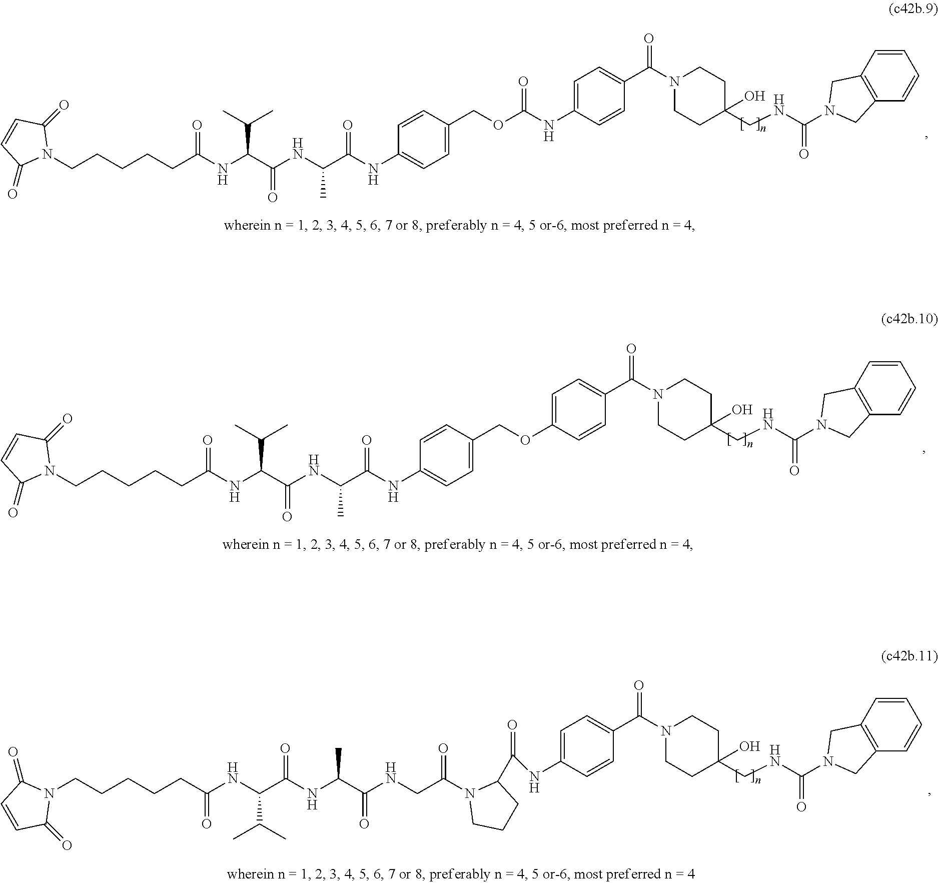

In particularly preferred embodiments, the compound of the present disclosure is selected from

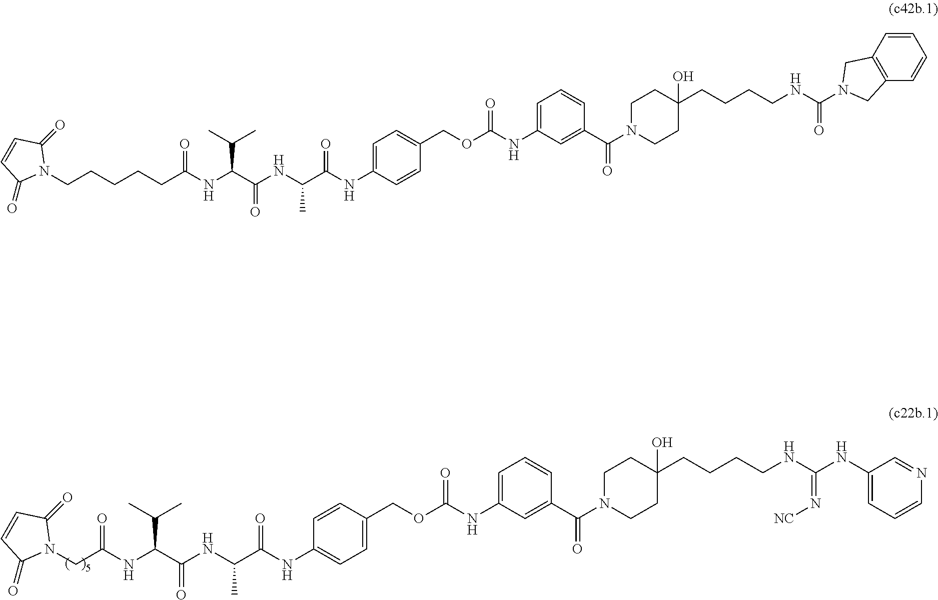

In one embodiment, the present disclosure pertains to the synthesis of the compounds of the present disclosure, the synthesis of which comprises reacting intermediate (i1) with intermediate (i2) in the presence of Mg, LaCl₃—LiCl, THF to yield intermediate (i3)

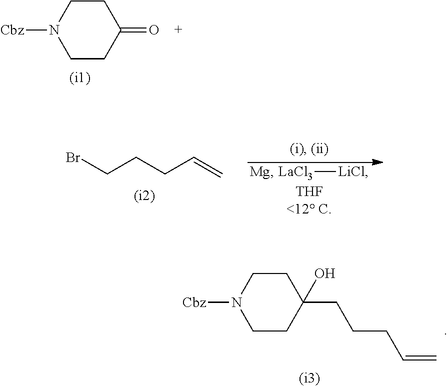

For example, the above intermediate (3) may be obtained by (i) a Grignard-formation and (ii) an alcohol synthesis, which may be done according to the below procedure:

### (i) Grignard-Formation

In the first step magnesium (solid) and I₂ are added to a dried three-neck flask equipped with a reflux condenser under argon atmosphere. In the second step tetrahydrofuran (THF) is added to a final concentration of about 0.4 M. In a third step, the mixture is heated to a temperature of about 60-70° C. (reflux) for about 15-60 minutes, followed by a fourth step in which the mixture is allowed to cool to room temperature. In a fifth step, 5-bromo-1-pentene (1-2 equivalent) is added dropwise over a period of 5-30 minutes. In a sixth step, the reaction mixture is incubated at room temperature until the reaction solution becomes less translucent accompanied by the formation of bubbles. The reaction mixture may then be heated to 60-70° C. for 20-45 minutes and allowed to cool to room temperature.

### (ii) Alcohol Synthesis

A solution of LaCl₃*2LiCl in THF is added to a dried two-neck flask, followed by addition of piperidone (1 eq.). The reaction mixture is stirred at room temperature for about 1 h, and subsequently cooled to <12° C. The Grignard compound from step (i) is added dropwise at a temperature of <12° C. The reaction progress may e.g. be followed by TLC and/or HPLC.

Following completion of the reaction the intermediate (i3) may be purified by quenching the reaction (ii) with NH₄Cl washing with H₂O and brine and dried over MgSO₄. Purification can be performed by flash chromatography.

In one embodiment, the present disclosure pertains to the use of the inventive compounds as disclosed above in the manufacture of an antibody-drug conjugate.

In one embodiment, the present disclosure pertains to an antibody-drug conjugate (ADC) represented by the structure

Ab-(Z′-L-T)ₖ,

wherein Ab is an antibody or antigen-binding fragment thereof, or antibody-like protein;
Z′ is a chemical moiety formed from a coupling reaction between a reactive substituent Z present on L and a reactive substituent present within an antibody, or an antigen-binding fragment thereof;
L is a linker selected from a cleavable or non-cleavable linker as disclosed herein;
T is a compound of the present disclosure, which is a NAMPT inhibitor, wherein k is from about 1 to about 12, e.g. 1, 2, 3, 4, 5, 6, 7, 8, 9, 10, 11, 12. (preferably 4 to 10, or 12).

The term “antibody” as used herein shall refer to a protein consisting of one or more polypeptide chains encoded by immunoglobulin genes or fragments of immunoglobulin genes or cDNAs derived from the same. Said immunoglobulin genes include the light chain kappa, lambda and heavy chain alpha, delta, epsilon, gamma and mu constant region genes as well as any of the many different variable region genes.

The basic immunoglobulin (antibody) structural unit is usually a tetramer composed of two identical pairs of polypeptide chains, the light chains (L, having a molecular weight of about 25 kDa) and the heavy chains (H, having a molecular weight of about 50-70 kDa). Each heavy chain is comprised of a heavy chain variable region (abbreviated as VH or V_{H}) and a heavy chain constant region (abbreviated as CH or C_{H}). The heavy chain constant region is comprised of three domains, namely CH1, CH2 and CH3. Each light chain contains a light chain variable region (abbreviated as VL or V_{L}) and a light chain constant region (abbreviated as CL or C_{L}). The VH and VL regions can be further subdivided into regions of hypervariability, which are also called complementarity determining regions (CDR) interspersed with regions that are more conserved called framework regions (FR). Each VH and VL region is composed of three CDRs and four FRs arranged from the amino terminus to the carboxy terminus in the order of FR1, CDR1, FR2, CDR2, FR3, CDR3, FR4. The variable regions of the heavy and light chains form a binding domain that interacts with an antigen. The constant regions (Fc regions) are not directly involved in the binding of the antibody to the antigen but exhibit various effector functions such as participation in antibody dependent cell-mediated cytotoxicity (ADCC), phagocytosis via binding to Fcg receptor, half-life/clearance rate via neonatal Fc receptor (FcRn) and complement activation via the C1q component, leading to the chemotactic, opsonic and, potentially in the case of a viable cellular antigen target, cytolytic actions of complement. Human antibodies of the IgG1 class are the most potent in activating the complement system and are therefore the desirable isotype for the therapeutic application of the antibodies of the present disclosure.

Human Fcg receptors include FcgR (I), FcgRlla, FcgRllb, FcVRllla and neonatal FcRn for which it was demonstrated that a common set of IgG1 residues is involved in binding all FcgRs, while FcgRII and FcgRIII utilize distinct sites outside of this common set (Shields et al. (2001) J. Biol. Chem 276: 6591-6604). One group of IgG1 residues reduced binding to all FcgRs when altered to alanine: Pro-238, Asp-265, Asp-270, Asn-297 and Pro-239 (numbering according to EU numbering system). All are in the IgG CH2 domain and clustered near the hinge joining CH1 and CH2. While FcgR1 utilizes only the common set of IgG1 residues for binding, FcgRII and FcgRIII interact with distinct residues in addition to the common set. Alteration of some residues reduced binding only to FcgRII (e.g. Arg-292) or FcgRIII (e.g. Glu-293). Some variants showed improved binding to FcgRII or FcgRIII but did not affect binding to the other receptor. The neonatal FcRn receptor is believed to be involved in both antibody clearance and the transcytosis across tissues (see: Junghans (1997) Immunol. Res 16: 29-57; and Ghetie et al. (2000) Annu. Rev. Immunol. 18: 739-766). Human IgG1 residues determined to interact directly with human FcRn include Ile253, Ser254, Lys288, Thr307, Gln311, Asn434 and His435.

The terms “CDR”, or any variation thereof as used herein follow the Kabat numbering convention (Kabat et al., Sequences of Proteins of Immunological Interest, 4th Ed., U.S. Department of Health and Human Services, National Institutes of Health (1987)). However, although the Kabat numbering convention for amino acid residues in variable domain sequences and full-length antibody sequences is used throughout this specification, it will be apparent to those skilled in the art that there are alternative numbering conventions for amino acid residues in variable domain sequences and full-length antibody sequences. There are also alternative numbering conventions for CDR sequences, for example those set out in Chothia et al. (1989) Nature 342: 877-883. The structure and protein folding of the antibody may mean that other residues based on the numbering system used are considered part of the CDR sequence and would be understood to be so by a skilled person, however, these differences functionally do not imply altered or different antigen-binding of the respective antibody. Other numbering conventions for CDR sequences available to a skilled person include “AbM” (University of Bath) and “contact” (University College London) methods.

The CDRs are most important for binding of the antibody or the antigen binding portion thereof. The FRs can be replaced by other sequences, provided the three-dimensional structure which is required for binding of the antigen is retained. Structural changes of the construct most often lead to a loss of sufficient binding to the antigen.

Antibodies typically bind specifically to their cognate antigen with high affinity, reflected by a dissociation constant (KD) of 10⁻⁵ to 10⁻¹¹ M or less. Any K_{D} greater than about 10⁻⁴ M is generally considered to indicate nonspecific binding. As used herein, an antibody that “binds specifically” to an antigen refers to an antibody that binds to the antigen and substantially identical antigens with high affinity, which means having a K_{D} of 10⁻⁷ M or less, preferably 10⁻⁸M or less, even more preferably 5×10⁻⁹ M or less, and most preferably from about 10⁻⁸, 10⁻⁹ M to about 10⁻¹⁰ M, 10⁻¹¹ or less, or e.g. from about 10⁻¹⁰ M to about 10⁻¹¹ M or less, but does not bind to unrelated (e.g. structurally or sequence unrelated) antigens with an affinity equal to the affinity for the specific target.

The antibody of the ADC according to the disclosure which may e.g. also be referred to as immunoglobulin may be from any of the commonly known isotypes, including but not limited to IgA, secretory IgA, IgG and IgM, preferably of the IgG isotype, more preferably the antibody of the ADC of the disclosure is one of an IgG1, IgG2, IgG3 or IgG4 isotype, most preferably the antibody is an IgG1, or IgG4 isotype. Throughout this disclosure, the antibody comprised in the ADC of the disclosure may also be referred to “antibody moiety” which may be used interchangeably. Both terms refer to the antibody part of the ADC of the disclosure.

The term “antibody fragment” or “antigen-binding fragment” as used herein refers to an antibody fragment or analog of an antibody which retains the binding specificity of the parent anti-GUCY2C antibody as disclosed herein and comprises a portion (for example, one or more CDRs) or variable region of the antigen binding region of the parent antibody. The antibody fragment is, for example, Fab, Fab′, F(ab′)2, Fv fragment, sc-Fv, unibody, diabody, linear antibody, nanobody, domain antibody, or multispecific antibody fragment formed from the antibody fragment. A Fab fragment consists of the CH1 and variable regions of one light chain and one heavy chain. The heavy chain of a Fab molecule cannot form a disulfide bond with another heavy chain molecule. A Fab′ fragment as contains a light chain and a portion of a heavy chain that contains the VH domain, the CH1 domain, and the region between the CH1 and CH2 domains. A F(ab′)2 fragment contains two light chains and two heavy chains containing a portion of the constant region between the CH1 and CH2 domains, thereby forming an interchain disulfide bond between the two heavy chains. A F(ab′)2 fragment is composed of two Fab′ fragments held together by the disulfide bond between the two heavy chains. A “Fv fragment” contains variable regions from both the heavy and light chains but lacks the constant region. The term “single-chain antibody” is a single-chain recombinant protein formed by connecting the heavy chain variable region VH and the light chain variable region VL of an antibody through a connecting peptide. It is the smallest antibody fragment with a complete antigen-binding site. The term “domain antibody fragment” is an immunoglobulin fragment with immunological functions that only contains a heavy chain variable region or a light chain variable region chain. In some cases, two or more VH regions are covalently linked to a peptide linker to form a bivalent domain antibody fragment. The two VH regions of the bivalent domain antibody fragment can target the same or different antigens.

Thus, according to some embodiments, the antibody of the ADC of the present disclosure is a humanized or human antibody. The term “humanized” antibody as used herein refers to an antibody that contains minimal sequences derived from non-human immunoglobulin. Thus, “humanized” forms of non-human (e.g., murine) antibodies are chimeric antibodies that contain minimal sequence derived from the non-human antibody. All or substantially all of the framework regions may also be those of a human immunoglobulin sequence. The humanized antibody may also contain at least a portion of an immunoglobulin constant region (Fc), typically that of a human immunoglobulin consensus sequence. Methods of antibody humanization are known in the art and have been described, for example, in Riechmann et al., Nature 332:323-7, 1988; U.S. Pat. Nos. 5,530,101; 5,585,089; 5,693,761; 5,693,762; and 6,180,370. Human antibodies such as those of the inventive ADC can be made by the hybridoma method using human myeloma or mouse-human heteromyeloma cells lines, see Kozbor (1984) J. Immunol 133, 3001. Alternative methods include the use of phage libraries or transgenic mice both of which utilize human variable region repertories (see e.g. Winter (1994) Annu. Rev. Immunol 12: 433-455; Green (1999) J. Immunol. Methods 231: 11-23). Several strains of transgenic mice are now available wherein their mouse immunoglobulin loci have been replaced with human immunoglobulin gene segments (see e.g. Tomizuka (2000) PNAS 97: 722-727; Fishwild (1996) Nature Biotechnol. 14: 845-851; Mendez (1997) Nature Genetics, 15: 146-156). Upon antigen challenge such mice are capable of producing a repertoire of human antibodies from which antibodies of interest can be selected.

In some embodiments, the antibody, or antibody fragment of the ADC according to the present disclosure is a monoclonal antibody. As used herein, the term “monoclonal antibody” (“mAb”) refers to a preparation of antibody molecules of single binding specificity and affinity for a particular epitope, representing a homogenous antibody population, i.e., a homogeneous population consisting of a whole immunoglobulin, or a fragment. Monoclonal antibodies (mAb) which are derived from e.g. mouse may cause unwanted immunological side-effects when administered to humans due to the fact that they contain a protein from another species which may elicit antibodies. To overcome this problem, antibody humanization and maturation methods have been designed to generate antibody molecules with minimal immunogenicity when applied to humans, while ideally still retaining specificity and affinity of the non-human parental antibody (for review see Almagro and Fransson 2008).

In some embodiments, the NAMPT inhibitor-linker conjugates (Z-L-T) as disclosed herein are conjugated to the antibody or antigen-binding fragment thereof via naturally occurring amine residues. Typically, such amine residues are e-amino groups on the antibody. Conjugation of the NAMPT inhibitor-linker conjugates (Z-L-T) of the present disclosure may be done using commercially available kits or as described in Ko et al MAbs. 2021 January-December; 13(1):1914885.

In a preferred embodiment, the NAMPT inhibitor-linker conjugates (Z-L-T) of the present disclosure as disclosed herein are conjugated to the antibody via naturally occurring reactive cysteine residues. Naturally occurring reactive cysteine residues are e.g. the cysteine residues that form the interchain disulfide bonds in an antibody upon reduction of said residues. Corresponding conjugation methods are known in the art and may e.g. be done according to the method disclosed in Neumann et al. Mol Cancer Ther (2018) 17 (12): 2633-2642. Briefly, the antibody may e.g. be subjected to buffer exchange into PBS, pH 7.4 followed by dilution of the antibody to a final concentration of 5 mg/mL in PBS, warmed to 37° C. A stock solution of tris(2-carboxyethyl)-phosphine TCEP (50 mM in water) freshly prepared in water may then be used in 2.5 molar excess (relative to the antibody concentration). The reduction reaction may e.g. be allowed to proceed for about 2 hours, after which the conjugation of the Linker-NAMPT inhibitor conjugate (Z-L-T) may be done by adding a stock solution of the conjugate in about 5 molar equivalents to the antibody may be added and incubated for about 1 hour after which the reaction mixture be subjected to a buffer exchanged buffer into PBS.

In some embodiments, the antibody of the inventive ADC comprises a heavy chain constant (Fc) region which comprises at least one amino acid substitution selected from L234A, L235A, A118C, S239C, D265C (according to EU numbering system, Edelman et al., 1969, Proc Natl Acad Sci USA 63:78-85.). As used herein, the term “amino acid substitution” or “mutation” both of which may be used interchangeably relates to modifications of the amino acid sequence of the protein, wherein one or more amino acids are replaced with the same number of different amino acids, producing a protein which contains a different amino acid sequence than the original protein.

In some embodiments, the Fc region of the antibody of the inventive ADC further comprises at least one cysteine amino acid substitution at sites where the engineered cysteines are available for conjugation but do not perturb immunoglobulin folding and assembly, such as e.g. A118C, S239C, D265C (according to EU numbering system). Cysteine-substituted or cysteine-engineered antibodies have been disclosed in WO2016040856A2, or Junutula, et al., 2008 Nature Biotech., 26(8):925-932; Doman et al (2009) Blood 114(13):2721-2729; U.S. Pat. Nos. 7,521,541; 7,723,485; WO2009/052249 and WO2016/142049) as well as method of obtaining the same. Preferred cysteine substitution in the Fc region of the antibody in the ADC of the present disclosure as disclosed herein is D265C alone, or in combination with A118C, or S239C (according to EU numbering system), whereby if the antibody has been engineered to comprise two cysteine amino acid substitutions, the combination of the cysteine substitutions A118C and D265C is preferred. The use of cysteine engineered antibodies may be advantageous to obtain ADCs such as those of the present disclosure with a DAR=8, 10 or a DAR of 12, which may e.g. be generated by conjugating the compound of the present disclosure to interchain cysteine residues and the engineered cysteine residues.

In some embodiments, the Fc region of the antibody of the ADC as disclosed herein comprises the mutations L234A and L235A (according to EU-numbering system) in addition to the cysteine mutations disclosed above. The use of antibodies comprising said mutations L234A, L235A may e.g. be particularly advantageous to reduce or ablate the interaction of the Fc region of said antibodies with FcγRs by at least 95%, 97.5%, 99% compared to a wild-type Fc region.

The chemical moiety Z′ in the inventive ADC is formed from a coupling reaction between a reactive substituent Z as disclosed above present on L and a reactive substituent present within an antibody, e.g. a reactive amine residue present on the antibody, or a reactive cysteine residue of the antibody. Reactive cysteine residues are for example the cysteine residues that form the interchain disulfide bonds within the antibody following reduction under suitable conditions, or the engineered cysteine residues in the Fc region of the antibody as disclosed above, or e.g. both the reduced interchain-forming cysteine residues and the engineered cysteine residues in the Fc region. It is preferred that Z is thiol-reactive as disclosed herein above, most preferably Z is a maleimidyl residue present on L such that Z′ is a succinimidyl residue present on L following conjugation of T-L-Z to the antibody having the structure

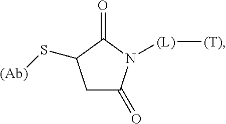

wherein L is a cleavable or non-cleavable linker as disclosed herein above.

Accordingly, the ADC as disclosed in the present disclosure above having the structure Ab-(Z′-L-T)ₖ, comprises from about 1, 2 Linker-NAMPT inhibitor conjugates (Z′-L-T, k=1 or 2) to about k=12 Linker-NAMPT inhibitor conjugates, preferably the ADC comprises n=4, 5, 6, 7, 8, 9, 10, 11, or 12 Linker-NAMPT inhibitor conjugates, more preferably the ADC comprises k=4, 6, 8, 10, or 12 linker-NAMPT inhibitor conjugates. Accordingly, the ADC of the present disclosure may e.g. have a Drug-Antibody-Ratio (DAR) of about 2, 4, 6 to about 8, 10, 12, preferably, from about 4, 6, 8 to about 10, 12, or from about 10 to about 12. The Drug-Antibody-Ratio (DAR) of ADCs according to the disclosure can be determined according to the methods as disclosed in e.g. Journal of Pharmaceutical Analysis 10 (2020) 209-220, the content of which is hereby incorporated in its entirety, or as disclosed herein further below.

In some embodiments, the ADC of the present disclosure has the structure of

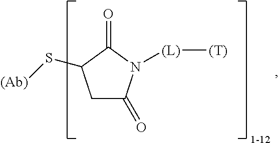

wherein L is a linker as disclosed herein, T is a NAMPT inhibitor (compound) of the present disclosure and wherein the ADC of the present disclosure comprises from about 1 to about 12 linker-payload (Z′-L-T) conjugates, preferably about 4, 6, 8, 10 12, more preferably about 8, 10 or 12.

According to some embodiments, the ADC of the present disclosure having the structure of Ab-(Z-L-T)ₖ comprises about at least one, two, 4, 6, 8, 10 or 12, e.g. 1, 2, 3, 4, 5, 6, 7, 8, 9, 10, 11, or 12 NAMPT inhibitor linker (L) conjugates of the present disclosure (e,g, k=1, 2, 3, 4, 5, 6, 7, 8, 9, 10, 11 or 12) selected from the compounds (1.75), (1.76), (1.77), (1.78), (1.79), (1.80), (1.81), (1.82), (1.83), (1.84), (1.85), (1.86), (1.87), (1.88), (1.89), (1.90), (1.91), (1.92), (1.93), (1.94), (1.95), (1.96), (1.97), (1.98), (1.99), (1.100), (1.101), (1.102), (1.103), (1.104), (1.105), (1.106), (1.107), (1.108), (1.109), (1.110), (1.111), (1.112), (1.113), (1.114), (1.115), (1.116), (1.117), (1.118), (1.119), (1.120), (1.121), (1.122), (1.123), (1.124), (1.125) as disclosed above wherein I_{N} is C₁-C₆ alkyl, R¹ is NR^{L} d R² is H for each compound. Preferably, for the NAMPT inhibitor linker (L) conjugates disclosed above, I_{N} is C₄ alkyl, R¹ is NR^{L} and R² is H for each compound.

According to preferred embodiments, the ADC of the present disclosure having the structure of Ab-(Z′-L-T)ₖ comprises about at least one, two, 4, 6, 8, 10 or 12, e.g. 1, 2, 3, 4, 5, 6, 7, 8, 9, 10, 11, or 12 NAMPT inhibitor linker (L) conjugates of the present disclosure selected from the compounds, e.g. k is 1, 2, 3, 4, 5, 6, 7, 8, 9, 10, 11, or 12, preferably, 6, 8, 10 or 12, more preferably 8, 10 or 12.

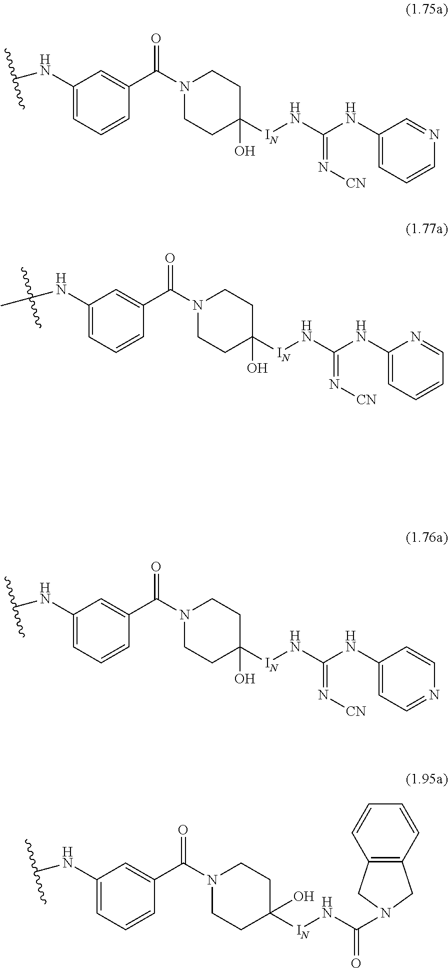

wherein I_{N} is C₁-C₆ alkyl, R¹ is NR^{L} as defined above and R² is H for each compound.

According to preferred embodiments, the ADC of the present disclosure having the structure of Ab-(Z′-L-T)ₖ comprises about at least one, two, 4, 6, 8, 10 or 12, e.g. 1, 2, 3, 4, 5, 6, 7, 8, 9, 10, 11, or 12 NAMPT inhibitor linker (L) conjugates of the present disclosure selected from the compounds

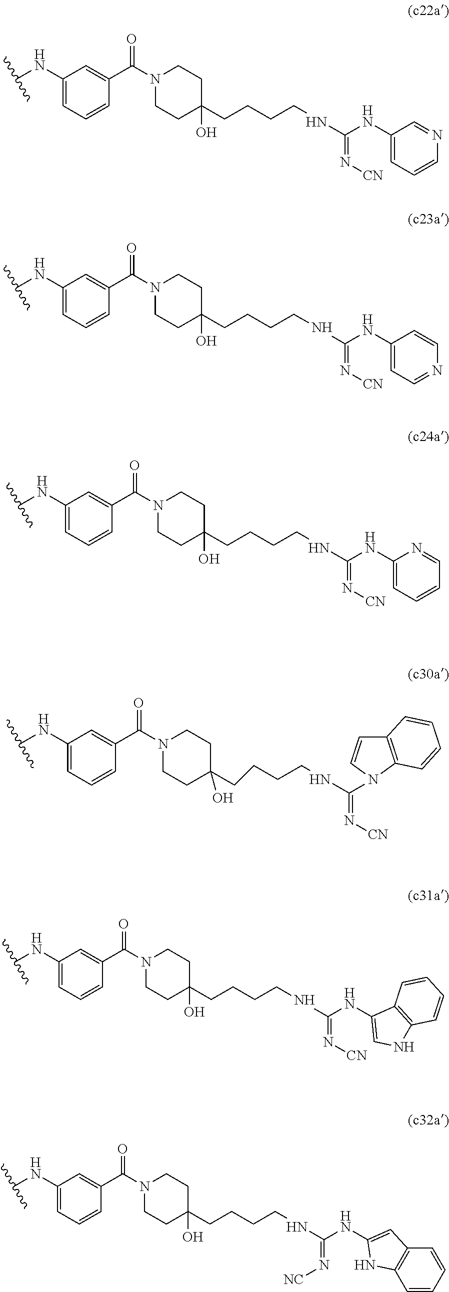 [SMILES: *Nc1cccc(C(=O)N2CCC(O)(CCCCN/C(=N/C#N)Nc3cccnc3)CC2)c1 ; *Nc1cccc(C(=O)N2CCC(O)(CCCCN/C(=N/C#N)Nc3ccncc3)CC2)c1 ; *Nc1cccc(C(=O)N2CCC(O)(CCCCN/C(=N/C#N)Nc3ccccn3)CC2)c1 ; *Nc1cccc(C(=O)N2CCC(O)(CCCCN/C(=N/C#N)n3ccc4ccccc43)CC2)c1 ; *Nc1cccc(C(=O)N2CCC(O)(CCCCN/C(=N/C#N)Nc3c[nH]c4ccccc34)CC2)c1 ; *Nc1cccc(C(=O)N2CCC(O)(CCCCN/C(=N\C#N)Nc3cc4ccccc4[nH]3)CC2)c1]

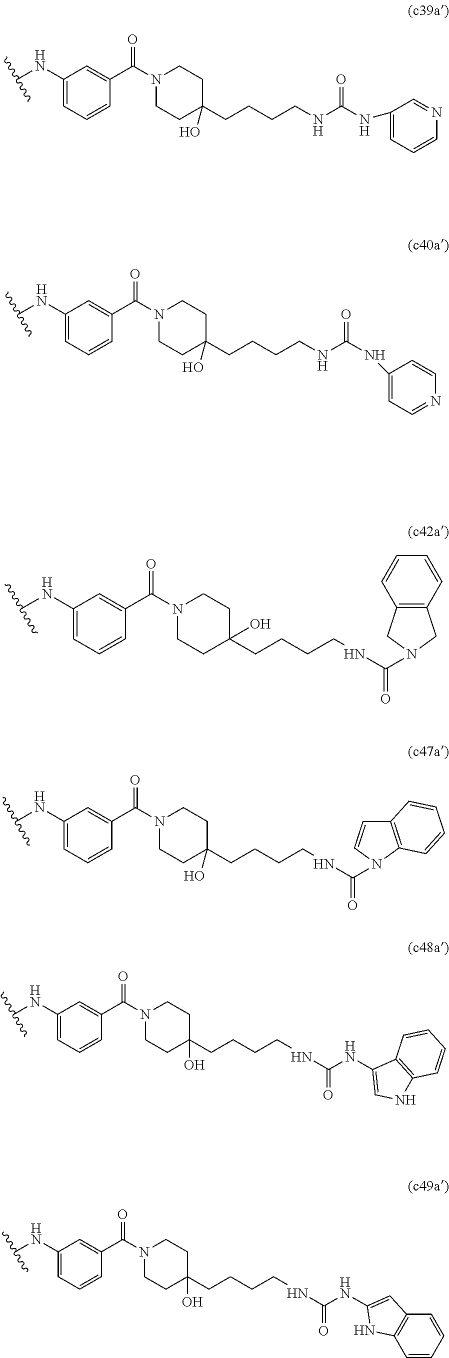 [SMILES: *Nc1cccc(C(=O)N2CCC(O)(CCCCNC(=O)Nc3cccnc3)CC2)c1 ; *Nc1cccc(C(=O)N2CCC(O)(CCCCNC(=O)Nc3ccncc3)CC2)c1 ; *Nc1cccc(C(=O)N2CCC(O)(CCCCNC(=O)N3Cc4ccccc4C3)CC2)c1 ; *Nc1cccc(C(=O)N2CCC(O)(CCCCNC(=O)n3ccc4ccccc43)CC2)c1 ; *Nc1cccc(C(=O)N2CCC(O)(CCCCNC(=O)Nc3c[nH]c4ccccc34)CC2)c1 ; *Nc1cccc(C(=O)N2CCC(O)(CCCCNC(=O)Nc3cc4ccccc4[nH]3)CC2)c1]

According to more preferred embodiments, the ADC of the present disclosure having the structure of Ab-(Z′-L-T)ₖ with k=1, 2, 3, 4, 5, 6, 8, 10 or 12, preferably, with k=2, 4, 6, 8, 10, or 12, more preferably, with k=6, 8, 10, 12, even more preferably with k=8, 10, or 12 NAMPT inhibitor linker conjugates of the present disclosure, whereby Z′-L-T is selected from the compounds

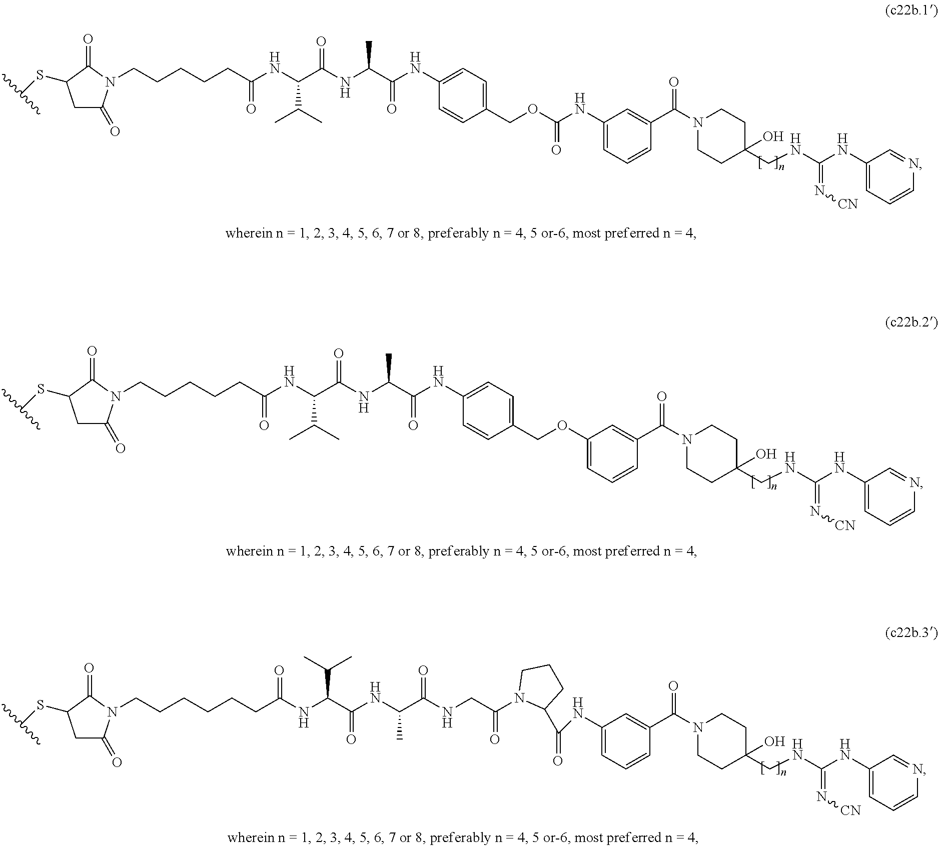

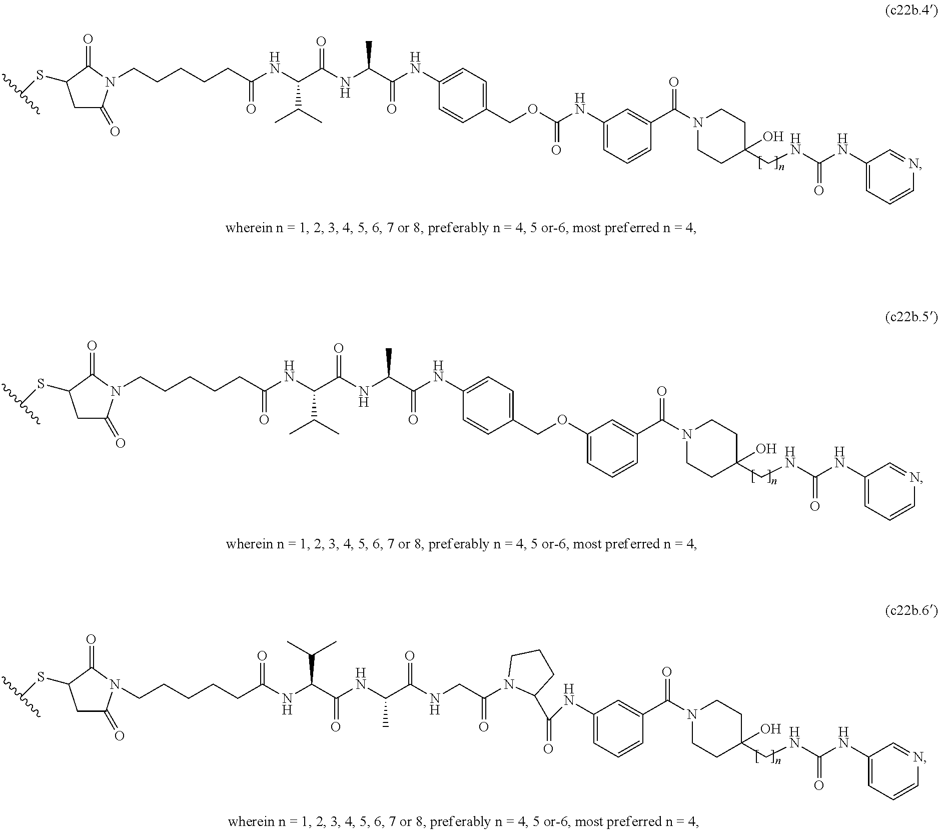

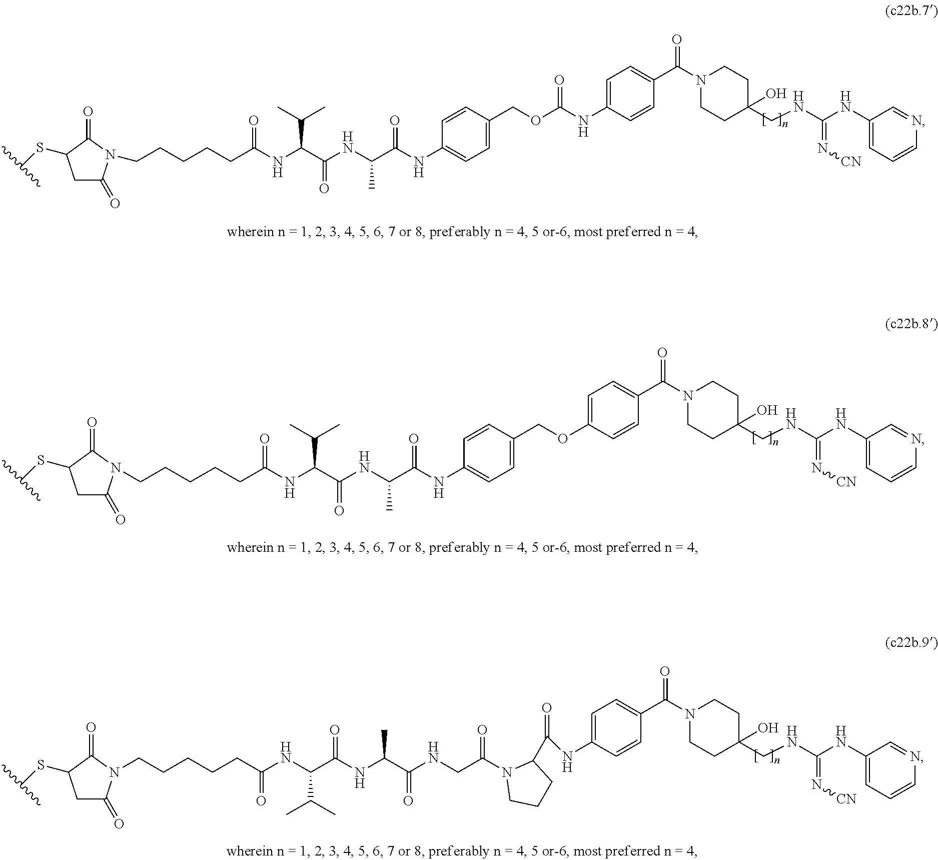

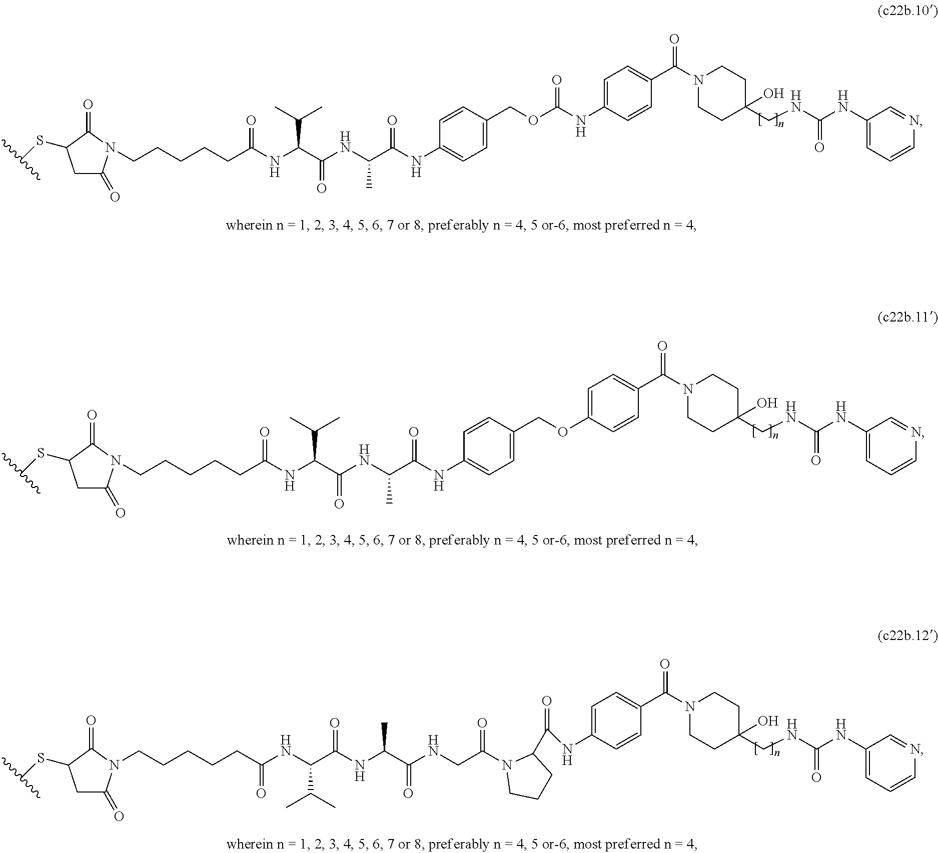

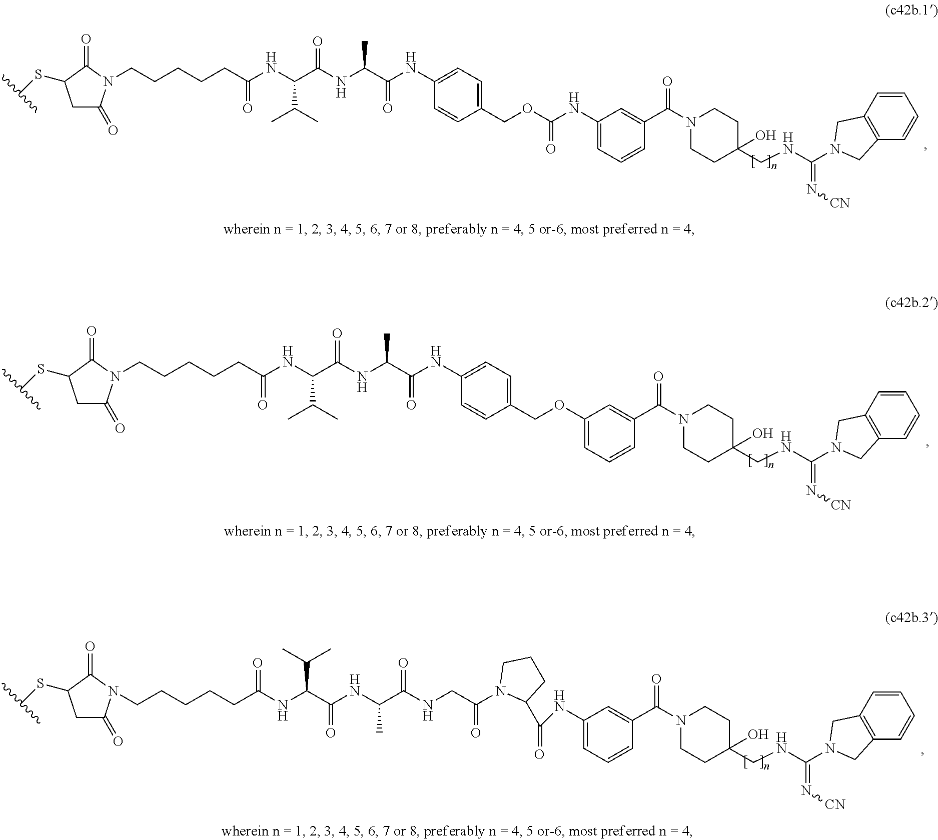

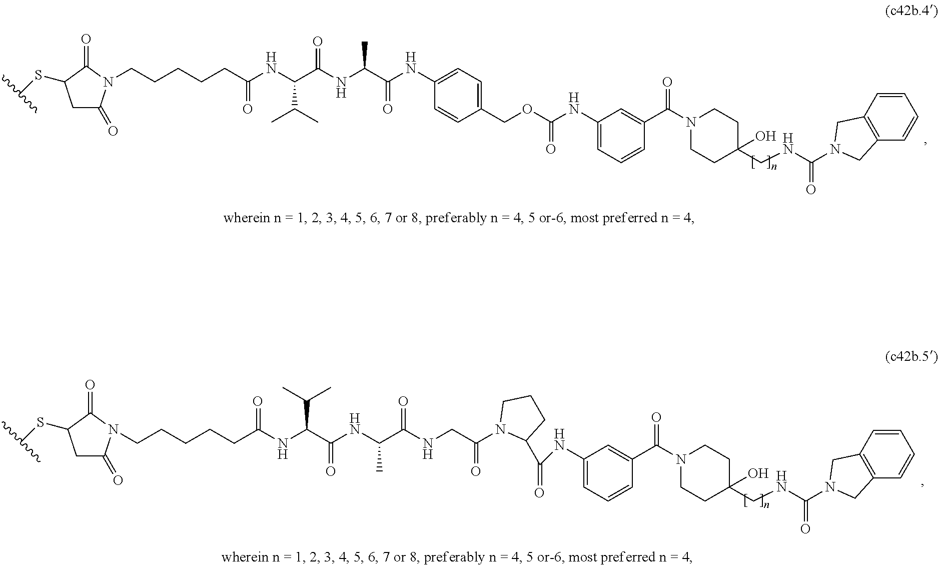

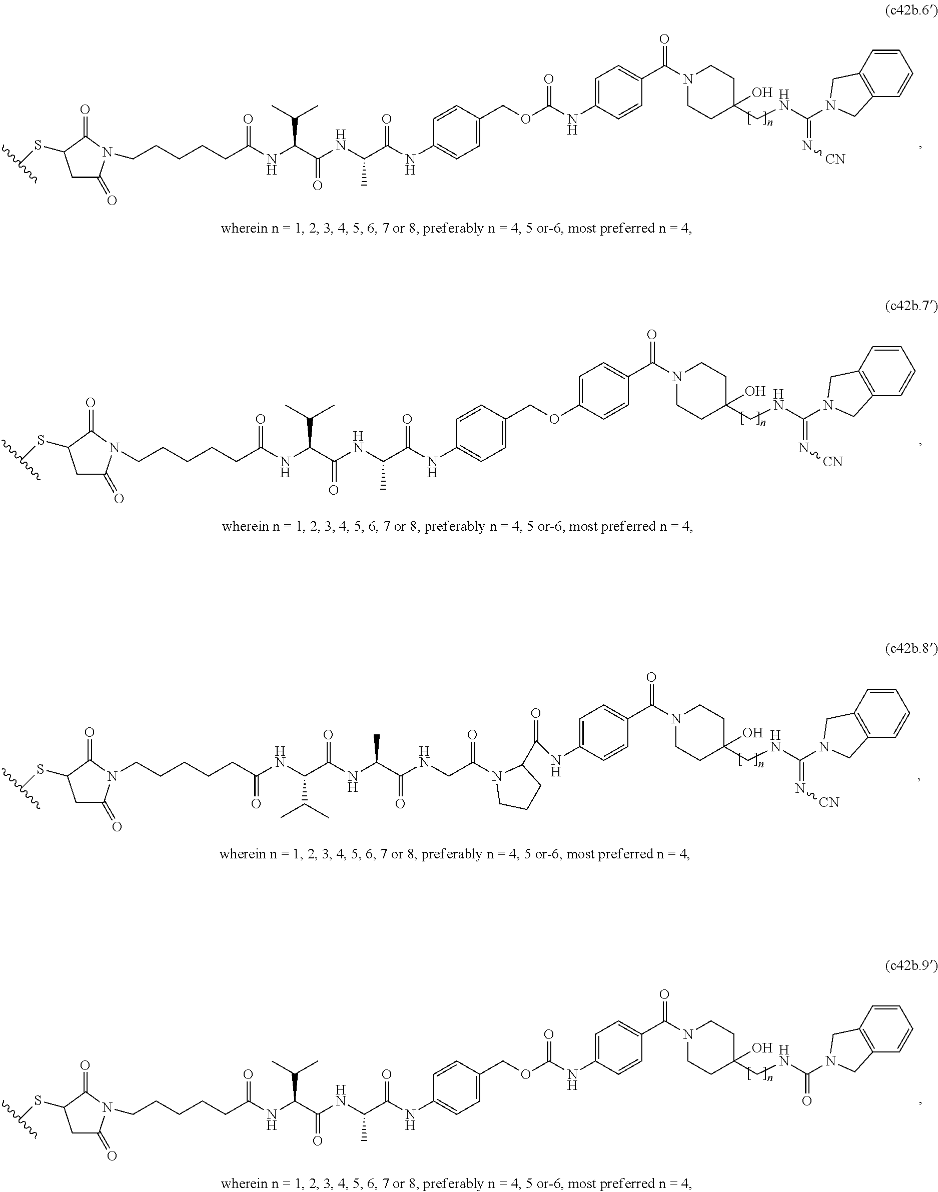

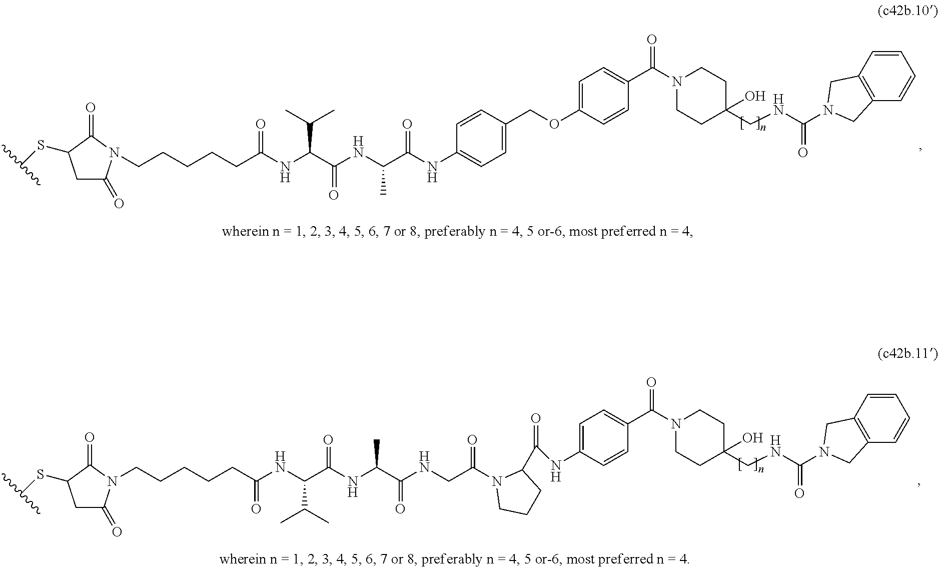

According to particularly preferred embodiments, the ADC of the present disclosure comprises a NAMPT inhibitor-linker conjugate (Z′-L-T) selected from (c42b.10′), (c22b.8′), wherein n is 4 and (Z′-L-T) has the structure

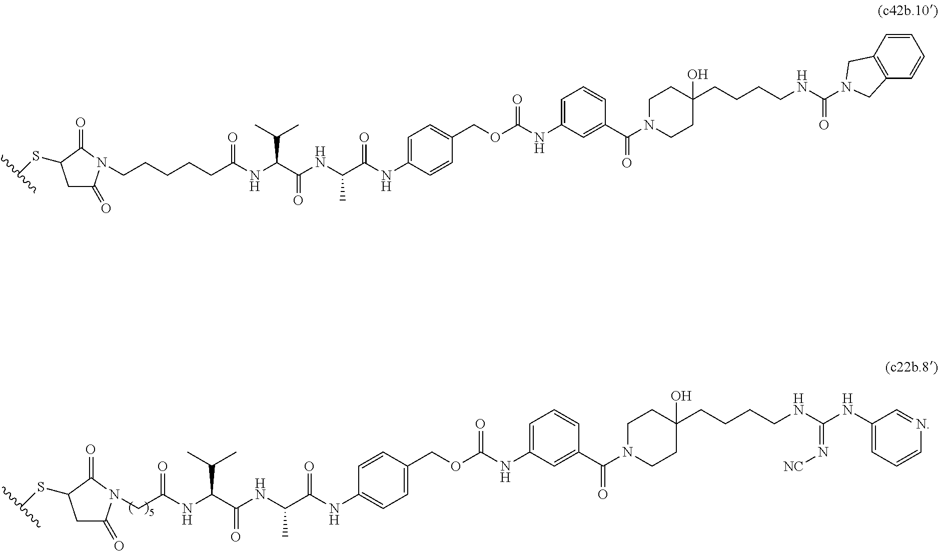

According to some embodiments, the ADC as disclosed above binds to a tumor antigen or tumor-associated antigen. The term “tumor antigen” shall refer to antigens specifically expressed by tumor cells and which are absent on non-cancerous cells of the host, e.g. a human. The term “tumor-associated antigen” (TAA) refers to any type of cancer antigen known in the art that can be associated with a tumor, including cell surface containing tumor cells, tumor-associated antigens are typically less tumor-specific and may also be present on a subset of non-cancerous cells of a host (e.g. a human).

According to some embodiments, the tumor-associated antigen to which the ADC of the present disclosure specifically binds is selected from the group comprising the following antigens: CD2, CD5, CD19, CD20, CD30, CD37, CD45, CD117, CD123, CD137, BCMA (CD269), HER3, NY-ESO-1, tyrosinase, Melan-A/MART-1, Her-2/neu, survivin, telomerase, WT1, CEA, gp100, Pmell7, mammaglobin-A, NY—BR-1, ERBB2, OA1, PAP, RAB 38/NY-MEL-1, TRP-1/gp75, TRP-2, BAGE-1, D393-CD20n, cyclin-A1, GAGE-1, GAGE-2, GAGE-8, GnTVf, HERV-K-MEL, KK-LC-1, KM-HN-1, LAGE-1, LY6K, MAGE-A1, MAGE-A2, MAGE-A3, MAGE-A4, MAGE-A6, MAGE-A9, MAGE-A10, MAGE-A12, MAGE-C1, MAGE-C2, mucin K, NA88-A, SAGE, spl7, SSX-2, SSX-4, TAG-1, TAG-3, TRAG-3, XAGE-lb, BCR-AB1, AIM-2, ALDH1A1, BCLX(L), BING-4, CALCA, CD274, CPSF, cyclin D1, DKK1, ENAH, EpCAM, EphA3, EZH2, FGF5, glypican-3, G250, HLA-DOB, hepsin, IDO1, IGF2B3, IL12Ralpha2, intestinal carboyxyl esterase, IGF1R, alpha-foetoprotein, kallikrein 4, KIF20A, Lengsin, M-CSF, M-CSP, mdm-2, MELOE-1, midkine, MMP-2, MMP-7, MUC1, MUC5AC, p53, PAX5, PBF, PRAME, prostate-specific membrane antigen (PSMA), RAGE-1, RGS5, RhoC, RNF43, RU2AS, SOX10, STEAP1, telomerase, TPBG, mesothelin, Axl, VEGF, EGFR, AFP, CA125, GUCY2C, TROP2, VEGFR-1, VEGFR-2, or VEGFR-3.

According to one embodiment, the ADC of the present disclosure as disclosed herein is for use as a medicament for the treatment of cancer, whereby the cancer is selected from the cancer types including but not limited to solid tumors and blood-borne cancers, such as acute and chronic leukemias, and lymphomas.

Solid tumors are exemplified, but not limited to, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer (particularly HER2-positive breast cancer), ovarian cancer (particularly HER2-positive ovarian cancer), triple-negative breast cancer (TNBC), prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, pharynx squamous cell carcinoma, medulloblastoma, craniopharyngioma, ependymoma, epidermoid carcinoma of the vulva, pinealoma, hemangioblastoma, acoustic neuroma, oligodendroglioma, meningioma, skin cancer, melanoma, neuroblastoma, and retinoblastoma.

Blood-borne cancers are exemplified, but not limited to, acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, and multiple myeloma.

Acute and chronic include lymphoblastic, myelogenous, lymphocytic, and myelocytic leukemias.

Lymphomas include Hodgkin's disease, non-Hodgkin's Lymphoma, Multiple myeloma, Waldenstrom's macroglobulinemia, Heavy chain disease and Polycythemia vera.

In one embodiment, the cancer is deficient in the nicotinic acid pathway. Nicotinate (niacin) and nicotinamide—more commonly known as vitamin B3—are precursors of the coenzymes nicotinamide-adenine dinucleotide (NAD+) and nicotinamide-adenine dinucleotide phosphate (NADP+). NAD+ synthesis occurs either de novo from amino acids, or a salvage pathway from nicotinamide. Most organisms use the de novo pathway whereas the savage pathway is only typically found in mammals. The specifics of the de novo pathway varies between organisms, but most begin by forming quinolinic acid (QA) from tryptophan (Trp) in animals, or aspartic acid in some bacteria (intestinal microflora) and plants. Nicotinate-nucleotide pyrophosphorylase converts QA into nicotinic acid mononucleotide (NaMN) by transfering a phosphoribose group. Nicotinamide mononucleotide adenylyltransferase then transfers an adenylate group to form nicotinic acid adenine dinucleotide (NaAD). Lastly, the nicotinic acid group is amidated to form a nicotinamide group, resulting in a molecule of nicotinamide adenine dinucleotide (NAD). Additionally, NAD can be phosphorylated to form NADP.

The salvage pathway involves recycling nicotinamide and nicotinamide-containing molecules such as nicotinamide riboside. The precursors are fed into the NAD+ biosynthetic pathway through adenylation and phosphoribosylation reactions. These compounds can be found in the diet, where the mixture of nicotinic acid and nicotinamide are called vitamin B3 or niacin. These compounds are also produced within the body when the nicotinamide group is released from NAD+ in ADP-ribose transfer reactions.

In cancers that are deficient in nicotinic acid pathway, administration of nicotinic acid will not replenish NAD⁺ and thereby will not reduce the toxicity of the NMPRT inhibitor to the cancer cell. Thus, the efficacy of the NMPRT inhibitor in cancer cells deficient in nicotinic acid pathway will not be reduced by administration of nicotinic acid. In contrast, administration of nicotinic acid to normal cells (which can synthesize NAD⁺ using the nicotinic acid pathway), reduces toxicity and side effects associated with administration of the NMPRT inhibitor to normal cells or uptake of the NMPRT inhibitor by normal cells. Therefore, a higher dose of the NMPRT inhibitor can be administered to the patient diagnosed with or suspected to have a cancer deficient in nicotinic acid when co-administered with nicotinic acid.4

One method that is useful for determination whether a cancer is deficient in nicotinic acid pathway is to add isotopically labeled nicotinic acid (e.g., with ²H, ³H, ¹³C, ¹⁴C, ¹⁵N, ¹⁷O, or ¹⁸O) to the tissue culture and monitor cellular production of isotopically labeled NAD+ in the tissue (see Hara et al. (2007), “Elevation of cellular NAD levels by nicotinic acid and involvement of nicotinic acid phosphoribosyltransferase in human cells”, Journal of Biological Chemistry 282 (34): 24574-24582, incorporated by reference herein in its entirety). Another method that is useful for determination whether a cancer is deficient in the nicotinic acid pathway is to administer an effective dose of a NMPRT inhibitor to cancer cells obtained from a patient followed by administration of nicotinic acid. If the cancer is deficient in nicotinic acid pathway, the cells will be rescued (i.e., the survival rate will increase). Another method is to determine NAPRT1 expression by immunohistochemical screening of tissue samples. Tissue sections can be scored for specific NAPRT1 expression by comparison of sections stained with anti-NAPRT1 compared to sequential sections stained with a pre-immune rabbit IgG to assess non-specific staining. These are just examples not limiting the scope of the disclosure and other methods for the determination of the nicotinic acid pathway can also be used.

It is to be understood that within the context of the present disclosure, the terms “cancer” or “tumor” include metastases and lesions.

In one embodiment, the present disclosure pertains to the use of the NAMPT inhibitors (compounds) as disclosed above for the manufacture of an antibody-drug conjugate (ADC), preferably for the manufacture of an ADC according to the present disclosure. Preferred compounds of the present disclosure to be used in the manufacture of an ADC are selected from (1.75), (1.76), (1.77), (1.78), (1.79), (1.80), (1.81), (1.82), (1.83), (1.84), (1.85), (1.86), (1.87), (1.88), (1.89), (1.90), (1.91), (1.92), (1.93), (1.94), (1.95), (1.96), (1.97), (1.98), (1.99), (1.100), (1.101), (1.102), (1.103), (1.104), (1.105), (1.106), (1.107), (1.108), (1.109), (1.110), (1.111), (1.112), (1.113), (1.114), (1.115), (1.116), (1.117), (1.118), (1.119), (1.120), (1.121), (1.122), (1.123), (1.124), (1.125), as disclosed above, wherein I_{N} is C₁-C₆ alkyl, R¹ is NR^{L} and R² is H for each compound as disclosed above, more preferably, wherein I_{N} is C₄ alkyl and R¹ is NR^{L} and R² is H for each compound.

In one embodiment, the present disclosure pertains to the use of NAMPT inhibitor-linker conjugate (Z-L-T) of the present disclosure as disclosed herein in the manufacture of an ADC. Preferably, the NAMPT inhibitor-linker conjugate (Z-L-T) is selected from the group consisting of (c22b.1), (c22b.2), (c22b.3), (c22b.4), (c22b.5), (c22b.6), (c22b.7), (c22b.8), (c22b.9), (c22b.10), (c22b.11), (c22b.12), (c42b.1), (c42b.2), (c42b.3), (c42b.4), (c42b.5), (c42b.6), (c42b.7), (c42b.8), (c42b.9), (c42b.10), and (c42b.11).

According to some embodiments, the NAMPT inhibitor-Linker conjugate (Z-L-T) of the present disclosure are conjugated to an antibody selected from Adecatumumab (anti-EpCam), Amatuximab, (anti-mesothelin), Amivantamab (anti-EGFR), Besilesomab (anti-CEA), blinatumomab (anti-CD19), brentuximab (anti-CD30), Cantuzumab, Cibisatamab (anti-CEACAM5), Cirmtuzumab (anti-RORI), cetuximab (anti-EGFR), Clivatuzumab (anti-MUC1), Gatipotuzumab (anti-MUC1), Coltuximab (anti-CD19), Daratumumab (anti-CD38), Duligotuzumab (anti-ERBB3), Edrecolomab (anti-EpCam), Enfortumab (anti-Nectin-4), Enoblituzumab (anti-CD276), Ensituximab (anti-MUC5AC), Epratuzumab (anti-CD22), Farletuzumab (anti-FOLR1), Igovomab (anti-CA-125), Inebilizumab (anti-CD19), Iratumumab (anti-CD30), Labetuzumab (anti-CEA), Margetuximab (anti-HER2/neu), Matuzumab (anti-EGFR), Modotuximab (anti-EGFR extracellular domain III), Naptumomab (anti-5T4), Naratuximab (anti-CD37), Necitumumab (anti-EGFR), Nimotuzumab (anti-EGFR), Obinutuzumab (anti-CD20), Ocaratuzumab (anti-CD20), Ocrelizumab (anti-CD20), Ofatumumab (anti-CD20), Otlertuzumab (anti-CD37), Ontuxizumab (anti-TEM1), Pertuzumab (anti-HER2/neu), Polatuzumab (anti-CD79b), Rituximab (anti-CD20), Rovalpituzumab (anti-DLL3), Sacituzumab (anti-TROP-2), Seribantumab (anti-ERBB3/HER3), Tafasitamab (anti-CD19), Tetulomab (anti-CD37), Timigutuzumab (anti-HER2), Trastuzumab (anti-HER2), Ublituximab (anti-CD20), Zolbetuximab (anti-Claudin 18.2), or Pulocimab (anti-VEGFR-2), whereby the NAMPT inhibitor-Linker conjugate (Z-L-T) of the present disclosure may e.g. be conjugated to naturally occurring cysteine residues of the respective antibody, or cysteine-engineered antibody variants of the above antibodies, which have been engineered to comprise at least one amino acid exchange in their heavy chain selected from A118C, S239C, or D265C, preferably D265C, whereby the amino acid numbering is according to the EU numbering system (Edelman, G. M. et al., Proc. Natl. Acad. USA, 63, 78-85 (1969)) and is used to refer to the corresponding amino acids in both, IgG1 and IgG4 isotype antibodies. The conjugation to the cysteine-engineered antibodies may e.g. be done as disclosed herein, see for example Lyon et al Methods in Enzymology, Volume 502, page 123-138, 2012 and the references cited within. For the generation of an antibody-drug conjugate which is characterized by comprising more than e.g. 4, 6, 8, 10 or more, preferably from about 6 to about 10, preferably 6, 8, or 10 NAMPT inhibitor-Linker conjugates (Z-L-T) as disclosed herein (high DAR, “hD” ADCs (see e.g. Antibody-Drug Conjugates, Methods in Molecular Biology 1045, (2013), chapter 18, Humana Press) the conjugation of NAMPT inhibitor-linker conjugates to naturally occurring cysteine residues of an antibody is preferred, e.g. the conjugation to an antibody as disclosed above.

According to preferred embodiments, the NAMPT inhibitor-linker conjugates as disclosed herein that are coupled to the antibodies as disclosed above are selected from the group comprising NAMPT inhibitor-linker conjugates (c22b.1), (c22b.2), (c22b.3), (c22b.4), (c22b.5), (c22b.6), (c22b.7), (c22b.8), (c22b.9), (c22b.10), (c22b.11), (c22b.12), (c42b.1), (c42b.2), (c42b.3), (c42b.4), (c42b.5), (c42b.6), (c42b.7), (c42b.8), (c42b.9), (c42b.10), and (c42b.11), e.g.

| Antibody | NAMPT inhibitor-Linker conjugate (Z-L-T) |
|---|---|
| Adecatumumab | (c22b.1), (c22b.2), (c22b.3), (c22b.4), (c22b.5), (c22b.6), |
| (anti-EpCam) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Amatuximab | (c22b.1), (c22b.2), (c22b.3), (c22b.4), (c22b.5), (c22b.6), |
| (anti-mesothelin) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Amivantamab | (c22b.1), (c22b.2), (c22b.3), (c22b.4), (c22b.5), (c22b.6), |
| (anti-EGFR) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Besilesomab | (c22b.1), (c22b.2), (c22b.3), (c22b.4), (c22b.5), (c22b.6), |
| (anti-CEA) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Blinatumomab | (c22b.1), (c22b.2), (c22b.3), (c22b.4), (c22b.5), (c22b.6), |
| (anti-CD19) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Brentuximab | (c22b.1), (c22b.2), (c22b.3), (c22b.4), (c22b.5), (c22b.6), |
| (anti-CD30) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Cantuzumab | (c22b.1), (c22b.2), (c22b.3), (c22b.4), (c22b.5), (c22b.6), |
| (anti-CanAg) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Cibisatamab | (c22b.1), (c22b.2), (c22b.3), (c22b.4), (c22b.5), (c22b.6), |
| (anti-CEACAM5) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Cirmtuzumab | (c22b.1), (c22b.2), (c22b.3), (c22b.4), (c22b.5), (c22b.6), |
| (anti-ROR1) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Cetuximab | (c22b.1), (c22b.2), (c22b.3), (c22b.4), (c22b.5), (c22b.6), |
| (anti-EGFR) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Clivatuzumab | (c22b.1), (c22b.2), (c22b.3), (c22b.4), (c22b.5), (c22b.6), |
| (anti-MUC1) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Gatipotuzumab | (c22b.1), (c22b.2), (c22b.3), (c22b.4), (c22b.5), (c22b.6), |
| (anti-MUC1) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Coltuximab | (c22b.1), (c22b.2), (c22b.3), (c22b.4), (c22b.5), (c22b.6), |
| (anti-CD19) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Daratumumab | (c22b.1), (c22b.2), (c22b.3), (c22b.4), (c22b.5), (c22b.6), |
| (anti-CD38) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Duligotuzumab | (c22b.1), (c22b.2), (c22b.3), (c22b.4), (c22b.5), (c22b.6), |
| (anti-ERBB3) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Edrecolomab | (c22b.1), (c22b.2), (c22b.3), (c22b.4), (c22b.5), (c22b.6), |
| (anti-EpCam) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Enfortumab | (c22b.1), (c22b.2), (c22b.3), (c22b.4), (c22b.5), (c22b.6), |
| (anti-Nectin-4) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Enoblituzumab | (c22b.1), (c22b.2), (c22b.3), (c22b.4), (c22b.5), (c22b.6), |
| (anti-CD276) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ensituximab | (c22b.1), (c22b.2), (c22b.3), (c22b.4), (c22b.5), (c22b.6), |
| (anti-MUC5AC) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Epratuzumab | (c22b.1), (c22b.2), (c22b.3), (c22b.4), (c22b.5), (c22b.6), |
| (anti-CD22) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Farletuzumab | (c22b.1), (c22b.2), (c22b.3), (c22b.4), (c22b.5), (c22b.6), |
| (anti-FOLR1) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Igovomab | (c22b.1), (c22b.2), (c22b.3), (c22b.4), (c22b.5), (c22b.6), |
| (anti-CA-125) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Inebilizumab | (c22b.1), (c22b.2), (c22b.3), (c22b.4), (c22b.5), (c22b.6), |
| (anti-CD19) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Iratumumab | (c22b.1), (c22b.2), (c22b.3), (c22b.4), (c22b.5), (c22b.6), |
| (anti-CD30) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Labetuzumab | (c22b.1), (c22b.2), (c22b.3), (c22b.4), (c22b.5), (c22b.6), |
| (anti-CEA) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Margetuximab | (c22b.1), (c22b.2), (c22b.3), (c22b.4), (c22b.5), (c22b.6), |
| (anti-HER2/neu) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Matuzumab | (c22b.1), (c22b.2), (c22b.3), (c22b.4), (c22b.5), (c22b.6), |
| (anti-EGFR) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Modotuximab | (c22b.1), (c22b.2), (c22b.3), (c22b.4), (c22b.5), (c22b.6), |
| (anti-EGFR | (c22b.7), (c22b.8), (c22b.9), (c22b.10), (c22b.11), (c22b.12), |
| extracellular | (c42b.1), (c42b.2), (c42b.3), (c42b.4), (c42b.5), (c42b.6), |
| domain III) | (c42b.7), (c42b.8), (c42b.9), (c42b.10), and (c42b.11) |
| Naptumomab | (c22b.1), (c22b.2), (c22b.3), (c22b.4), (c22b.5), (c22b.6), |
| (anti-5T4) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Naratuximab | (c22b.1), (c22b.2), (c22b.3), (c22b.4), (c22b.5), (c22b.6), |
| (anti-CD37) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Necitumumab | (c22b.1), (c22b.2), (c22b.3), (c22b.4), (c22b.5), (c22b.6), |
| (anti-EGFR) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Nimotuzumab | (c22b.1), (c22b.2), (c22b.3), (c22b.4), (c22b.5), (c22b.6), |
| (anti-EGFR) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Obinutuzumab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ocaratuzumab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ocrelizumab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ofatumumab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Otlertuzumab | (c22b.1), (c22b.2), (c22b.3), (c22b.4), (c22b.5), (c22b.6), |
| (anti-CD37) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ontuxizumab | (c22b.1), (c22b.2), (c22b.3), (c22b.4), (c22b.5), (c22b.6), |
| (anti-TEM1) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Pertuzumab | (c22b.1), (c22b.2), (c22b.3), (c22b.4), (c22b.5), (c22b.6), |
| (anti-HER2/neu) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Polatuzumab | (c22b.1), (c22b.2), (c22b.3), (c22b.4), (c22b.5), (c22b.6), |
| (anti-CD79b) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Rituximab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Rovalpituzumab | (c22b.1), (c22b.2), (c22b.3), (c22b.4), (c22b.5), (c22b.6), |
| (anti-DLL3) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Sacituzumab | (c22b.1), (c22b.2), (c22b.3), (c22b.4), (c22b.5), (c22b.6), |
| (anti-TROP-2) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Seribantumab | (c22b.1), (c22b.2), (c22b.3), (c22b.4), (c22b.5), (c22b.6), |
| (anti- | (c22b.7), (c22b.8), (c22b.9), (c22b.10), (c22b.11), (c22b.12), |
| ERBB3/HER3) | (c42b.1), (c42b.2), (c42b.3), (c42b.4), (c42b.5), (c42b.6), |
| | (c42b.7), (c42b.8), (c42b.9), (c42b.10), and (c42b.11) |
| Tafasitamab | (c22b.1), (c22b.2), (c22b.3), (c22b.4), (c22b.5), (c22b.6), |
| (anti-CD19) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Tetulomab | (c22b.1), (c22b.2), (c22b.3), (c22b.4), (c22b.5), (c22b.6), |
| (anti-CD37) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Timigutuzumab | (c22b.1), (c22b.2), (c22b.3), (c22b.4), (c22b.5), (c22b.6), |
| (anti-HER2) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Trastuzumab | (c22b.1), (c22b.2), (c22b.3), (c22b.4), (c22b.5), (c22b.6), |
| (anti-HER2) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Ublituximab | (c22b.1), (c22b.2), (c22b.3), (c22b.4), (c22b.5), (c22b.6), |
| (anti-CD20) | (c22b.7), (c22b.8), (c22b.9), (c22b.10), (c22b.11), (c22b.12), |
| | (c42b.1), (c42b.2), (c42b.3), (c42b.4), (c42b.5), (c42b.6), |
| | (c42b.7), (c42b.8), (c42b.9), (c42b.10), and (c42b.11) |
| Zolbetuximab | (c22b.1), (c22b.2), (c22b.3), (c22b.4), (c22b.5), (c22b.6), |
| (anti-Claudin | (c22b.7), (c22b.8), (c22b.9), (c22b.10), (c22b.11), (c22b.12), |
| 18.2) | (c42b.1), (c42b.2), (c42b.3), (c42b.4), (c42b.5), (c42b.6), |
| | (c42b.7), (c42b.8), (c42b.9), (c42b.10), and (c42b.11) |

In one embodiment, the present disclosure pertains to a composition which comprises a compound of the present disclosure as disclosed above, or the ADC of the present disclosure as disclosed above. For example, the composition comprises a compound selected from the group consisting of (c22a′), (c23a′), (c24a′), (c30a′), (c31a′), (c32a′), (c39a′), (c40a′), (c42a′), (c47a′), (c48a′), and (c49a′), or an ADC of the present disclosure comprising one of the NAMPT inhibitors as linker-payload conjugates (Z′-L-T) as disclosed above.

In a preferred embodiment, the composition of the present disclosure is a pharmaceutical composition.

The pharmaceutical composition of the present disclosure may optionally further comprise one or more pharmaceutically acceptable buffers, surfactants, diluents, carriers, excipients, fillers, binders, lubricants, glidants, disintegrants, adsorbents, and/or preservatives.

In aqueous form, said pharmaceutical formulation may be ready for administration, while in lyophilised form said formulation can be transferred into liquid form prior to administration, e.g., by addition of water for injection which may or may not comprise a preservative such as for example, but not limited to, benzyl alcohol, antioxidants like vitamin A, vitamin E, vitamin C, retinyl palmitate, and selenium, the amino acids cysteine and methionine, citric acid and sodium citrate, synthetic preservatives like the parabens methyl paraben and propyl paraben.

The pharmaceutical formulation may further comprise one or more stabilizer, which may be, for example an amino acid, a sugar polyol, a disaccharide and/or a polysaccharide. Said pharmaceutical formulation may further comprise one or more surfactant, one or more isotonizing agents, and/or one or more metal ion chelator, and/or one or more preservative.

The pharmaceutical formulation as described herein can be suitable for at least intravenous, intramuscular or subcutaneous administration.

In some embodiments, the pharmaceutical composition of the present disclosure as disclosed herein is for use in the treatment of cancer, wherein the cancer types are selected from the cancer types disclosed herein above.

In some embodiments, the present disclosure pertains to a method of treating a patient afflicted with cancer, wherein the method comprises administering to said patient a pharmacologically effective amount of the ADC of the present disclosure, or of the pharmaceutical composition as disclosed above. In some embodiments, the cancer is selected from the list of cancer types as disclosed herein above.

In some embodiments, the present disclosure pertains to the NAMPT inhibitors as disclosed herein for use as a medicament in the treatment in the treatment of cancer, preferably said NAMPT inhibitor of the disclosure is one of (c22a), (c23a), (c24a), (c30a), (c31a), (c32a), (c39a), (c40a), (c42a), (c47a), (c48a) or (c49a). Said NAMPT inhibitors are preferably formulated into a pharmaceutical composition and may e.g. be used in the treatment of cancer whereby the cancer is selected from the cancer types disclosed herein.

In some embodiments, the present disclosure pertains to a method of treating cancer in a patient, whereby the method comprises administering to said patient a NAMTP inhibitor of the disclosure as disclosed herein, preferably said NAMPT inhibitor of the disclosure is one of (c22a), (c23a), (c24a), (c30a), (c31a), (c32a), (c39a), (c40a), (c42a), (c47a), (c48a) or (c49a).

Further disclosed is an improved synthesis of tert-Butyl 4-hydroxy-4-(4-oxobutyl)piperidine-1-carboxylate (116) with RuCl₃*3H₂O:

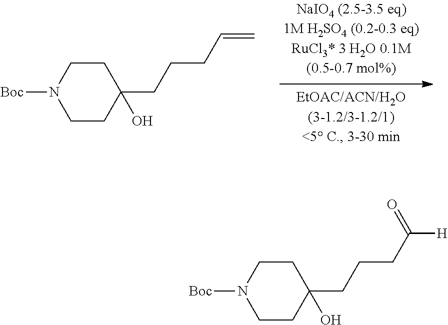 [SMILES: C=CCCCC1(O)CCN(C(=O)OC(C)(C)C)CC1 ; CC(C)(C)OC(=O)N1CCC(O)(CCCC=O)CC1]

This improved synthesis which employs NaIO₄, H₂SO₄ and RuCl₃*3H₂O obviates the use of the highly toxic catalyst OsO₄ and circumvents the necessity to work-up and purify the dihydroxylation intermediate. The above method results in overall yields of about 80% of the product tert-Butyl 4-hydroxy-4-(4-oxobutyl)piperidine-1-carboxylate in comparison to an overall yield of about 63% of the two-step synthesis using OsO₄. In addition to avoiding the use of the toxic OsO₄, the above method reduces reaction time from about 2 days to about 1 hour. Thus, the above synthesis provides a novel method to synthesize aldehydes from terminal polyfunctionalized olefins, e.g. olefins with two or more functional groups such as amino-, or hydroxy-groups.

### EXAMPLES

While the present disclosure has been illustrated and described in detail in the drawings and foregoing description, such illustrations and description are to be considered illustrative or exemplary and not restrictive; the present disclosure is not limited to the disclosed embodiments. Other variations to the disclosed embodiments can be understood and effected by those skilled in the art in practicing the claimed present disclosure, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

### NAMPT Inhibitor Payload Conjugation to Ab; ADC Production

Two methods for the conjugation of maleimide-containing payloads to mAb-reducing cysteines were used.

### a) High DAR Conjugation: Conjugation of Maleimide-Containing Payloads to Fully Reduce mAb

### 1) Reduction of Native Interchain Cysteines and Additionally Engineered Cysteines (Thiomab)

For this method, as an example, a 5 mg/mL solution of mAb in PBS was used. The pH and EDTA concentration were adjusted to 7.4 and 1 mM, respectively, using a 100×100 mM EDTA PBS solution at pH 8.0. The mAb solution was then reduced by adding 40 eq. of TCEP from a freshly made 50 mM TCEP solution (with 1 mM EDTA, pH 7.4), and incubated for 2 h at 37° C. The solution was then dialysed twice at 4° C. in a Slide-A-Lyzer Dialysis Cassette with a 20,000 MWCO (Thermo Scientific, different capacity cassettes were used depending on the sample volume: 3, 12, 30 mL), first for 4 h and then overnight. The dialysis was performed in 500 times the volume of the sample in 1×PBS, 1 mM EDTA, and pH 7.4.

2) Conjugation with Maleimide Containing Payload

The NAMPT inhibitor payload or amanitin payload (as a control) was conjugated to the working solution by adding 20 eq. of the respective payload (diluted to 10 μg/μL in DMSO) and incubated for 2 h at room temperature on a shaking platform. For hydrophobic payloads, fresh DMSO was added to the solution to a final concentration of 10% V/V, considering the DMSO from the payload solution. The remaining thiols were capped by incubating the solution with 12 eq. of a freshly prepared 100 mM solution of N-ethylmaleimide in DMSO for 1 hour on a shaking platform. Unreacted maleimide-containing molecules were quenched by incubating the solution with 40 eq. of a freshly prepared 100 mM solution of N-acetyl-L-cysteine in water for 15 min on a shaking platform at room temperature.

### b) Site-Specific Conjugation of Maleimide-Containing Payloads to Reduced Thiomab Cysteines

### 1) Reduction of Native Interchain Cysteines and Thiomab

For this method, as an example, a 5 mg/mL solution of mAb in PBS was used. The pH and EDTA concentrations were adjusted to 7.4 and 1 mM, respectively, using a 100×100 mM EDTA PBS solution at pH 8.0. The mAb solution was then reduced by adding 40 eq. of TCEP from a freshly made 50 mM TCEP solution (with 1 mM EDTA, pH 7.4) and incubated for 2 h at 37° C. The solution was then dialysed twice at 4° C. in a Slide-A-Lyzer Dialysis Cassette with a 20,000 MWCO (Thermo Scientific); different capacity cassettes were used depending on the sample volume: 3, 12, 30 mL), first for 4 h and then overnight. The dialysis was performed in 500 times the volume of the sample in 1×PBS, 1 mM EDTA, and pH 7.4.

### 2) Reoxidation of Interchain Cysteines and Disulfide Bonds Regeneration

To conjugate the payload only to the thiomab cysteines, an oxidation reaction of the interchain cysteines was performed by adding 20 eq. of dhAA (a freshly made 50 mM solution in DMSO) and incubating for 3 h at room temperature on a shaking platform.

3) Conjugation with Maleimide Containing Payload

In this study, the NAMPT inhibitor payload or amanitin payload (as a) was conjugated to the working solution by adding 8 eq. of the respective payload (diluted to 10 μg/μL in DMSO) and incubating for 2 h at room temperature on a shaking platform. For hydrophobic payloads, fresh DMSO was added to the solution to a final concentration of 10% V/V, considering the DMSO from the payload solution. Unreacted maleimide-containing molecules were quenched by incubating the solution with 25 eq. of a freshly prepared 100 mM solution of N-acetyl-L-cysteine in water for 15 min on a shaking platform at room temperature.

c) Purification and Aggregate Removal were Performed by Two Means:

### 1) PD-10 Column (GE Healthcare Life Sciences; Ref: 17-0851-01) Purification:

This method can only be used for up to 2 mL of ADC reaction mix and does not remove endotoxins.

To purify the ADC reaction mix, PD-10 columns were equilibrated with PBS, pH 7.4 by filling the column completely with the buffer and allowing it to enter the packed bed, then the process was repeated for four times and the flow-through was discarded. Afterwards, the sample was added, up to a maximum volume of 2.0 mL, to the column. Once the sample had entered the packed bed completely, 2.5 mL of PBS, pH7.4 was added and the flow-through was discarded. For sample collection 1.5 mL tubes were prepared and 3.5 mL of PBS was added to the column to elute the 500 μL sample fractions. Then, Bradford reagent (5 μL sample+5 μL Bradford reagent) was placed on parafilm to identify protein-containing fractions. To accurately determine the protein containing fractions, those fractions that were weakly stained with Bradford reagent were measured in NanoDrop at 280 nm and all the fractions with a protein content of >0.1 mg/mL were combined.

### 2) Purification by Gel Filtration in FPLC AKTA Start, Using HiLoad 16/600 (Cytiva Healthcare; Ref: 28-9893-35) or 26/600 Superdex 200 μg: Prepacked XK26-Column

To remove endotoxins and aggregates from the ADC/Ab solution, an isocratic method using PBS as mobile phase was used. The detection and automatic fractionation of proteins were assessed by UV absorption at 280 nm with a 16/600 mm size column for up to 50 mg of ADC/Ab, and a 26/600 mm size column for larger amounts. After purification, the ADC solution was dialysed at 4° C. overnight using 500 times the sample volume of 1×PBS, pH 7.4, and Slide-A-Lyzer Dialysis Cassettes with a 20,000 MWCO. Afterwards, the concentration was adjusted to 5 mg/mL using Amicon Ultra Centrifugal Filters with a 50,000 MWCO. Then, the concentration was determined by measuring absorbance at 280 nm (background 390 nm) with a Nanodrop One C and calculating the extinction factor using the amino acid sequence with the ProtParam tool on Expasy. Finally, the solution was sterile filtered with a MillexGV syringe filter with a 0.22 μm pore size.

### Quality Analysis:

1) Aggregates quantification was performed by HPLC-SEC in an Agilent 1260 Infinity HPLC system (Agilent) using a Tosoh UP SW 3000 4.6 mm×15 cm, 2 μm gel filtration column (Tosoh Bioscience, Ref: 0023449) and isocratic method during 8 min at 0.357 mL/min. UV detection of signals was performed at 280 nm. Running buffer: 0.05% NaN₃+0.1 mol/l Na₂SO₄ in 0.1 mol/1 NaPO₄, pH 6.7.
2) Endotoxin was determined by means of commercial kit, EndoZyme® II Recombinant Factor C (rFC) Assay (Hyglos, Ref: 890030), following manufacturer indications.
3) ADC integrity and qualitative determination of conjugation by SDS-Page.

The sample were loaded along with a protein ladder standard and two controls (a positive control of a pre-determined ADC of known DAR and a negative control of free Ab used for conjugation) onto Mini-PROTEAN TGX Stain-Free Gels with a 4-20% gradient and a 12-well comb (BioRad, Ref: 456-8095) to analyse. In all cases, the first lane was loaded with Precision Plus Protein Unstained Standard (BioRad, Ref 161-0363). The samples and controls were both measured in reduced and non-reduced conditions.

The samples were diluted to 1.5 mg/mL in order to be loaded in the gel. For reducing conditions, 4 μL of 4× Roti Load (Carl Roth GmbH, Ref: K929.1) was added to 12 μL of the 1.5 mg/mL sample, then, the mix was heated for 5 minutes at 95° C., and 13.33 μL were loaded onto the gel. For non-reducing conditions, 12 μL of 2× loading buffer without mercaptoethanol (composition of loading buffer without mercaptoethanol: 5526.6 μL H₂O, 657.9 μL 1M Tris-HCl pH 6.8, 1052.6 μL glycerol, 2105.3 μL 10% SDS, and 657.9 μL 0.05% bromophenol blue) were added to 12 μL of the 1.5 mg/mL sample, then, the mix was incubated for 60 min at 37° C., and 20 μL were loaded onto the gel. The electrophoresis was run in a Mini-PROTEAN Tetra system (BioRad, Ref: 165-8000) at 85 V for 10 min until the samples passes the stacking gel, then the voltage was raised to 140 V for 45 min. After electrophoresis, protein signals were monitored in an Azure C400 Imaging System (Azure biosystems) with UV-light. The signal was generated thanks to the trihalo compounds in Mini-PROTEAN TGX Stain-Free Gels that reacts with tryptophan residues in the proteins in a UV-induced 1-min reaction to produce fluorescence signals.

### 4) DAR Analysis by Mass Spectrometry

The samples were first deglycosylated by incubation at 50° C. for 5 min using Rapid PNGase F (NEB, Ref: P0711S). Then, a UPLC-MS tandem device consisting of an ACQUITY UPLC I Class Plus (Waters) and a BioACCord with RDa detector (Waters) as mass spectrometers was used. The samples were ionised by ESI and measured in positive mode.

### Synthesis of Compounds

### General Procedure A. Synthesis of Boc-Protected Amine Intermediates

### Procedure:

The amine intermediate (1 eq.) was dissolved in DCM (resulting concentration=0.1 M), triethylamine (1-2 eq.) was added, and the reaction mixture was set to 0° C. A solution of di-tert-butyl dicarbonate (1-2 eq.) dissolved in DCM at a concentration of 0.67 M was added dropwise. The reaction mixture was stirred at 20° C. for 3 h. The reaction progress was followed by TLC (Hex/EtOAc). The proportion was indicated in each case.

### Work Up:

After completion, the volatiles were evaporated under reduced pressure, and the crude product was adsorbed on diatomaceous earth and purified by flash chromatography using an isocratic method (Hex/EtOAc).

### General Procedure B. Coupling of Butyl-Amine with Phenyl-Cyano-Carbamimidate for N-Cyano-3-Pyridin-Guanidin Intermediates

### Procedure:

The amine intermediate (1 eq.) and phenyl (Z)—N′-cyano-N-(pyridin-3-yl)carbamimidate (1-1.5 eq.) were dissolved in 1,4-dioxane or DCM (resulting concentration=47 mM). The reaction mixture was set under argon, and Et₃N (1-3 eq.) was added. The progress of the reaction was followed by TLC (CHCl₃/MeOH).

### Work Up:

After completion, the volatiles are evaporated under reduced pressure, and the crude product was adsorbed on diatomaceous earth and purified by flash chromatography using an isocratic method (DCM/MeOH).

### General Procedure C. Boc Deprotection of Primary and Secondary Amines

For Boc-deprotection, two variations were applied depending on the stability of the intermediates against acid.

### Variation 1 (Acid-Resistant Intermediates):

### Procedure:

The Boc-protected intermediate was dissolved in pure TFA and the solution was rotated for 5 min at 400 mbar, followed by TFA evaporation lowering pressure to 80 mbar.

### Work Up:

After completion, the crude product was three times co-evaporated with MeOH. If required, the crude product was purified after co-evaporation.

### Variation 2 (Acid-Labile Intermediates):

### Procedure:

Boc-protected intermediates were dissolved in a solution of 10-20% TFA in DCM or DMF. The reaction mixture was stirred at room temperature (r.t.) for 2 h. The reaction progress was followed by TLC and/or HPLC, as specified for each molecule.

### Work Up:

After completion, the crude product was three times co-evaporated with MeOH. If required, the crude product was purified after co-evaporation.

### General Procedure D. Cbz-Protection of Amines

### Procedure:

The amine intermediate (1 eq.) was dissolved in THF (resulting concentration=0.33 M), benzyl chloroformate (1-2 eq.) was added, followed by base (NaOH or NaHCO₃, 2 eq.). The reaction mixture was stirred at r.t. for 3 h. The progress of the reaction was followed by TLC (CHCl₃/MeOH).

### Work Up:

After completion, the reaction mixture was acidified until a pH-value of 2 and extracted with EtOAc (3×). The organic layers were combined and washed with brine. The volatiles were evaporated under reduced pressure, and the crude product was adsorbed on diatomaceous earth and purified by flash chromatography applying a 15-min linear method gradient from 100% CHCl₃ to CHCl₃/MeOH.

### General Procedure E. Cbz-Deprotection by Hydrogenation

### Procedure:

In a three-neck flask equipped with an argon balloon, a hydrogen balloon and an adaptor for connection to a vacuum pump, the Cbz-protected intermediate was dissolved in EtOAc (resulting concentration=80 mM), followed by the addition of EtOH (same volume as EtOAc) and Pd/C (10-12 mol-%). Five cycles of vacuum/argon, followed by five cycles of vacuum/hydrogen were applied, and the reaction mixture vigorously stirred under hydrogen at r.t. for 3 h.

### Work Up:

After completion, the system was degassed from hydrogen by applying five vacuum/argon cycles. The reaction crude was filtered through celite, and volatiles were evaporated under reduced pressure. The reaction endpoint was determined by TLC or HPLC. Methods for detection and purification requirements (if needed) are specified for each molecule.

### General Procedure F. DCC Promoted Amide Bond Formation

### Procedure:

The carboxylic acid intermediate (1-2 eq.) and amine intermediate (1 eq.) were dissolved in DCM (abs.) or DMF (abs., resulting concentration: 80 mM). In the following order, DIPEA (1-2.2 eq.), HOBt (1-2 eq.) and DCC (1-2 eq.) were added. The reaction mixture was stirred at r.t. overnight. The reaction progress was followed by TLC and/or HPLC. The exact conditions and methods are described for each individual compound.

### Work Up:

After completion, the precipitated urea was removed from the reaction crude by filtration through celite, and the liquid layer was collected and washed with an aqueous 5% citric acid solution. The organic layer was collected and washed with water and brine. The organic layer was dried over MgSO₄, and volatiles were removed under reduced pressure. The crude product was adsorbed on diatomaceous earth and purified by flash chromatography using a linear solvent gradient specified for each molecule.

### General Procedure G. Synthesis of Mesylate

### Procedure:

An alcohol intermediate (1 eq.) was dissolved in DCM (abs., resulting concentration=0.14 M). The reaction mixture was then cooled down to 0° C., and methanesulfonyl chloride (1.5 eq.) was added, followed by triethylamine (2 eq.). The reaction mixture was stirred at r.t. for 1-2 h. The reaction progress was followed by TLC and/or HPLC. The individual conditions and methods applied are described for each intermediate.

### Work Up:

After completion, the crude product was dissolved in CHCl₃, neutralised with an aqueous 5% citric acid solution, dried over MgSO₄ and the volatiles evaporated under reduced pressure. Purification conditions were described for each molecule.

### General Procedure H. Gabriel Synthesis of Primary Amines (2-Steps)

### Step 1:

### Procedure:

The mesylate intermediate (1 eq.) was dissolved in DMF (abs., resulting concentration=96 mM), potassium phthalimide (1.2 eq.) was added, and the reaction mixture was stirred at 50° C. overnight. The reaction progress was followed by TLC and/or HPLC. The exact conditions and methods are described for each individual compound.

### Work Up:

After completion, reaction crude was dissolved in DCM (10× volume of DMF used in the reaction), and the organic layer was washed 2× with same volume of H₂O. The organic layer was collected and dried over MgSO₄. The volatiles were evaporated under reduced pressure. The exact purification conditions were described for each molecule.

### Step 2:

### Procedure:

The phthalimide from step 1 (1 eq.) was dissolved in EtOH (resulting concentration: 90 mM). After setting the system under an argon atmosphere, hydrazine monohydrate (4-6 eq.) was added. The reaction mixture was stirred at r.t. for 6 h or overnight. The reaction progress was followed by TLC and/or HPLC. The exact conditions and methods are described for each individual compound.

### Work Up:

After completion, the crude product was filtered to remove precipitates, and the volatiles were evaporated under reduced pressure. The crude product was dried in vacuo overnight to remove traces of hydrazine. The exact purification conditions were described for each molecule.

### General Procedure I. Coupling of NHS-Activated Carboxylic Acids Containing Maleimides to Amino Groups

### Procedure:

The amine intermediate (1-1.2 eq.) was dissolved in DMF (abs., resulting concentration: 10 mM), EMCS, BMPS or Mal-dPEG4-NHS (1-1.5 eq.) was added, followed by DIPEA (2-3.3 eq.). The reaction progress was followed by TLC and/or HPLC. The exact conditions and methods are described for each individual compound.

### Work Up:

After completion, volatiles were evaporated under reduced pressure. The exact purification conditions are described for each molecule.

### General Procedure J. Coupling of Aniline-Containing NAMPT Inhibitor to PNP-Activated Linkers

### Procedure:

The PNP-activated Linker or free carboxylic acid linker (1-2 eq.) was dissolved in DMF (resulting concentration: 25 mM), then PyAOP (1.5-2 eq.) or HATU (1.1 eq.) was added, followed by DIPEA (5-6 eq., 4 eq. in case of use HATU) until a pH-value of 10 was reached. The linker was activated for 30 min, then an aniline-containing intermediate (1 eq.) was added. The reaction mixture was stirred at r.t. overnight. The reaction progress was followed by TLC and/or HPLC. The exact conditions and methods are described for each individual compound.

### Work Up:

After completion, the solvent was evaporated under reduced pressure and purified by RP-HPLC, the method is specified for each molecule.

### General Procedure K. Biotinylation of Aniline-Containing Free NAMPT Inhibitors

### Procedure:

Biotin (1 eq.), DIPEA (2 eq.) and HATU (1.1 eq.) were dissolved together in DMF (abs., resulting concentration referred to biotin: 35.5 mM). After 15 min for biotin activation, a solution of the aniline-containing in DMF (abs.) was added to the reaction mixture, followed by DMF (abs.) until the limiting reagent final concentration of 14.2 mM was reached. The reaction progress was followed by TLC and/or HPLC. The exact conditions and methods are described for each individual compound.

### Work Up:

After completion, volatiles were evaporated under reduced pressure. The crude product was purified by HPLC using a method specified for each molecule.

### General Procedure L. Activation of PAB Alcohols with Bis(p-Nitrophenyl) Carbonate

### Procedure:

The linker containing PAB alcohol (1 eq.) was dissolved in DMF (abs., resulting concentration: 43.5 mM), bis(p-nitrophenyl) carbonate (2 eq.) was added, and after dissolution, DIPEA (1.5-3 eq.) was added. The reaction progress was followed by TLC and/or HPLC. The exact conditions and methods are described for each individual compound.

### Work Up:

After completion, the crude product was cooled on ice for 10 min. Then, a solution of 1:1 hexane/MTBE (20× the initial volume of DMF used in the reaction) was added; the reaction mixture was shaken until a solid was formed and centrifuged at 4,500 RPM for 5 min in a precooled (0° C.) centrifuge. The generated pellet was washed twice with pure MTBE. The pellet obtained was dried under vacuum and purified by HPLC, using the method specified for each molecule.

### General Procedure M. Synthesis of 1-Boc or 1-Cbz-4-hydroxy-4(pent-4-en-1-yl)piperidine

### Procedure (Part 1, Grignard-Formation):

Magnesium (solid, turnings; 2 eq.) and 12 (tip of a spatula) were added to a dried three-neck flask equipped with a reflux condenser. An inert atmosphere was set up by flushing the system with argon. Then, THF (abs., resulting concentration: 0.43 M) was added, and the temperature was raised to 70° C. (reflux) for 20 min. The mixture was cooled down to r.t. and afterwards, 5-bromo-1-pentene (1.5 eq.) was added dropwise over a period of 10 min. The reaction mixture was kept at r.t. until it got cloudy, and the formation of bubbles was observed. The reaction mixture was then set at 70° C. for 30 min and then cooled to r.t. and kept under argon atmosphere until it was used in the next step.

### Procedure (Part 2, Alcohol Synthesis):

A solution of LaCl₃*2LiCl in THF (1 eq.) was added to a dried two-neck flask, followed by piperidone (1 eq.). The reaction mixture was stirred at rt. for 1 h, and then, the system was cooled to 8° C. with a chiller, and the Grignard from step 1 was added dropwise. The reaction mixture was kept at 8° C. The reaction progress was followed by TLC and/or HPLC. The exact conditions and methods are described for each individual compound.

### Work Up:

After completion, the reaction was quenched with NH₄Cl (sat.), washed once with H₂O and once with brine, and dried over MgSO₄. Purification was performed by flash chromatography and the solvent(gradient) is specified for each intermediate.

### General Procedure N. OsO₄-Catalysed Dihydroxylation of Alkenes

### Procedure:

The alkene (1 eq.) was dissolved in acetone (resulting concentration=0.17 M). Then, NMO (1.2 eq.), water (16 eq.), and a 2.5 wt. % solution of OsO₄ in tert-butanol (0.47 mol-%) were added. The reaction progress was followed by TLC and/or HPLC. The exact conditions and methods are described for each individual compound.

### Work Up:

After completion, the reaction mixture was quenched with saturated aqueous Na₂SO₃ solution and extracted with CHCl₃ (3×). The organic layers were collected and washed once with water and once with brine. The organic layer was dried over MgSO₄, and volatiles were removed under reduced pressure. Purification was performed by flash chromatography and the solvent(gradient) is specified for each intermediate.

### General Procedure O. Oxidative Cleavage of 1,2-Diol Intermediates

### Procedure:

The diol (1 eq.) was dissolved in THF (resulting concentration: 0.16 M). Sodium periodate (2 eq.) was pre-dissolved in water (resulting concentration 0.08 M). The THF reaction mixture was cooled to 0° C., and both solutions were then mixed in a ratio of THF/H₂O: 2/1 v/v. The reaction progress was followed by TLC and/or HPLC. The exact conditions and methods are described for each individual compound.

### Work Up:

After completion, volatiles were evaporated, the crude product re-dissolved in EtOAc and washed once with water and once with brine. The organic layer was dried over MgSO₄ and volatiles were evaporated under reduced pressure. Purification was performed by RP-HPLC, using a method specified for each molecule.

### General Procedure P. Amine Synthesis by Reductive Amination of Aldehydes

### Procedure:

The aldehyde (1 eq.) was dissolved in MeOH (abs., resulting concentration: 0.23 M). Ammonium acetate (10 eq.) and sodium cyanoborohydride (2 eq.) were dissolved in MeOH (abs., 0.7 times volume used to dissolve the aldehyde). Then, the ammonium acetate-sodium cyanoborohydride-solution was added to the aldehyde solution. The pH-value was adjusted to 6 with acetic acid. The reaction progress was followed by TLC and/or HPLC. The exact conditions and methods are described for each individual compound.

### Work Up:

After completion, the reaction mixture was quenched with an aqueous 5% citric acid solution, followed by neutralisation with a sodium hydroxide-solution (5M) until pH=12. Then, the quenched crude product was extracted with EtOAc (3×), the organic layers were combined and washed once with brine. The organic layer was then dried over MgSO₄, and volatiles were evaporated under reduced pressure. The crude product was purified by RP-HPLC, using a method specified for each molecule.

### General Procedure Q. Fmoc and Acetyl Deprotection of Glucuronic Acid-Containing Linkers

### Procedure

N-Fmoc-O—Ac-β-D-glucuronide-linker-payload-intermediate (1 eq.) was dissolved in methanol (resulting concentration: 14 mM). The solution was cooled to 0° C., and then an aqueous solution of LiOH monohydrate (concentration of solution=0.23M) was added (same volume as MeOH). The reaction mixture was stirred at 0° C. for 15 min. The reaction progress was followed by HPLC. Methods are described for each individual intermediate.

### Work Up:

After completion, the reaction mixture was neutralised using acetic acid (1.1 eq.); volatiles were then evaporated under reduced pressure. Fmoc removal was afforded by dissolving the crude product in piperidine (200 eq.) and incubate it for 5 min at r.t. The volatiles were evaporated under reduced pressure, and the crude product was purified by HPLC, using the method specified for each individual molecule.

### General Procedure R. Urea Bond Formation Between Free Aliphatic Amine and 4-Nitrophenyl Isoindoline-2-Carbamate

### Procedure:

The amine intermediate (1 eq.) and carbamate (2 eq.) were dissolved in DMF (abs., resulting concentration: 23 mM). Then, N-methylimidazole (10 eq.) and DIPEA (1.5 eq.) were added. The reaction progress was followed by TLC and/or HPLC. The exact conditions and methods are described for each individual compound.

### Work Up:

After completion, the crude product was directly injected into the preparative RP-HPLC machine and purified using method 1.

### General Procedure S. Ru (VIII) Catalysed, One-Pot Synthesis of Aldehydes from Terminal Aliphatic Olefines

### Procedure:

Sodium metaperiodate (2.5 eq.) was dissolved in H₂O (Resulting concentration=0.6 M). Next, a 1M solution of sulfuric acid in water (0.33 eq.) was added and after dissolution of all solids, the temperature was lowered to 0° C. Next, a 0.1 M solution of ruthenium trichloride trihydrate in water (0.5 mol %) was added. The reaction mixture was stirred for 5 min until the colour changed to bright yellow. Then, an equimolar mixture of acetonitrile and ethyl acetate was added, and the reaction mixture was stirred for additional 5 min. A solution of the olefin (1 eq.) in ethyl acetate pre-cooled to 0° C. was added to the reaction mixture (resulting concentration of olefin 114=79 mM) resulting in a final solvent mixture composition of ethyl acetate, acetonitrile and water of 1.2/1.2/1. Then the resulting reaction mixture was stirred for 30 min at 0° C. The progress of the reaction was monitored by thin layer chromatography (TLC) (SiO₂, DCM/MeOH: 9/1 and DCM/MeOH: 30/1). Upon completion, the reaction mixture was poured into an aqueous mixture of a saturated sodium hydrogen carbonate solution and a saturated sodium thiosulfate solution at room temperature and stirred until the crude colour turned from yellow to violet. The organic layer was retained while the aqueous phase was extracted with ethyl acetate (3×). The organic layers were combined and washed once more with brine, dried over MgSO₄ and the volatiles were evaporated under reduced pressure. Purification was performed by flash chromatography on silica phase. The exact conditions and methods are described for each individual compound.

### Product Synthesis and Purification: Exact Conditions and Methods for Each Individual Compound.

### Phenyl (Z)—N′-cyano-N-(pyridin-3-yl)carbamimidate (1)

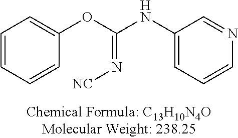 [SMILES: N#C/N=C(/Nc1cccnc1)Oc1ccccc1]

### Procedure:

3-aminopyridine (1 eq., 34.9 mmol, 3.28 g) and diphenyl N-cyanocarbonimidate (1.1 eq., 34.9 mmol, 8.31 g) were charged into a two-neck-flask. Then, DCM (105 mL) was added to the mixture, followed by triethylamine (1 eq., 34.9 mmol, 4.6 mL). The mixture was allowed to react overnight at room temperature and the reaction monitored by TLC (SiO₂, CHCl₃/MeOH: 21/1).

After completion, the volatiles were evaporated under reduced pressure, and the crude product was adsorbed on diatomaceous earth. Purification by flash chromatography applying an isocratic method (CHCl₃/MeOH: 21/1) afforded carbamimidate 1 (90% yield, 31.4 mmol, 7.48 g) as white to pale yellow crystals. MS (ESI, negative mode): found m/z=237.3 [M−H]⁻, calculated m/z=237.1 [M−H]⁻.

### N-Boc-4-(4-hydroxy-butyl)-piperidine (2)

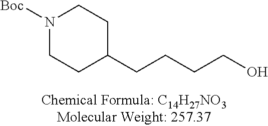 [SMILES: CC(C)(C)OC(=O)N1CCC(CCCCO)CC1]

Following General Procedure A, 4-(4-hydroxy-butyl)piperidine (1 eq., 3.18 mmol, 500 mg) and triethylamine (1.05 eq., 3.34 mmol, 466 μL) were dissolved in DCM (35 mL) and cooled to 0° C. Then, di-tert-butyl dicarbonate (1.05 eq., 3.34 mmol, 729 mg) was added, the reaction mixture temperature allowed to warm to r.t. and was stirred for 3 h. The reaction was monitored by TLC (SiO₂, hexane/EtOAc: 3/1). After completion, the volatiles were evaporated under reduced pressure, and the crude product was adsorbed on diatomaceous earth. Purification by flash chromatography applying an isocratic method (hexane/EtOAc: 4/1) afforded Boc-protected piperidine 2 (82% yield, 2.61 mmol, 671 mg) as clear oil. MS (ESI, negative mode): found m/z=256.4 [M−H]⁻, calculated m/z=256.2 [M−H]⁻.

### N-Boc-4-(piperidinyl)butyl-1-azide(3)

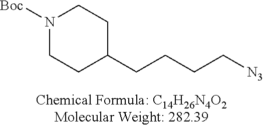 [SMILES: CC(C)(C)OC(=O)N1CCC(CCCCN=[N+]=[N-])CC1]

### Procedure:

Intermediate 2 (1 eq., 0.4 mmol, 103 mg) was dissolved in DMF (abs., 5 mL, resulting concentration=77.8 mM) at 0° C. under argon. Then, DPPA (3 eq., 1.17 mmol, 252 μL) and subsequently, DBU (3 eq., 1.17 mmol, 174 μL) were added. The reaction mixture was kept for 30 min at 0° C. Afterwards, it was heated to 100° C., and sodium azide (3 eq., 1.17 mmol, 76 mg) was added. The reaction mixture was stirred for 90 min and then cooled to r.t. The reaction was monitored by TLC (SiO₂, hexane/EtOAc: 4/1).

After completion, the crude product was dissolved in DCM and washed with water (2×) and brine (2×). Then, the volatiles were evaporated under reduced pressure and, and the crude product was adsorbed on diatomaceous earth. Purification by flash chromatography, applying an isocratic method (hexane/EtOAc: 4/1) afforded azide 3 (40% yield, 0.16 mmol, 45.2 mg) as yellow oil. MS (ESI, negative mode): found m/z=281.6 [M−H]⁻, calculated m/z=281.2 [M−H]⁻.

### N-Boc-4-(4-piperidinyl)butanoic acid (4)

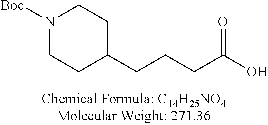 [SMILES: CC(C)(C)OC(=O)N1CCC(CCCC(=O)O)CC1]

### Procedure:

4-(4-piperidinyl)butanoic acid hydrochloride (1 eq., 24 mmol, 4.98 g) was dissolved in a mixture of THF/H₂O (17 mL THF and 32 mL H₂O, resulting concentration=0.75 M). Then, sodium bicarbonate (2.23 eq., 53.6 mmol, 4.5 g) was added slowly, and gas formation was observed. After the gas formation stopped, di-tert-butyl dicarbonate (1.12 eq., 27 mmol, 5.9 g) was added, and the reaction mixture was heated to 50° C. for 4 h. The reaction was monitored by TLC (SiO₂, MeOH/DCM/AcOH: 1/20/2%).

After completion, the volatiles were evaporated under reduced pressure until one third of the initial volume was left. EtOAc was added and the pH-value adjusted to 3 by adding an aqueous 20 wt-% potassium bisulfate solution dropwise. The layers were separated, and the aqueous layer was extracted with EtOAc (2×). The combined organic layers were mixed and washed once with water and once with brine. The volatiles were evaporated under reduced pressure. The crude product was adsorbed on diatomaceous earth. Purification by flash chromatography, applying an isocratic method (MeOH/DCM/AcOH 1/20/2%) afforded intermediate 4 (63% yield, 15.12 mmol, 4.1 g) as a turbid white oil. ¹H-NMR (500 MHz, CDCl₃): δ/ppm=4.06 (s, 4H), 2.65 (s, 2H), 2.33 (t, J=7.5 Hz, 2H), 1.69-1.59 (m, 2H), 1.44 (s, 14H), 1.06 (qd, J=12.7, 4.5 Hz, 2H). ¹³C-NMR (126 MHz, CDCl₃): δ/ppm=179.37, 154.93, 79.36, 35.77, 35.69, 34.10, 31.95, 28.42, 21.77, 20.82), MS (ESI, positive mode): found m/z=272.2 [M+H]⁺, calculated m/z=272.2 [M+H]⁺.

### N-Boc-4-(piperidinyl)butanamide (5)

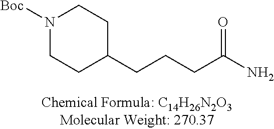 [SMILES: CC(C)(C)OC(=O)N1CCC(CCCC(N)=O)CC1]

### Procedure:

Under an argon atmosphere, intermediate 4 (1 eq., 0.37 mmol, 100 mg) was charged in a two-neck flask and dissolved in EtOAc (5 mL). Then, N—N-carbonyl diimidazole (1.25 eq., 0.46 mmol, 75 mg) was added, and the resulting mixture was stirred for 2.5 h at r.t. Afterwards, the reaction mixture was cooled down to 0° C., and a concentrated aqueous ammonia solution (1.5 mL) added. The mixture was then allowed to warm to r.t. and was stirred overnight. The reaction was monitored by TLC (SiO₂, CHCl₃/MeOH: 15/1).

After completion, the reaction mixture was washed with a saturated, aqueous solution of sodium bicarbonate (3×) and once with brine. Then, the combined organic layers were collected and dried over MgSO₄, and the volatiles were evaporated under reduced pressure. Without further purification, intermediate 5 (40% yield, 0.15 mmol, 40 mg) was obtained as oil. MS (ESI, negative mode): found m/z=269.6 [M−H]⁻, calculated m/z=269.2 [M−H]⁻.

### N-Boc-4-(piperidinyl)butan-1-amine (6)

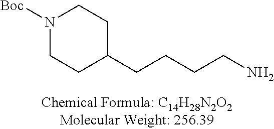 [SMILES: CC(C)(C)OC(=O)N1CCC(CCCCN)CC1]

### Procedure:

Intermediate 5 (1 eq., 7.85 mmol, 2.1 g) was dissolved in THF (70 mL, resulting concentration=0.11 M) and transferred to a three-neck flask equipped with a reflux condenser. Then, the solution was cooled to 0° C., and a 1M BH₃-THF-solution (7.8 eq., 60 mmol, 60 mL) was added. Next, the system was heated to 73° C. and refluxed overnight. The reaction was monitored by TLC (SiO₂, CHCl₃/MeOH/Et₃N: 15/1/1%).

After completion, the mixture was cooled to 0° C. and MeOH (250 mL) was added. Then, the volatiles were evaporated under reduced pressure. The residue was dissolved in methanol (100 mL) and refluxed at 70° C. for 1 h. Afterwards, the volatiles were evaporated under reduced pressure, and crude product was dissolved in EtOAc, and washed once with an aqueous, saturated sodium bicarbonate solution, followed by brine. Next, the combined organic layers were dried over MgSO₄, and the obtained crude product was adsorbed on diatomaceous earth. Purification by flash chromatography, applying an isocratic method (CHCl₃/MeOH/Et₃N: 15/1/1%) afforded intermediate 6 (10% yield, 0.785 mmol, 200 mg) as a pale-yellow oil. MS (ESI, positive mode): found m/z=257.5 [M+H]⁺, calculated m/z=257.2 [M+H]⁺.

### tert-butyl (E)-4-(4-(2-cyano-3-(pyridin-3-yl)guanidino)butyl)piperidine-1-carboxylate (7)

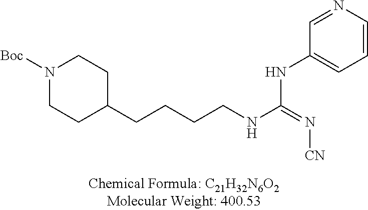 [SMILES: CC(C)(C)OC(=O)N1CCC(CCCCN/C(=N\C#N)Nc2cccnc2)CC1]

Following General Procedure B, intermediate 6 (1 eq., 0.4 mmol, 102 mg) and intermediate 1 (1 eq., 0.4 mmol, 95.3 mg) were dissolved in dioxane (4 mL), followed by the addition of triethylamine (1.2 eq., 0.4 mmol, 55.8 μL). Then, the reaction mixture was stirred for 36 h. The reaction was monitored by TLC (SiO2, hexane/EtOAc: 1/1 for phenol side product detection and CHCl₃/MeOH: 15/1 for product formation). The crude product was adsorbed on diatomaceous earth. Purification by flash chromatography, applying an isocratic method (CHCl₃/MeOH: 15/1) afforded intermediate 7 (55% yield, 0.22 mmol, 88.1 mg) as clear sticky oil. MS (ESI, positive mode): found m/z=401.4 [M+H]⁺, calculated m/z=401.3 [M+H]⁺.

### (E)-2-cyano-1-(4-(piperidin-4-yl)butyl)-3-(pyridin-3-yl)guanidine chloride (8)

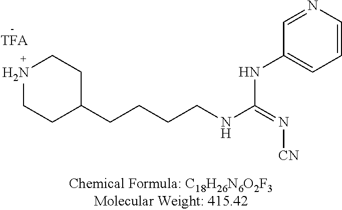 [SMILES: N#C/N=C(\NCCCCC1CCNCC1)Nc1cccnc1]

Following General Procedure C, intermediate 7 (1 eq., 0.27 mmol, 108.1 mg) was dissolved in pure TFA as a solvent (4 mL). The reaction mixture was stirred for 15 min at 400 mbar. The reaction was monitored by TLC (SiO₂, CHCl₃/MeOH/Et₃N: 10/1/1%). After completion, the crude product was worked up as reported in General Procedure C, and intermediate 8 (80% yield, 0.22 mmol, 89.7 mg) was afforded as sticky white crystals and used without further purification in the next reaction step.

### (E)-4-(4-(4-(2-carbamoyl-3-(pyridin-3-yl)guanidino)butyl)piperidine-1-carbonyl)phenylacetate (9)

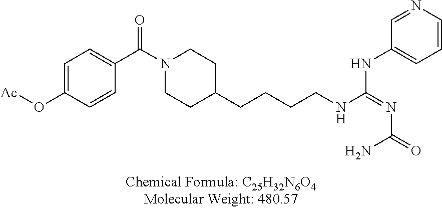 [SMILES: CC(=O)Oc1ccc(C(=O)N2CCC(CCCCN/C(=N\C(N)=O)Nc3cccnc3)CC2)cc1]

### Procedure:

Intermediate 8 (1 eq., 0.26 mmol, 108 mg) was dissolved under an argon atmosphere in DMF (abs., 7 mL, resulting concentration=37 mM). Then, p-acetoxybenzoic acid succinimide ester premade in HDP (1 eq., 0.35 mmol, 97 mg) was added, followed by DIPEA (4 eq., 1.4 mmol, 244 μL). The reaction was monitored by TLC (SiO₂, hexane/EtOAc/Et₃N: 1/1/1%).

After completion, the crude product was diluted with CHCl₃, washed once with H₂O and then rinsed once with brine. The combined organic layers were dried over MgSO₄. Then, the obtained crude oil was adsorbed on diatomaceous earth. Purification by flash chromatography, applying an isocratic method (hexane/EtOAc/Et₃N: 1/1/1%) afforded the guanyl urea intermediate 9 (30% yield, 0.078 mmol, 37 mg) as white crystals. MS (ESI, positive mode): found m/z=481.7 [M+H]⁺, calculated m/z=481.2 [M+H]⁺.

### Benzyl 4-(4-amino-4-oxobutyl)piperidine-1-carboxylate (10)

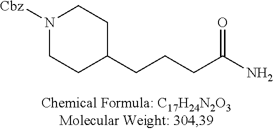 [SMILES: NC(=O)CCCC1CCN(C(=O)OCc2ccccc2)CC1]

Following General Procedure C, variation 1, intermediate 5 was Boc-deprotected (1 eq., 17.64 mmol, 4.8 g), and the obtained crude product was mixed with a solution of benzyl chloroformate (2 eq., 35.28 mmol, 6.0 g) in THF (53 mL) and an aqueous 2M NaOH (53 mL) solution. The reaction mixture was stirred for 3 h at r.t. The reaction was monitored by TLC (SiO₂, CHCl₃/MeOH: 15/1). After completion, the crude product was worked up as reported in General Procedure D and was adsorbed on diatomaceous earth. Purification by flash chromatography, applying an isocratic method (CHCl₃/MeOH: 15/1), afforded intermediate 10 (40% yield, 7.1 mmol, 2.1 g) as pale-yellow oil. MS (ESI, positive mode): found m/z=305.4 [M+H]⁺, calculated m/z=305.2 [M+H]⁺.

### N-Cbz-4-(piperidinyl)butan-1-amine (11)

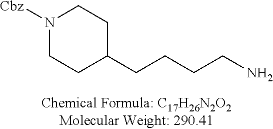 [SMILES: NCCCCC1CCN(C(=O)OCc2ccccc2)CC1]

### Procedure:

Intermediate 10 (1 eq., 3.29 mmol, 1 g) was dissolved under argon atmosphere in THF (20 mL). Then, a 1M Borane dimethyl sulphide complex solution in THF (1.5 eq., 4.93 mmol, 4.93 mL) was added to the solution, which is then heated up to reflux and left overnight. The reaction was followed by TLC (SiO₂, CHCl₃/MeOH/Et₃N: 5/1/1%).

After completion, the reaction mixture was cooled to 0° C., and an aqueous 2N NaOH solution (10 mL, 20 mmol) was slowly added. The reaction mixture was maintained under stirring conditions at r.t for 1 h more. The crude product was then diluted with water (10 mL) and extracted with EtOAc (3×). The combined organic layers were washed once with brine and dried over MgSO₄. The volatiles were evaporated under reduced pressure and the obtained crude product was adsorbed on diatomaceous earth. Purification by flash chromatography, applying an isocratic method (CHCl₃/MeOH/Et₃N: 5/1/1%), afforded intermediate 11 (15% yield, 0.494 mmol, 144 mg) as clear oil. MS (ESI, positive mode): found m/z=291.4 [M+H]⁺, calculated m/z=291.2 [M+H]⁺.

### benzyl(E)-4-(4-(2-cyano-3-(pyridin-3-yl)guanidino)butyl)piperidine-1-carboxylate (12)

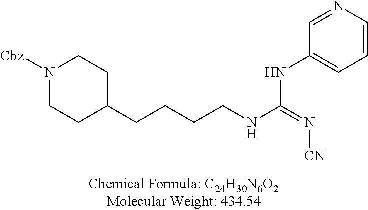 [SMILES: N#C/N=C(\NCCCCC1CCN(C(=O)OCc2ccccc2)CC1)Nc1cccnc1]

Following General Procedure B, intermediate 11 (1 eq., 1.18 mmol, 343 mg) and intermediate 1 (1.5 eq., 1.76 mmol, 419 mg) were charged in a two-neck flask and dissolved in DCM (30 mL). Then, triethylamine (3 eq., 3.58 mmol, 500 μL) was added, and the reaction mixture was kept stirring for 36 h at r.t. The reaction was monitored by TLC (hexane/EtOAc: 1/1 for phenol side product detection and CHCl₃/MeOH: 15/1 for product formation). The crude product was adsorbed on diatomaceous earth. Purification by flash chromatography, applying an isocratic method (CHCl₃/MeOH: 15/1), afforded intermediate 12 (40% yield, 0.472 mmol, 205 mg) as sticky oil. ¹H-NMR (500 MHz, CDCl₃): δ/ppm=8.58 (s, 1H), 8.50 (s, 1H), 7.94 (s, 1H), 7.71 (s, 1H), 7.42-7.27 (m, 6H), 5.11 (s, 2H), 3.31 (q, J=6.7 Hz, 2H), 2.74 (s, 2H), 1.57-1.50 (m, 2H), 1.37 (s, 1H), 1.30 (s, 2H), 1.30 (d, J=15.8 Hz, 1H), 1.24 (q, J=6.7 Hz, 2H), 1.08 (d, J=12.8 Hz, 3H). ¹³C-NMR (126 MHz, CDCl₃): δ/ppm=155.14, 136.80, 132.78, 128.32, 127.77, 127.65, 66.80, 44.05, 42.05, 35.80, 35.58, 29.29, 23.57). MS (ESI, positive mode): found m/z=435.1 [M+H]⁺, calculated m/z=435.2 [M+H]⁺.

### (E)-2-cyano-1-(4-(piperidin-4-yl)butyl)-3-(pyridin-3-yl)guanidine (13)

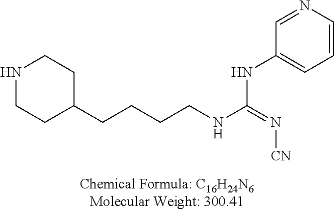 [SMILES: N#C/N=C(\NCCCCC1CCNCC1)Nc1cccnc1]

Following General Procedure E, intermediate 12 (1 eq., 0.4 mmol, 174 mg) was dissolved in a solvent mixture of EtOAc (5 mL, resulting concentration in EtOAc=80 mM) and EtOH (5 mL EtOH). Then, a Pd/C catalyst (10% in Pd, 12 mol-%, 50 mg) was added. Afterwards, the system was sealed, and hydrogenation was started according to General Procedure E. The reaction takes 3 h, and was monitored by TLC (SiO₂, CHCl₃/MeOH/Et₃N: 15/1/1%). After workup, the product is ready to use without further purification, affording intermediate 13 (85% yield, 0.34 mmol, 102 mg) as a white powder. MS (ESI, positive mode): found m/z=301.3 [M+H]⁺, calculated m/z=301.2 [M+H]⁺.

### N-(4-(4-hydroxy)-butyl)piperidinyl-4-methoxyphenyl-benzamide (14)

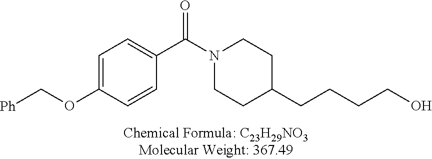 [SMILES: O=C(c1ccc(OCc2ccccc2)cc1)N1CCC(CCCCO)CC1]

Following General Procedure F, 4-(4-(hydroxy)-butyl)-piperidine (1 eq., 3.18 mmol, 500 mg) and 4-benzyloxybenzoic acid (1 eq., 3.18 mmol, 726 mg) were dissolved in DCM (abs., 40 mL, resulting concentration=80 mM). Then, DIPEA (1 eq., 3.18 mmol, 554 μL), HOBt (1 eq., 3.18 mmol, 430 mg) and DCC (1 eq., 3.18 mmol, 656 mg) were sequentially charged to the reaction mixture. The resulting mixture was stirred overnight at r.t., and the reaction monitored by TLC (SiO₂, hexane/EtOAc/MeOH: 10/10/1). After completion, the work-up was performed as described in General Procedure F, and the crude product was adsorbed on diatomaceous earth. Purification by flash chromatography, applying an isocratic method (Hex/EtOAc/MeOH: 10/10/1), afforded intermediate 14 (50% yield, 1.59 mmol, 584 mg) as oil. ¹H-NMR (500 MHz, CDCl₃): δ/ppm=7.46-7.30 (m, 7H), 6.97 (d, J=8.7 Hz, 2H), 5.09 (s, 2H), 3.64 (t, J=6.6 Hz, 2H), 1.88-0.99 (m, 15H). ¹³C-NMR (126 MHz, CDCl₃): δ/ppm=170.18, 159.68, 136.52, 132.04, 128.87, 128.72, 128.60, 128.06, 127.42, 114.49, 114.38, 69.99, 62.77, 36.14, 32.80, 22.79. MS (ESI, positive mode): found m/z=368.1 [M+H]⁺, calculated m/z=368.2 [M+H]⁺.

### N-(4-(4-methanesulfonyl)-butyl)piperidinyl-4-methoxyphenyl-benzamide (15)

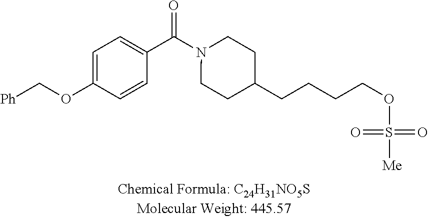 [SMILES: CS(=O)(=O)OCCCCC1CCN(C(=O)c2ccc(OCc3ccccc3)cc2)CC1]

Following General Procedure G, intermediate 14 (1 eq., 2.99 mmol, 1 g) was dissolved in DCM (abs., 22 mL). Then, the reaction mixture was cooled to 0° C. and methanesulfonyl chloride (1.5 eq., 4.49 mmol, 347 μL) was added. Triethylamin (2 eq., 6 mmol, 837 μL) was added and the reaction stirred for 2 h at r.t. The reaction was monitored by TLC (SiO₂, DCM/MeOH: 30/1). After completion, the work-up was performed as described in General Procedure G, and the crude product was adsorbed on diatomaceous earth. Purification by flash chromatography, applying an isocratic method (DCM/MeOH: 30/1), afforded intermediate 15 (73% yield, 2.18 mmol, 971 mg) as pale-yellow oil. ¹H-NMR (500 MHz, d6-DMSO): δ/ppm=7.48-7.39 (m, 2H), 7.39-7.29 (m, 5H), 7.08-7.00 (m, 2H), 5.14 (s, 2H), 4.19 (t, J=6.4 Hz, 2H), 3.15 (s, 3H), 2.88-2.84 (m, 2H), 1.72-1.60 (m, 4H), 1.55-1.45 (m, 1H), 1.42-1.32 (m, 2H), 1.30-1.21 (m, 2H), 1.05 (qd, J=12.4, 4.1 Hz, 2H). ¹³C-NMR (126 MHz, d6-DMSO): δ/ppm=169.05, 159.42, 137.14, 129.04, 128.98, 128.76, 128.20, 128.01, 114.75, 70.65, 69.66, 36.92, 35.56, 35.47, 28.97, 22.28. MS (ESI, positive mode): found m/z=446.1 [M+H]⁺, calculated m/z=446.2 [M+H]⁺.

### N-(4-(4-phthalimido)-butyl)piperidinyl-4-methoxyphenyl-benzamide (16)

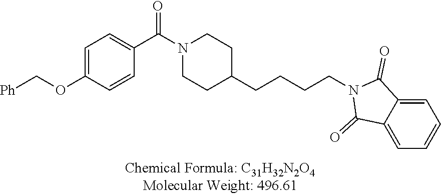 [SMILES: O=C(c1ccc(OCc2ccccc2)cc1)N1CCC(CCCCN2C(=O)c3ccccc3C2=O)CC1]

Following General Procedure H part 1, intermediate 15 (1 eq., 1.15 mmol, 512 mg) and potassium phthalimide (1.2 eq., 1.38 mmol, 255 mg) were dissolved in DMF (abs. 12 mL). The reaction mixture was stirred overnight at 50° C. The reaction was monitored by TLC (SiO₂, hexane/EtOAc/MeOH: 13/7/1). After completion, the work-up was performed as described in General Procedure H part 1, and the crude product was adsorbed on diatomaceous earth. Purification by flash chromatography, applying an isocratic method (Hex/EtOAc/MeOH: 13/7/1), afforded intermediate 16 (60% yield, 0.69 mmol, 343 mg) as pale-yellow oil. ¹H-NMR (500 MHz, d6-DMSO): δ/ppm=7.92-7.79 (m, 6H), 7.49-7.42 (m, 1H), 7.46-7.36 (m, 1H), 7.40-7.28 (m, 3H), 7.07-7.00 (m, 2H), 5.14 (s, 2H), 3.57 (t, J=7.1 Hz, 2H), 2.85-2.80 (m, 2H), 1.64-1.53 (m, 4H), 1.50-1.42 (m, 1H), 1.35-1.22 (m, 4H), 1.03 (qd, J=12.2, 4.1 Hz, 2H). ¹³C-NMR (126 MHz, d6-DMSO): δ/ppm=169.03, 168.24, 159.41, 137.14, 135.03, 134.66, 131.92, 129.03, 128.97, 128.75, 128.19, 128.01, 123.63, 123.28, 114.74, 69.66, 37.64, 35.60, 28.39, 23.62. MS (ESI, negative mode): found m/z=495.0 [M−H]⁻, calculated m/z=495.2 [M−H]⁻.

### N-(4-(4-amino)-butyl)piperidinyl-4-methoxyphenyl-benzamide (17)

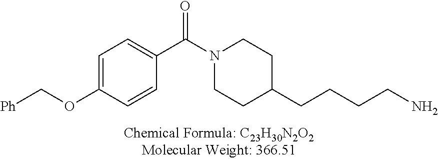 [SMILES: NCCCCC1CCN(C(=O)c2ccc(OCc3ccccc3)cc2)CC1]

Following General Procedure H part 2, intermediate 16 (1 eq., 0.9 mmol, 447 mg) was dissolved in EtOH (10 mL). Then, hydrazine monohydrate (4 eq., 3.6 mmol, 177 μL) was added, and the reaction stirred for 6 h at r.t. The reaction was monitored by TLC (SiO₂, CHCl₃/MeOH/Et₃N: 10/1/1%). After completion, the work-up was performed as described in General Procedure H part 2, and the crude product was adsorbed on diatomaceous earth. Purification by flash chromatography, applying an isocratic method (CHCl₃/MeOH/Et₃N: 10/1/1%), afforded intermediate 17 (40% yield, 0.36 mmol, 132 mg) as white crystals. ¹H-NMR (500 MHz, d6-DMSO): δ/ppm=7.45 (d, J=6.9 Hz, 2H), 7.40 (t, J=7.3 Hz, 2H), 7.36-7.30 (m, 3H), 7.04 (d, J=8.6 Hz, 2H), 5.14 (s, 2H), 4.17-4.13 (m, 2H), 2.57 (t, J=7.0 Hz, 2H), 1.69-1.59 (m, 2H), 1.53-1.42 (m, 1H), 1.38 (p, J=7.1 Hz, 2H), 1.29 (dq, J=17.6, 5.0, 3.5 Hz, 2H), 1.21 (q, J=6.4, 5.9 Hz, 2H), 1.05 (qd, J=12.5, 4.3 Hz, 2H). ¹³C-NMR (126 MHz, d6-DMSO): δ/ppm=168.63, 136.72, 128.65, 128.56, 128.33, 127.77, 127.58, 114.34, 69.26, 40.61, 35.54, 35.27, 31.88, 31.73, 23.51, 23.21. MS (ESI, negative mode): found m/z=365.0 [M−H]⁻, calculated m/z=365.2 [M−H]⁻.

### (E)-1-(4-(1-(benzyloxycarbonyl)-piperidin-4-yl)butyl)-2-cyano-3-(pyridin-3-yl)guanidine (18)

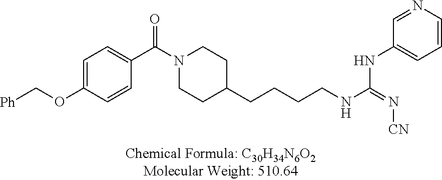 [SMILES: N#C/N=C(\NCCCCC1CCN(C(=O)c2ccc(OCc3ccccc3)cc2)CC1)Nc1cccnc1]

Following General Procedure B, intermediate 17 (1 eq., 0.41 mmol, 209 mg) and intermediate 1 (1 eq., 0.41 mmol, 98 mg) were dissolved in 1,4-dioxane (18 mL). Triethylamine (3 eq., 1.18 mmol, 164 μL) is then added, and the reaction end was determined after 48 h by TLC (SiO₂, CHCl₃/MeOH: 15/1). After completion, the work-up was performed as described in General Procedure B, and the crude product was adsorbed on diatomaceous earth. Purification by flash chromatography, applying an isocratic method (CHCl₃/MeOH: 15/1), afforded intermediate 18 (36% yield, 0.148 mmol, 75 mg) as sticky crystals. ¹H-NMR (500 MHz, d6-DMSO): δ/ppm=9.02 (s, 1H), 8.46 (d, J=2.5 Hz, 1H), 8.32 (dd, J=4.7, 1.5 Hz, 1H), 7.66 (ddd, J=8.2, 2.6, 1.5 Hz, 1H), 7.49-7.29 (m, 9H), 7.08-7.01 (m, 2H), 5.14 (s, 2H), 3.27 (s, 2H), 3.26-3.20 (m, 3H), 1.66 (d, J=11.3 Hz, 2H), 1.51 (p, J=7.3 Hz, 3H), 1.38-1.20 (m, 4H), 1.06 (qd, J=12.2, 4.2 Hz, 2H). ¹³C-NMR (126 MHz, d6-DMSO): δ/ppm 168.64, 159.01, 157.95, 145.27, 144.72, 136.72, 134.60, 130.70, 128.64, 128.56, 128.34, 127.78, 127.59, 123.47, 116.85, 114.34, 69.26, 66.26, 41.44, 35.28, 35.21, 31.86, 28.86, 23.07). MS (ESI, negative mode): found m/z=509.7 [M−H]⁻, calculated m/z=509.2 [M−H]⁻.

### (E)-2-cyano-1-(4-(1-(4-hydroxybenzoyl)piperidin-4-yl)butyl)-3-(pyridin-3-yl)guanidine (19)

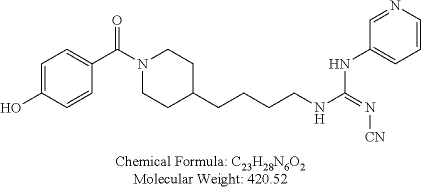 [SMILES: N#C/N=C(\NCCCCC1CCN(C(=O)c2ccc(O)cc2)CC1)Nc1cccnc1]

Following General Procedure E, intermediate 18 (1 eq., 0.23 mmol, 117 mg) was dissolved in a solvent mixture of EtOAc (9.6 mL) and EtOH (9.6 mL EtOH). Then, a Pd/C catalyst (10% in Pd, 12 mol-%, 40 mg) was added and the hydrogenation started according to General Procedure E. The reaction was performed for 3.5 h and monitored by TLC (SiO₂, CHCl₃/MeOH/Et₃N: 15/1/1%). After work-up, the product was ready to use without further purification, affording intermediate 19 (85% yield, 0.20 mmol, 82 mg) as a white powder. ¹H-NMR (500 MHz, d6-DMSO): δ/ppm=9.37 (s, 1H), 8.66 (d, J=2.5 Hz, 1H), 8.45 (dd, J=5.1, 1.4 Hz, 1H), 7.98 (d, J=8.4 Hz, 1H), 7.68-7.60 (m, 2H), 7.25-7.17 (m, 2H), 6.82-6.75 (m, 2H), 3.28 (q, J=6.9 Hz, 2H), 1.67 (d, J=12.9 Hz, 2H), 1.53 (p, J=7.2 Hz, 3H), 1.38-1.21 (m, 4H), 1.05 (qd, J=12.3, 4.2 Hz, 2H). ¹³C-NMR (126 MHz, d6-DMSO): δ/ppm=169.09, 158.42, 141.61, 140.73, 136.16, 133.61, 128.73, 126.67, 124.98, 114.76, 114.60, 66.28, 41.62, 35.33, 35.28, 31.92, 28.87, 23.09. MS (ESI, negative mode): found m/z=419.4 [M−H]⁻, calculated m/z=419.2 [M−H]⁻.

### tert-butyl((9S,12S)-7-(4-((4-(4-(4-((E)-2-cyano-3-(pyridin-3-yl)guanidino)butyl)piperidine-1-carbonyl)phenoxy)methyl)phenyl)-2,2,9,13-tetramethyl-8,11-dioxo-5-oxa-7,10-diaza-2-silatetradecan-12-yl)carbamate (20)

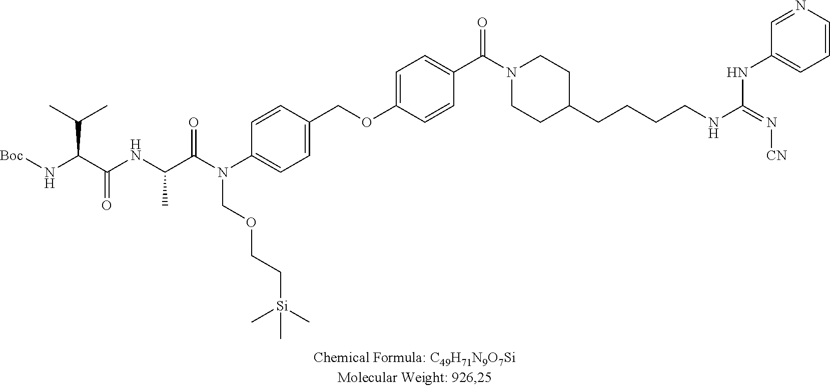 [SMILES: CC(C)[C@H](NC(=O)OC(C)(C)C)C(=O)N[C@@H](C)C(=O)N(COCC[Si](C)(C)C)c1ccc(COc2ccc(C(=O)N3CCC(CCCCN/C(=N\C#N)Nc4cccnc4)CC3)cc2)cc1]

### Procedure:

Intermediate 19 (1 eq., 0.12 mmol, 50.46 mg) was dissolved under an argon atmosphere in DMF (abs., 2.5 mL, resulting concentration=48 mM). Then, caesium carbonate (1 eq., 0.12 mmol, 39.1 mg) was charged to the mixture, and after 30 min, a solution of tert-butyl ((9R,12R)-7-(4-(bromomethyl)phenyl)-2,2,9,13-tetramethyl-8,11-dioxo-5-oxa-7,10-diaza-2-silatetradecan-12-yl)carbamate (1.7 eq., 0.2 mmol, 118 mg) in DMF (abs., 0.6 mL) was added. The reaction was stirred overnight and monitored by HPLC (method 4). After completion, the reaction was quenched with acetic acid until a pH-value of 5 was reached. The volatiles were evaporated under reduced pressure. The crude product was purified by preparative HPLC (method 4, t_{R}=15.5 min), affording intermediate 20 (40% yield, 0.048 mmol, 44.46 mg) as sticky oil. MS (ESI, positive mode): found m/z=927.3 [M+H]⁺, calculated m/z=927.5 [M+H]⁺.

### (S)-2-amino-N—((S)-1-((4-((4-(4-(4-((E)-2-cyano-3-(pyridin-3-yl)guanidino)butyl)piperidine-1-carbonyl)phenoxy)methyl)phenyl)amino)-1-oxopropan-2-yl)-3-methylbutanamide (21)

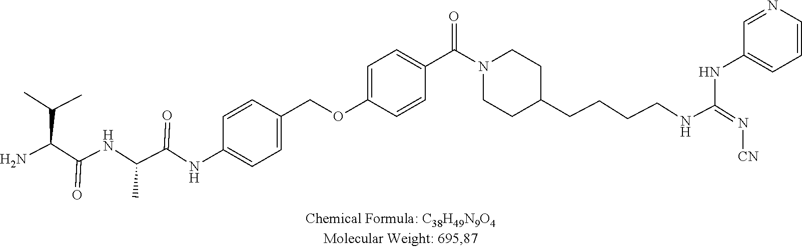 [SMILES: CC(C)[C@H](N)C(=O)N[C@@H](C)C(=O)Nc1ccc(COc2ccc(C(=O)N3CCC(CCCCN/C(=N\C#N)Nc4cccnc4)CC3)cc2)cc1]

Following General Procedure C, intermediate 20 (1 eq., 0.026 mmol, 24 mg) was dissolved in pure TFA (3 mL). The reaction mixture was stirred under 400 mbar for 5 min. Then, the crude product was worked up as described in General Procedure C. Without further purification, the product was used directly in a second deprotection step for SEM-group removal. For this purpose, the crude product was dissolved in ACN (5 mL) and then, an aqueous ammonia solution was added dropwise until a pH-value of 10 was reached. The mixture was stirred for 5 min and the reaction monitored by HPLC (method 4), elution time. After completion, crude is neutralised and used without purification in the production of intermediate 27. t_{R} (HPLC)=9.88 min.

### 4-(piperidin-4-yl)-1-butanol (22)

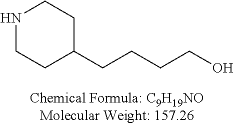 [SMILES: OCCCCC1CCNCC1]

4-(piperidin-4-yl)-butanoic acid (1 eq., 52.55 mmol, 9 g) was dissolved under an argon atmosphere in THF (abs., 150 mL, resulting concentration=0.35 M) and cooled down to 0° C. Lithium aluminium hydride (4 eq., 174 mmol, 6.6 g) was added slowly to the reaction mix, controlling the temperature with a thermometer. Afterwards, the reaction mixture was allowed to warm to r.t. and was kept stirring for 15 min, followed by heating to reflux overnight. The reaction was monitored by TLC (SiO₂, EtOAc/Et₃N/MeOH: 18/2/80). After completion, the reaction mixture was cooled to 0° C. and an aqueous 30 wt-% KOH-solution (30 mL) was added dropwise under argon via dropping funnel. Subsequently, the suspension was warmed to r.t. and stirred for 1 h. Then, the white suspension was filtered through celite, and the remaining solids were washed with fresh THF. The combined liquids were concentrated under reduced pressure. The crude product was dissolved in DCM, filtered, and the volatiles evaporated under reduced pressure, affording intermediate 22 (95% yield, 49.9 mmol, 7.8 g) as a white melting solid ready to use without further purification. MS (ESI, positive mode): found m/z=158.4 [M+H]⁺, calculated m/z=158.2 [M+H]⁺.

### 4-(N-Boc)-N-(4-(4-hydroxy)-butyl)-piperidinyl)benzamide (23)

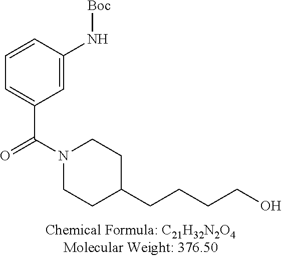 [SMILES: CC(C)(C)OC(=O)Nc1cccc(C(=O)N2CCC(CCCCO)CC2)c1]

Following General Procedure F, intermediate 22 (1 eq., 18.36 mmol, 3.2 g) and 3-(N-Boc)-amino)benzoic acid (1 eq., 18.36 mmol, 4.4 g) were dissolved in DCM (abs., 230 mL). Then, DIPEA (1 eq., 18.36 mmol, 3.2 mL), HOBt (1 eq., 18.36 mmol, 2.5 g) and DCC (1 eq., 18.36 mmol, 3.8 g) were added sequentially and stirred overnight at r.t. The reaction was monitored by TLC (SiO₂, hexane/EtOAc/MeOH: 10/10/1). After completion, the work-up was performed as described in General Procedure F, and the crude product was adsorbed on diatomaceous earth. Purification by flash chromatography, applying an isocratic method (Hex/EtOAc/MeOH: 10/10/1), afforded intermediate 23 (57% yield, 10.5 mmol, 3.9 g) as oil. MS (ESI, positive mode): found m/z=377.2 [M+H]⁺, calculated m/z=377.2 [M+H]⁺.

### 4-(N-Boc)-N-(4-(4-methanesulfonyl)-butyl)-piperidinyl)benzamide (24)

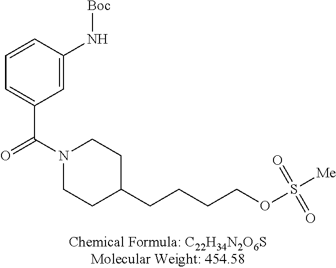 [SMILES: CC(C)(C)OC(=O)Nc1cccc(C(=O)N2CCC(CCCCOS(C)(=O)=O)CC2)c1]

Following General Procedure G, intermediate 23 (1 eq., 16.08 mmol, 6 g) is dissolved in DCM (abs., 118 mL). The solution was cooled to 0° C. and then, methane sulfonyl chloride (1.5 eq., 24.12 mmol, 2.8 g) was added, followed by triethylamine (2 eq., 32.16 mmol, 4.5 mL). The reaction was stirred for 2 h at r.t. and monitored by TLC (SiO₂, DCM/MeOH: 30/1). After completion, the work-up was performed as described in General Procedure G, and the crude product was adsorbed on diatomaceous earth. Purification by flash chromatography, applying an isocratic method (DCM/MeOH: 30/1), afforded intermediate 24 (75% yield, 12.06 mmol, 5.5 g) as an oil. MS (ESI, positive mode): found m/z=455.2 [M+H]⁺, calculated m/z=455.2 [M+H]⁺.

### 3-((N-Boc)-amino)-N-(4-(4-phthalimidyl)-butyl)-piperidinyl)benzamide (25)

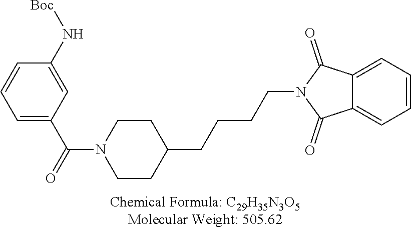 [SMILES: CC(C)(C)OC(=O)Nc1cccc(C(=O)N2CCC(CCCCN3C(=O)c4ccccc4C3=O)CC2)c1]

Following General Procedure H part 1, intermediate 24 (1 eq., 14.3 mmol, 6.5 g) and potassium phthalimide (1.2 eq., 17.16 mmol, 3.2 g) were dissolved in DMF (abs. 150 mL) and stirred at 50° C. overnight. The reaction was monitored by TLC (SiO₂, hexane/EtOAc/MeOH: 13/7/1). After completion, the work-up was performed as described in General Procedure H part 1, and the crude product was adsorbed on diatomaceous earth. Purification by flash chromatography, applying an isocratic method (hexane/EtOAc/MeOH: 13/7/1), afforded intermediate 25 (96% yield, 13.7 mmol, 6.9 g) as a melting solid. MS (ESI, positive mode): found m/z=506.4 [M+H]⁺, calculated m/z=506.2 [M+H]⁺.

### 3-(N-Boc)-N-(4-(4-amino)-butyl)-piperidinyl)benzamide (26)

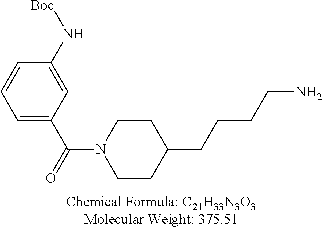 [SMILES: CC(C)(C)OC(=O)Nc1cccc(C(=O)N2CCC(CCCCN)CC2)c1]

Following General Procedure H part 2, intermediate 25 (1 eq., 13.84 mmol, 7 g) was dissolved in EtOH (180 mL). Then, hydrazine monohydrate (4 eq., 55.38 mmol, 2.7 mL) was added and the reaction mixture stirred for 6 h at r.t. The reaction was monitored by TLC (SiO₂, CHCl₃/MeOH/Et₃N: 10/1/1%). After completion, the work-up was performed as described in General Procedure H part 2, and the crude product was adsorbed on diatomaceous earth. Purification by flash chromatography, applying an isocratic method (CHCl₃/MeOH/Et₃N: 10/1/1%), afforded intermediate 26 (60% yield, 8.3 mmol, 3.1 g) as colourless oil. MS (ESI, positive mode): found m/z=375.9 [M+H]⁺, calculated m/z=376.2 [M+H]⁺.

### (S)—N—((S)-1-((4-((4-(4-(4-((E)-2-cyano-3-(pyridin-3-yl)guanidino)butyl)piperidine-1-carbonyl)phenoxy)methyl)phenyl)amino)-1-oxopropan-2-yl)-2-(3-(2,5-dioxo-2,5-dihydro-1H-pyrrol-1-yl)propanamido)-3-methylbutanamide (27)

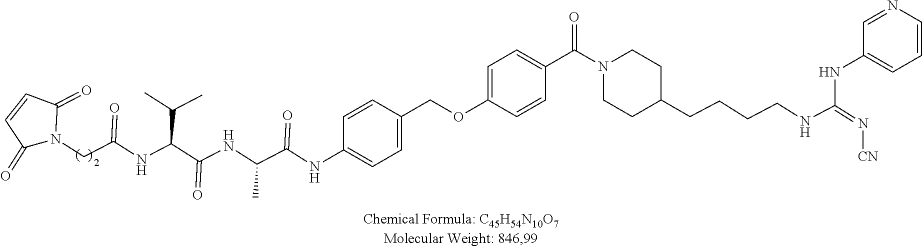 [SMILES: CC(C)[C@H](NC(=O)CN1C(=O)C=CC1=O)C(=O)N[C@@H](C)C(=O)Nc1ccc(COc2ccc(C(=O)N3CCC(CCCCN/C(=N\C#N)Nc4cccnc4)CC3)cc2)cc1]

Following General Procedure I, intermediate 21 (1 eq., 0.01 mmol, 7 mg) and BMPS (1 eq., 0.01 mmol, 2.66 mg) were dissolved in DMF (1 mL). Then, DIPEA (2 eq., 0.02 mmol, 3.48 μL) was and the reaction was stirred for 4 h at r.t. The reaction was monitored by HPLC (method 2). After completion, the work-up was performed as described in General Procedure I and purified by preparative HPLC (method 2, t_{R}=12.49 min), affording intermediate 27 (40% yield, 0.004 mmol, 3.4 mg) as white powder after lyophilisation. MS (ESI, positive mode): found m/z=848.1 [M+H]⁺, calculated m/z=846.4 [M+H]⁺.

### tert-butyl(E)-(3-(4-(4-(2-cyano-3-(pyridin-3-yl)guanidino)butyl)piperidine-1-carbonyl)phenyl)carbamate (28)

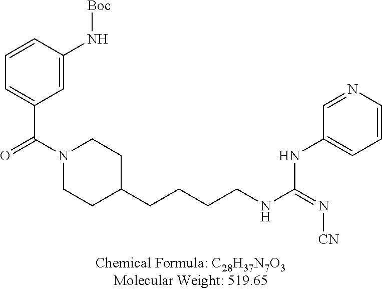 [SMILES: CC(C)(C)OC(=O)Nc1cccc(C(=O)N2CCC(CCCCN/C(=N\C#N)Nc3cccnc3)CC2)c1]

Following General Procedure B, intermediate 26 (1 eq., 6.7 mmol, 2.5 g) and intermediate 1 (1 eq., 6.7 mmol, 1.6 g) were dissolved in 1,4-dioxane (143 mL). Then, triethylamine (1.2 eq., 8.05 mmol, 1.1 mL) was added and the mixture stirred for 36 h at r.t. The reaction was monitored by TLC (SiO₂, CHCl₃/MeOH: 15/1). After completion, the work-up was performed as described in General Procedure B, and the crude product adsorbed on diatomaceous earth. Purification by flash chromatography, applying an isocratic method (CHCl₃/MeOH: 15/1), afforded intermediate 28 (40% yield, 2.68 mmol, 1.4 g) as sticky oil. ¹H-NMR (500 MHz, d6-DMSO): δ/ppm=9.00 (s, 1H), 8.46 (dd, J=2.6, 0.8 Hz, 1H), 8.33 (dd, J=4.7, 1.5 Hz, 1H), 7.69-7.62 (m, 1H), 7.47 (dq, J=4.7, 1.9, 1.5 Hz, 1H), 7.39-7.32 (m, 2H), 7.32-7.25 (m, 1H), 6.92 (dt, J=7.6, 1.3 Hz, 1H), 3.28 (s, 2H), 3.23 (q, J=6.7 Hz, 2H), 1.56-1.42 (m, 3H), 1.48 (s, 9H), 1.37-1.24 (m, 2H), 1.10-1.01 (m, 2H). ¹³C-NMR (126 MHz, d6-DMSO): δ/ppm=168.96, 158.29, 152.98, 145.63, 145.07, 139.83, 137.19, 134.90, 131.07, 128.92, 123.81, 120.28, 118.95, 116.38, 79.51, 79.40, 41.80, 35.64, 35.50, 29.19, 28.34, 23.42, 18.77. MS (ESI, positive mode): found m/z=521.0 [M+H]⁺, calculated m/z=520.3 [M+H]⁺.

### 3-(N-Cbz)-amino-benzoic Acid (30)

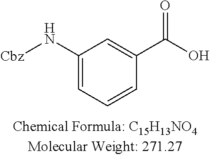 [SMILES: O=C(Nc1cccc(C(=O)O)c1)OCc1ccccc1]

Following General Procedure D, 3-aminobenzoic acid (1 eq., 4.79 mmol, 657 mg) and benzyl chloroformate (1 eq., 4.79 mmol, 683 μL) were dissolved in THF (150 mL), followed by the addition of sodium bicarbonate (2 eq., 9.6 mmol, 806 mg). The reaction system was stirred for 7 h and was monitored by TLC (SiO₂, CHCl₃/MeOH/AcOH: 15/1/1%). After completion, work-up was performed as described in General Procedure D, and the crude product was ready to use without further purification. Intermediate 30 (90% yield, 4.3 mmol, 1.2 g) was afforded as pale-orange crystals. ¹H-NMR (500 MHz, d6-Acetone): δ/ppm=9.09 (s, 1H), 8.43 (t, J=2.0 Hz, 1H), 7.98 (ddd, J=8.2, 2.3, 1.1 Hz, 1H), 7.83 (ddd, J=7.8, 1.7, 1.0 Hz, 1H), 7.56 (t, J=8.0 Hz, 1H), 7.45-7.27 (m, 5H), 5.19 (s, 2H). ¹³C-NMR (126 MHz, d6-Acetone): δ/ppm=162.92, 154.30, 141.06, 137.51, 130.56, 130.27, 129.26, 128.99, 128.91, 125.27, 124.98, 120.51, 67.19. MS (ESI, negative mode): found m/z=270.1 [M−H]⁻, calculated m/z=270.1 [M−H]⁻.

### 3-(N-Cbz)-amino-N′-(4-(4-hydroxy)-butyl)-piperidinyl-benzamide (31)

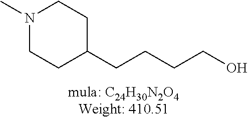

Following General Procedure F, intermediate 22 (1 eq., 3.49 mmol, 549 mg) and intermediate 30 (1 eq., 3.49 mmol, 947 mg) were dissolved in DCM (abs., 43.67 mL). Then, DIPEA (1 eq., 3.49 mmol, 608 μL), HOBt (1 eq., 3.49 mmol, 475 mg) and DCC (1 eq., 3.49 mmol, 722 mg) were added sequentially and the reaction mixture stirred overnight. The reaction was monitored by TLC (SiO₂, hexane/EtOAc/MeOH: 10/10/1). After completion, the work-up was performed as described in General Procedure F, and the crude product was adsorbed on diatomaceous earth. Purification by flash chromatography, applying an isocratic method (Hex/EtOAc/MeOH: 10/10/1), afforded intermediate 31 (40% yield, 1.4 mmol, 573 mg) as oil. MS (ESI, positive mode): found m/z=411.2 [M+H]⁺, calculated m/z=411.2 [M+H]⁺.

### 3-(N-Cbz)-N-(4-(4-methanesulfonyl)-butyl)-piperidinyl)benzamide (32)

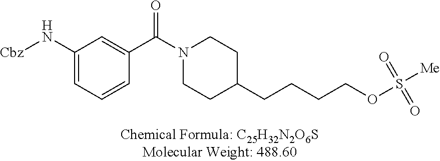 [SMILES: CS(=O)(=O)OCCCCC1CCN(C(=O)c2cccc(NC(=O)OCc3ccccc3)c2)CC1]

Following General Procedure G, intermediate 31 (1 eq., 1.04 mmol, 427 mg) was dissolved in DCM (abs. 10 mL). The solution was cooled to 0° C. and methanesulfonyl chloride (1.5 eq., 1.56 mmol, 182 mg) was added. Afterwards, triethylamine (2 eq., 2.08 mmol, 291 μL) was charged into the reaction flask and the reaction stirred for 2 h at r.t. The reaction was monitored by TLC, DCM/MeOH: 30/1. After completion, the work-up was performed as described in General Procedure G, and the crude product was adsorbed on diatomaceous earth. Purification by flash chromatography, applying an isocratic method (DCM/MeOH: 30/1), afforded intermediate 32 (75% yield, 0.78 mmol, 381 mg) as an oil. MS (ESI, positive mode): found m/z=489.2 [M+H]⁺, calculated m/z=489.2 [M+H]⁺.

### 3-(N-Cbz)-N-(4-(4-phthalimidyl)-butyl)-piperidinyl)benzamide (33)

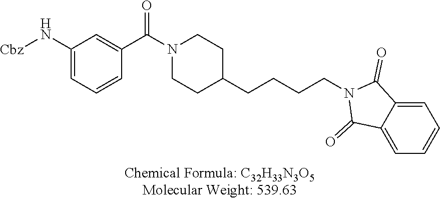 [SMILES: O=C(Nc1cccc(C(=O)N2CCC(CCCCN3C(=O)c4ccccc4C3=O)CC2)c1)OCc1ccccc1]

Following General Procedure H part 1, intermediate 32 (1 eq., 13.67 mmol, 6.7 g) and potassium phthalimide (1.2 eq., 16.4 mmol, 3.1 g) were dissolved in dry-DMF (145 mL) and the reaction mixture was stirred overnight at 50° C. The reaction was monitored by TLC (hexane/EtOAc/MeOH: 13/7/1). After completion, work-up was performed as described in General Procedure H part 1, and the crude product was adsorbed on diatomaceous earth. Purification by flash chromatography, applying an isocratic method (Hex/EtOAc/MeOH: 13/7/1), afforded intermediate 33 (80% yield, 10.9 mmol, 5.9 g) as oil. MS (ESI, positive mode): found m/z=540.4 [M+H]⁺, calculated m/z=540.2 [M+H]⁺.

### 3-(N-Cbz)-N-(4-(4-phthalimidyl)-butyl)-piperidinyl)benzamide (34)

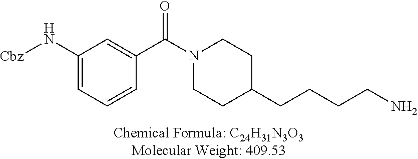 [SMILES: NCCCCC1CCN(C(=O)c2cccc(NC(=O)OCc3ccccc3)c2)CC1]

Following General Procedure H part 2, intermediate 33 (1 eq., 10.56 mmol, 5.7 g) was dissolved in EtOH (120 mL). Then, hydrazine monohydrate (4 eq., 42.2 mmol, 2.1 mL) was added and the reaction mixture was stirred overnight at r.t. The reaction was monitored by TLC (SiO₂, CHCl₃/MeOH/Et₃N: 10/1/1%). After completion, work-up was performed as described in General Procedure H part 2, and the crude product was adsorbed on diatomaceous earth. Purification by flash chromatography, applying an isocratic method (CHCl₃/MeOH/Et₃N: 10/1/1%), afforded intermediate 34 (50% yield, 5.28 mmol, 2.2 g) as white crystals. MS (ESI, positive mode): found m/z=410.5 [M+H]⁺, calculated m/z=410.2 [M+H]⁺.

### benzyl(E)-(3-(4-(4-(2-cyano-3-(pyridin-3-yl)guanidino)butyl)piperidine-1-carbonyl)phenyl)carbamate (35)

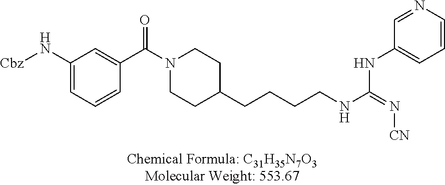 [SMILES: N#C/N=C(\NCCCCC1CCN(C(=O)c2cccc(NC(=O)OCc3ccccc3)c2)CC1)Nc1cccnc1]

Following General Procedure B, intermediate 34 (1 eq., 8.55 mmol, 3.5 g) and intermediate 1 (1 eq., 8.55 mmol, 2.0 g) were dissolved in 1,4-dioxane (185 mL). Then, triethylamine (2.4 eq., 20.5 mmol, 2.86 mL) was added and the reaction mixture stirred overnight at r.t. The reaction was monitored by TLC (SiO₂, CHCl₃/MeOH: 15/1). After completion, the work-up was performed as described in General Procedure B, and the crude product was adsorbed on diatomaceous earth. Purification by flash chromatography, applying an isocratic method (CHCl₃/MeOH: 15/1), afforded intermediate 35 (41% yield, 3.5 mmol, 1.9 g) as sticky oil. MS (ESI, positive mode): found m/z=554.4 [M+H]⁺, calculated m/z=554.2 [M+H]⁺.

### (E)-1-(4-(1-(3-aminobenzoyl)piperidin-4-yl)butyl)-2-cyano-3-(pyridin-3-yl)guanidine (36)

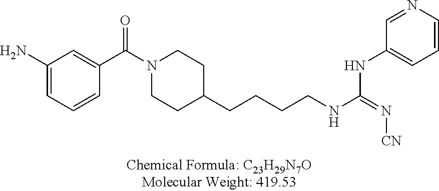 [SMILES: N#C/N=C(\NCCCCC1CCN(C(=O)c2cccc(N)c2)CC1)Nc1cccnc1]

Following General Procedure E, intermediate 35 (1 eq., 5.06 mmol, 2.8 g) was dissolved in a solvent mixture of EtOAc (50 mL) and EtOH (50 mL). Then, Pd/C (10% in Pd, 12 mol-%) was added, and the hydrogenation was started according to General Procedure E. The reaction mixture was stirred overnight and the progress monitored by TLC (SiO₂, CHCl₃/MeOH/Et₃N: 15/1/1%) and HPLC (method 1). After workup, the product is ready to use without further purification, affording intermediate 36 (85% yield, 4.3 mmol, 1.8 g) as white powder. t_{R}/min (HPLC, method 1)=8.96 min. ¹H-NMR and ¹³C-NMR as described in the literature,¹²² MS (ESI, negative mode): found m/z=418.4 [M−H]⁻, calculated m/z=418.2 [M−H]⁻.

### 4-((S)-2-((S)-2-(6-(2,5-dioxo-2,5-dihydro-1H-pyrrol-1-yl)hexanamido)-3-methylbutanamido)propanamido)benzyl (3-(4-(4-((E)-2-cyano-3-(pyridin-3-yl)guanidino)butyl) piperidine-1-carbonyl)phenyl)carbamate (37)

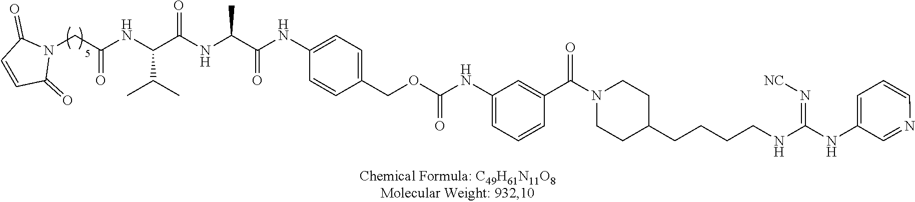

Following General Procedure J, mc-Val-Ala-PAB-PNP (CAS-No. 1639939-40-4, 1.5 eq., 0.023 mmol, 15 mg) was dissolved in DMF (612 μL), followed by the addition of PyAOP (1.5 eq., 0.023 mmol, 12 mg) and DIPEA (6 eq., 0.092 mmol, 16 μL). The mixture was stirred for 30 min at r.t. and then, intermediate 36 (1 eq., 0.015 mmol, 6.3 mg) was added. The reaction mixture was stirred overnight at R.T and the reaction progress monitored by RP-HPLC (method 1). After completion, the mixture was directly purified by preparative HPLC (method 1, t_{R}=15.09 min), affording intermediate 37 (30% yield, 0.004 mmol, 4.2 mg) as a white solid. MS (ESI, negative mode): found m/z=931.4 [M−H]⁻, calculated m/z=931.4 [M−H]⁻.

### Benzyl 4-(4-((tert-butoxycarbonyl)amino)butanoyl)piperazine-1-carboxylate (38)

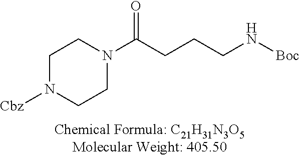 [SMILES: CC(C)(C)OC(=O)NCCCC(=O)N1CCN(C(=O)OCc2ccccc2)CC1]

Following General Procedure F, Boc-GABA-OH (1 eq., 24.6 mmol, 5 g) and 1-Cbz-piperazine (1 eq., 24.6 mmol, 5.4 g) were dissolved in DCM (abs., 385 mL). Then, DIPEA (1 eq., 24.6 mmol, 4.3 mL), HOBt (1 eq., 24.6 mmol, 3.3 g) and DCC (1 eq., 24.6 mmol, 5.1 g) were added sequentially and the reaction mixture stirred overnight at r.t. The reaction was monitored by TLC (SiO₂, hexane/EtOAc/MeOH: 13/7/1). After completion, the work-up was performed as described in General Procedure F, and the crude product was adsorbed on diatomaceous earth. Purification by flash chromatography, applying an isocratic method (Hex/EtOAc/MeOH: 13/7/1), afforded intermediate 38 (60% yield, 14.8 mmol, 6 g) as oil. MS (ESI, positive mode): found m/z=406.2 [M+H]⁺, calculated m/z=406.2 [M+H]⁺.

### Benzyl 4-(4-aminobutanoyl)piperazine-1-carboxylate (39)

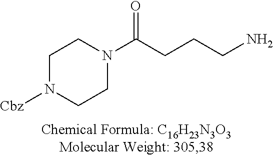 [SMILES: NCCCC(=O)N1CCN(C(=O)OCc2ccccc2)CC1]

Following General Procedure C variation 2, intermediate 38 (1 eq., 2 mmol, 811 mg) was dissolved in solution of TFA/DCM (50 mL, 1:10). The reaction mixture was stirred for 2 h at r.t. The reaction was monitored by TLC (SiO₂, CHCl₃/MeOH/Et₃N: 10/1/1%). After completion, the work-up was performed as described in General Procedure C affording intermediate 39 (90% yield, 1.8 mmol, 550 mg) as colourless melting crystals. Without further purification, the product was directly used in the next reaction step.

### Benzyl (E)-4-(4-(2-cyano-3-(pyridin-3-yl)guanidino)butanoyl)piperazine-1-carboxylate (40)

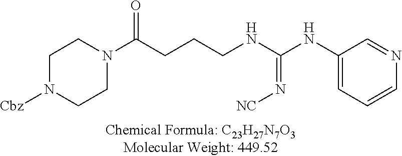 [SMILES: N#C/N=C(\NCCCC(=O)N1CCN(C(=O)OCc2ccccc2)CC1)Nc1cccnc1]

Following General Procedure B, intermediate 39 (1 eq., 6.55 mmol, 2 g) and intermediate 1 (1 eq., 6.55 mmol, 1.5 g) were dissolved in 1,4-dioxane (142 mL). Triethylamine (3 eq., 19.65 mmol, 2.7 mL) was added and the reaction mixture stirred overnight at r.t. The reaction was monitored by TLC (SiO₂, CHCl₃/MeOH: 15/1). After completion, the work-up was performed as described in General Procedure B, and the crude product was adsorbed on diatomaceous earth. Purification by flash chromatography, applying an isocratic method (CHCl₃/MeOH: 15/1), afforded intermediate 40 (50% yield, 3.3 mmol, 1.5 g) as pale-yellow oil. MS (ESI, positive mode): found m/z=450.3 [M+H]⁺, calculated m/z=450.2 [M+H]⁺.

### (E)-2-cyano-1-(4-oxo-4-(piperazin-1-yl)butyl)-3-(pyridin-3-yl)guanidine (41)

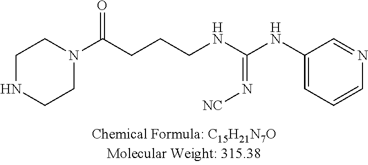 [SMILES: N#C/N=C(\NCCCC(=O)N1CCNCC1)Nc1cccnc1]

Following General Procedure E, intermediate 40 (1 eq., 1.69 mmol, 760 mg) was dissolved in a solvent mixture of EtOAc (50 mL) and EtOH (50 mL EtOH). Then, Pd/C (10% in Pd, 12 mol-%) was added (12% mol, 200 mg), and the hydrogenation was started according to General Procedure E. The reaction was stirred overnight and monitored by TLC (SiO₂, CHCl₃/MeOH/Et₃N: 10/1/1%). The work-up was performed as described in General Procedure E, and the product was ready to use without further purification. Intermediate 41 (90% yield, 1.52 mmol, 480 mg) was obtained as a white powder. MS (ESI, positive mode): found m/z=316.0 [M+H]⁺, calculated m/z=316.2 [M+H]⁺.

### Benzyl(E)-(3-(4-(4-(2-cyano-3-(pyridin-3-yl)guanidino)butanoyl)piperazine-1carbonyl)phenyl)carbamate (42)

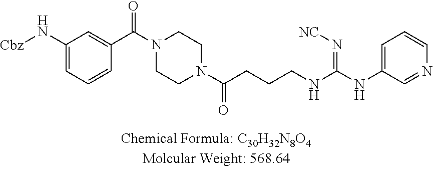 [SMILES: N#C/N=C(\NCCCC(=O)N1CCN(C(=O)c2cccc(NC(=O)OCc3ccccc3)c2)CC1)Nc1cccnc1]

Following General Procedure F, intermediate 41 (1 eq., 1.78 mmol, 561 mg) and intermediate 30 (1 eq., 1.78 mmol, 483 mg) were dissolved in DCM (abs., 30 mL). Then, DIPEA (1 eq., 1.78 mmol, 311 μL), HOBt (1 eq., 1.78 mmol, 239 mg) and DCC (1 eq., 1.78 mmol, 369 mg) were sequentially added and stirred overnight at room temperature. The reaction progress was monitored TLC (SiO₂, CHCl₃/MeOH: 15/1). After completion, the work-up was performed as described in General Procedure F, and the crude product was adsorbed on diatomaceous earth. Purification by flash chromatography, applying an isocratic method (CHCl₃/MeOH: 15/1), afforded intermediate 42 (40% yield, 0.71 mmol, 405 mg) as oil. MS (ESI, positive mode): found m/z=569.7 [M+H]⁺, calculated m/z=569.2 [M+H]⁺.

### (E)-1-(4-(4-(3-aminobenzoyl)piperazin-1-yl)-4-oxobutyl)-2-cyano-3-(pyridin-3-yl)guanidine (43)

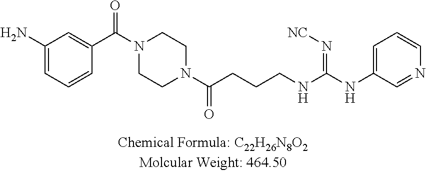 [SMILES: N#C/N=C(\NCCCC(=O)N1CCN(C(=O)c2cccc(N)c2)CC1)Nc1cccnc1]

Following General Procedure E, intermediate 42 (1 eq., 0.98 mmol, 557 mg) was dissolved in a solvent mixture of EtOAc (50 mL) and EtOH (50 mL). Then, Pd/C (10% in Pd, 12% mol, 350 mg) was added, and the hydrogenation was started according to General Procedure E. The reaction was stirred overnight and monitored by TLC (SiO₂, CHCl₃/MeOH/Et₃N: 10/1/1%). The work-up was performed as described in General Procedure E, and the product is ready to use without further purification. Intermediate 43 (80% yield, 0.78 mmol, 341 mg) was obtained as a white powder. ¹H-NMR (500 MHz, d6-DMSO): δ/ppm=9.17 (s, 1H), 8.51 (d, J=2.7 Hz, 1H), 8.32 (dd, J=4.7, 1.5 Hz, 1H), 7.72 (d, J=8.2 Hz, 1H), 7.36 (ddd, J=8.3, 4.7, 0.8 Hz, 1H), 7.10-7.02 (m, 1H), 6.62 (ddd, J=8.1, 2.4, 1.0 Hz, 1H), 6.59-6.53 (m, 1H), 6.49 (ddd, J=7.4, 1.6, 1.0 Hz, 1H), 5.21 (s, 2H), 3.48 (s, 2H), 3.30-3.20 (m, 6H), 2.39 (s, 2H), 1.77 (p, J=7.0 Hz, 2H). ¹³C-NMR (126 MHz, d6-DMSO): δ/ppm=170.65, 169.80, 158.07, 148.65, 145.23, 144.59, 136.24, 134.63, 130.55, 128.73, 123.45, 116.83, 114.78, 113.78, 111.99, 41.12, 29.23, 24.21. MS (ESI, positive mode): found m/z=435.2 [M+H]⁺, calculated m/z=435.2 [M+H]⁺.

### 4-((S)-2-((S)-2-(6-(2,5-dioxo-2,5-dihydro-1H-pyrrol-1-yl)hexanamido)-3-methylbutanamido) propanamido)benzyl (3-(4-(4-((E)-2-cyano-3-(pyridin-3-yl)guanidino)butanoyl)piperazine-1-carbonyl)phenyl)carbamate (44)

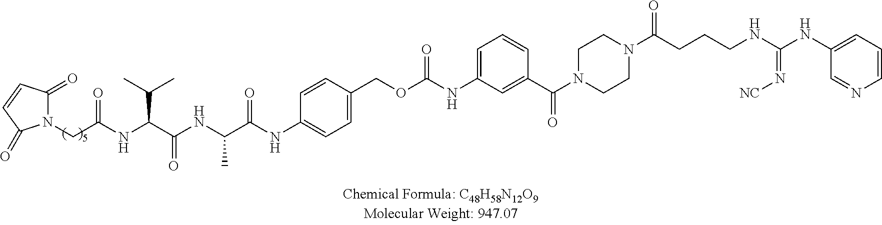

Following General Procedure J, mc-Val-Ala-PAB-PNP (CAS-No. 1639939-40-4, 1.5 eq., 0.005 mmol, 3.26 mg) was dissolved in DMF (200 μL) and then, PyAOP (1.5 eq., 0.005 mmol, 2.6 mg) and DIPEA (6 eq., 0.021 mmol, 3.7 μL) were added. The mixture was stirred for 30 min at r.t. and next, intermediate 43 (1 eq., 0.0035 mmol, 1.5 mg) was added, and the reaction mix was stirred overnight at r.t. After completion, reaction mixture was directly purified by preparative HPLC (method 1, t_{R}=13.77 min), affording intermediate 44 (30% yield, 0.001 mmol, 1 mg) as a white solid. MS (ESI, positive mode): found m/z=948.1 [M+H]⁺, calculated m/z=947.4 [M+H]⁺. MS (ESI, negative mode): found m/z=946.8 [M−H]⁻, calculated m/z=946.4 [M−H]⁻.

### (E)-N-(3-(4-(4-(2-cyano-3-(pyridin-3-yl)guanidino)butanoyl)piperazine-1-carbonyl)phenyl)-3-(2,5-dioxo-2,5-dihydro-1H-pyrrol-1-yl)propenamide (45)

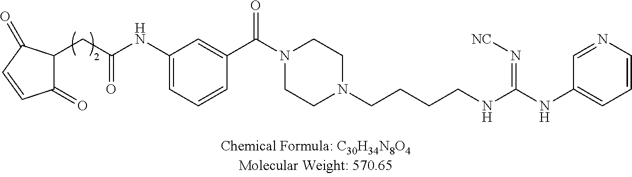

Following General Procedure, I, intermediate 36 (1 eq., 0.024 mmol, 10 mg) and BMPS (1 eq., 0.05 mmol, 13.3 mg) were dissolved in DMF (abs., 2.4 mL). Then, DIPEA (2 eq., 0.05 mmol, 8.7 μL) was added and the reaction stirred for 4 h at r.t. The reaction was monitored by HPLC (method 2). After completion, the work-up was performed as described in General Procedure I and purified by preparative HPLC using (method 2, t_{R}=9.00 min). Intermediate 45 (38% yield, 0.009 mmol, 5.2 mg) was afforded as white powder after lyophilisation. MS (ESI, positive mode): found m/z=571.4 [M+H]⁺, calculated m/z=571.2 [M+H]⁺.

### (E)-N-(3-(4-(4-(2-cyano-3-(pyridin-3-yl)guanidino)butanoyl)piperazine-1-carbonyl)phenyl) biotinamide (46)

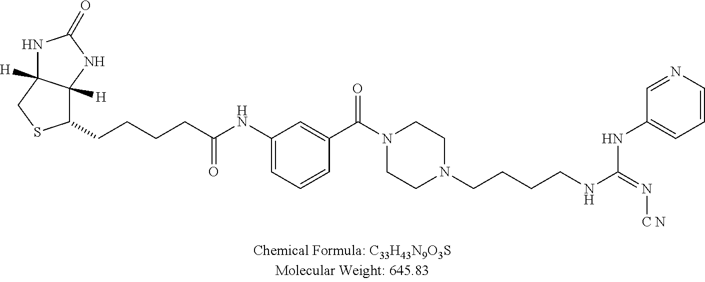

Following General Procedure K, biotin (1 eq., 0.071 mmol, 17.3 mg) and HATU (1.1 eq., 0.08 mmol, 30.4 mg) were dissolved in DMF (abs., 5 mL) and stirred at r.t. After 20 min, DIPEA (2 eq., 0.14 mmol, 24 μL) and intermediate 36 (1 eq., 0.071 mmol, 30 mg) were added, and the reaction mixture was stirred over the weekend. The reaction was monitored by TLC (SiO₂, CHCl₃/MeOH 10/1) and HPLC (method 2). After completion, the work-up was performed as described in General Procedure K and purification by preparative HPLC (method 2, t_{R}=12.05 min) afforded intermediate 46 (45% yield, 0.03 mmol, 22 mg) as a white solid after lyophilisation. ¹H-NMR (500 MHz, d6-DMSO): δ/ppm=9.95 (s, 1H), 9.25 (s, 1H), 8.61 (d, J=2.5 Hz, 1H), 8.42 (dd, J=5.0, 1.4 Hz, 1H), 7.91 (d, J=8.4 Hz, 1H), 7.66 (t, J=1.9 Hz, 1H), 7.62-7.53 (m, 2H), 7.33 (t, J=7.9 Hz, 1H), 6.99 (dt, J=7.6, 1.3 Hz, 1H), 6.31 (s, 2H), 3.26 (q, J=6.7 Hz, 2H), 2.83 (dd, J=12.4, 5.1 Hz, 1H), 2.63-2.50 (m, 2H), 2.32 (t, J=7.8 Hz, 2H), 1.78-1.19 (m, 17H), 1.05 (d, J=13.7 Hz, 2H). ¹³C-NMR (126 MHz, d6-DMSO): δ/ppm=171.29, 168.55, 162.59, 157.64, 142.38, 141.57, 139.20, 136.82, 135.80, 133.09, 128.65, 124.67, 120.92, 119.57, 117.05, 116.55, 66.26, 60.97, 59.15, 55.25, 41.57, 39.72, 36.15, 35.30, 35.18, 28.84, 28.09, 27.99, 24.93, 23.09. MS (ESI, positive mode): found m/z=646.3 [M+H]⁺, calculated m/z=646.3 [M+H]⁺.

### Mal-β-Ala-PEG(4)-Val-Ala-PAB-OH (CAS-No. 2417003-93-9) (47)

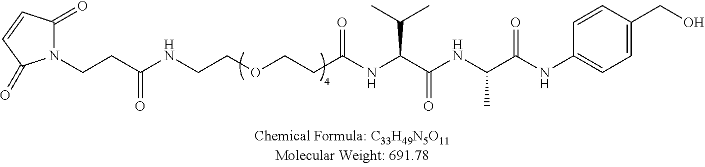

Following General Procedure I, H-Val-Ala-PAB-OH (CAS-No. 1343476-44-7, 1 eq., 0.034 mmol, 10 mg) and Mal-dPEG(4)-NHS (CAS-No. 756525-99-2, 1 eq., 0.034 mmol, 17 mg) were dissolved in DMF (abs., 2 mL). Then, DIPEA (2 eq., 0.07 mmol, 12.2 μL) was added and the reaction stirred for 2.5 h at r.t. The reaction was monitored by HPLC (method 5). After completion, the work-up was performed as described in General Procedure I and purification by preparative HPLC (method 5, t_{R}=11.56 min) afforded intermediate 47 (33% yield, 0.01 mmol, 7.8 mg) as oil. MS (ESI, negative mode): found m/z=690.1 [M−H]⁻, calculated m/z=690.3 [M−H]⁻.

### Mal-β-Ala-PEG(4)-Val-Ala-PAB-PNP (CAS-No. 2417003-94-0) (48)

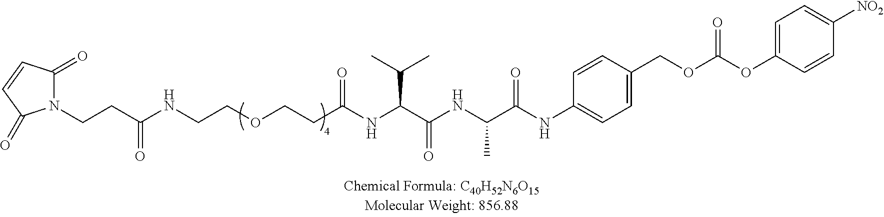

Following General Procedure L, Intermediate 47 (1 eq., 0.087 mmol, 60 mg) and bis-(p-nitro-phenyl)carbonate (2 eq., 0.17 mmol, 52 mg) were dissolved in DMF (abs., 2 mL). Then, DIPEA (1.5 eq., 0.13 mmol, 23 μL) was added and the reaction mixture stirred for 3 h at r.t. The reaction was monitored by HPLC (method 6). After completion, the work-up was performed following General Procedure L, followed by purification by preparative HPLC (method 6, t_{R}=12.99 min) affording intermediate 48 (30% yield, 0.03 mmol, 22 mg) as a white solid after lyophilisation. MS (ESI, positive mode): found m/z=857.8 [M+H]⁺, calculated m/z=857.4 [M+H]⁺.

### Mal-β-Ala-PEG(8)-Val-Ala-PAB-OH (CAS-No. 756525-93-6) (49)

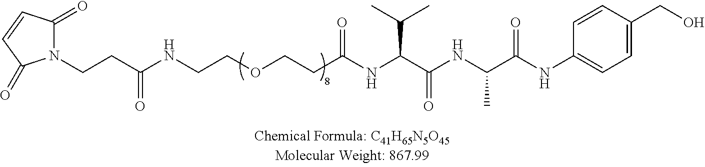

Following General Procedure I, H-Val-Ala-PAB-OH (CAS-No. 1343476-44-7, 1.2 eq., 0.087 mmol, 26 mg) and Mal-dPEG(8)-NHS (CAS-No. 756525-93-6, 1 eq., 0.072 mmol, 50 mg) were dissolved in DMF (abs., 4.28 mL). Then, DIPEA (2 eq., 0.15 mmol, 26 μL) was added and the reaction stirred for 3 h at r.t. The reaction was monitored by HPLC (method 5). After completion, the work-up was performed as described in General Procedure I and purification by preparative HPLC (method 5, t_{R}=15.24 min) afforded intermediate 49 (22% yield, 0.02 mmol, 14 mg) as oil. MS (ESI, positive mode): found m/z=868.7 [M+H]⁺, calculated m/z=868.4 [M+H]⁺.

### 4-((2S,5S)-25-(2,5-dioxo-2,5-dihydro-1H-pyrrol-1-yl)-5-isopropyl-2-methyl-4,7,23-trioxo-10,13,16,19-tetraoxa-3,6,22-triazapentacosanamido)benzyl (3-(4-(4-((E)-2-cyano-3-(pyridin-3-yl)guanidino)butyl)piperidine-1-carbonyl)phenyl)carbamate (50)

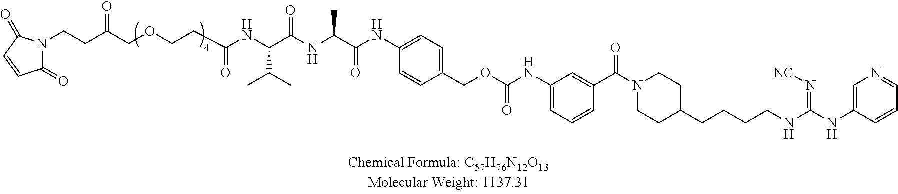

Following General Procedure J, Intermediate 48 (1 eq., 0.043 mmol, 37 mg) and HATU (1.1 eq., 0.05 mmol, 19 mg) were dissolved in DMF (abs., 3.03 mL). Then, DIPEA (4 eq., 0.17 mmol, 29 μL) was added and stirred at r.t. After 20 min, intermediate 36 (1 eq., 0.043 mmol, 18 mg) was added and the reaction stirred over the weekend. The reaction was monitored by TLC (SiO₂, CHCl₃/MeOH 10/1) and HPLC (method 7). After completion, the work-up was performed as described in General Procedure J and purified by preparative HPLC (method 7 t_{R}=12.82 min), affording intermediate 50 (20% yield, 0.009 mmol, 10 mg) as a white solid after lyophilisation. ¹H-NMR (500 MHz, d6-DMSO): δ/ppm=9.90 (s, 1H), 9.82 (s, 1H), 9.06 (s, 1H), 8.35 (s, 2H), 8.11 (d, J=7.0 Hz, 1H), 7.83 (d, J=8.6 Hz, 1H), 7.65-7.58 (m, 1H), 7.53-7.45 (m, 1H), 7.42-7.28 (m, 4H), 7.01-6.92 (m, 2H), 5.09 (s, 2H), 4.40 (p, J=6.9 Hz, 2H), 4.20 (dd, J=8.6, 6.7 Hz, 1H), 3.68-3.54 (m, 4H), 3.56-3.43 (m, 11H), 3.36 (t, J=5.9 Hz, 2H), 3.27-3.06 (m, 4H), 1.97 (h, J=6.7 Hz, 1H), 1.68-1.62 (m, 1H), 1.39-1.12 (m, 15H), 0.86 (dd, J=17.7, 6.8 Hz, 6H). ¹³C-NMR (126 MHz, d6-DMSO): δ/ppm=170.70, 170.55, 170.22, 169.33, 153.25, 139.01, 138.75, 136.90, 134.39, 131.02, 128.77, 128.73, 120.36, 118.89, 69.64, 69.57, 69.41, 69.34, 68.84, 66.77, 65.50, 57.40, 53.47, 48.88, 43.62, 41.69, 41.43, 35.80, 35.26, 35.12, 33.93, 33.79, 30.37, 30.30, 28.82, 23.05, 17.95, 17.76, 16.61. MS (ESI, negative mode): found m/z=1136.2 [M−H]⁻, calculated m/z=1136.6 [M−H]⁻.

### Mal-β-Ala-PEG(8)-Val-Ala-PAB-PNP (51)

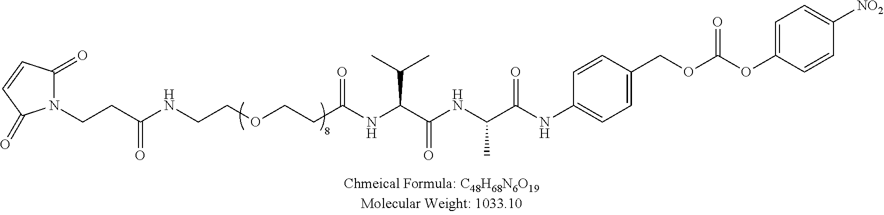

Following General Procedure L, intermediate 49 (1 eq., 0.044 mmol, 38 mg) and bis-(p-nitro-phenyl) carbonate (2 eq., 0.09 mmol, 27 mg) were dissolved in DMF (abs., 1 mL). Then, DIPEA (3 eq., 0.13 mmol, 23 μL) was added and the reaction mixture stirred for 3 h at r.t. The reaction progress was monitored by HPLC (method 6). After completion, the work-up was performed following General Procedure L, and purification by preparative HPLC (method 6, t_{R}=12.89 min) afforded intermediate 51 (10% yield, 0.004 mmol, 4.5 mg) as a white solid after lyophilisation. MS (ESI, positive mode): found m/z=1034.0 [M+H]⁺, calculated m/z=1034.4 [M+H]⁺.

### Mal-Peg(8)-Val-Ala-Pab-36 (52)

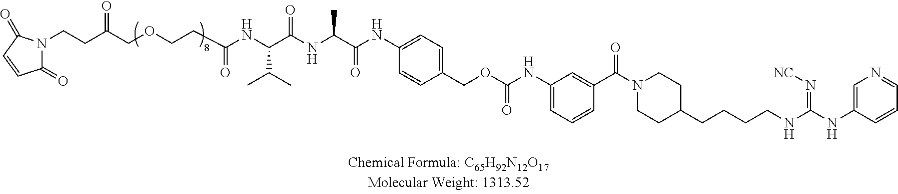

Following General Procedure J, Intermediate 51 (1 eq., 0.023 mmol, 24 mg) and HATU (1.1 eq., 0.025 mmol, 9.5 mg) were dissolved in DMF (abs., 1.6 mL). Then, DIPEA (4 eq., 0.09 mmol, 15 μL) was added and the reaction stirred at r.t. After 20 min, intermediate 36 (1 eq., 0.023 mmol, 9.6 mg) was added and the reaction stirred over the weekend. The reaction was monitored by TLC (SiO₂, CHCl₃/MeOH 10/1) and HPLC (method 7). After completion, the work-up was performed as described in General Procedure J and purified by preparative HPLC (method 7, t_{R}=9.03 min), affording intermediate 52 (1% yield, 0.23 μmol, 0.3 mg) as a white solid after lyophilisation. MS (ESI, positive mode): found m/z=1314.5 [M+H]⁺, calculated m/z=1314.7 [M+H]⁺.

### N-Cbz-4-(pent-4-(en)-yl)-4-hydroxy-piperidine (53)

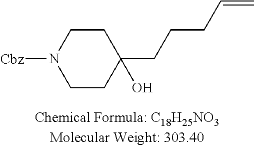 [SMILES: C=CCCCC1(O)CCN(C(=O)OCc2ccccc2)CC1]

Following General Procedure M, magnesium (turnings; 2 eq., 13 mmol, 316 mg) and a spatula tip of 12 were charged into a dried three-neck dried flask under Ar atmosphere, followed by the addition of THF (abs. 15 mL). After the activation of magnesium following the instructions from General Procedure M, 5-bromo-1-pentene (1.5 eq., 9.9 mmol, 1.2 mL) was added. In parallel, N-Cbz-4-piperidone (1 eq., 6.5 mmol, 1.5 g) was dissolved in a 0.6 M solution of LaCl_{3*2}LiCl in THF (10.8 mL) in a second dried two-neck flask, and the mixture was activated at for 1 h at r.t., followed by cooling down to 8° C. Next, the Grignard solution was added at 8° C. dropwise to the activated piperidone solution, and the resulting reaction mixture was stirred overnight at 8° C. The reaction was monitored by TLC (SiO₂, hexane/EtOAc: 1/1) and HPLC (method 8). After completion, the work-up was performed as described in General Procedure M, and the crude product was adsorbed on diatomaceous earth. Purification by flash chromatography, applying an isocratic method (Hex/EtOAc: 1/1), afforded intermediate 53 (47% yield, 3.1 mmol, 927 mg) as oil. t_{R}/min (HPLC, method 8)=11.75. ¹H-NMR (500 MHz, d6-DMSO): δ/ppm=7.40-7.28 (m, 5H), 5.80 (ddt, J=16.9, 10.2, 6.6 Hz, 1H), 5.06 (s, 2H), 5.00 (dq, 1H), 4.94 (ddt, J=10.2, 2.3, 1.2 Hz, 1H), 4.16 (s, 1H), 3.75-3.65 (m, 2H), 1.99 (dtd, J=7.3, 6.2, 5.8, 1.5 Hz, 2H), 1.48-1.29 (m, 8H). ¹³C-NMR (126 MHz, d6-DMSO): δ/ppm=154.30, 138.75, 137.03, 128.26, 127.61, 127.34, 114.48, 67.50, 65.88, 41.88, 33.63, 21.54. MS (ESI, negative mode): found m/z=302.1 [M−H]⁻, calculated m/z=302.2 [M−H]⁻.

### 5-(N-Cbz-1-hydroxy-piperidinyl)-1,2-pentandiol (54)

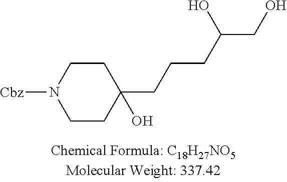 [SMILES: O=C(OCc1ccccc1)N1CCC(O)(CCCC(O)CO)CC1]

Following General Procedure N, Intermediate 53 (1 eq., 0.988 mmol, 300 mg) was dissolved in acetone (6 mL). Then, NMO (1.2 eq., 1.19 mmol, 139 mg) was added, followed by H₂O (16 eq., 16.7 mmol, 300 μL). Finally, an OsO₄-solution (0.47% mol, 4.6 μmol, 1 μL) was added and the reaction stirred overnight. The reaction progress was monitored by TLC (SiO₂, hexane/EtOAc: 1/1 for olefine consumption, CHCl₃/MeOH: 10/1 for diol formation) and HPLC (method 9). After completion, the work-up was performed as described in General Procedure M, and the crude product was adsorbed on diatomaceous earth. Purification by flash chromatography, applying an isocratic method (CHCl₃/MeOH: 10/1), afforded intermediate 53 (55% yield, 0.54 mmol, 183 mg) as oil. t_{R}/min (HPLC, method 8)=13.50. MS (ESI, positive mode): found m/z=338.17 [M+H]⁺ 360.25 [M+Na]+, calculated m/z=338.4 [M+H]⁺.

### 4-(N-Cbz-1-hydroxy-piperidinyl)-butanal (55)

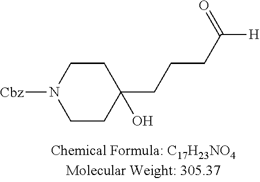 [SMILES: O=CCCCC1(O)CCN(C(=O)OCc2ccccc2)CC1]

Following General Procedure O, intermediate 54 (1 eq., 0.377 mmol, 127 mg) was dissolved in THF (2.4 mL). In parallel, sodium periodate (2 eq., 0.754 mmol, 161 mg) was dissolved in H₂O (1.2 mL). Then, both solutions are mixed following instructions from General Protocol O. The reaction mixture was stirred for 1 h. The reaction was monitored by HPLC (method 7). After completion, reaction the work-up was performed following General Procedure O. Purification by preparative HPLC (method 7, t_{R}=6.94 min) afforded intermediate 55 (38% yield, 0.14 mmol, 44 mg) as a white solid after lyophilisation. ¹H-NMR (500 MHz, d6-DMSO): δ/ppm=9.67 (s, 1H), 7.41-7.29 (m, 5H), 5.07 (s, 2H), 4.85 (ddd, J=8.0, 5.8, 2.3 Hz, 1H), 3.67 (d, J=13.1 Hz, 2H), 3.28 (s, 1H), 1.73-1.52 (m, 4H), 1.49-1.14 (m, 6H). ¹³C-NMR (126 MHz, d6-DMSO): δ/ppm=203.38, 154.40, 137.01, 128.29, 127.65, 127.35, 70.83, 65.93, 59.61, 43.38, 41.58, 39.23, 37.62, 34.22, 32.53, 31.47, 30.33, 20.63, 16.86, 15.17, 13.97, 1.83). MS (ESI, positive mode): found m/z=306.25 [M+H]⁺ 328.25 [M+Na]+, calculated m/z=306.2 [M+H]⁺.

### Tert-butyl(E)-(3-(4-(4-(3-(pyridin-3-yl)acrylamido)butyl)piperidine-1-carbonyl)phenyl)carbamate (56)

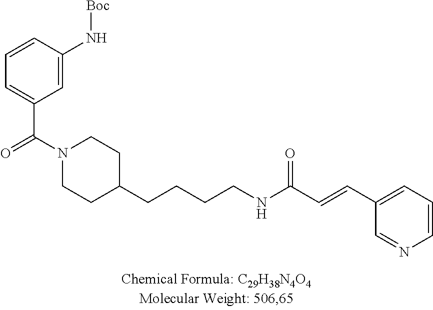 [SMILES: CC(C)(C)OC(=O)Nc1cccc(C(=O)N2CCC(CCCCNC(=O)/C=C/c3cccnc3)CC2)c1]

Following General Procedure F, intermediate 26 (1 eq., 2.09 mmol, 785 mg) and (E)-3-(3-pyridyl)-acrylic acid (1 eq., 2.09 mmol, 312 mg) were dissolved in DCM (abs., 230 mL). Then, DIPEA (1 eq., 2.09 mmol, 365 μL), HOBt (1 eq., 2.09 mmol, 281 mg) and DCC (1 eq., 2.09 mmol, 433 mg) were added sequentially and the reaction mixture stirred overnight at r.t. The reaction was monitored by TLC (SiO₂, DCM/MeOH: 30/1). After completion, the work-up was performed as described in General Procedure F, and the crude product was adsorbed on diatomaceous earth. Purification by flash chromatography, applying an isocratic method (DCM/MeOH: 30/1), afforded intermediate 56 (37% yield, 0.77 mmol, 392 mg) as oil. MS (ESI, positive mode): found m/z=507.3 [M+H]⁺, calculated m/z=507.3 [M+H]⁺.

### (E)-N-(4-(1-(3-aminobenzoyl)piperidin-4-yl)butyl)-3-(pyridin-3-yl)acrylamide (57)

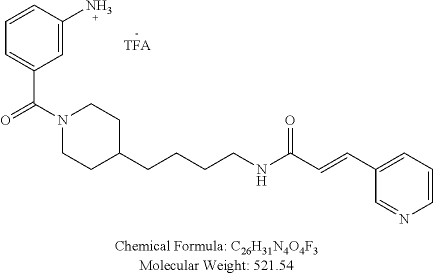 [SMILES: Nc1cccc(C(=O)N2CCC(CCCCNC(=O)/C=C/c3cccnc3)CC2)c1]

Following General Procedure C variation 1, Intermediate 56 (1 eq., 2.37 mmol, 1.2 g) was dissolved in pure TFA (15 mL) as a solvent. The reaction mixture was stirred at 400 mbar for 5 min. The reaction was monitored by HPLC (method 2). After completion, the work-up was performed as described in General Procedure C. The crude product was purified by preparative HPLC (method 2, t_{R}=9.06 min), affording intermediate 57 (60% yield, 1.4 mmol, 742 mg) as oil. ¹H-NMR (500 MHz, d6-DMSO): δ/ppm=9.20 (s, 2H), 8.94 (d, J=2.1 Hz, 1H), 8.72 (dd, J=5.2, 1.5 Hz, 1H), 8.36 (dt, J=8.2, 1.8 Hz, 1H), 8.21 (t, J=5.6 Hz, 1H), 7.77 (dd, J=8.1, 5.2 Hz, 1H), 7.51 (d, J=16.0 Hz, 1H), 7.39 (t, J=7.7 Hz, 1H), 7.09 (d, J=8.0 Hz, 2H), 6.84 (d, J=15.9 Hz, 1H), 3.19 (q, J=6.6 Hz, 2H), 1.56-1.21 (m, 8H), 1.09-1.02 (m, 2H). ¹³C-NMR (126 MHz, d6-DMSO): δ/ppm=168.02, 163.95, 145.66, 144.92, 138.44, 137.64, 133.36, 129.54, 126.44, 125.58, 122.10, 120.88, 118.32, 116.61, 114.30, 38.68, 35.42, 35.22, 29.16, 26.78, 23.41. MS (ESI, positive mode): found m/z=407.3 [M+H]⁺ calculated m/z=407.2 [M+H]⁺.

### (E)-N-(4-(1-(3-(3-(2,5-dioxo-2,5-dihydro-1H-pyrrol-1-yl)propanamido)benzoyl)piperidin-4-yl)butyl)-3-(pyridin-3-yl)acrylamide (58)

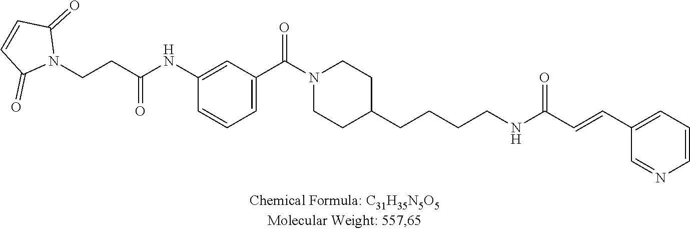 [SMILES: O=C(/C=C/c1cccnc1)NCCCCC1CCN(C(=O)c2cccc(NC(=O)CCN3C(=O)C=CC3=O)c2)CC1]

Following General Procedure J, 3-maleimidyl-propionic acid (1.5 eq., 0.07 mmol, 12 mg) was dissolved in DMF (1.6 mL). Then, PyAOP (1.5 eq., 0.07 mmol, 38 mg) and DIPEA (6 eq., 0.3 mmol, 53 μL) were added and stirred for 30 min at r.t. Next, intermediate 57 (1 eq., 0.05 mmol, 26 mg) was added, and the reaction mixture was stirred overnight at r.t. The reaction progress was monitored by HPLC (method 2). After completion, the reaction mixture was directly purified by preparative HPLC (method 2, t_{R}=11.83 min), affording intermediate 58 (34% yield, 0.017 mmol, 9.5 mg) as a white solid. ¹H-NMR (500 MHz, d6-DMSO): δ/ppm=10.07 (s, 1H), 8.85 (d, J=2.2 Hz, 1H), 8.63 (dd, J=5.0, 1.5 Hz, 1H), 8.21-8.12 (m, 2H), 7.66-7.59 (m, 2H), 7.53 (ddd, J=8.1, 2.2, 1.1 Hz, 1H), 7.48 (d, J=15.9 Hz, 1H), 7.33 (t, J=7.9 Hz, 1H), 7.04-6.97 (m, 3H), 6.78 (d, J=15.9 Hz, 1H), 3.72 (t, J=7.1 Hz, 2H), 3.19 (q, J=6.8 Hz, 2H), 2.64-2.52 (m, 2H), 1.56-1.15 (m, 7H), 1.09-1.02 (m, 2H). ¹³C-NMR (126 MHz, d6-DMSO): δ/ppm 171.20, 169.20, 169.06, 164.65, 148.22, 147.38, 139.43, 136.77, 135.05, 134.66, 129.23, 125.98, 125.28, 121.79, 120.34, 117.82, 39.19, 35.97, 35.79, 35.55, 34.30, 29.70, 23.97. MS (ESI, positive mode): found m/z=558.3 [M+H]⁺, calculated m/z=558.3 [M+H]⁺.

### 4-((S)-2-((S)-2-(6-(2,5-dioxo-2,5-dihydro-1H-pyrrol-1-yl)hexanamido)-3-methylbutanamido)propanamido)benzyl(3-(4-(4-((E)-3-(pyridin-3-yl)acrylamido)butyl)piperidine-1-carbonyl)phenyl)carbamate (59)

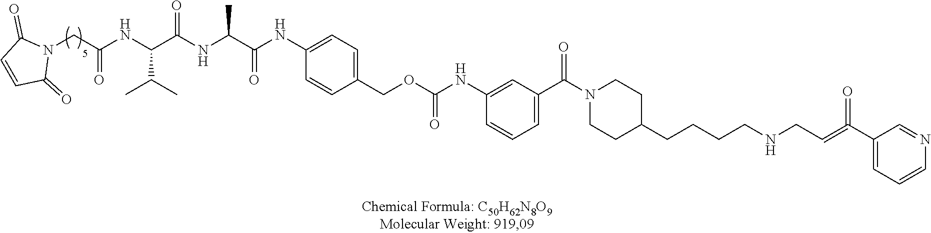

Following General Procedure J, mc-Val-Ala-PAB-PNP (CAS-No. 1639939-40-4, 1.5 eq., 0.037 mmol, 24 mg) was dissolved in DMF (1.5 mL). Then, PyAOP (1.5 eq., 0.037 mmol, 19 mg) and DIPEA (6 eq., 0.15 mmol, 26 μL) were added and stirred for 30 min at r.t. Next, intermediate 57 (1 eq., 0.025 mmol, 13 mg) was added and the reaction mixture stirred overnight at r.t. The reaction progress was monitored by HPLC (method 2). After completion, the reaction mixture was directly purified by preparative HPLC (method 2, t_{R}=15.37 min), affording intermediate 59 (20% yield, 0.005 mmol, 4.5 mg) as white solid. ¹H-NMR (500 MHz, d6-DMSO): δ/ppm=9.91 (s, 1H), 9.83 (s, 1H), 8.75 (d, J=2.2 Hz, 1H), 8.55 (dd, J=4.7, 1.7 Hz, 1H), 8.39-8.31 (m, 3H), 8.17-8.06 (m, 1H), 7.77 (d, J=8.6 Hz, 1H), 7.68-7.58 (m, 5H), 7.53-7.39 (m, 4H), 6.74 (d, J=15.9 Hz, 1H), 5.09 (s, 2H), 4.40 (p, J=6.9 Hz, 1H), 4.16 (dd, J=8.6, 6.8 Hz, 1H), 3.37 (t, J=7.1 Hz, 2H), 3.02 (dtd, J=6.6, 3.9, 2.4 Hz, 3H), 1.91-1.81 (m, 24H), 1.79-1.68 (m, 2H), 0.86 (d, J=6.8 Hz, 3H), 0.83 (d, J=6.8 Hz, 3H). ¹³C-NMR (126 MHz, d6-DMSO): δ/ppm=172.21, 171.00, 170.90, 170.88, 168.50, 164.25, 153.29, 149.60, 148.63, 139.05, 138.79, 136.95, 134.79, 134.32, 134.07, 131.05, 130.82, 128.79, 126.20, 124.47, 123.93, 122.16, 122.13, 120.39, 118.92, 65.53, 57.51, 48.92, 47.17, 38.59, 36.91, 35.40, 35.21, 34.83, 29.15, 27.63, 25.81, 25.75, 25.69, 25.64, 25.57, 24.76, 17.82. MS (ESI, positive mode): found m/z=920.2 [M+H]⁺, calculated m/z=920.5 [M+H]⁺.

### 4-(N-Boc-amino)-phenylacetic acid (60)

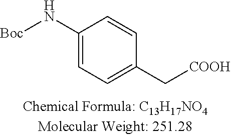 [SMILES: CC(C)(C)OC(=O)Nc1ccc(CC(=O)O)cc1]

Following General Procedure A, 4-amino-phenylacetic acid (1 eq., 6.62 mmol, 1 g) and di-tert-butyl dicarbonate (2 eq., 3.34 mmol, 3 g) were dissolved in DCM (60 mL) and cooled to 0° C. Then, triethylamin (2 eq., 3.34 mmol, 1.9 mL) was added, the mixture was allowed to warm to r.t. and stirred overnight. The reaction progress was monitored by TLC (SiO₂, CHCl₃/MeOH/AcOH: 30/1/1%). After completion, the volatiles were evaporated under reduced pressure, and the crude product was adsorbed on diatomaceous earth. Purification by flash chromatography, applying an isocratic method (CHCl₃/MeOH/AcCOOH: 30/1/1%), afforded intermediate 60 (80% yield, 5.3 mmol, 1.3 g) as pale orange crystals. MS (ESI, negative mode): found m/z=250.2 [M−H]⁻, calculated m/z=250.1 [M−H]⁻.

### Tert-butyl(E)-(4-(2-oxo-2-((3-(4-(4-(3-(pyridin-3-yl)acrylamido)butyl)piperidine-1-carbonyl)phenyl)amino)ethyl)phenyl)carbamate (61)

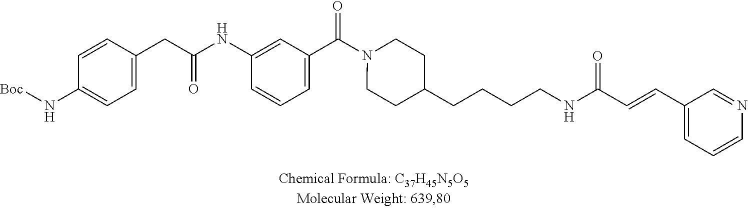 [SMILES: CC(C)(C)OC(=O)Nc1ccc(CC(=O)Nc2cccc(C(=O)N3CCC(CCCCNC(=O)/C=C/c4cccnc4)CC3)c2)cc1]

Following General Procedure J, intermediate 60 (1 eq., 0.098 mmol, 24.6 mg) was dissolved in DMF (2 mL). Then, PyAOP (1.5 eq., 0.15 mmol, 77 mg) and DIPEA (5 eq., 0.49 mmol, 85 μL) were added and stirred for 30 min at r.t. Next, intermediate 57 (1.5 eq., 0.15 mmol, 78 mg) was added, and the reaction mix is stirred overnight at r.t. The reaction progress was monitored by HPLC (method 10). After completion, the reaction mixture was directly purified by preparative HPLC (method 10, t_{R}=13.07 min), affording intermediate 61 (20% yield, 0.02 mmol, 13 mg) as a white solid. MS (ESI, positive mode): found m/z=640.3 [M+H]⁺, calculated m/z=640.3 [M+H]⁺.

### (E)-N-(4-(1-(3-(2-(4-aminophenyl)acetamido)benzoyl)piperidin-4-yl)butyl)-3-(pyridin-3-yl)acrylamide (62)

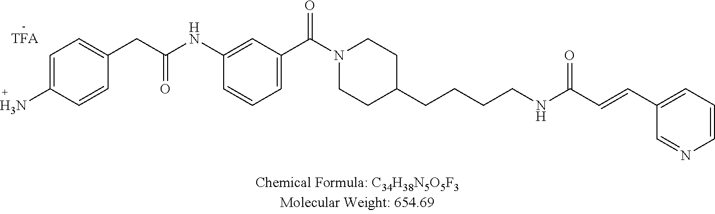 [SMILES: Nc1ccc(CC(=O)Nc2cccc(C(=O)N3CCC(CCCCNC(=O)/C=C/c4cccnc4)CC3)c2)cc1]

Following General Procedure C variation 1, Intermediate 57 (1 eq., 0.011 mmol, 5.73 mg) was dissolved in pure TFA (2 mL). The reaction mixture was stirred at 400 mbar for 5 min the progress monitored by HPLC (method 2). After completion, the work-up was performed as described in General Procedure C. Purification by preparative HPLC (method 2, t_{R}=7.40 min) afforded intermediate 62 (55% yield, 0.006 mmol, 4 mg) as oil. MS (ESI, positive mode): found m/z=540.3 [M+H]⁺, calculated m/z=540.3 [M+H]⁺.

### Tert-butyl4-(4-(4-(4-hydroxybutyl)piperidine-1-carbonyl)phenyl)piperazine-1-carboxylate (63)

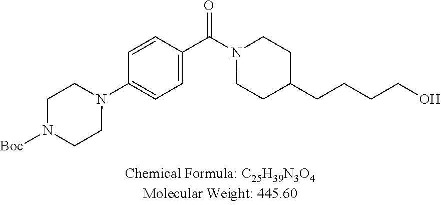 [SMILES: CC(C)(C)OC(=O)N1CCN(c2ccc(C(=O)N3CCC(CCCCO)CC3)cc2)CC1]

Following General Procedure F, intermediate 22 (1 eq., 1.63 mmol, 256 mg) and 4-(4-carboxy-phenyl)-piperazine-1-carboxylic acid tert-butyl ester (1 eq., 1.63 mmol, 500 mg) were dissolved in DCM (abs., 20.5 mL). Then, DIPEA (1 eq., 1.63 mmol, 285 μL), HOBt (1 eq., 1.63 mmol, 219 mg), and DCC (1 eq., 1.63 mmol, 338 mg) were added sequentially and the reactions mixture was stirred overnight at r.t. The reaction was monitored by TLC (SiO₂, hexane/EtOAc/MeOH: 10/10/1). After completion, the work-up was performed as described in General Procedure F, and the crude product was adsorbed on diatomaceous earth. Purification by flash chromatography, applying an isocratic method (hexane/EtOAc/MeOH: 10/10/1), afforded intermediate 63 (40% yield, 0.65 mmol, 290 mg) as orange oil. MS (ESI, positive mode): found m/z=446.4 [M+H]⁺, calculated m/z=446.3 [M+H]⁺.

### Tert-butyl-4-(4-(4-(4-methanesulfonylbutyl)piperidine-1-carbonyl)phenyl)piperazine-1-carboxylate (64)

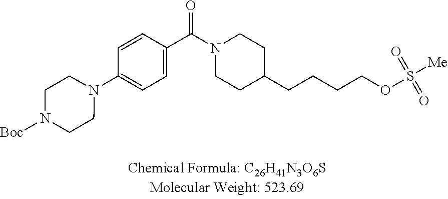 [SMILES: CC(C)(C)OC(=O)N1CCN(c2ccc(C(=O)N3CCC(CCCCOS(C)(=O)=O)CC3)cc2)CC1]

Following General Procedure G, intermediate 63 (1 eq., 1.29 mmol, 575 mg) was dissolved in DCM (abs., 10 mL) and cooled to 0° C. Then, methanesulfonyl chloride (1.5 eq., 1.93 mmol, 152 μL) and DIPEA (2 eq., 2.57 mmol, 448 μL) were added and stirred for 2 h at r.t. The reaction progress was monitored by TLC (SiO₂, DCM/MeOH: 30/1). After completion, the work-up was performed as described in General Procedure G, and the crude product was adsorbed on diatomaceous earth. Purification by flash chromatography, applying an isocratic method (DCM/MeOH: 30/1), afforded intermediate 64 (73% yield, 0.94 mmol, 493 mg) as an orange oil. MS (ESI, positive mode): found m/z=524.4 [M+H]⁺, calculated m/z=524.3 [M+H]⁺.

### Tert-butyl-4-(4-(4-(4-phtalimidylbutyl)piperidine-1-carbonyl)phenyl)piperazine-1-carboxylate (65)

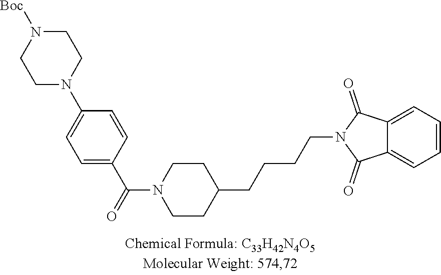 [SMILES: CC(C)(C)OC(=O)N1CCN(c2ccc(C(=O)N3CCC(CCCCN4C(=O)c5ccccc5C4=O)CC3)cc2)CC1]

Following General Procedure H part 1, intermediate 64 (1 eq., 0.83 mmol, 434 mg) and potassium phthalimide (1.2 eq., 0.997 mmol, 188 mg) were dissolved in DMF (abs. 9 mL). Following the temperature conditions described in General Procedure H part 1, the reaction mixture was stirred overnight. The reaction was monitored by TLC (SiO₂, hexane/EtOAc/MeOH: 13/7/1). After completion, the work-up was performed as described in General Procedure H part 1, and the crude product was adsorbed on diatomaceous earth. Purification by flash chromatography, applying an isocratic method (hexane/EtOAc/MeOH: 13/7/1), afforded intermediate 65 (81% yield, 0.67 mmol, 386 mg) as brown oil. MS (ESI, positive mode): found m/z=575.6 [M+H]⁺, calculated m/z=575.3 [M+H]⁺.

### Tert-butyl-4-(4-(4-(4-aminobutyl)piperidine-1-carbonyl)phenyl)piperazine-1-carboxylate (66)

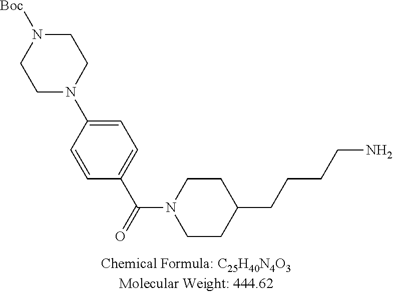 [SMILES: CC(C)(C)OC(=O)N1CCN(c2ccc(C(=O)N3CCC(CCCCN)CC3)cc2)CC1]

Following General Procedure H part 2, intermediate 65 (1 eq., 0.7 mmol, 402 mg) was dissolved in EtOH (13 mL). Then, hydrazine monohydrate (4 eq., 4.18 mmol, 208 μL) was added and the reaction mixture stirred overnight at r.t. The reaction progress was monitored by TLC (SiO₂, CHCl₃/MeOH/Et₃N: 10/1/1%). After completion, the work-up was performed as described in General Procedure H part 2, and the crude product was adsorbed on diatomaceous earth. Purification by flash chromatography, applying an isocratic method (CHCl₃/MeOH/Et₃N: 10/1/1%), afforded intermediate 66 (52% yield, 0.36 mmol, 162 mg) as pale-yellow crystals. MS (ESI, positive mode): found m/z=445.4 [M+H]⁺, calculated m/z=445.2 [M+H]⁺.

### Tert-butyl(E)-4-(4-(4-(4-(3-(pyridin-3-yl)acrylamido)butyl)piperidine-1-carbonyl)phenyl)piperazine-1-carboxylate (67)

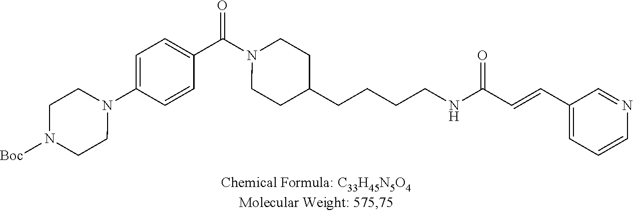 [SMILES: CC(C)(C)OC(=O)N1CCN(c2ccc(C(=O)N3CCC(CCCCNC(=O)/C=C/c4cccnc4)CC3)cc2)CC1]

Following General Procedure F, intermediate 66 (1 eq., 0.373 mmol, 166 mg) and (E)-3-(3-pyridyl)-acrylic acid (1 eq., 0.37 mmol, 56 mg) were dissolved in DCM (abs., 5 mL). Then, DIPEA (1 eq., 0.37 mmol, 65 μL), HOBt (1 eq., 0.37 mmol, 50 mg) and DCC (1 eq., 0.37 mmol, 77 mg) were added sequentially, and the resulting reaction mixture was stirred overnight at r.t. The reaction progress was monitored by TLC (SiO₂, DCM/MeOH: 10/1). After completion, the work-up was performed as described in General Procedure F, and the crude product was adsorbed on diatomaceous earth. Purification by flash chromatography, applying an isocratic method (DCM/MeOH: 10/1), afforded intermediate 67 (35% yield, 0.13 mmol, 75 mg) as oil. MS (ESI, positive mode): found m/z=577.1 [M+H]⁺, calculated m/z=576.4 [M+H]⁺.

### (E)-N-(4-(1-(4-(piperazin-1-yl)benzoyl)piperidin-4-yl)butyl)-3-(pyridin-3-yl)acrylamide (68)

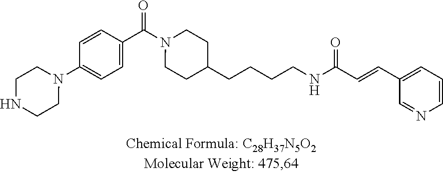 [SMILES: O=C(/C=C/c1cccnc1)NCCCCC1CCN(C(=O)c2ccc(N3CCNCC3)cc2)CC1]

Following General Procedure C variation, intermediate 67 (1 eq., 0.13 mmol, 75 mg) was dissolved in pure TFA (2 mL). The solution was stirred at 400 mbar for 5 min. The reaction progress was monitored by HPLC (method 2). After completion, the work-up was performed as described in General Procedure C. The crude product was purified by preparative HPLC using (method 2, t_{R}=9.12 min), affording intermediate 68 (60% yield, 0.078 mmol, 37 mg) as brown oil. ¹H-NMR (500 MHz, d6-DMSO): δ/ppm=8.87 (s, 1H), 8.83 (d, J=2.2 Hz, 1H), 8.62 (dd, J=5.0, 1.6 Hz, 1H), 8.14 (dt, J=8.0, 1.9 Hz, 1H), 7.58 (dd, J=8.0, 5.0 Hz, 1H), 7.47 (d, J=15.9 Hz, 1H), 7.29 (d, J=8.8 Hz, 2H), 7.00 (d, J=8.8 Hz, 2H), 6.78 (d, J=15.9 Hz, 1H), 3.42 (dd, J=6.6, 4.0 Hz, 4H), 3.26-3.15 (m, 8H), 2.86 (s, 2H), 1.67 (d, J=12.7 Hz, 2H), 1.47 (dq, J=14.6, 7.4 Hz, 3H), 1.38-1.21 (m, 3H), 1.05 (qd, J=12.4, 4.2 Hz, 2H). ¹³C-NMR (126 MHz, d6-DMSO): δ/ppm=168.96, 164.22, 150.46, 148.10, 147.19, 135.87, 134.31, 131.58, 128.35, 127.06, 125.26, 124.63, 114.87, 114.80, 44.83, 42.60, 38.67, 35.44, 35.33, 31.98, 29.17, 23.43. MS (ESI, positive mode): found m/z=476.3 [M+H]⁺, calculated m/z=476.3 [M+H]⁺.

### 4-((S)-2-((S)-2-(6-(2,5-dioxo-2,5-dihydro-1H-pyrrol-1-yl)hexanamido)-3-methylbutanamido)propanamido)benzyl4-(4-(4-(4-((E)-3-(pyridin-3-yl)acrylamido)butyl)piperidine-1-carbonyl)phenyl) piperazine-1-carboxylate (69)

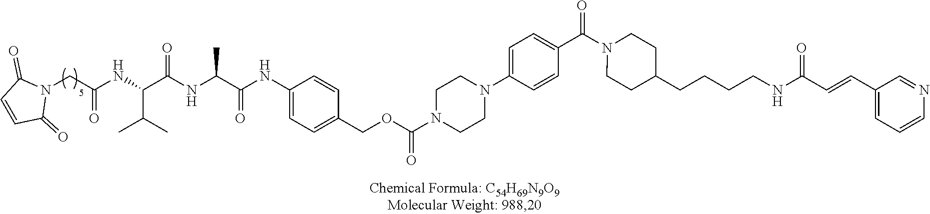

Following General Procedure J, mc-Val-Ala-PAB-PNP (CAS-No. 1639939-40-4, 1.5 eq., 0.023 mmol, 15 mg) was dissolved in DMF (1 mL). Then, PyAOP (1.5 eq., 0.032 mmol, 16 mg) and DIPEA (6 eq., 0.13 mmol, 23 μL) were added and at stirred for 30 min at r.t. Next, intermediate 68 (1 eq., 0.05 mmol, 24 mg) was added, and the resulting mixture was stirred overnight at r.t. The reaction was monitored by HPLC (method 2). After completion, the reaction mixture was directly purified by preparative HPLC (method 2, t_{R}=15.37 min), affording intermediate 69 (12% yield, 0.006 mmol, 6 mg) as white solid. ¹H-NMR (500 MHz, d6-DMSO): δ/ppm 9.88 (s, 1H), 8.78 (d, J=2.2 Hz, 1H), 8.58 (dd, J=4.9, 1.6 Hz, 1H), 8.10 (t, J=5.7 Hz, 1H), 8.10-8.01 (m, 2H), 7.74 (d, J=8.5 Hz, 1H), 7.59 (d, J=8.5 Hz, 2H), 7.51 (dd, J=8.0, 4.9 Hz, 1H), 7.45 (d, J=15.9 Hz, 1H), 7.32 (d, J=8.6 Hz, 2H), 7.25 (d, J=8.7 Hz, 2H), 6.98 (s, 2H), 6.94 (d, J=8.8 Hz, 2H), 6.74 (d, J=15.9 Hz, 1H), 5.04 (s, 2H), 4.16 (dd, J=8.6, 6.7 Hz, 1H), 3.37 (t, J=7.1 Hz, 2H), 3.30 (h, J=3.7 Hz, 4H), 3.24-3.14 (m, 7H), 2.84 (s, 2H), 2.24-2.06 (m, 2H), 1.97 (h, J=6.8 Hz, 1H), 1.87 (td, J=6.2, 3.3 Hz, 4H), 1.66 (d, J=12.8 Hz, 2H), 1.48 (ddd, J=21.1, 11.7, 5.3 Hz, 8H), 1.39-1.14 (m, 13H), 1.05 (q, J=10.3 Hz, 2H), 0.85 (dd, J=16.5, 6.8 Hz, 6H). ¹³C-NMR (126 MHz, d6-DMSO): δ/ppm=172.19, 170.96, 170.88, 169.00, 164.16, 154.36, 148.93, 147.98, 138.59, 134.80, 134.54, 134.30, 131.42, 131.10, 128.34, 128.28, 126.18, 126.11, 124.77, 124.18, 118.99, 114.37, 66.03, 57.49, 48.89, 47.32, 47.21, 47.17, 43.03, 38.57, 36.91, 35.39, 35.30, 34.82, 31.93, 30.32, 30.19, 29.14, 27.61, 25.67, 25.62, 25.55, 24.74, 23.37, 19.06, 18.03, 17.81. MS (ESI, positive mode): found m/z=989.5 [M+H]⁺, calculated m/z=989.5 [M+H]⁺.

### (E)-N-(4-(1-(4-(4-(3-(2,5-dioxo-2,5-dihydro-1H-pyrrol-1-yl)propanoyl)piperazin-1-yl)benzoyl)piperidin-4-yl)butyl)-3-(pyridin-3-yl)acrylamide (70)

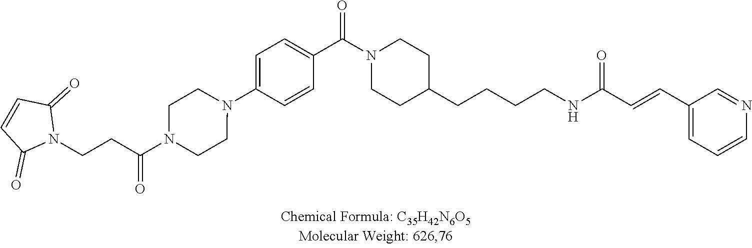 [SMILES: O=C(/C=C/c1cccnc1)NCCCCC1CCN(C(=O)c2ccc(N3CCN(C(=O)CCN4C(=O)C=CC4=O)CC3)cc2)CC1]

Following General Procedure J, 3-maleimidopropionic acid (1.5 eq., 0.08 mmol, 13.5 mg) was dissolved in DMF (1 mL). Then, PyAOP (1.5 eq., 0.032 mmol, 16 mg) and DIPEA (6 eq., 0.13 mmol, 23 μL) were added and stirred for 30 min at r.t. Next, intermediate 68 (1 eq., 0.05 mmol, 24 mg) was added and the reaction mixture stirred overnight at r.t. The reaction progress was monitored by HPLC (method 2). After completion, reaction mixture was directly purified by preparative HPLC (method 2, t_{R}=12.18 min), affording intermediate 70 (34% yield, 0.017 mmol, 10.6 mg) as a white solid. ¹H-NMR (500 MHz, d6-DMSO): δ/ppm=8.81 (s, 1H), 8.60 (d, J=4.0 Hz, 1H), 8.15-8.06 (m, 2H), 7.54 (dd, J=8.0, 4.9 Hz, 1H), 7.46 (d, J=15.9 Hz, 1H), 7.26 (d, J=8.7 Hz, 2H), 7.00 (s, 2H), 6.95 (d, J=8.9 Hz, 2H), 6.75 (d, J=15.9 Hz, 1H), 4.05 (s, 8H), 3.69-3.61 (m, 2H), 3.56 (t, J=5.2 Hz, 4H), 3.27-3.16 (m, 2H), 2.69-2.61 (m, 2H), 1.70-1.63 (m, 2H), 1.46 (q, J=6.9 Hz, 3H), 1.35-1.20 (m, 4H), 1.11-0.99 (m, 2H). ¹³C-NMR (126 MHz, d6-DMSO): δ/ppm 170.67, 169.02, 168.14, 164.13, 151.11, 148.51, 147.61, 135.25, 134.51, 134.39, 131.30, 128.30, 126.03, 124.98, 124.36, 114.25, 47.55, 47.14, 44.37, 40.53, 38.58, 35.40, 35.31, 33.55, 31.94, 30.91, 29.14, 23.37. MS (ESI, positive mode): found m/z=627.5 [M+H]⁺, calculated m/z=627.3 [M+H]⁺.

### Benzyl 4-(4-aminobutyl)-4-hydroxypiperidine-1-carboxylate (71)

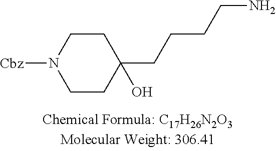 [SMILES: NCCCCC1(O)CCN(C(=O)OCc2ccccc2)CC1]

Following General Procedure P, intermediate 55 (1 eq., 4.27 mmol, 1.3 g) was dissolved in MeOH (abs., 18.24 mL). In parallel, ammonium acetate (10 eq., 42.66 mmol, 3.3 g) and sodium cyanoborohydride (2 eq., 8.53 mmol, 536 mg) were charged in a two-neck flask and dissolved in MeOH (abs. 13 mL). Then, following the conditions described in General Procedure P, ammonium acetate/cyanoborohydride solution was added to the intermediate 55-solution and stirred for 8 h at r.t. The reaction progress was monitored by TLC (SiO₂, CHCl₃/MeOH/Et₃N: 10/1/1%) and HPLC (method 11). After completion, the work-up was performed as described in General Procedure P. The crude product was purified by preparative HPLC (method 11, t_{R}=15.57 min), affording intermediate 71 (20% yield, 0.854 mmol, 262 mg) as a white solid. ¹H-NMR (500 MHz, d6-DMSO): δ/ppm=7.41-7.27 (m, 5H), 5.06 (s, 2H), 3.71 (dt, J=13.2, 4.0 Hz, 2H), 2.82-2.75 (m, 2H), 1.56-1.32 (m, 10H). ¹³C-NMR (126 MHz, d6-DMSO): δ/ppm=154.33, 137.05, 128.29, 127.65, 127.36, 67.55, 66.28, 65.92, 41.66, 39.78, 38.84, 35.97, 27.57, 19.35. MS (ESI, positive mode): found m/z=307.33 [M+H]⁺, calculated m/z=306.2 [M+H]⁺.

### 6-(Pyridin-4-yl)hex-5-yn-1-ol (72)

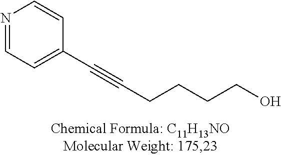 [SMILES: OCCCCC#Cc1ccncc1]

### Step 1:

4-Bromopyridine hydrochloride (1 eq., 10.28 mmol, 2 g) was dissolved in an aqueous 2 M NaOH-solution (8 mL). The reaction mixture was then stirred for 5 min. The aqueous solution was extracted with EtOAc (3×), and the combined organic layers were washed once with H₂O and once with brine and dried over MgSO₄. The volatiles were evaporated under reduced pressure.

### Step 2:

The product from step 1 was charged into a three-neck flask equipped with a reflux condenser, dissolved in triethylamine (2.8 eq., 28.7 mmol, 4 mL) and the solution degassed with argon. Next, Hex-5-yn-1-ol (1 eq., 10.28 mmol, 1.13 mL), Bis(triphenylphosphine)palladium chloride (1.1 mol-%, 0.114 mmol, 80 mg) and copper(I) iodide (2.04% mol, 0.21 mmol, 40 mg) were added sequentially. The reaction mixture was heated to reflux for 30 min and the reaction progress was monitored by TLC (SiO₂, EtOAc 100%). After completion, the reaction mixture was cooled to r.t. and extracted with EtOAc (100 mL) and the organic layer washed with H₂O (25 mL). Next, the organic layer was collected and washed once with H₂O and once with brine, dried over MgSO₄, and the volatiles were evaporated under reduced pressure. The crude product was adsorbed on diatomaceous earth. Purification by flash chromatography, applying an isocratic method (EtOAC=100%), afforded intermediate 72 (30% yield, 3.1 mmol, 540 mg) as a white solid. MS (ESI, positive mode): found m/z=176.2 [M+H]⁺, calculated m/z=176.1 [M+H]⁺.

### 6-(Piperidin-4-yl)hexan-1-ol (73)

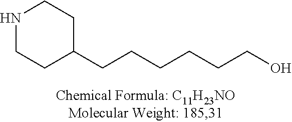 [SMILES: OCCCCCCC1CCNCC1]

Intermediate 72 (1 eq., 3.32 mmol, 582 mg) was charged in a three-neck flask and dissolved in acetic acid (15 mL). Two of the three necks were then equipped with a balloon filled with argon and a balloon filled with hydrogen, respectively. Afterwards, PtO₂ (20% mol, 0.664 mmol, 81 mg) was added through the central neck, and the system connected to a vacuum pump. Without stirring, the system was degassed with five cycles comprising evacuation and flushing with argon followed by five evacuation/flushing cycles with hydrogen in the same manner as for argon. Then, vigorous stirring was started, and the reaction mixture was kept at r.t. for 3 h. The reaction progress was monitored by TLC (SiO₂, CHCl₃/MeOH/Et₃N: 10/1/1%). After completion, the work-up was performed as described in General Procedure E, affording intermediate 73 (85% yield, 2.8 mmol, 523 mg) as a white solid. MS (ESI, positive mode): found m/z=186.5 [M+H]⁺, calculated m/z=187.1 [M+H]⁺.

### 6-(N-Boc-piperidin-4-yl)hexan-1-ol (74)

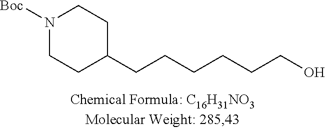 [SMILES: CC(C)(C)OC(=O)N1CCC(CCCCCCO)CC1]

Following General Procedure A, intermediate 73 (1 eq., 3.48 mmol, 645 mg) and di-tert-butyl dicarbonate (2 eq., 6.97 mmol, 6.3 g) were dissolved in DCM (90 mL) and cooled to 0° C. Then, triethylamine (2 eq., 6.97 mmol, 4 mL) was added and the mixture allowed to warm to r.t. The mixture was stirred overnight at r.t. and the progress monitored by TLC (SiO₂, hexane/EtOAc: 4/1). After completion, the volatiles are evaporated under reduced pressure, and the crude product was adsorbed on diatomaceous earth. Purification by flash chromatography, applying an isocratic method (Hex/EtOAc: 4/1), afforded intermediate 74 (70% yield, 2.44 mmol, 695 mg) as clear oil. MS (ESI, positive mode): found m/z=308.33 [M+Na]⁺, calculated m/z=286.2 [M+H]⁺.

### Benzyl (E)-4-(4-(2-cyano-3-(pyridin-3-yl)guanidino)butyl)-4-hydroxypiperidine-1-carboxylate (75)

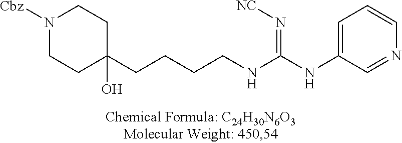 [SMILES: N#C/N=C(\NCCCCC1(O)CCN(C(=O)OCc2ccccc2)CC1)Nc1cccnc1]

Following General Procedure B, intermediate 71 (1 eq., 0.161 mmol, 49 mg) and intermediate 1 (1.1 eq., 0.178 mmol, 41 mg) were dissolved in 1,4-dioxane (3.8 mL). Triethylamine (2.4 eq., 19.65 mmol, 54 μL) was added and the resulting mixture stirred overnight at r.t. The reaction progress was monitored by TLC (SiO₂, CHCl₃/MeOH: 15/1). After completion, the work-up was performed as described in General Procedure B, and purification by preparative HPLC (method 2, t_{R}=12.94 min), afforded intermediate 75 (40% yield, 0.06 mmol, 29 mg) as sticky oil. MS (ESI, positive mode): found m/z=451.4 [M+H]⁺, calculated m/z=451.2 [M+H]⁺.

### N-Boc-4-(6-methanesulfonyl-hexyl)-piperidine (76)

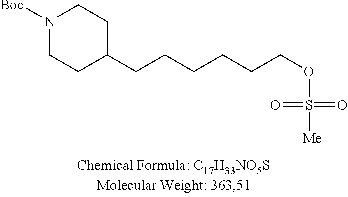 [SMILES: CC(C)(C)OC(=O)N1CCC(CCCCCCOS(C)(=O)=O)CC1]

Following General Procedure G, intermediate 74 (1 eq., 1.52 mmol, 434 mg) was dissolved in DCM (abs., 11.3 mL). The solution was cooled to 0° C. and methanesulfonyl chloride (1.5 eq., 2.28 mmol, 180 μL) was added, followed by triethylamine (2 eq., 3.04 mmol, 424 μL). The reaction was stirred for 1.5 h at r.t. The reaction was monitored by TLC (SiO₂, DCM/MeOH: 30/1) and after completion, the work-up was performed as described in General Procedure G. The crude product was adsorbed on diatomaceous earth, and purification by flash chromatography, applying an isocratic method (DCM/MeOH: 30/1), afforded intermediate 76 (70% yield, 1.1 mmol, 387 mg) as an oil. MS (ESI, positive mode): found m/z=364.1 [M+H]⁺, calculated m/z=364.2 [M+H]⁺.

### (E)-2-cyano-1-(4-(4-hydroxypiperidin-4-yl)butyl)-3-(pyridin-3-yl)guanidine (77)

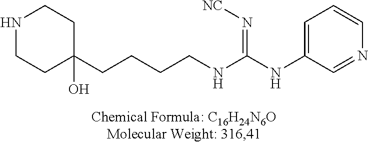 [SMILES: N#C/N=C(\NCCCCC1(O)CCNCC1)Nc1cccnc1]

Following General Procedure E, intermediate 75 (1 eq., 0.07 mmol, 32 mg) was dissolved in a solvent mixture of EtOAc (5 mL) and EtOH (5 mL). Then, a Pd/C (10% in Pd, 12 mol-%, 80 mg) was added and the hydrogenation started according to General Procedure E. The reaction mixture was stirred overnight at r.t and the progress monitored by TLC (SiO₂, CHCl₃/MeOH/Et₃N: 15/1/1%). After work-up according to General Procedure E, the product was ready to use without further purification. Intermediate 77 (73% yield, 0.05 mmol, 16 mg) was afforded as a white powder. MS (ESI, positive mode): found m/z=317.2 [M+H]⁺, calculated m/z=317.2 [M+H]⁺.

### Benzyl(E)-(3-(4-(4-(2-cyano-3-(pyridin-3-yl)guanidino)butyl)-4-hydroxypiperidine-1-carbonyl)phenyl)carbamate (78)

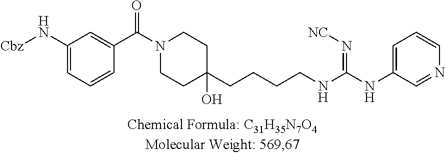 [SMILES: N#C/N=C(\NCCCCC1(O)CCN(C(=O)c2cccc(NC(=O)OCc3ccccc3)c2)CC1)Nc1cccnc1]

Following General Procedure F, intermediate 77 (1 eq., 1.78 mmol, 563 mg) and intermediate 30 (1 eq., 1.78 mmol, 483 mg) were dissolved in DCM (abs., 30 mL). Then, DIPEA (1 eq., 1.78 mmol, 313 μL), HOBt (1 eq., 1.78 mmol, 241 mg) and DCC (1 eq., 1.78 mmol, 370 mg) were sequentially added and the resulting mixture stirred overnight at r.t. The reaction progress was monitored by TLC (SiO₂, CHCl₃/MeOH: 15/1) and HPLC (method 11). After completion, the work-up was performed as described in General Procedure F and the crude product purified by preparative HPLC (method 11, t_{R}=13.74 min), affording intermediate 78 (37% yield, 0.66 mmol, 375 mg) as oil. MS (ESI, positive mode): found m/z=570.3 [M+H]⁺, calculated m/z=570.3 [M+H]⁺.

### N-Boc-4-(6-phthalimidyl-hexyl)-piperidine (79)

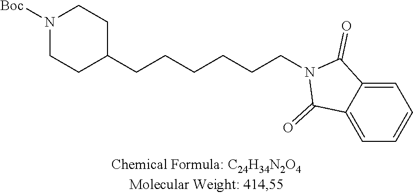 [SMILES: CC(C)(C)OC(=O)N1CCC(CCCCCCN2C(=O)c3ccccc3C2=O)CC1]

Following General Procedure H part 1, intermediate 76 (1 eq., 0.998 mmol, 363 mg) and potassium phthalimide (1.2 eq., 1.2 mmol, 226 mg) were dissolved in DMF (abs., 10.7 mL). Following the temperature conditions described in General Procedure H part 1, the reaction mixture was stirred overnight. The reaction progress was monitored by TLC (SiO₂, hexane/EtOAc/MeOH: 13/7/1). After completion, the work-up was performed as described in General Procedure H part 1, and the crude product was adsorbed on diatomaceous earth. Purification by flash chromatography, applying an isocratic method (Hex/EtOAc/MeOH: 13/7/1), afforded intermediate 79 (75% yield, 0.75 mmol, 310 mg) as oil. MS (ESI, positive mode): found m/z=415.2 [M+H]⁺, calculated m/z=415.2 [M+H]⁺.

### N-Boc-4-(6-amino-hexyl)-piperidine (80)

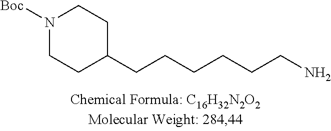 [SMILES: CC(C)(C)OC(=O)N1CCC(CCCCCCN)CC1]

Following General Procedure H part 2, Intermediate 79 (1 eq., 0.81 mmol, 336 mg) was dissolved in EtOH (12 mL). Then, hydrazine monohydrate (4 eq., 3.25 mmol, 162 μL) was added and the reaction mixture stirred overnight at r.t. The reaction progress was monitored by TLC (SiO₂, CHCl₃/MeOH/Et₃N: 10/1/1%). After completion, the work-up was performed as described in General Procedure H part 2, and the crude product was adsorbed on diatomaceous earth. Purification by flash chromatography, applying an isocratic method (CHCl₃/MeOH/Et₃N: 10/1/1%), afforded intermediate 80 (50% yield, 0.4 mmol, 115 mg) as oil. MS (ESI, positive mode): found m/z=285.3 [M+H]⁺, calculated m/z=285.2 [M+H]⁺.

### 3-((Benzyloxy)carbonyl)benzoic acid (81)

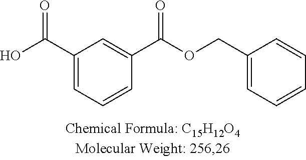 [SMILES: O=C(O)c1cccc(C(=O)OCc2ccccc2)c1]

### Part 1:

Isophthalic acid (1 eq., 20 mmol, 3.3 g) was dissolved in MeOH (resulting concentration=0.66 M, 30 mL). Then, H₂O (2 mL) and Et₃N (1 eq., 20 mmol, 2.8 mL) were added, and the reaction mixture was stirred overnight at r.t. After completion, the volatiles were evaporated under reduced pressure.

### Part 2:

The residue from Part 1 was dissolved in DMF (resulting concentration=0.66 M, 30 mL) and set under an argon atmosphere. Then, benzyl bromide (1.1 eq., 22 mmol, 2.62 mL) was added slowly added to the reaction mixture. The reaction was stirred for 2 h at 100° C. The reaction progress was monitored by TLC (SiO₂, CHCl₃/MeOH/AcOH: 15/1/1%). After completion, the reaction was stopped by dropping the temperature to 0° C. After the formation of a precipitate, an aqueous 5% NaHCO₃ solution (100 mL) was added and extracted EtOAc (3×60 mL). The aqueous layer was acidified with 2M HCl until a pH-value of 3 was reached, followed by an extraction with EtOAc (3×). The combined organic layers were dried over MgSO₄, and the volatiles were evaporated under reduced pressure. The crude product was adsorbed on diatomaceous earth and purification by flash chromatography, applying an isocratic method (CHCl₃/MeOH/AcOH: 18/1/1%), afforded intermediate 81 (40% yield, 8 mmol, 2 g) as pale brown crystals. ¹H-NMR (500 MHz, CDCl₃): δ/ppm=8.80 (s, 1H), 8.31 (d, J=7.7 Hz, 1H), 8.29 (d, J=7.7 Hz, 1H), 7.57 (td, J=7.7, 0.5 Hz, 1H), 7.51-7.44 (m, 2H), 7.44-7.32 (m, 3H), 5.41 (s, 2H). ¹³C-NMR (126 MHz, CDCl₃): δ/ppm=171.12, 165.49, 135.69, 134.78, 134.44, 131.45, 130.79, 129.84, 128.80, 128.67, 128.43, 128.34, 67.15. MS (ESI, negative mode): found m/z=255.1 [M−H]⁻, calculated m/z=253.2 [M−H]⁻.

### Tert-butyl (E)-4-(6-(3-(pyridin-3-yl)acrylamido)hexyl)piperidine-1-carboxylate (82)

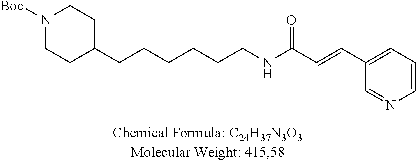 [SMILES: CC(C)(C)OC(=O)N1CCC(CCCCCCNC(=O)/C=C/c2cccnc2)CC1]

Following General Procedure F, intermediate 80 (1 eq., 2.09 mmol, 594 mg) and trans-3-(3-pyridyl)acrylic acid (1 eq., 2.09 mmol, 312 mg) were dissolved in DCM (abs. 1.7 mL). Then, DIPEA (1 eq., 2.09 mmol, 368 μL), HOBt (1 eq., 2.09 mmol, 283 mg) and DCC (1 eq., 2.09 mmol, 434 mg) were added sequentially and the resulting mixture stirred overnight at r.t. The reaction progress was monitored by (SiO₂, TLC DCM/MeOH: 10/1) and HPLC (method 11). After completion, the work-up was performed as described in General Procedure F and the crude product purified by preparative HPLC (method 11, t_{R}=17.34 min) affording intermediate 82 (35% yield, 0.73 mmol, 304 mg) as oil. MS (ESI, positive mode): found m/z=416.33 [M+H]⁺, calculated m/z=416.2 [M+H]⁺.

### (E)-N-(6-(piperidin-4-yl)hexyl)-3-(pyridin-3-yl)acrylamide (83)

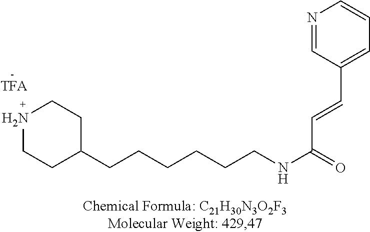 [SMILES: O=C(/C=C/c1cccnc1)NCCCCCCC1CCNCC1]

Following General Procedure C variation 1, intermediate 82 (1 eq., 0.73 mmol, 303 mg) was dissolved in pure TFA (2 mL) and the solution stirred at 400 mbar for 5 min. The reaction was monitored by TLC (SiO₂, CHCl₃/MeOH/Et₃N: 10/1/1%). After completion, the work-up was performed as described in General Procedure C affording intermediate 83 (80% yield, 0.584 mmol, 251 mg) as oil without further purification. MS (ESI, positive mode): found m/z=316.4 [M+H]⁺, calculated m/z=316.2 [M+H]⁺.

### Tert-butyl(E)-(3-(4-(6-(3-(pyridin-3-yl)acrylamido)hexyl)piperidine-1-carbonyl)phenyl)carbamate (84)

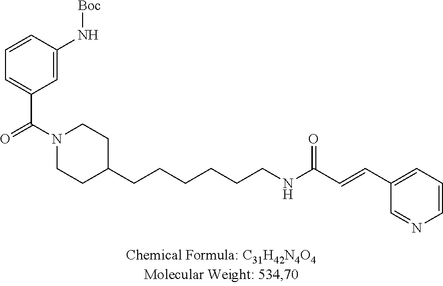 [SMILES: CC(C)(C)OC(=O)Nc1cccc(C(=O)N2CCC(CCCCCCNC(=O)/C=C/c3cccnc3)CC2)c1]

Following General Procedure F, intermediate 83 (1 eq., 0.132 mmol, 57 mg) and Boc-3-aminobenzoic acid (1 eq., 0.132 mmol, 31.3 mg) were dissolved in DCM (abs., 1.7 mL). Then, DIPEA (1 eq., 0.132 mmol, 23.2 μL), HOBt (1 eq., 0.132 mmol, 18 mg) and DCC (1 eq., 0.132 mmol, 27 mg) were added sequentially, and the resulting reaction mixture was stirred overnight at r.t. The reaction progress was monitored by TLC (SiO₂, hexane/EtOAc/MeOH: 10/10/1) and HPLC (method 2). After completion, the work-up was performed as described in General Procedure F and the crude product purified by preparative HPLC (method 2, t_{R}=16.76 min) affording intermediate 84 (40% yield, 0.05 mmol, 28 mg) as oil. MS (ESI, positive mode): found m/z=535.5 [M+H]⁺, calculated m/z=535.2 [M+H]⁺.

### (E)-N-(6-(1-(3-aminobenzoyl)piperidin-4-yl)hexyl)-3-(pyridin-3-yl)acrylamide (85)

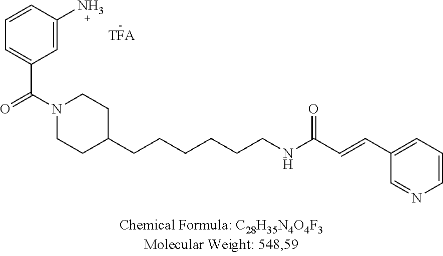 [SMILES: Nc1cccc(C(=O)N2CCC(CCCCCCNC(=O)/C=C/c3cccnc3)CC2)c1]

Following General Procedure C variation 1, intermediate 84 (1 eq., 0.73 mmol, 390 mg) was dissolved in pure TFA (2 mL) and the reaction stirred at 400 mbar for 5 min. The reaction was monitored by TLC (SiO₂, CHCl₃/MeOH/Et₃N: 10/1/1%). After completion, the work-up was performed as described in General Procedure C affording intermediate 85 (80% yield, 0.584 mmol, 320 mg) as oil without further purification. MS (ESI, positive mode): found m/z=435.4 [M+H]⁺, calculated m/z=435.2 [M+H]⁺.

### Benzyl 3-(4-(4-hydroxybutyl)piperidine-1-carbonyl)benzoate (86)

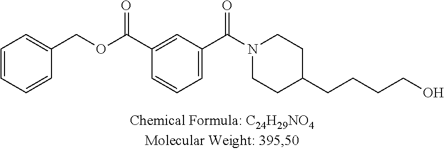 [SMILES: O=C(OCc1ccccc1)c1cccc(C(=O)N2CCC(CCCCO)CC2)c1]

Following General Procedure F, intermediate 81 (1 eq., 3.38 mmol, 866 mg) and intermediate 22 (1 eq., 3.38 mmol, 532 mg) were dissolved in DCM (abs., 43 mL). Then, DIPEA (1 eq., 3.38 mmol, 594 μL), HOBt (1 eq., 3.38 mmol, 461 mg) and DCC (1 eq., 3.38 mmol, 691 mg) were added sequentially and the resulting reaction mixture stirred overnight at r.t. The reaction progress was monitored by TLC (hexane/EtOAc/MeOH: 10/10/1). After completion, the work-up was performed as described in General Procedure F and the crude product adsorbed on diatomaceous earth. Purification by flash chromatography, applying an isocratic method (hexane/EtOAc/MeOH: 10/10/1), afforded intermediate 86 (38% yield, 1.28 mmol, 508 mg) as oil. MS (ESI, positive mode): found m/z=396.2 [M+H]⁺, calculated m/z=396.4 [M+H]⁺.

### Benzyl 3-(4-(4-((methylsulfonyl)oxy)butyl)piperidine-1-carbonyl)benzoate (87)

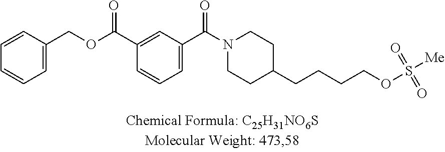 [SMILES: CS(=O)(=O)OCCCCC1CCN(C(=O)c2cccc(C(=O)OCc3ccccc3)c2)CC1]

Following General Procedure G, intermediate 86 (1 eq., 2.05 mmol, 811 mg) was dissolved in DCM (abs., 15 mL). The solution was cooled to 0° C. and methanesulfonyl chloride (1.5 eq., 3.1 mmol, 245 μL) was added followed by triethylamine (2 eq., 4.1 mmol, 572 μL). The reaction mixture was stirred for 1.5 h at r.t and the progress monitored by TLC (SiO₂, DCM/MeOH: 30/1). After completion, the work-up was performed as described in General Procedure G, and the crude product adsorbed on diatomaceous earth. Purification by flash chromatography, applying an isocratic method (DCM/MeOH: 30/1), afforded intermediate 87 (80% yield, 1.64 mmol, 777 mg) as an oil. MS (ESI, positive mode): found m/z=475.1 [M+H]⁺, calculated m/z=474.8 [M+H]⁺.

### Benzyl 3-(4-(4-((phthalimidyl)oxy)butyl)piperidine-1-carbonyl)benzoate (88)

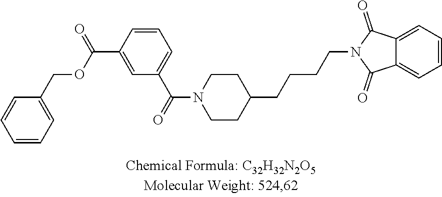 [SMILES: O=C(OCc1ccccc1)c1cccc(C(=O)N2CCC(CCCCN3C(=O)c4ccccc4C3=O)CC2)c1]

Following General Procedure H part 1, intermediate 87 (1 eq., 1.84 mmol, 871 mg) and potassium phthalimide (1.2 eq., 2.2 mmol, 414 mg) were dissolved in DMF (abs., 20 mL). Following the temperature conditions described in General Procedure H part 1, the reaction mixture was stirred overnight. The reaction progress was monitored by TLC (hexane/EtOAc/MeOH: 13/7/1). After completion, the work-up was performed as described in General Procedure H part 1 and the crude product adsorbed on diatomaceous earth. Purification by flash chromatography, applying an isocratic method (Hex/EtOAc/MeOH: 13/7/1), afforded intermediate 88 (65% yield, 1.2 mmol, 630 mg) as oil. ¹H-NMR (500 MHz, d6-DMSO): δ/ppm=8.05 (dt, J=7.7, 1.5 Hz, 1H), 7.92 (t, J=1.7 Hz, 1H), 7.92-7.77 (m, 5H), 7.60 (t, J=7.6 Hz, 1H), 7.53-7.32 (m, 5H), 5.37 (s, 2H), 3.57 (t, J=7.1 Hz, 2H), 1.78-0.97 (m, 14H). ¹³C-NMR (126 MHz, d6-DMSO): δ/ppm=167.80, 167.58, 164.90, 136.89, 135.85, 134.22, 131.50, 131.33, 129.84, 129.71, 128.97, 128.40, 128.04, 127.91, 127.20, 122.84, 66.33, 37.22, 35.13, 35.06, 27.97, 23.21. MS (ESI, positive mode): found m/z=525.5 [M+H]⁺, calculated m/z=525.2 [M+H]⁺.

### Benzyl 3-(4-(4-((amino)oxy)butyl)piperidine-1-carbonyl)benzoate (89)

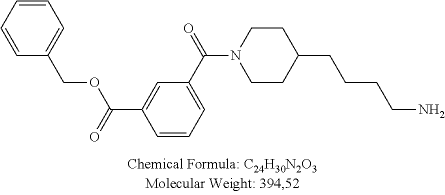 [SMILES: NCCCCC1CCN(C(=O)c2cccc(C(=O)OCc3ccccc3)c2)CC1]

Following General Procedure H part 2, Intermediate 88 (1 eq., 1.72 mmol, 902 mg) was dissolved in EtOH (32 mL). Then, hydrazine monohydrate (6 eq., 10.3 mmol, 513 μL) was added and the resulting reaction mixture stirred overnight at r.t. The reaction progress was monitored by TLC (SiO₂, CHCl₃/MeOH/Et₃N: 10/1/1%). After completion, the work-up was performed as described in General Procedure H part 2 and the crude product adsorbed on diatomaceous earth. Purification by flash chromatography, applying an isocratic method (CHCl₃/MeOH/Et₃N: 10/1/1%), afforded intermediate 89 (55% yield, 0.95 mmol, 373 mg) as pale-yellow crystals. ¹H-NMR (500 MHz, d6-DMSO): δ/ppm=8.05 (dt, J=7.7, 1.5 Hz, 1H), 7.93-7.91 (m, 1H), 7.66 (dt, J=7.6, 1.5 Hz, 1H), 7.60 (td, J=7.6, 0.6 Hz, 1H), 7.45-7.27 (m, 5H), 5.37 (s, 2H), 2.90-2.58 (m, 4H), 2.42 (q, J=7.1 Hz, 2H), 1.41-1.14 (m, 11H). ¹³C-NMR (126 MHz, d6-DMSO): δ/ppm=167.61, 164.92, 131.33, 129.85, 128.99, 128.41, 128.06, 127.92, 127.88, 127.86, 127.20, 66.34, 41.39, 35.60, 35.36, 35.19, 35.12, 33.16, 29.06, 23.39, 23.29, 11.69. MS (ESI, positive mode): found m/z=395.4 [M+H]⁺, calculated m/z=395.4 [M+H]⁺.

### Benzyl(E)-3-(4-(4-(2-cyano-3-(pyridin-3-yl)guanidino)butyl)piperidine-1-carbonyl)benzoate (90)

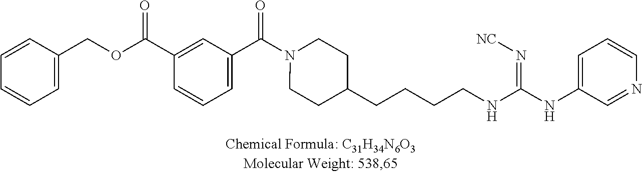 [SMILES: N#C/N=C(\NCCCCC1CCN(C(=O)c2cccc(C(=O)OCc3ccccc3)c2)CC1)Nc1cccnc1]

Following General Procedure B, intermediate 89 (1 eq., 0.51 mmol, 201 mg) and intermediate 1 (1.1 eq., 0.56 mmol, 135 mg) were dissolved in 1,4-dioxane (12 mL). Triethylamine (2.4 eq., 1.22 mmol, 3.3 μL) was added and the resulting reaction mixture stirred overnight at r.t. The reaction progress was monitored by TLC (SiO₂, CHCl₃/MeOH: 15/1). After completion, the work-up was performed as described in General Procedure B and the crude product adsorbed on diatomaceous earth. Purification by flash chromatography, applying an isocratic method (DCM/MeOH: 9/1), afforded intermediate 90 (40% yield, 0.2 mmol, 110 mg) as sticky oil. ¹H-NMR (500 MHz, d6-DMSO): δ/ppm=9.01 (s, 1H), 8.46 (dd, J=2.6, 0.8 Hz, 1H), 8.33 (dd, J=4.7, 1.5 Hz, 1H), 8.05 (dt, J=7.7, 1.5 Hz, 1H), 7.92 (td, J=1.8, 0.6 Hz, 1H), 7.66 (dt, J=7.6, 1.5 Hz, 2H), 7.60 (td, J=7.7, 0.6 Hz, 1H), 7.48 (d, J=6.7 Hz, 1H), 7.44-7.32 (m, 5H), 5.37 (s, 2H), 3.45 (qd, J=7.0, 5.1 Hz, 2H), 3.23 (q, J=7.0 Hz, 2H), 1.51 (p, J=7.1 Hz, 3H), 1.36-1.23 (m, 5H), 1.06 (t, J=7.0 Hz, 5H). ¹³C-NMR (126 MHz, d6-DMSO): δ/ppm=167.62, 164.92, 157.95, 145.29, 144.72, 136.91, 135.86, 134.56, 131.33, 130.72, 129.86, 129.74, 128.99, 128.41, 128.07, 127.92, 127.21, 123.47, 116.83, 66.35, 55.92, 41.45, 35.23, 35.09, 28.85, 23.07, 18.42. MS (ESI, positive mode): found m/z=539.5 [M+H]⁺, calculated m/z=539.4 [M+H]⁺.

### N-(3-(4-(4-((E)-3-(pyridin-3-yl)acrylamido)butyl)piperidine-1-carbonyl)phenyl)biotinamide (91)

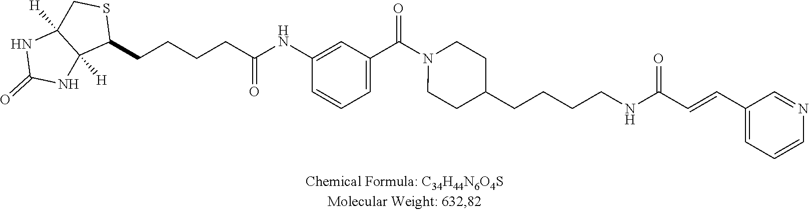

Following General Procedure K, biotin (1 eq., 0.071 mmol, 17.3 mg) and HATU (1.1 eq., 0.08 mmol, 30.4 mg) were dissolved in DMF (abs., 5 mL). DIPEA (2 eq., 0.14 mmol, 24 μL) was added and the resulting reaction mixture stirred for 20 min at r.t. Then, intermediate 57 (1 eq., 0.071 mmol, 37 mg) was added and the reaction stirred for 48 h. The reaction progress was monitored by TLC (SiO₂, CHCl₃/MeOH 10/1) and HPLC (method 2). After completion, the work-up was performed as described in General Procedure K and purified by preparative HPLC (method 2, t_{R}=12.86 min), affording intermediate 91 (42% yield, 0.03 mmol, 20 mg) as a white solid after lyophilisation. ¹H-NMR (500 MHz, d6-DMSO): δ/ppm=10.16 (s, 1H), 9.77 (s, 1H), 8.73 (d, J=2.2 Hz, 1H), 8.53 (d, J=4.6 Hz, 1H), 8.07 (s, 1H), 7.95 (d, J=8.1 Hz, 1H), 7.64 (s, 1H), 7.57 (d, J=8.0 Hz, 1H), 7.51 (d, J=8.3 Hz, 2H), 7.47-7.38 (m, 2H), 7.33 (t, J=7.8 Hz, 1H), 7.22 (d, J=8.3 Hz, 2H), 7.00 (d, J=7.5 Hz, 1H), 6.70 (d, J=15.9 Hz, 1H), 6.35 (s, 1H), 6.29 (s, 1H), 3.57 (s, 2H), 3.18-2.52 (m, 11H), 1.72-0.82 (m, 28H). MS (ESI, positive mode): found m/z=633.7 [M+H]⁺, calculated m/z=633.4 [M+H]⁺.

### (E)-3-(4-(4-(2-cyano-3-(pyridin-3-yl)guanidino)butyl)piperidine-1-carbonyl)benzoic acid (92)

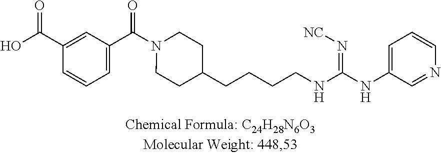 [SMILES: N#C/N=C(\NCCCCC1CCN(C(=O)c2cccc(C(=O)O)c2)CC1)Nc1cccnc1]

Following General Procedure E, intermediate 90 (1 eq., 0.18 mmol, 97 mg) was dissolved in a solvent mixture of EtOAc (5 mL) and EtOH (5 mL). Then, Pd/C (10% in Pd, 12 mol-%, 50 mg) was added and the hydrogenation process started according to General Procedure E. The reaction proceeded overnight and the progress was monitored by (SiO₂, TLC CHCl₃/MeOH: 10/1). The work-up was performed as described in General Procedure E. Without further purification, intermediate 92 (80% yield, 0.14 mmol, 65 mg) was afforded as a white powder. MS (ESI, positive mode): found m/z=449.3 [M+H]⁺, calculated m/z=449.4 [M+H]⁺.

### 4-((S)-2-((S)-2-(6-(2,5-dioxo-2,5-dihydro-1H-pyrrol-1-yl)hexanamido)-3-methylbutanamido) propanamido)benzyl 3-(4-(4-((E)-2-cyano-3-(pyridin-3-yl)guanidino)butyl)piperidine-1-carbonyl) benzoate (93)

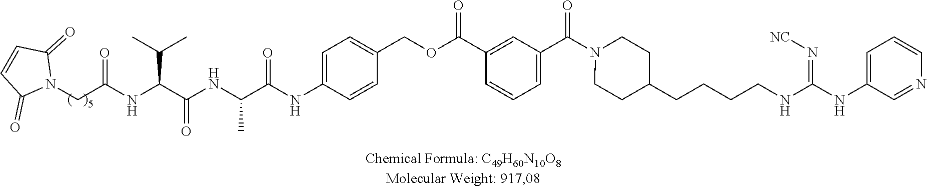

Intermediate 92 (1 eq., 10 μmol, 4.5 mg) was dissolved in DMF (resulting concentration=50 mM, 0.2 mL), followed by the addition of 0.1 mL of a stock solution containing 1.1 M DIC and 1.1 M HOBt in DMF (10 eq. each, 0.111 mmol). The reaction mixture was pre-activated for 40 min. Next, mc-Val-Ala-PAB-OH (CAS-No. 1870916-87-2, 81.26 mg, 0.17 mmol) and DIPEA (48.34 μL, 0.28 mmol) were dissolved in 1 mL DMF (=master mix two). Master mix two (0.2 mL containing 0.03 mmol=3 eq. mc-Val-Ala-PAB-OH and 0.055 mmol=5 eq. DIPEA) was added to the reaction mixture containing intermediate 92, DIC and HOBt. The reaction mixture was stirred for 24 h and the progress was monitored by TLC (SiO₂, DCM/MeOH 9/1) and HPLC (method 11). After completion, the volatiles were evaporated under reduced pressure, and the crude product was purified by preparative HPLC (method 11, t_{R}=13.43 min) affording intermediate 93 (20% yield, 0.002 mmol, 1.83 mg) as white solid. MS (ESI, positive mode): found m/z=917.7 [M+H]⁺, calculated m/z=917.4 [M+H]⁺.

### (E)-1-(4-(1-(3-aminobenzoyl)-4-hydroxypiperidin-4-yl)butyl)-2-cyano-3-(pyridin-3-yl)guanidine (94)

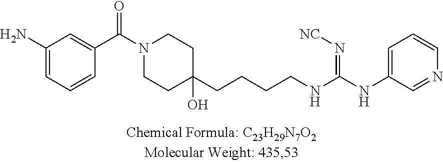 [SMILES: N#C/N=C(\NCCCCC1(O)CCN(C(=O)c2cccc(N)c2)CC1)Nc1cccnc1]

Following General Procedure E, intermediate 78 (1 eq., 0.02 mmol, 11.4 mg) was dissolved in a solvent mixture of EtOAc (1 mL) and EtOH (1 mL). Then, Pd/C (10% in Pd, 12 mol-%, 15 mg) was added and the hydrogenation process was started according to General Procedure E. The reaction proceeded for 4 h and the progress was monitored by TLC (SiO₂, CHCl₃/MeOH/Et₃N: 15/1/1%). The work-up was performed as described in General Procedure E. Without further purification, intermediate 94 (80% yield, 0.016 mmol, 7 mg) was afforded as a white powder. ¹H-NMR (500 MHz, d6-DMSO): δ/ppm=8.38 (d, J=2.6 Hz, 1H), 8.28 (dd, J=4.8, 1.5 Hz, 1H), 7.64 (dt, J=8.1, 2.1 Hz, 1H), 7.44-7.30 (m, 1H), 7.05 (t, J=7.8 Hz, 1H), 6.62 (ddd, J=8.1, 2.3, 1.0 Hz, 1H), 6.51 (t, J=1.9 Hz, 1H), 6.46 (dt, J=7.5, 1.3 Hz, 1H), 3.98-3.93 (m, 4H), 3.56-3.52 (m, 6H), 1.57-1.14 (m, 10H). ¹³C-NMR (126 MHz, d6-DMSO): δ/ppm=169.36, 157.91, 148.51, 145.24, 144.68, 137.13, 134.59, 130.66, 128.63, 123.45, 118.45, 116.83, 114.37, 113.52, 111.68, 66.25, 41.95, 41.55, 29.40, 19.53. MS (ESI, positive mode): found m/z=436.33 [M+H]⁺, calculated m/z=436.4 [M+H]⁺.

### tert-butyl (2-(1,3-dioxoisoindolin-2-yl)ethoxy)carbamate (95)²¹¹

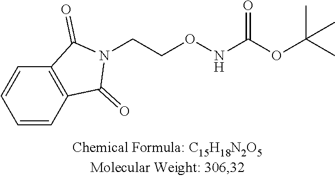 [SMILES: CC(C)(C)OC(=O)NOCCN1C(=O)c2ccccc2C1=O]

Following WO2006069246, potassium carbonate (2.33 eq., 87.6 mmol, 12 g) and N-(2-bromoethyl)phthalimide (1.05 eq., 39.7 mmol, 10 g) were added to a solution of tert-butyl N-hydroxycarbamate (1 eq., 37.6 mmol, 5 g) in DMF (resulting concentration=1.2 M, 30 mL) at 0° C. The reaction mixture was stirred for 3 h at r.t and the progress was monitored by TLC (SiO₂, hexane/EtOAc 1:1). After completion, the mixture was diluted in H₂O (200 mL) and extracted with EtOAC (200 mL). The organic layer was collected and washed once with H₂O and once with brine. The organic layer was dried over MgSO₄, and the volatiles were evaporated under reduced pressure. The crude product was adsorbed on diatomaceous earth, and purification by flash chromatography, applying a gradient (20:1-1:1 hexane:EtOAc, 30 min), afforded intermediate 95 (55% yield, 21 mmol, 6.3 g) as white solid. MS (ESI, positive mode): found m/z=307.0 [M+H]⁺ and 324.2 [M+NH₄, calculated m/z=307.4 [M+H]⁺.

### tert-Butyl (2-aminoethoxy)carbamate (96)

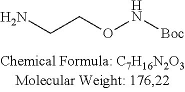 [SMILES: CC(C)(C)OC(=O)NOCCN]

Following General Procedure H part 2, Intermediate 95 (1 eq., 3.69 mmol, 1.1 g) was dissolved in EtOH (23 mL). Then, hydrazine monohydrate (5 eq., 10.3 mmol, 921 μL) was added and the reaction mixture stirred for 5 h at r.t. The progress was monitored by TLC (SiO₂, CHCl₃/MeOH/Et₃N: 10/1/1%). After completion, the work-up was performed as described in General Procedure H part 2, and the crude product adsorbed on diatomaceous earth. Purification by flash chromatography, applying an isocratic method (CHCl₃/MeOH/Et₃N: 9/1/1%), afforded intermediate 96 (50% yield, 1.8 mmol, 325 mg) as crystals. MS (ESI, positive mode): found m/z=177.2 [M+H]⁺, calculated m/z=177.4 [M+H]⁺.

### 4-((S)-2-((S)-2-(6-(2,5-dioxo-2,5-dihydro-1H-pyrrol-1-yl)hexanamido)-3-methylbutanamido)propanamido)benzyl(3-(4-(4-((E)-2-cyano-3-(pyridin-3-yl)guanidino)butyl)-4-hydroxypiperidine-1-carbonyl)phenyl)carbamate (97)

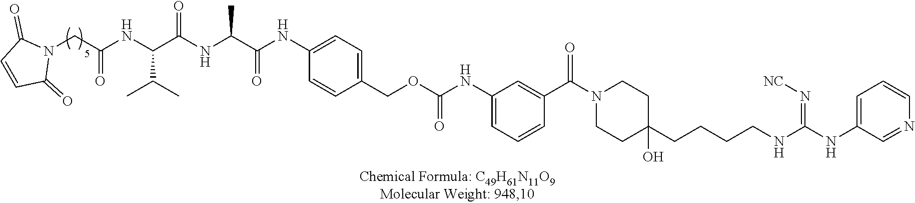

Following General Procedure J, mc-Val-Ala-PAB-PNP (CAS-No. 1639939-40-4, 2 eq., 0.014 mmol, 9 mg) was dissolved in DMF (0.5 mL). Then, PyAOP (2 eq., 0.014 mmol, 7 mg) and DIPEA (4 eq., 0.028 mmol, 5 μL) were added and stirred for 30 min at r.t. Then, intermediate 94 (1 eq., 0.007 mmol, 3 mg) was added and the resulting reaction mixture stirred overnight at r.t. The reaction progress was monitored by HPLC (method 2). After completion, reaction mixture is directly purified by preparative HPLC (method 2, t_{R}=14.57 min) affording intermediate 97 (12% yield, 0.8 μmol, 0.8 mg) as a white solid. MS (ESI, negative mode): found m/z=946.5 [M−H]⁻, calculated m/z=946.4 [M−H]⁻.

### tert-butyl (E)-(2-(3-(pyridin-3-yl)acrylamido)ethoxy)carbamate (98)

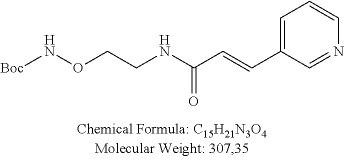 [SMILES: CC(C)(C)OC(=O)NOCCNC(=O)/C=C/c1cccnc1]

Following General Procedure F, intermediate 96 (1 eq., 1.95 mmol, 344 mg) and trans-3-(3-pyridyl)acrylic acid (1 eq., 1.95 mmol, 291 mg) were dissolved in DCM (abs., 25 mL). Then, DIPEA (1 eq., 1.95 mmol, 340 μL), HOBt (1 eq., 1.95 mmol, 263 mg) and DCC (1 eq., 1.95 mmol, 402 mg) were sequentially added and the resulting reaction mixture stirred overnight at r.t. The reaction progress was monitored by TLC (SiO₂, DCM/MeOH: 10/1). After completion, the work-up was performed as described in General Procedure F, and the crude product adsorbed on diatomaceous earth. Purification by flash chromatography, applying an isocratic method (DCM/MeOH: 10/1), afforded intermediate 98 (35% yield, 0.68 mmol, 210 mg) as oil. MS (ESI, positive mode): found m/z=308.2 [M+H]⁺, calculated m/z=308.4 [M+H]⁺.

### (E)-N-(2-(aminooxy)ethyl)-3-(pyridin-3-yl)acrylamide (99)

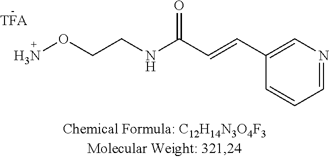 [SMILES: NOCCNC(=O)/C=C/c1cccnc1 ; "TFA"]

Following General Procedure C variation 2, Intermediate 98 (1 eq., 0.52 mmol, 160 mg) was dissolved in a 15% TFA solution in DCM (15 mL). The reaction mixture was stirred for 2 h at r.t. and the progress monitored by TLC (SiO₂, CHCl₃/MeOH/Et₃N: 10/1/1%). After completion, the work-up was performed as described in General Procedure C, and without further purification, intermediate 99 (83% yield, 0.43 mmol, 139 mg) was afforded as oil.

### tert-butyl (E)-4-((2-(3-(pyridin-3-yl)acrylamido)ethoxy)imino)piperidine-1-carboxylate (100)

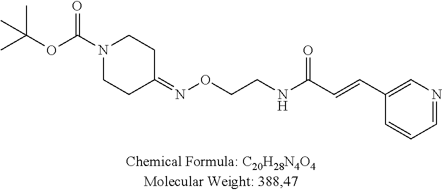 [SMILES: CC(C)(C)OC(=O)N1CCC(=NOCCNC(=O)/C=C/c2cccnc2)CC1]

Intermediate 99 (1 eq., 0.52 mmol, 167 mg) and N-Boc-piperid-4-one (1 eq., 0.52 mmol, 104 mg) were dissolved in ethanol (resulting concentration=0.13 M, 4 mL). After solubilisation, sodium acetate (2 eq., 1.04 mmol, 85 mg) was added and the resulting reaction mixture stirred for 6 h at r.t. The reaction was monitored by TLC (SiO₂, DCM/MeOH: 10/1). After completion, the volatiles were evaporated under reduced pressure. The residue was then redissolved in chloroform (100 mL) and the organic layer once washed with an aqueous, saturated sodium bicarbonate solution (50 mL) and once with water (50 mL). Then, the combined organic layers were dried over MgSO₄ and the volatiles evaporated under reduced pressure. The crude product was adsorbed on diatomaceous earth, and purification by flash chromatography, applying a gradient method (100% DCM to 100% DCM/MeOH: 9/1 in 15 min), afforded intermediate 100 (70% yield, 0.43 mmol, 168 mg) as oil. MS (ESI, positive mode): found m/z=389.2 [M+H]⁺, calculated m/z=389.4 [M+H]⁺.

### (E)-N-(2-((piperidin-4-ylideneamino)oxy)ethyl)-3-(pyridin-3-yl)acrylamide, TFA salt (101)

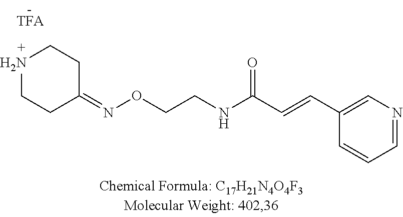 [SMILES: O=C(/C=C/c1cccnc1)NCCON=C1CCNCC1]

Following General Procedure C variation 2, intermediate 100 (1 eq., 0.31 mmol, 120 mg) was dissolved in a 15% TFA solution in DCM (15 mL). The reaction mixture was stirred at r.t. for 2 h. The progress was monitored by TLC (SiO₂, CHCl₃/MeOH/Et₃N: 10/1/1%). After completion, the work-up was performed as described in General Procedure C, and without further purification, intermediate 101 (72% yield, 0.22 mmol, 90 mg) was afforded as oil.

### tert-butyl(E)-(3-(4-((2-(3-(pyridin-3-yl)acrylamido)ethoxy)imino)piperidine-1-carbonyl)phenyl)carbamate (102)

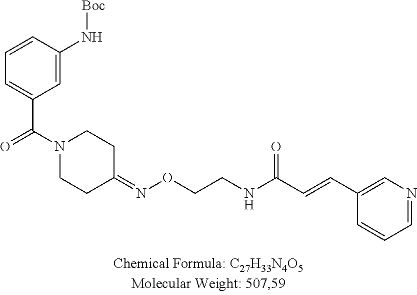 [SMILES: CC(C)(C)OC(=O)Nc1cccc(C(=O)N2CCC(=NOCCNC(=O)/C=C/c3cccnc3)CC2)c1]

Following General Procedure F, intermediate 101 (1 eq., 0.33 mmol, 133 mg) and Boc-3-aminobenzoic acid (2 eq., 0.66 mmol, 157 mg) were dissolved in dry-DCM (5.5 mL). Then, DIPEA (2.2 eq., 0.73 mmol, 127 μL), HOBt (2 eq., 0.66 mmol, 89 mg) and DCC (2 eq., 0.66 mmol, 136 mg) were added sequentially and the resulting reaction mixture stirred overnight at r.t. The reaction progress was monitored by TLC (SiO₂, DCM/MeOH: 9/1). After completion, the work-up was performed as described in General Procedure F and the crude product adsorbed on diatomaceous earth. Purification by flash chromatography, applying an isocratic method (DCM/MeOH: 10/1), afforded intermediate 102 (35% yield, 0.12 mmol, 59 mg) as oil. MS (ESI, positive mode): found m/z=508.2 [M+H]⁺, calculated m/z=508.4 [M+H]⁺.

### (2S,3R,4S,5S,6S)-2-(2-(3-((((9H-fluoren-9-yl)methoxy)carbonyl)amino)propanamido)-4-((((3-(4-(4-((E)-2-cyano-3-(pyridin-3-yl)guanidino)butyl)piperidine-1-carbonyl)phenyl)carbamoyl)oxy)methyl)phenoxy)-6-(methoxycarbonyl)tetrahydro-2H-pyran-3,4,5-triyl triacetate (103)

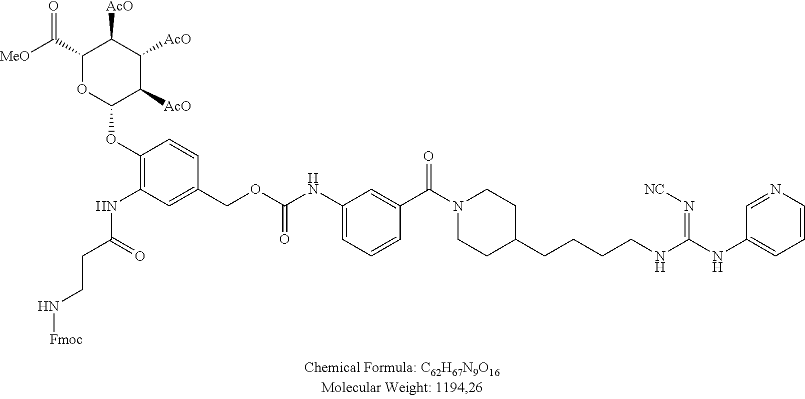

Following General Procedure J, N-Fmoc-O—Ac-β-D-glucuronide-PNP-carbonate (CAS-Nr. 894095-98-8, 1.5 eq., 0.036 mmol, 33 mg) was dissolved in DMF (1.5 mL). Then, PyAOP (1.5 eq., 0.036 mmol, 18 mg) and DIPEA (6 eq., 0.14 mmol, 25 μL) were added and stirred for 30 min at r.t. Next, intermediate 36 (1 eq., 0.024 mmol, 10 mg) was added, and the reaction mixture was stirred overnight at r.t. The reaction progress was monitored by HPLC (method 11). After completion, the reaction mixture was directly purified by preparative HPLC (method 11, t_{R}=18.32 min), affording intermediate 103 (10% yield, 0.0024 mmol, 3 mg) as a white solid. MS (ESI, positive mode): found m/z=1194.4 [M+H]⁺, calculated m/z=1194.5 [M+H]⁺.

### (2S,3R,4S,5S,6S)-2-(2-(3-((((9H-fluoren-9-yl)methoxy)carbonyl)amino)propanamido)-4-((((3-(4-(4-(€-2-cyano-3-(pyridine-3-yl)guanidino)butyl)-4-hydroxypiperidine-1-carbonyl)phenyl)carbamoyl)oxy)methyl)phenoxy)-6-(methoxycarbonyl)tetrahydro-2H-pyran-3,4,5-triyl triacetate (104)

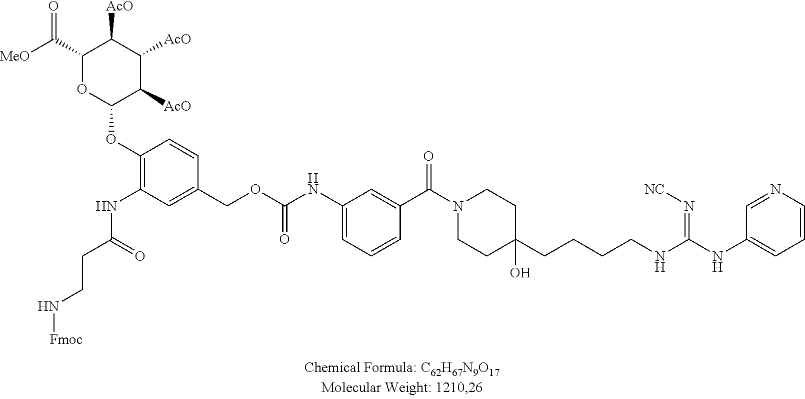

Following General Procedure J, N-Fmoc-O—Ac-β-D-glucuronide-PNP-carbonate (CAS-Nr. 894095-98-8, 1.5 eq., 0.036 mmol, 31 mg) was dissolved in DMF (1.4 mL). PyAOP (1.5 eq., 0.036 mmol, 17 mg) and DIPEA (6 eq., 0.13 mmol, 23 μL) were added and stirred for 30 min at r.t. Next, intermediate 94 (1 eq., 0.023 mmol, 10 mg) was added and the reaction mixture stirred overnight at r.t. The reaction end point was followed by HPLC (method 11). After completion, the reaction mixture was directly purified by preparative HPLC (method 11, t_{R}=18.54 min), affording intermediate 104 (11% yield, 0.0025 mmol, 3 mg) as a white solid. MS (ESI, positive mode): found m/z=1210.4 [M+H]⁺, calculated m/z=1210.5 [M+H]⁺.

### (E)-N-(2-(((1-(3-aminobenzoyl)piperidin-4-ylidene)amino)oxy)ethyl)-3-(pyridin-3-yl)acrylamide, TFA salt (105)

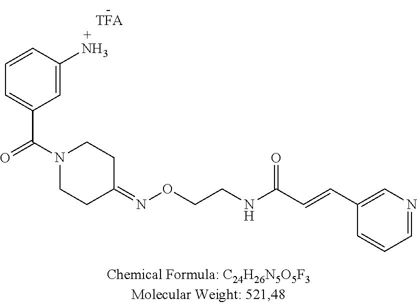 [SMILES: Nc1cccc(C(=O)N2CCC(=NOCCNC(=O)/C=C/c3cccnc3)CC2)c1]

Following General Procedure C variation 2, Intermediate 102 (1 eq., 0.04 mmol, 20 mg) was dissolved in a solution of 15% TFA in DCM (15 mL). The reaction mixture was stirred at r.t. for 1.5 h. The reaction progress was monitored by TLC (SiO₂, CHCl₃/MeOH/Et₃N: 10/1/1%). After completion, the work-up was performed as described in General Procedure C, affording intermediate 105 (73% yield, 0.03 mmol, 15 mg) as oil without further purification. MS (ESI, positive mode): found m/z=408.3 [M+H]⁺, calculated m/z=408.4 [M+H]⁺.

### (2S,3S,4S,5R,6S)-6-(2-(3-aminopropanamido)-4-((((3-(4-(4-((E)-2-cyano-3-(pyridin-3-yl)guanidino)butyl)piperidine-1-carbonyl)phenyl)carbamoyl)oxy)methyl)phenoxy)-3,4,5-trihydroxytetrahydro-2H-pyran-2-carboxylic acid (106)

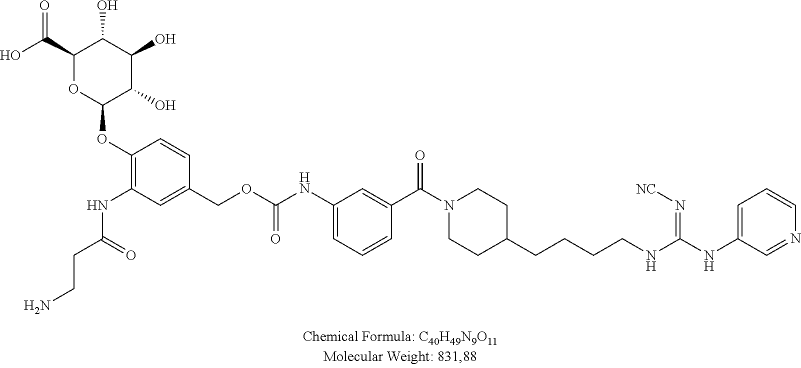 [SMILES: N#C/N=C(\NCCCCC1CCN(C(=O)c2cccc(NC(=O)OCc3ccc(O[C@H]4O[C@@H](C(=O)O)[C@H](O)[C@@H](O)[C@@H]4O)c(NC(=O)CCN)c3)c2)CC1)Nc1cccnc1]

Following General Procedure Q, intermediate 103 (1 eq., 0.01 mmol, 12 mg) was dissolved in MeOH (0.67 mL) followed by the addition of an aqueous 0.23 M LiOH solution (0.67 mL). The solution was stirred for 15 min and the reaction progress monitored by HPLC (method 2). After completion, the work-up was performed as described in General Procedure Q. Purification by RP-HPLC (method 2, t_{R}=9.12 min) afforded intermediate 106 (60% yield, 0.006 mmol, 5 mg) as an oil.

### (2S,3S,4S,5R,6S)-6-(2-(3-aminopropanamido)-4-((((3-(4-(4-((E)-2-cyano-3-(pyridin-3-yl)guanidino)butyl)-4-hydroxypiperidine-1-carbonyl)phenyl)carbamoyl)oxy)methyl)phenoxy)-3,4,5-trihydroxytetrahydro-2H-pyran-2-carboxylic acid (107)

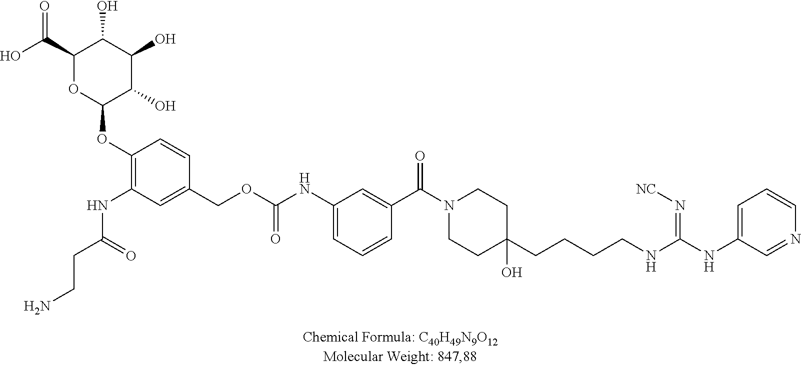 [SMILES: N#C/N=C(\NCCCCC1(O)CCN(C(=O)c2cccc(NC(=O)OCc3ccc(O[C@H]4O[C@@H](C(=O)O)[C@H](O)[C@@H](O)[C@@H]4O)c(NC(=O)CCN)c3)c2)CC1)Nc1cccnc1]

Following General Procedure Q, Intermediate 104 (1 eq., 0.007 mmol, 8.5 mg) was dissolved in MeOH (0.5 mL) followed by the addition of an aqueous 0.23 M LiOH solution (0.5 mL). The solution was stirred for 15 min at r.t. and the reaction progress was monitored by HPLC (method 2). After completion, the work-up was performed as described in General Procedure Q. Purification by HPLC (method 2, t_{R}=8.50 min) afforded intermediate 107 (58% yield, 4 μmol, 3 mg) as an oil.

### (2S,3S,4S,5R,6S)-6-(4-((((3-(4-(4-((E)-2-cyano-3-(pyridin-3-yl)guanidino)butyl)piperidine-1-carbonyl)phenyl)carbamoyl)oxy)methyl)-2-(3-(6-(2,5-dioxo-2,5-dihydro-1H-pyrrol-1-yl)hexanamido)propanamido)phenoxy)-3,4,5-trihydroxytetrahydro-2H-pyran-2-carboxylic acid (108)

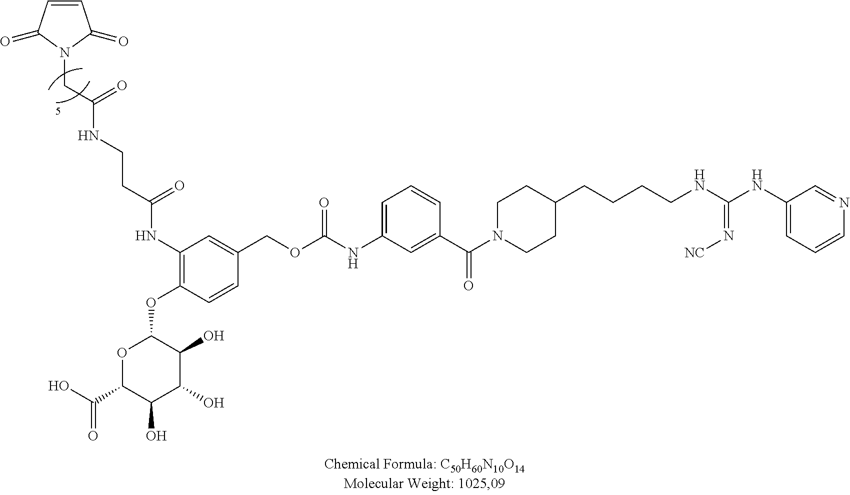

Following General Procedure I, intermediate 106 (1.2 eq., 0.001 mmol, 0.83 mg) and EMCS (1.5 eq., 0.015 mmol, 4.6 mg) were dissolved in DMF (abs., 0.8 mL). DIPEA (3.3 eq., 0.033 mmol, 5.7 μL) was added and the reaction stirred for 15 min at r.t. The reaction progress was monitored by HPLC (method 2). After completion, the work-up was performed as described in General Procedure I and purification by preparative HPLC (method 2, t_{R}=7.10 min) afforded intermediate 108 (50% yield, 5×10⁻⁴ mmol, 0.51 mg) as a white solid. MS (ESI, positive mode): found m/z=1025.3 [M+H]⁺, calculated m/z=1025.4 [M+H]⁺.

### (2S,3S,4S,5R,6S)-6-(4-((((3-(4-(4-((E)-2-cyano-3-(pyridin-3-yl)guanidino)butyl)-4-hydroxypiperidine-1-carbonyl)phenyl)carbamoyl)oxy)methyl)-2-(3-(6-(2,5-dioxo-2,5-dihydro-1H-pyrrol-1-yl)hexanamido)propanamido)phenoxy)-3,4,5-trihydroxytetrahydro-2H-pyran-2-carboxylic acid (109)

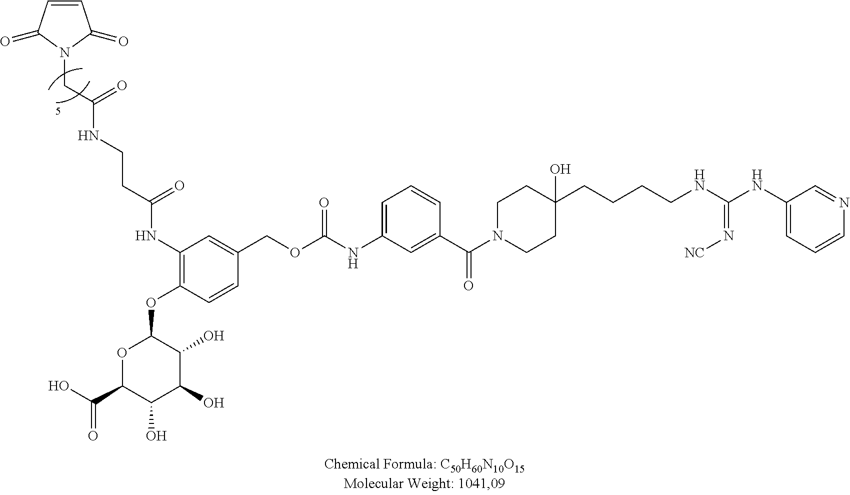

Following General Procedure I, intermediate 107 (1 eq., 0.007 mmol, 6 mg) and EMCS (1.5 eq., 0.011 mmol, 3.37 mg) were dissolved in DMF (abs., 0.6 mL). Then, DIPEA (3.3 eq., 0.025 mmol, 4.3 μL) was added and the reaction mixture stirred for 30 min at r.t. The reaction progress was monitored by HPLC (method 2). After completion, the work-up was performed as described in General Procedure I and purification by preparative HPLC (method 2, t_{R}=5.40 min) afforded intermediate 109 (51% yield, 4 μmol, 3.7 mg) as a white solid. MS (ESI, positive mode): found m/z=1041.4 [M+H]⁺, calculated m/z=1041.4 [M+H]⁺.

### 4-((S)-2-((S)-2-(6-(2,5-dioxo-2,5-dihydro-1H-pyrrol-1-yl)hexanamido)-3-methylbutanamido)propanamido)benzyl(3-(4-((2-((E)-3-(pyridin-3-yl)acrylamido)ethoxy)imino) piperidine-1-carbonyl)phenyl)carbamate (110)

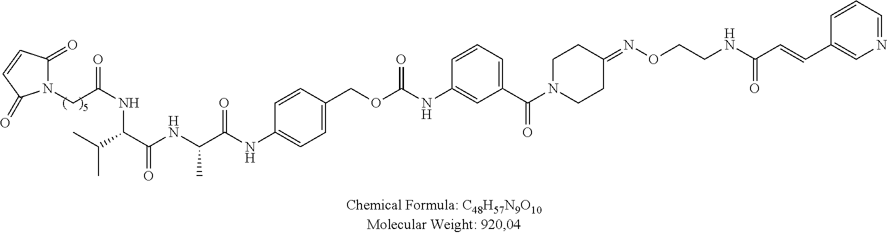

Following General Procedure J, mc-Val-Ala-PAB-PNP (CAS-No. 1639939-40-4, 2 eq., 40 μmol, 26 mg) was dissolved in DMF (1.4 mL). PyAOP (2 eq., 0.039 mmol, 18 mg) and DIPEA (5 eq., 0.1 mmol, 18 μL) were added and stirred for 30 min at r.t. Next, intermediate 105 (1 eq., 0.02 mmol, 10.4 mg) was added, and the reaction mixture was stirred overnight at r.t. The reaction progress was monitored by HPLC (method 2). After completion, the reaction mixture was directly purified by preparative HPLC (method 2, t_{R}=16.97 min), affording intermediate 110 (17% yield, 0.003 mmol, 3 mg) as a white solid. MS (ESI, positive mode): found m/z=920.4 [M+H]⁺, calculated m/z=920.4 [M+H]⁺.

### (E)-1-(((4-(1-(3-aminobenzoyl)piperidin-4-yl)butyl)amino)(pyridin-3-ylamino)methylene)urea (113)

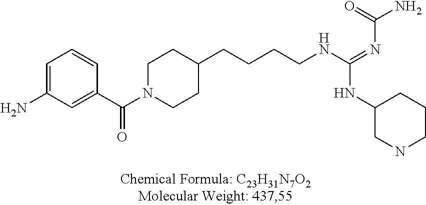 [SMILES: NC(=O)/N=C(\NCCCCC1CCN(C(=O)c2cccc(N)c2)CC1)NC1CCCNC1]

Intermediate 36 (1 eq., 0.01 mmol, 4.2 mg) was dissolved in a premixed solution of MeOH/H₂O (4/1) and TFA (until pH=1) (5 mL). The reaction mixture was stirred overnight at 37° C. and the progress monitored by HPLC (method 2). After completion, the reaction mixture was directly purified by preparative HPLC (method 2, t_{R}=9.76 min), affording intermediate 113 (95% yield, 0.009 mmol, 4.2 mg) as a white solid. ¹H-NMR (500 MHz, CD₃OD): δ/ppm=8.66-8.58 (m, 2H), 7.90 (ddd, J=8.2, 2.6, 1.5 Hz, 1H), 7.62 (ddd, J=8.2, 4.9, 0.8 Hz, 1H), 7.47 (t, J=7.8 Hz, 1H), 7.26 (ddd, J=8.1, 2.3, 1.0 Hz, 1H), 7.22 (dt, J=7.6, 1.3 Hz, 1H), 7.18 (t, J=1.9 Hz, 1H), 4.61 (d, J=13.0 Hz, 1H), 3.75-3.41 (m, 4H), 3.34 (s, 1H), 1.93-1.07 (m, 11H). ¹³C-NMR (126 MHz, CD₃OD): δ/ppm=171.15, 162.44, 162.15, 156.54, 149.68, 148.12, 138.93, 137.06, 131.25, 126.57, 124.28, 122.71, 119.72, 118.99, 43.72, 43.32, 33.88, 32.98, 29.58, 24.73, 18.36. MS (ESI, positive mode): found m/z=438.3 [M+H]⁺, calculated m/z=438.4 [M+H]⁺.

### N-Boc-4-(pent-4-(en)-yl)-4-hydroxy-piperidine (114)

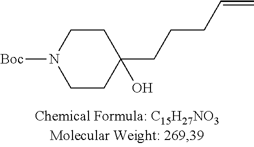 [SMILES: C=CCCCC1(O)CCN(C(=O)OC(C)(C)C)CC1]

Following General Procedure M, magnesium (turnings; 2 eq., 46.6 mmol, 1.1 g) and the tip of a spatula of 12 were charged in a dried three-neck flask under an argon atmosphere. THF (abs., 46.3 mL) was added and activation of magnesium performed as described in General Procedure M. Then, 5-bromopentene (1.5 eq., 34.9 mmol, 4.2 mL) was added as described in General Procedure M (solution A). In parallel, N-Boc-4-piperidone (1 eq., 23.3 mmol, 4.6 g) was dissolved in a LaCl₃*2LiCl solution in THF (0.6 M, 38.7 mL) in a dried two-neck flask. The mixture was activated for 1 h at r.t. followed by cooling to 8° C. (solution B). Next, solution A was added dropwise to solution B at 0° C., and the resulting reaction mixture was stirred overnight at 8° C. The reaction end point was monitored by TLC (SiO₂, hexane/EtOAc: 1/1). After completion, the work-up was performed as described in General Procedure M and the crude product adsorbed on diatomaceous earth. Purification by flash chromatography, applying an isocratic method (hexane/EtOAc: 1/1), afforded intermediate 114 (50% yield, 11.65 mmol, 3.1 g) as oil. MS (ESI, positive mode): found m/z=270.0 [M+H]⁺, calculated m/z=270.0 [M+H]⁺.

### tert-Butyl 4-(4,5-dihydroxypentyl)-4-hydroxypiperidine-1-carboxylate (115)

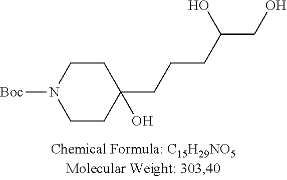 [SMILES: CC(C)(C)OC(=O)N1CCC(O)(CCCC(O)CO)CC1]

Following General Procedure N, intermediate 114 (1 eq., 7.8 mmol, 2.1 g) was dissolved in acetone (47.4 mL). Then, NMO (1.2 eq., 9.35 mmol, 1.1 g) was added followed by water (16 eq., 124.8 mmol, 2.2 mL). Finally, OsO₄-solution in tert-butanol (0.47% mol, 468.8 μmol, 102 μL) was added and the resulting reaction mixture stirred overnight at r.t. The reaction progress was monitored by TLC (SiO₂, hexane/EtOAc: 1/1 for olefine consumption, CHCl₃/MeOH: 10/1 for diol formation). After completion, the work-up was performed as described in General Procedure M and the crude product adsorbed on diatomaceous earth. Purification by flash chromatography, applying an isocratic method (CHCl₃/MeOH: 10/1), afforded intermediate 115 (66% yield, 5.1 mmol, 1.6 mg) as oil. MS (ESI, positive mode): found m/z=304.1 [M+H]⁺, calculated m/z=304.4 [M+H]⁺.

### tert-Butyl 4-hydroxy-4-(4-oxobutyl)piperidine-1-carboxylate (116)

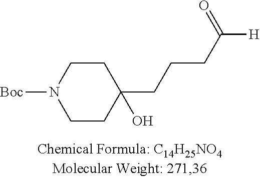 [SMILES: CC(C)(C)OC(=O)N1CCC(O)(CCCC=O)CC1]

Following General Procedure O, Intermediate 115 (1 eq., 5.83 mmol, 1.8 g) was dissolved in THF (36.8 mL). In parallel, sodium periodate (2 eq., 11.7 mmol, 2.5 g) was dissolved in water (18.4 mL). Then, both solutions were mixed following instructions from General procedure O. The reaction mixture was stirred for 1 h and the progress monitored by TLC (SiO₂, DCM/MeOH: 9/1). After completion, the work-up was performed as described in General Procedure O, and without further purification, intermediate 116 (50% yield, 2.9 mmol, 791 mg) was afforded as a white solid after lyophilisation. ¹H-NMR (500 MHz, d6-DMSO). δ/ppm=9.66 (t, J=1.7 Hz, 1H), 3.64-3.51 (m, 2H), 1.69-1.08 (m, 17H). ¹³C-NMR (126 MHz, d6-DMSO): δ/ppm=203.35, 153.96, 89.72, 78.26, 70.89, 40.25, 34.18, 32.55, 31.50, 28.00, 16.90. MS (ESI, positive mode): found m/z=272.0 [M+H]⁺, calculated m/z=272.4 [M+H]⁺.

Alternative method for the one pot synthesis of 116:

Following General Procedure S, sodium metaperiodate (2.5 eq., 9.3 mmol, 1.99 g) was dissolved in H₂O (15.7 mL). Next, a 1M solution of sulfuric acid (0.33 eq., 1.1 mmol, 107.8 mg) in water (1.1 mL) was added. Then, temperature was lowered and according to General Procedure S, 186 μL (0.5 mol %, 18.6 μmol, 4.86 mg) of a 0.1 M solution of ruthenium trichloride trihydrate in water was added. After adding the organic phase composed of acetonitrile and ethyl acetate as described in General Procedure S, a pre-cooled solution of olefin 114 (1 eq., 3.72 mmol, 1 g) in ethyl acetate (5.16 mL) was added. The progress of the reaction was monitored by thin layer chromatography (TLC) (SiO₂, DCM/MeOH: 9/1 and DCM/MeOH: 30/1). After stirring for 30 minutes at 0° C. the reaction mixture was worked up as described in General Procedure S. Purification by flash chromatography on silica phase (100% DCM to 10% MeOH 90% DCM in 15 min) afforded intermediate 116 (79% yield, 2.944 mmol, 798.8 mg) in a one-pot synthesis as transparent oil. ¹H NMR (500 MHz, dmso-d6): δ/ppm 5.97 (d, J=5.9 Hz, 3H), 4.83 (ddd, J=8.2, 5.9, 2.4 Hz, 1H), 3.56 (d, J=13.0 Hz, 2H), 3.08-3.04 (m, 2H), 1.68-1.51 (m, 2H), 1.44-1.32 (m, 11H), 1.32-1.14 (m, 6H). ¹³C NMR (126 MHz, dmso-d6): δ/ppm 153.95, 89.71, 78.26, 70.89, 37.73, 34.16, 32.55, 31.48, 28.00, 16.89. MS (ESI, positive mode): found m/z=272.0 [M+H]⁺ and 291.7 (mass of water acetal), calculated m/z=272.18 [M+H]⁺ and 290.19 [M+H]⁺ (water acetal of 116) [M+H]⁺.

### tert-butyl 4-(4-aminobutyl)-4-hydroxypiperidine-1-carboxylate (117)

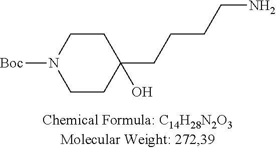 [SMILES: CC(C)(C)OC(=O)N1CCC(O)(CCCCN)CC1]

Following General Procedure P, intermediate 116 (1 eq., 3.28 mmol, 890 mg) was dissolved in MeOH (abs., 14.02 mL). In parallel, ammonium acetate (10 eq., 32.8 mmol, 2.5 g) and sodium cyanoborohydride (2 eq., 6.56 mmol, 412 mg) were added into a two-neck flask and dissolved in MeOH (abs., 10 mL). Then, following the conditions described in General Procedure P, the ammonium acetate/sodium cyanoborohydride solution is poured into the intermediate 116 solution and stirred for 8 h at r.t. The reaction was monitored by TLC (SiO₂, CHCl₃/MeOH/Et₃N: 10/1/1%). After completion, the work-up was performed as described in General Procedure P. Purification by preparative HPLC (method 11) afforded intermediate 117 (12% yield, 0.39 mmol, 107 mg) as an oil. MS (ESI, positive mode): found m/z=273.2 [M+H]⁺, calculated m/z=273.4 [M+H]⁺.

### 4-nitrophenyl isoindoline-2-carboxylate (118)

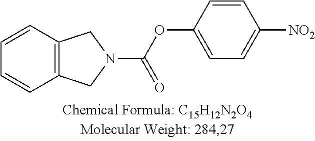 [SMILES: O=C(Oc1ccc([N+](=O)[O-])cc1)N1Cc2ccccc2C1]

Isoindoline (1 eq., 0.42 mmol, 48 μL) was dissolved in DMF (abs. resulting concentration=0.11 M, 3.7 mL). Then, bis-p-nitrophenyl carbonate (1.1 eq., 0.462 mmol, 141 mg) was added. After solubilisation, DIPEA (1.5 eq., 0.63 mmol, 110 μL) was added and the resulting reaction mixture stirred for 30 min at r.t. The reaction progress was monitored by HPLC (method 1). After completion, the reaction mixture was directly purified by preparative HPLC system (method 1, t_{R}=13.78 min), affording intermediate 118 (90% yield, 0.38 mmol, 107 mg) as white crystals. ¹H-NMR (500 MHz, CDCl₃): δ/ppm=8.27 (d, J=9.1 Hz, 2H), 7.39 (d, J=9.1 Hz, 2H), 7.35-7.28 (m, 4H), 4.96 (s, 2H), 4.85 (s, 2H). ¹³C-NMR (126 MHz, CDCl₃) δ/ppm=156.09, 151.65, 144.81, 136.06, 135.83, 127.84, 127.78, 125.10, 122.81, 122.66, 122.15, 52.81, 52.66. MS (ESI, negative mode): found m/z=283.3 [M−H]⁻, calculated m/z=283.4 [M−H]⁻.

### tert-butyl 4-hydroxy-4-(4-(isoindoline-2-carboxamido)butyl)piperidine-1-carboxylate (119)

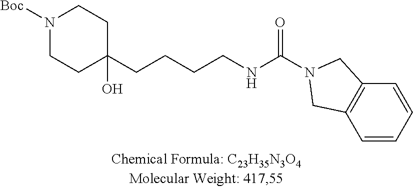 [SMILES: CC(C)(C)OC(=O)N1CCC(O)(CCCCNC(=O)N2Cc3ccccc3C2)CC1]

Following General Procedure R, intermediate 118 (2 eq., 0.84 mmol, 239 mg) and intermediate 117 (1 eq., 0.42 mmol, 114 mg) were dissolved in DMF (abs., resulting concentration=26.7 mmol, 18.4 mL). Then, N-methylimidazole (10 eq., 4.22 mmol, 336 μL) were as added and the resulting reaction mixture was stirred for three weeks at 45° C. The reaction end point was followed by HPLC (method 19. After completion, the work-up was performed as described in General Procedure R. Purification by preparative HPLC (method 1, t_{R}=14.01 min) afforded intermediate 119 (8% yield, 30 μmol, 14 mg) as an oil. MS (ESI, positive mode): found m/z=418.3 [M+H]⁺, calculated m/z=418.4 [M+H]⁺.

### 3-(Boc-hydroxy)-4-(Boc-amino)benzoic Acid (120)

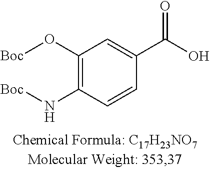 [SMILES: CC(C)(C)OC(=O)Nc1ccc(C(=O)O)cc1OC(=O)OC(C)(C)C]

44-amino-3-hydroxybenzoic acid (1 eq., 6.53 mmol, 1 g) and triethylamine (3.5 eq., 22.9 mmol, 3.19 mL) were dissolved in DCM (abs., resulting concentration=0.17 M, 39.2 mL) at 0° C. Then, a solution of di-tert-butyl dicarbonate (3.5 eq., 22.9 mmol, 5 g) in DCM (abs., resulting concentration=0.33 M, 20 mL) was added dropwise at 0° C. The reaction mixture was stirred overnight at 20° C. The reaction progress was monitored by TLC (SiO₂, hexane/EtOAc: 2/1). After completion, the volatiles were evaporated under reduced pressure. Then, the crude product was adsorbed on diatomaceous earth. Purification by flash chromatography, applying an isocratic method (hexane/EtOAc: 2/1) afforded intermediate 120 (36% yield, 2.35 mmol, 831 mg) as a white solid. ¹H-NMR (500 MHz, d6-DMSO): δ/ppm=10.51 (s, 1H), 8.29 (s, 1H), 7.59 (dd, J=8.6, 2.0 Hz, 1H), 7.51 (d, J=2.1 Hz, 1H), 6.81 (d, J=8.6 Hz, 1H), 1.50 (d, J=1.2 Hz, 18H). ¹³C-NMR (126 MHz, d6-DMSO): δ/ppm=160.64, 150.79, 147.25 (d, J=6.2 Hz), 135.74, 129.59, 124.63, 114.77, 112.34, 84.92, 83.24, 27.16, 26.91. MS (ESI, negative mode): found m/z=352.1 [M−H]⁻, calculated m/z=352.4 [M−H]⁻.

### N-(4-(4-hydroxypiperidin-4-yl)butyl)isoindoline-2-carboxamide, TFA salt (122)

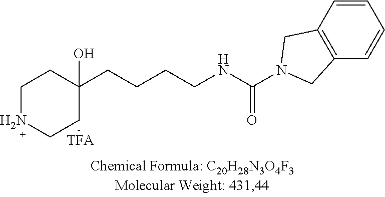

Following General Procedure C variation 2, intermediate 119 (1 eq., 0.05 mmol, 21 mg) was dissolved in a solution of 20% TFA in DCM (6 mL). The reaction mixture was stirred at r.t. for 30 min. The reaction progress was monitored by TLC (SiO₂, DCM/MeOH: 10/1) and HPLC (method 2). After completion, the work-up was performed as described in General Procedure C, and without further purification, intermediate 122 (80% yield, 0.04 mmol, 17.3 mg) was afforded as oil. HPLC: t_{R}/min=9.89 min.

### tert-butyl(2-((tert-butoxycarbonyl)oxy)-4-(4-hydroxy-4-(4-(isoindoline-2-carboxamido)butyl)piperidine-1-carbonyl)phenyl)carbamate (123)

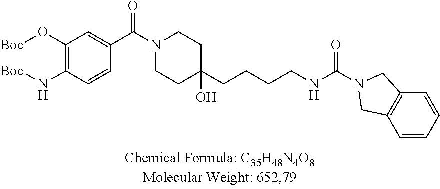 [SMILES: CC(C)(C)OC(=O)Nc1ccc(C(=O)N2CCC(O)(CCCCNC(=O)N3Cc4ccccc4C3)CC2)cc1OC(=O)OC(C)(C)C]

Following General Procedure F, intermediate 122 (1 eq., 0.05 mmol, 22 mg) and intermediate 120 (2 eq., 0.1 mmol, 35 mg) were dissolved in DCM (abs., 1 mL). Then, DIPEA (2.2 eq., 0.11 mmol, 19 μL), HOBt (2 eq., 0.1 mmol, 13 mg) and DCC (2 eq., 0.1 mmol, 21 mg) were added sequentially to the reaction mixture and stirred overnight at r.t. The reaction progress was monitored by TLC (SiO₂, DCM/MeOH: 9/1). After completion, the work-up was performed as described in General Procedure F, and purification by preparative HPLC (method 1, t_{R}=13.82 min) afforded intermediate 123 (30% yield, 0.02 mmol, 9.8 mg) as white powder after lyophilisation. MS (ESI, positive mode): found m/z=653.2 [M+H]⁺, calculated m/z=653.4 [M+H]⁺.

### N-(4-(1-(4-amino-3-hydroxybenzoyl)-4-hydroxypiperidin-4-yl)butyl)isoindoline-2-carboxamide TFA salt (124)

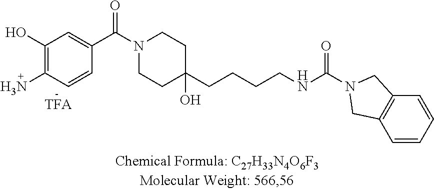 [SMILES: Nc1[c-]cc(C(=O)N2CCC(O)(CCCCNC(=O)N3Cc4ccccc4C3)CC2)cc1O]

Following General Procedure C variation 2, Intermediate 123 (1 eq., 0.02 mmol, 13 mg) was dissolved in a solution of 20% TFA in DCM (4 mL). The reaction mixture was stirred for 20 min at r.t. The reaction progress was monitored by TLC (SiO₂, DCM/MeOH: 10/1) and HPLC (method 2). After completion, the work-up was performed as described in General Procedure C, and without further purification intermediate 124 (65% yield, 0.01 mmol, 7.4 mg) was afforded as a white solid. ¹H-NMR (500 MHz, d6-DMSO): δ/ppm=7.33-7.23 (m, 4H), 6.73 (d, J=1.8 Hz, 1H), 6.68-6.58 (m, 2H), 6.23 (d, J=5.6 Hz, 1H), 4.56 (s, 4H), 1.48-1.33 (m, 10H). HPLC: t_{R}/min=8.14. MS (ESI, positive mode): found m/z=453.3 [M+H]⁺, calculated m/z=453.4 [M+H]⁺.

Synthesis of c42a

### tert-butyl 4-hydroxy-4-(4-(isoindoline-2-carboxamido)butyl)piperidine-1-carboxylate (119)

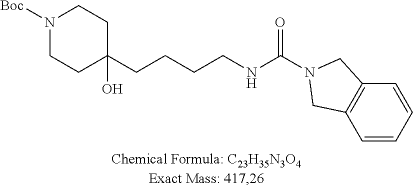 [SMILES: CC(C)(C)OC(=O)N1CCC(O)(CCCCNC(=O)N2Cc3ccccc3C2)CC1]

2,3-dihydro-1H-isoindole-2-carbonyl chloride (65.84 mg, 0.36 mmol, 1 eq.) was dissolved in DCM (abs., 243 μL) under argon. Then, a solution of amine (117) (1 eq., 0.24 mmol, 66 mg) and triethylamine (118.2 μL, 0.85 mmol, 3.5 eq.) in DCM (abs. 1.3 mL) was added and stirred overnight at room temperature. The reaction progress was monitored by HPLC (method 12) and TLC (SiO₂, CHCl₃/MeOH: 10/1). After completion, the reaction mixture was diluted with DCM (3.3 mL) and then washed once with saturated aqueous solution of sodium bicarbonate and twice with brine. The organic phase was collected and dried over MgSO₄, the volatiles were evaporated under reduced pressure, and the crude product was purified by preparative HPLC system (method 12), affording intermediate 119 (45.31 mg, 44%) as orange oil. HPLC (method 12): t_{R}=12.7 min MS (ESI, positive mode): found m/z=418.3 [M+H]⁺, calculated m/z=418.26 [M+H]⁺.

### N-(4-(4-hydroxypiperidin-4-yl)butyl)isoindoline-2-carboxamide, TFA salt (intermediate 122 alternative synthesis route)

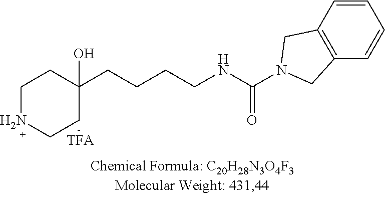

According to general procedure C, variant 2, Boc-protected intermediate (166a) (1 eq., 0.109 mmol, 45.31 mg) was dissolved in a solution of 20% TFA in DCM (5 mL). The reaction mixture was stirred at room temperature for 1 h. The reaction progress was monitored by TLC (SiO₂, CHCl₃/MeOH: 10/1) and HPLC (method 13). After completion, the reaction mixture was diluted with MeOH (10 mL) and the volatiles were evaporated under reduced pressure. This procedure was repeated twice. Then, the crude product was dried overnight under high vacuum. Without further purification, intermediate (166a) (47.02 mg, quant.) was afforded as oil. HPLC (method 13): t_{R}/min=7.79 min.

### tert-butyl (3-(4-hydroxy-4-(4-(isoindoline-2-carboxamido)butyl)piperidine-1-carbonyl)phenyl)carbamate (167a)

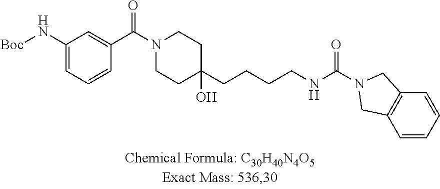 [SMILES: CC(C)(C)OC(=O)Nc1cccc(C(=O)N2CCC(O)(CCCCNC(=O)N3Cc4ccccc4C3)CC2)c1]

TFA salt intermediate (122) (51.38 mg, 0.12 mmol, 1 eq.) was dissolved in DMF (abs., 2 mL). Then, 3-(Boc-amino)benzoic acid (45.31 mg, 0.14 mmol, 1.2 eq.) was added and stirred until complete dissolution. DIPEA (49 μL, 0.26 mmol, 2.2 eq.), HOBt (19.4 mg, 0.14 mmol, 1.2 eq.) and DCC (33.0 mg, 0.16 mmol, 1.3 eq.) were added sequentially to the reaction mixture and stirred overnight at room temperature. The reaction progress was monitored by TLC (SiO₂, CHCl₃/MeOH: 10/1). After completion, the urea precipitate was removed by filtration, the volatiles evaporated under reduced pressure and the crude product purified by preparative HPLC (method 14). Intermediate 167a (22.4 mg, 35%) was afforded as a yellow powder after lyophilization. HPLC (method 14): t_{R}=9.1 min. MS (ESI, positive mode): found m/z=537.4 [M+H]⁺, calculated m/z=537.3 [M+H]⁺.

### N-(4-(1-(3-aminobenzoyl)-4-hydroxypiperidin-4-yl)butyl)isoindoline-2-carboxamide TFA salt (c42a)

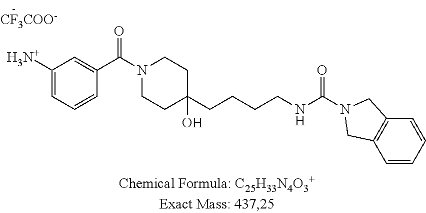 [SMILES: "CF3COO-" ; [NH3+]c1cccc(C(=O)N2CCC(O)(CCCCNC(=O)N3Cc4ccccc4C3)CC2)c1]

According to general procedure C, variant 2, Boc-protected intermediate (167a) (22.4 mg, 41.8 μmol, 1 eq.) was dissolved in a solution of 20% TFA in DCM (5 mL). The reaction mixture was stirred at room temperature for 1 h. The reaction progress was monitored by TLC (SiO₂, CHCl₃/MeOH: 10/1) and HPLC (method 14). After completion, the reaction mixture was diluted with MeOH (10 mL) and the volatiles were evaporated under reduced pressure. This procedure was repeated twice. The crude product was dried overnight under high vacuum. Without further purification, NAMPT inhibitor c42a (21.28 mg, 92%) was afforded as oil. HPLC (method 14): t_{R}/min=6.5 min. MS (ESI, positive mode): found m/z=437.2 [M+H]⁺, calculated m/z=437.2 [M+H]⁺.

### 4-((R)-2-((R)-2-(6-(2,5-dioxo-2,5-dihydro-1H-pyrrol-1-yl)hexanamido)-3-methylbutanamido) propanamido)benzyl (3-(4-hydroxy-4-(4-(isoindoline-2-carboxamido)butyl)piperidine-1-carbonyl) phenyl)carbamate (c42b.10)

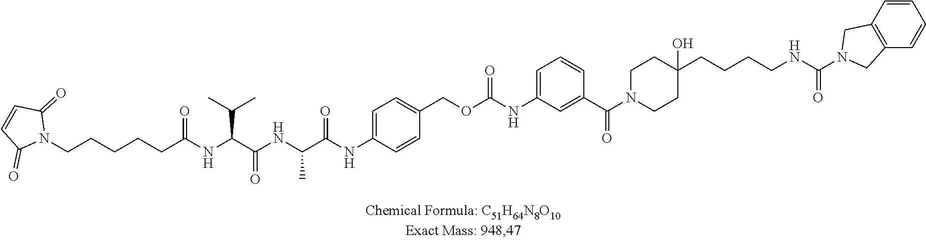 [SMILES: CC(C)[C@H](NC(=O)CCCCCN1C(=O)C=CC1=O)C(=O)N[C@@H](C)C(=O)Nc1ccc(COC(=O)Nc2cccc(C(=O)N3CCC(O)(CCCCNC(=O)N4Cc5ccccc5C4)CC3)c2)cc1]

mc-Val-Ala-PAB-PNP (21.49 mg, 33.0 μmol, 1.5 eq.) was dissolved in DMF (abs., 1 mL) under argon. Then, PyAOP (17.2 mg, 33.0 μmol, 1.5 eq.) was dissolved in DMF (abs., 648 μL) and added to the linker solution. DIPEA (2.5 eq., 0.054 mmol, 9.6 μL) was added to the reaction mixture and the pH checked to be over 8. The resulting solution was stirred at room temperature for 30 min followed by the addition of NAMPT inhibitor (c42a) (12.0 mg, 21.8 μmol, 1 eq.) and stirred at room temperature overnight. The reaction progress was monitored by analytical HPLC (method 15). After completion, the volatiles were evaporated under reduced pressure and the crude product was purified by preparative HPLC (method 15). Linker-payload (c42b.10) (5.37 mg, 26%) was afforded as white solid after lyophilization. HPLC (method 15): t_{R}=14.65 min. MS (ESI, positive mode): found m/z=949.7 [M+H]⁺, calculated m/z=949.5 [M+H]⁺.

Synthesis of Compounds c42a and c42b.10′

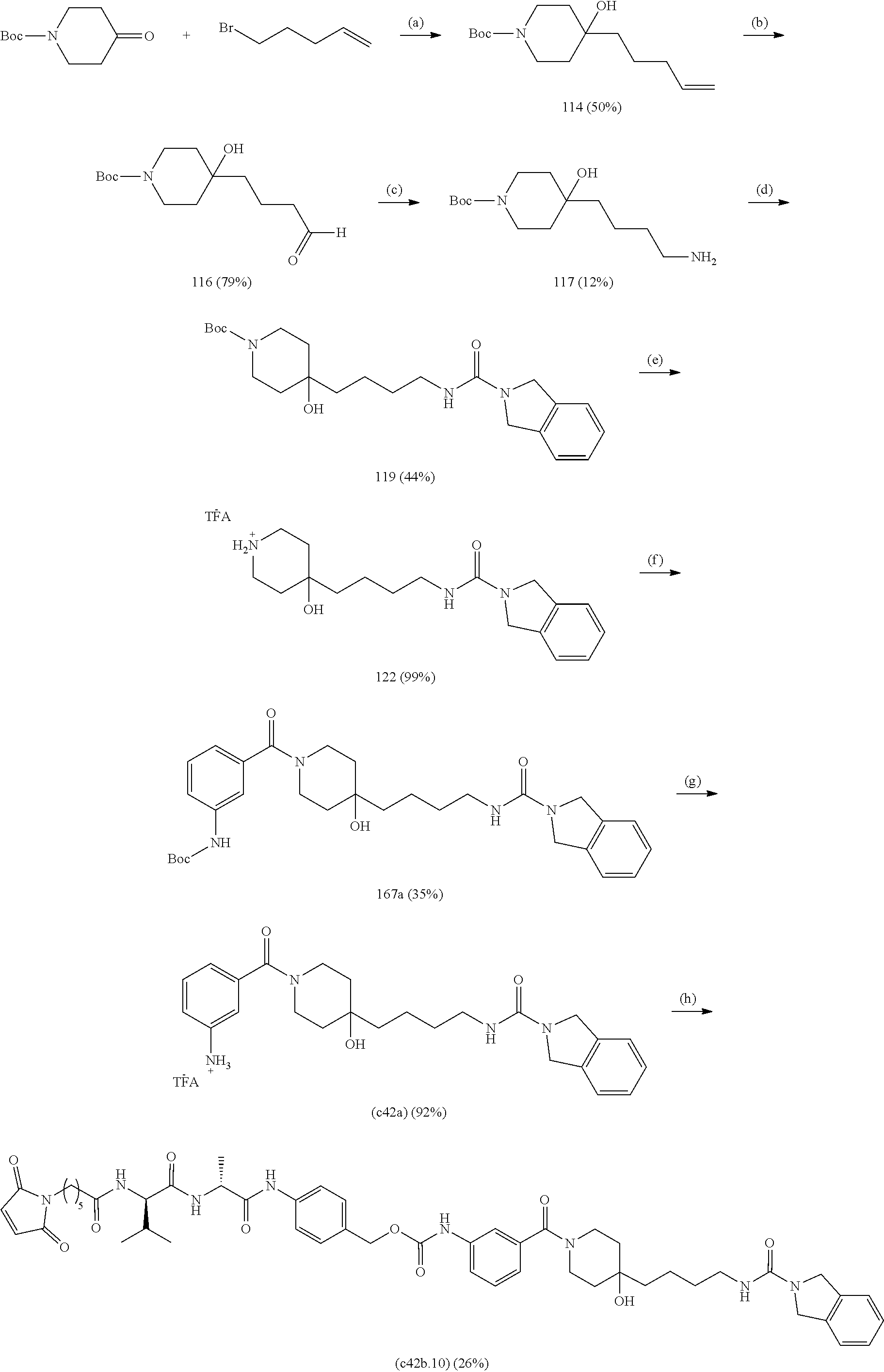

Tertiary alcohol isoindoline urea payload c42a and linker payload c42b.10. Reagents and conditions: (a) Mg, LaCl₃—LiCl, THF (abs.), <12° C., overnight; (b) RuCl₃*3 H₂O (cat), NaIO₄, H₂SO₄, H₂O, Acetonitrile, Ethyl acetate, <5° C., 30 min; (c) ammonium acetate (pH=6), NaCNBH₄, MeOH (abs.), r.t, 4.5 h; (d) 2,3-dihydro-1H-isoindole-2-carbonyl chloride, Et₃N, DCM (abs.), r.t, overnight; (e) 20% TFA DCM solution, r.t, 1 h; (f) Boc-3-ABZ-OH, DCC/HOBt, DIPEA, DCM (abs.); r.t., overnight; (g) 20% TFA DCM solution, r.t, 1 h; (h) Mal-Val-Ala-PAB-PNP, PyAOP, DIPEA, DMF (abs.), r.t., overnight.

### HPLC Methods:

TABLE 1
HPLC method 1

| | Solvent 1: H₂O 0.05% | Solvent 2: |
|---|---|---|
| Time (min) | TFA | ACN |
| 0 | 95 | 5 |
| 14.8 | 0 | 100 |
| 18 | 0 | 100 |
| 18.5 | 95 | 5 |
| 22 | 95 | 5 |

TABLE 2
HPLC method 2

| | Solvent 1: H₂O 0.05% | Solvent 2: |
|---|---|---|
| Time (min) | TFA | ACN |
| 0 | 95 | 5 |
| 14.8 | 50 | 50 |
| 15 | 0 | 100 |
| 18 | 0 | 100 |
| 18.5 | 95 | 5 |
| 22 | 95 | 5 |

TABLE 3
HPLC method 3

| | Solvent 1: H₂O 0.05% | Solvent 2: |
|---|---|---|
| Time (min) | TFA | ACN |
| 0 | 95 | 5 |
| 10.8 | 30 | 70 |
| 15 | 20 | 80 |
| 33.5 | 0 | 100 |
| 34.5 | 95 | 5 |
| 35 | 95 | 5 |

TABLE 4
HPLC method 4

| | Solvent 1: H₂O 0.05% | |
|---|---|---|
| Time (min) | TFA | Solvent 2: ACN |
| 0 | 95 | 5 |
| 10.8 | 30 | 70 |
| 15 | 20 | 80 |
| 26 | 0 | 100 |
| 27.5 | 95 | 5 |
| 28 | 95 | 5 |

TABLE 5
HPLC method 5

| | Solvent 1: H₂O 0.05% | |
|---|---|---|
| Time (min) | TFA | Solvent 2: ACN |
| 0 | 95 | 5 |
| 15 | 30 | 70 |
| 15.1 | 0 | 100 |
| 18 | 0 | 100 |
| 18.5 | 95 | 5 |
| 22 | 95 | 5 |

TABLE 6
HPLC method 6

| | Solvent 1: H₂O 0.05% | |
|---|---|---|
| Time (min) | TFA | Solvent 2: MeOH |
| 0 | 95 | 5 |
| 14.8 | 20 | 80 |
| 15 | 0 | 100 |
| 18 | 0 | 100 |
| 18.5 | 95 | 5 |
| 22 | 95 | 5 |

TABLE 7
HPLC method 7

| | Solvent 1: H₂O 0.05% | |
|---|---|---|
| Time (min) | TFA | Solvent 2: ACN |
| 0 | 95 | 5 |
| 2 | 75 | 25 |
| 35 | 66.3 | 33.7 |
| 36 | 0 | 100 |
| 41 | 0 | 100 |
| 41.5 | 95 | 5 |
| 43 | 95 | 5 |

TABLE 8
HPLC method 8

| Time (min) | Solvent 1: H₂O | Solvent 2: ACN |
|---|---|---|
| 0 | 95 | 5 |
| 2 | 50 | 50 |
| 15 | 35 | 65 |
| 15.2 | 0 | 100 |
| 18 | 0 | 100 |
| 18.5 | 95 | 5 |
| 22 | 95 | 5 |

TABLE 9
HPLC method 9

| | Solvent 1: H₂O 0.05% | |
|---|---|---|
| Time (min) | TFA | Solvent 2: ACN |
| 0 | 95 | 5 |
| 2 | 50 | 50 |
| 13 | 0 | 100 |
| 18 | 0 | 100 |
| 18.5 | 95 | 5 |
| 22 | 95 | 5 |

TABLE 10
HPLC method 10

| | Solvent 1: H₂O 0.05% | |
|---|---|---|
| Time (min) | TFA | Solvent 2: ACN |
| 0 | 95 | 5 |
| 14.8 | 35 | 65 |
| 15 | 0 | 100 |
| 18 | 0 | 100 |
| 18.5 | 95 | 5 |
| 22 | 95 | 5 |

TABLE 11
HPLC method 11

| | Solvent 1: H₂O 0.05% | |
|---|---|---|
| Time (min) | TFA | Solvent 2: ACN |
| 0 | 95 | 5 |
| 14.8 | 50 | 50 |
| 21 | 0 | 100 |
| 24 | 0 | 100 |
| 24.5 | 95 | 5 |
| 28 | 95 | 5 |

TABLE 12
HPLC method 12

| Time (min) | Solvent 1: H₂O | Solvent 2: ACN |
|---|---|---|
| 0 | 95 | 5 |
| 14.8 | 15 | 85 |
| 16 | 0 | 100 |
| 18 | 0 | 100 |
| 18.5 | 95 | 5 |
| 22 | 95 | 5 |

TABLE 13
HPLC method 13

| | Solvent 1: H₂O 0.05% | |
|---|---|---|
| Time (min) | TFA | Solvent 2: ACN |
| 0 | 95 | 5 |
| 14.8 | 20 | 80 |
| 16 | 0 | 100 |
| 18 | 0 | 100 |
| 18.5 | 95 | 5 |
| 22 | 95 | 5 |

TABLE 14
HPLC method 14

| | Solvent 1: H₂O 0.05% | |
|---|---|---|
| Time (min) | TFA | Solvent 2: ACN |
| 0 | 95 | 5 |
| 7 | 30 | 70 |
| 14.8 | 18 | 82 |
| 16 | 0 | 100 |
| 18 | 0 | 100 |
| 18.5 | 95 | 5 |
| 22 | 95 | 5 |

TABLE 15
HPLC method 15

| | Solvent 1: H₂O 0.05% | |
|---|---|---|
| Time (min) | TFA | Solvent 2: ACN |
| 0 | 95 | 5 |
| 12 | 45.5 | 54.5 |
| 14.8 | 31 | 69 |
| 16 | 0 | 100 |
| 18 | 0 | 100 |
| 18.5 | 95 | 5 |
| 22 | 95 | 5 |

TABLE 16
HPLC method 16

| | Solvent 1: H₂O 0.05% | |
|---|---|---|
| Time (min) | TFA | Solvent 2: ACN |
| 0 | 95 | 5 |
| 7 | 48.6 | 51.4 |
| 14.8 | 26.9 | 73.1 |
| 16 | 0 | 100 |
| 18 | 0 | 100 |
| 18.5 | 95 | 5 |
| 22 | 95 | 5 |

Synthesis of Compounds c22a (94) and c22b.8 (97)

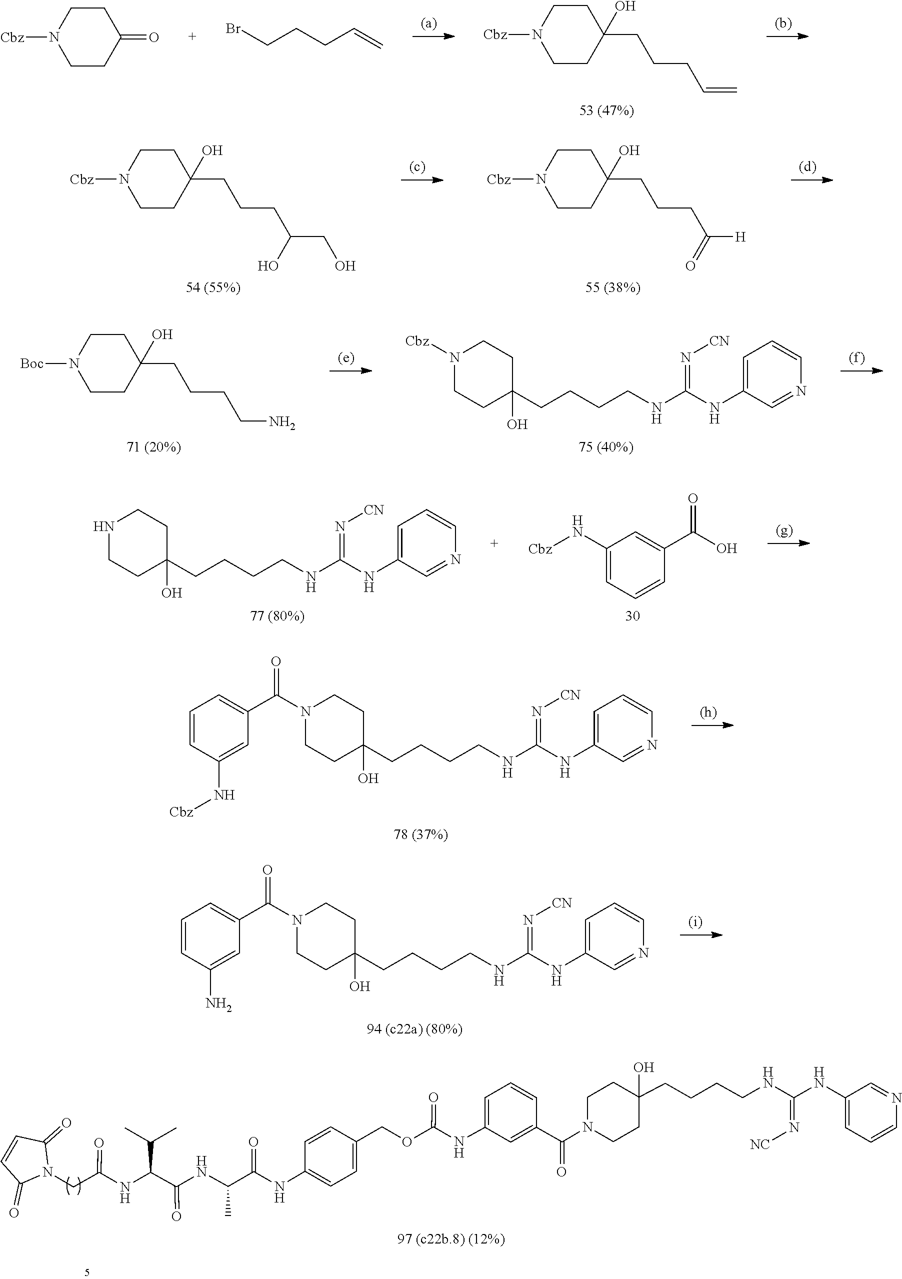

Tertiary alcohol payload c22a (94) and linker payload c22b.8 (97). Reagents and conditions: (a) Mg, LaCl₃—LiCl, THF (abs.), <12° C., overnight; (b) OsO₄ (cat), N-methyl morpholine N-oxide (NMO), H₂O, acetone, r.t, overnight; (c) NaIO₄, THF/H₂O (2:1), 0° C., 30 min; (d) ammonium acetate (pH=6), NaCNBH₄, MeOH (abs.), r.t, 4.5 h; (e) Phenyl N-cyano-N′-3-pyridinylcarbamimidate; Et₃N; 1,4-dioxane; r.t., overnight; (f) H₂, Pd/C, EtOH/EtOAc (1:1), r.t., overnight; (g) DCC/HOBt, DIPEA, DCM (abs.); r.t., overnight; (h) H₂, Pd/C, EtOH/EtOAc (1:1), r.t., 4 h; (i) Mal-Val-Ala-PAB-PNP, PyAOP, DIPEA, DMF (abs.), r.t., overnight.

### Improved synthesis of tert-Butyl 4-hydroxy-4-(4-oxobutyl)piperidine-1-carboxylate (116)

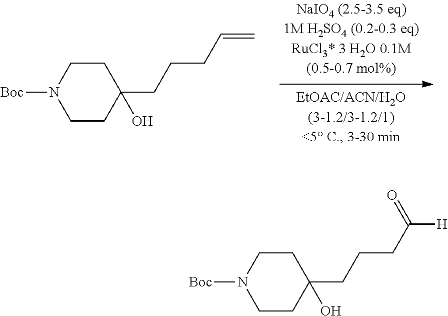 [SMILES: C=CCCCC1(O)CCN(C(=O)OC(C)(C)C)CC1 ; CC(C)(C)OC(=O)N1CCC(O)(CCCC=O)CC1]

According to general procedure S, sodium metaperiodate (2.5 eq., 9.3 mmol, 1.99 g) was dissolved in H₂O (15.7 mL) in a 100 mL one-necked flask. Next, a 1M solution of sulfuric acid (0.33 eq., 1.1 mmol, 107.8 mg) in water (1.1 mL) was added and after dissolution of all solids, the temperature was lowered to 0° C. Next, a 0.1M solution of ruthenium trichloride trihydrate (0.5 mol %, 18.6 μmol, 4.86 mg) in water (186 μL) was added. The reaction mixture was stirred for 5 minutes until the colour changed to bright yellow. Then, a mixture of acetonitrile (16.63 mL) and ethyl acetate (11.47 mL) was added, and the reaction mixture was stirred for 5 minutes. A solution of olefin 114 (1 eq. 3.72 mmol, 1 g) in ethyl acetate (5.16 mL) pre-cooled to 0° C. was added to the reaction mixture (resulting concentration of olefin 114=79 mM) and the resulting reaction mixture stirred for 30 min at 0° C. The progress of the reaction was monitored by thin layer chromatography (TLC) (SiO₂, DCM/MeOH: 9/1 and DCM/MeOH: 30/1). Upon completion, the reaction mixture was poured into an aqueous mixture of a saturated sodium hydrogen carbonate solution (40 mL) and a saturated sodium thiosulfate solution (54 mL) at room temperature and stirred until the crude colour turned from yellow to violet. The organic layer was retained while the aqueous phase was extracted with ethyl acetate (3×60 mL). The organic layers were combined and washed once more with brine, dried over MgSO₄ and the volatiles were evaporated under reduced pressure. Purification by flash chromatography on silica phase (100% DCM to 10% MeOH 90% DCM in 15 min) afforded intermediate 116 (79% yield, 2.944 mmol, 798.8 mg) in a one-pot synthesis as transparent oil. ¹H NMR (500 MHz, dmso-d6): δ/ppm 5.97 (d, J=5.9 Hz, 3H), 4.83 (ddd, J=8.2, 5.9, 2.4 Hz, 1H), 3.56 (d, J=13.0 Hz, 2H), 3.08-3.04 (m, 2H), 1.68-1.51 (m, 2H), 1.44-1.32 (m, 11H), 1.32-1.14 (m, 6H). ¹³C NMR (126 MHz, dmso-d6): δ/ppm 153.95, 89.71, 78.26, 70.89, 37.73, 34.16, 32.55, 31.48, 28.00, 16.89. MS (ESI, positive mode): found m/z=272.0 [M+H]⁺ and 291.7 (mass of water acetal), calculated m/z=272.18 [M+H]⁺ and 290.19 [M+H]⁺ (water acetal of 116) [M+H]⁺.

In Vitro Testing of c22a and c22b.8′ According to the Disclosure
Comparison of the Free Inhibitor c22a and High DAR ADC Loaded with c22b.8′ in L540 CD30⁺ Cell Line

Cytotoxic profile of free inhibitor c22a compared with anti-CD30 mAb (Brentuximab)-c22b.8 ADC showing a 10⁴ fold higher toxicity of the ADC in comparison to the free inhibitor. Using as free inhibitor positive control 36 (not according to the disclosure) and method positive control Alpha-Amanitin. As positive control ADC a high DAR brentuximab ADC loaded with linker-payload 37 (not according to the disclosure) and unconjugated Brentuximab monoclonal antibody were used as negative control.

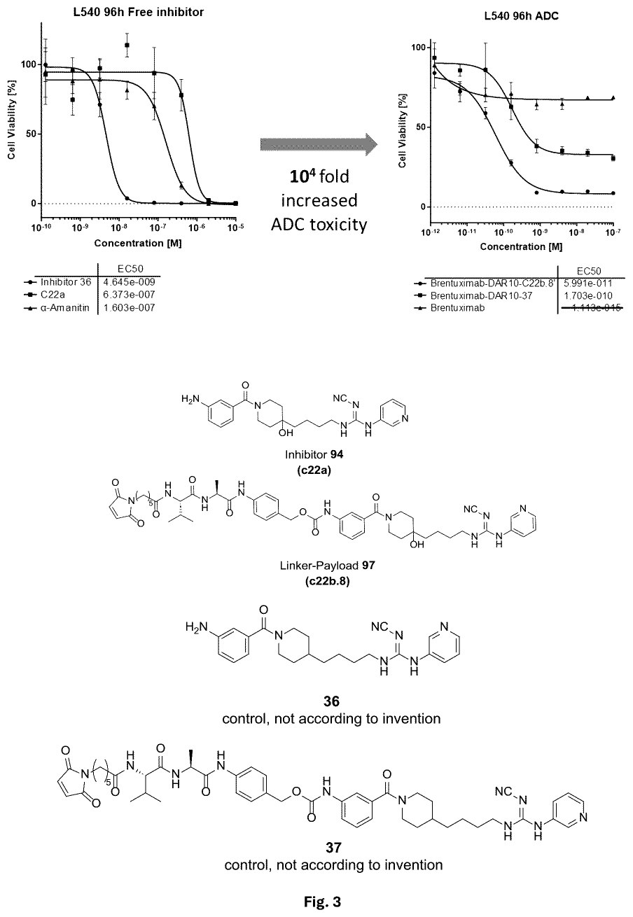

L540 CD30⁺ cell lines were used for the study. 2×10³ Cells were added to each well of a 96-well plate, but for the blank wells. Test compounds and controls were added to their corresponding lanes in a 7-step 1:5 dilution series starting with the concentration of 1×10⁻⁵ M for non-conjugated inhibitors and 1×10⁻⁷ M for ADCs and free monoclonal antibody. The dilution scheme was prepared in triplicates for each sample. Cells were incubated at 37° C. at 5% CO₂ for 96 h in an evaporation chamber. Readout was performed using CellTiter-Glo 2.0 assay, according to the kit instructions (Promega; Cat #G9242). (See FIG. 3)

In Vitro Testing of c42a and c42b.10′ According to the Disclosure
Comparison of the Free Inhibitor c42a Tested in L540 CD30⁺ Cell Line Vs Alpha-Amanitin

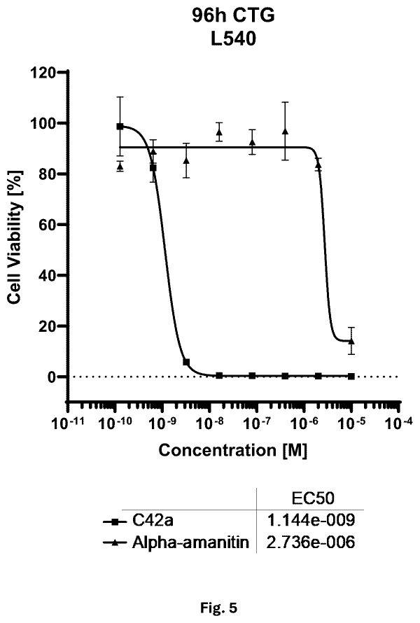

L540 (Hodgkin lymphoma) cells were used for an analysis of the cytotoxic potential HDP c42a. 2×10³ Cells were added to each well of a 96-well plate, but for the blank wells. Compound c42a and control (alpha-Amanitin) were added to their corresponding lanes in a 7-step 1:5 dilution series starting with the concentration of 1×10⁻⁵ M. The dilution scheme was prepared in triplicates for each sample. Cells were incubated at 37° C. at 5% CO₂ for 96 h in an evaporation chamber. Readout was performed using CellTiter-Glo 2.0 assay, according to the kit instructions (Promega; Cat #G9242). (See FIG. 5)

### Comparison of ADC DAR≈10, Linker Payload C42b.10′ Vs Control Linker-Payload 37 (not According to the Disclosure).

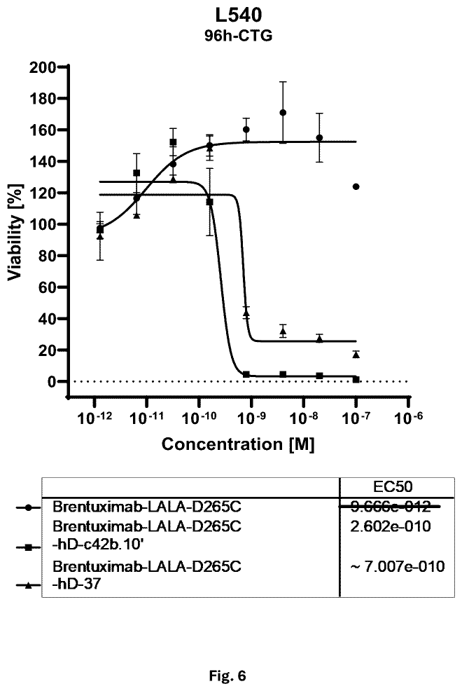

L540 (Hodgkin lymphoma) cells were used for an analysis of the cytotoxic potential of ADC Brentuximab-LALA-D265C-hD-c42b.10′ (anti-CD30 antibody conjugated to payload-linker c42b.10′). 2×10³ Cells were added to each well of a 96-well plate, but for the blank wells. The test compound (Brentuximab-LALA-D265C-hD-c42b.10′) and the controls (Brentuximab-LALA-D265C and Brentuximab-LALA-D265C-hD-37) were added to their corresponding lanes in a 7-step 1:5 dilution series starting with the concentration of 1×10⁻⁷ M. The dilution scheme was prepared in triplicates for each sample. Cells were incubated at 37° C. at 5% CO₂ for 96 h in an evaporation chamber. Readout was performed using CellTiter-Glo® 2.0 assay, according to kit instructions (Promega; Cat #G9242). (See FIG. 6)

Comparison of the First Generation Free Inhibitor 36 vs DAR≈10 (hD) Trastuzumab ADC Loaded to Linker Payload c42b.10′, Tested in MDA-MB-453 Using Bayer Cell Medium Conditions.

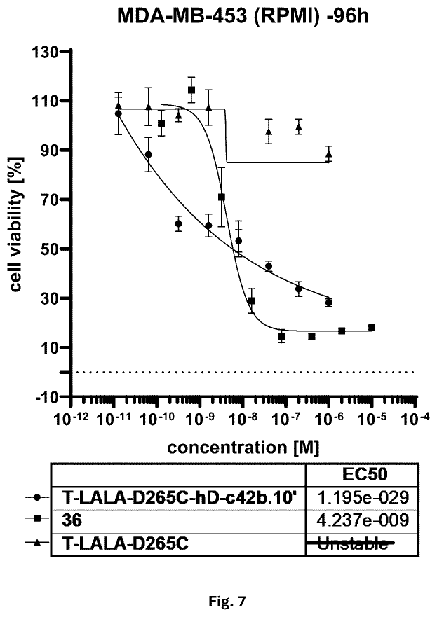

MDA-MB-453 (breast cancer) cells were used for an analysis of the cytotoxic potential of ADC T-LALA-D265C-hD-c42b.10′ (anti-HER2 antibody conjugated to payload-linker c42b.10′). 2×10³ Cells were added to each well of a 96-well plate, but for the blank wells. Test article (T-LALA-D265C-hD-c42b.10′) and controls (T-LALA-D265C and 36) were added to their corresponding lanes in a 7-step 1:5 dilution series starting with the concentration of 1×10⁻⁷ M. The dilution scheme was prepared in triplicates for each sample. Cells were incubated at 37° C. at 5% CO₂ for 96 h in an evaporation chamber. Readout was performed using BrdU-ELISA according to kit instructions (Roche, Cat #11669915001). (See FIG. 7)

### In Vivo Testing of NAMPTi

### I. NAMPTi-ADC Significantly Reduces Tumor Growth of L540 Subcutaneous Tumors in CB-17 SCID Mice at Single Dose

Female CB17-SCID mice were inoculated subcutaneously with 1×10⁷ L540 tumor cells in 200 μL RPMI medium containing 50% GFR-Matrigel into their right flanks. Once a mean tumor volume of 154.64 mm³ was reached, animals were allocated to control or treatment groups according to tumor size (day 0; n=10 animals/group). On the day after (day 1), the animals of groups 2-4 were treated with a single intravenous dose of Brentuximab-LALA-D265C-hD-c22b.8′. The animals of groups 5-7 were treated with a single intravenous dose of Brentuximab-LALA-D265C-hD-37. The tumor volume was measured twice per week by caliper and body weights were determined in parallel. Clinical signs and survival were monitored daily. The animals were sacrificed, and necropsy was performed when one or more termination criteria arose or at study termination.

TABLE 17
Experimental groups

| | | Dose | Dose | | | |
|---|---|---|---|---|---|---|
| | | Protein | Toxin | | | Animals |
| Group | Compound | [mg/kg] | [μg/kg] | Route | Schedule | (n) |
| 1 | PBS (Control) | — | — | i.v. | 1x | 10 |
| 2 | Brentuximab-LALA- | 7.5 | 374.6 | i.v. | 1x | 10 |
| | D265C- hD-c22b.8′ | | | | | |
| 3 | Brentuximab-LALA- | 12.5 | 624.3 | i.v. | 1x | 10 |
| | D265C- hD-c22b.8′ | | | | | |
| 4 | Brentuximab-LALA- | 25 | 1248.6 | i.v. | 1x | 10 |
| | D265C- hD-c22b.8′ | | | | | |
| 5 | Brentuximab-LALA- | 7.5 | 467.2 | i.v. | 1x | 10 |
| | D265C-hD -37 | | | | | |
| 6 | Brentuximab-LALA- | 12.5 | 778.6 | i.v. | 1x | 10 |
| | D265C-hD -37 | | | | | |
| 7 | Brentuximab-LALA- | 25 | 1557.3 | i.v. | 1x | 10 |
| | D265C-hD -37 | | | | | |

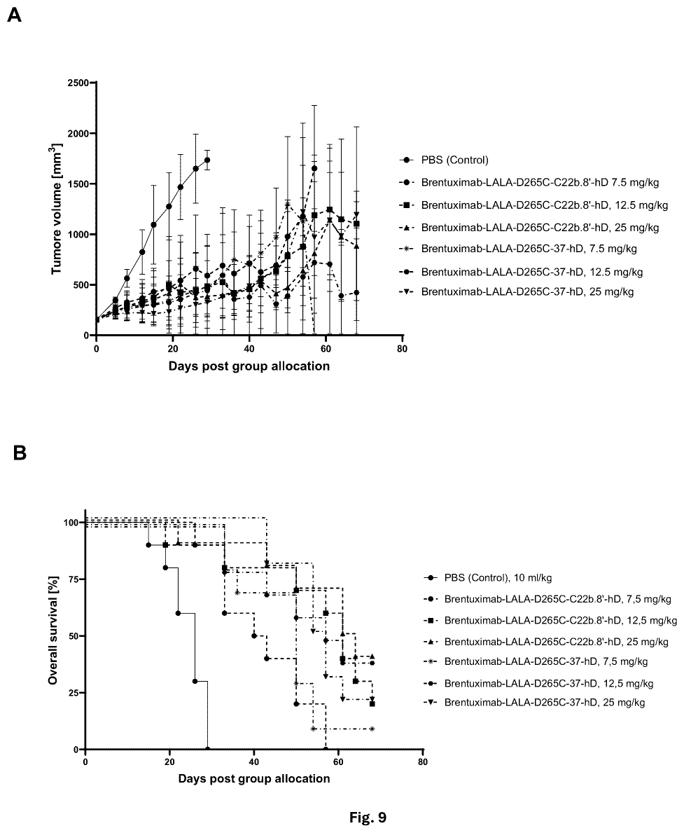

FIG. 9 shows: (A) Mean tumor volume [mm³] depicted from day 0 to day 71 post group allocation, measured by caliper. Mice were treated either with vehicle control or ADCs given as single intravenous dose (n=10 animals/group, mean depicted±SD). Treatment with ADC significantly reduced tumor growth in vivo. Both ADCs showed similar anti-tumor efficacy. (B) Survival of animals treated with ADCs was significantly prolonged in a dose-dependent manner as compared to vehicle control.

### II. NAMPTi-ADC Significantly Reduces Tumor Growth of L540 Subcutaneous Tumors in CB-17 SCID Mice

Female CB17-SCID mice were inoculated subcutaneously with 1×107 L540 tumor cells in 200 μL RPMI medium containing 50% GFR-Matrigel into their right flanks. Once a mean tumor volume of 137.90 mm3 was reached, animals were allocated to control or treatment groups according to tumor size (day 0; n=10 animals/group). On the day after (day 1), the animals of groups 2-4 were treated with Brentuximab-LALA-D265C-hD-c22b.8′ either as single intravenous dose or multiple dose treatment once a week for four weeks. The animals of groups 5 and 6 were treated with Brentuximab-A118C-LALA-D265C-c22b.8′ either as single intravenous dose or multiple dose treatment once a week for four weeks. Animals of negative control groups 7 and 8 were either treated with non-targeting ADC DIG-LALA-D265C-hD-c22b.8′ or naked antibody Brentuximab-LALA-D265C once a week for four weeks. The tumor volume was measured twice per week by calliper and body weights were determined in parallel. Clinical signs and survival were monitored daily. The animals were sacrificed, and necropsy was performed when one or more termination criteria arose or at study termination.

TABLE 18
Experimental groups

| | | Dose | | | | |
|---|---|---|---|---|---|---|
| | | Protein | Dose Toxin | | | |
| Group | Compound | [mg/kg] | [μg/kg] | Route | Schedule | Animals (n) |
| 1 | PBS (Control) | — | — | i.v. | 1x | 10 |
| 2 | Brentuximab- | 6.25 | 344.7 | i.v. | 1x/week | 10 |
| | LALA-D265C- | | | | for 4 weeks | |
| | hD-c22b.8′ | | | | | |
| 3 | Brentuximab- | 12.5 | 689.4 | i.v. | 1x/week | 10 |
| | LALA-D265C- | | | | for 4 weeks | |
| | hD-c22b.8′ | | | | | |
| 4 | Brentuximab- | 12.5 | 689.4 | i.v. | 1x | 10 |
| | LALA-D265C- | | | | | |
| | hD-c22b.8′ | | | | | |
| 5 | Brentuximab- | 12.5 | 251.1 | i.v. | 1x/week | 10 |
| | A118C-LALA- | | | | for 4 weeks | |
| | D265C-c22b.8′ | | | | | |
| 6 | Brentuximab- | 12.5 | 251.1 | i.v. | 1x | 10 |
| | A118C-LALA- | | | | | |
| | D265C-c22b.8′ | | | | | |
| 7 | DIG-LALA- | 12.5 | 697.3 | i.v. | 1x/week | 10 |
| | D265C-hD- | | | | for 4 weeks | |
| | c22b.8′ | | | | | |
| 8 | Brentuximab- | 12.5 | — | i.v. | 1x/week | 10 |
| | LALA-D256C | | | | for 4 weeks | |

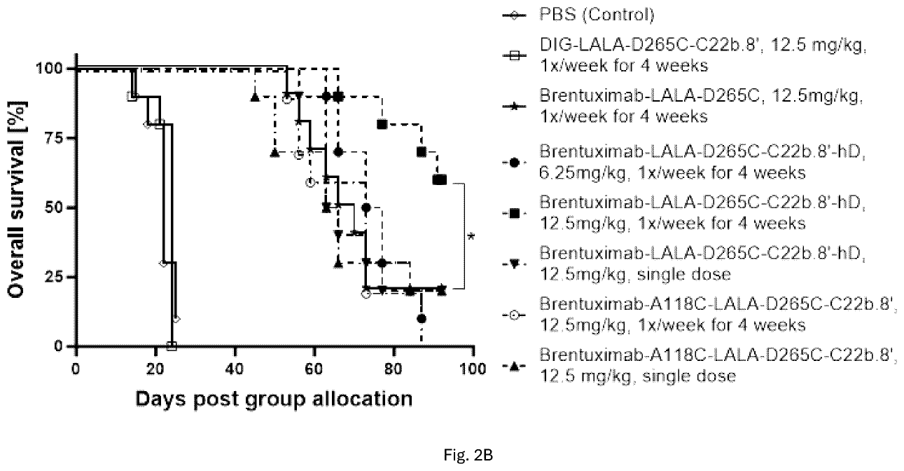

FIG. 2 shows: (A) Mean tumor volume [mm3] depicted from day 0 to day 91 post group allocation, measured by caliper. Mice were treated either with vehicle control, non-targeting ADC, naked antibody control, or ADC, as single or multiple doses (n=10 animals/group, mean depicted±SD). Treatment with ADC or naked antibody control significantly reduced tumor growth in vivo. Non-targeting ADC did not show any effect on tumor growth as compared with vehicle control. Naked antibody showed a similar anti-tumor efficacy as ADC with NAMPT inhibitor as payload. (B) Survival of animals treated with Brentuximab-LALA-D265C-hD-c22b.8′ at multiple doses of 12.5 mg/kg was significantly prolonged as compared to naked antibody control.

### III. NAMPTi-ADC Significantly Prolongs Overall Survival of L540 Disseminated Tumor-Bearing NXG Mice

Female NXG mice were inoculated intravenously with 5×10⁶ L540 tumor cells in 200 μL RPMI medium without phenol red into their tail vein on day −3. Treatment was started on day 0 (n=10 animals/group). The animals of groups 2-4 were treated with Brentuximab-LALA-D265C-hD-c22b.8′ either as single intravenous dose or multiple dose treatment once a week for four weeks. The animals of groups 5 and 6 were treated with Brentuximab-A118C-LALA-D265C-c22b.8′ either as single intravenous dose or multiple dose treatment once a week for four weeks. Animals of negative control groups 7 and 8 were either treated with non-targeting ADC DIG-LALA-D265C-hD-c22b.8′ or naked antibody Brentuximab-LALA-D265C once a week for four weeks. Body weights were determined twice per week. Clinical signs and survival were monitored daily. The animals were sacrificed, and necropsy was performed when one or more termination criteria arose or at study termination.

TABLE 19
Experimental groups

| | | Dose | *Dose | | | |
|---|---|---|---|---|---|---|
| | | Protein | Toxin | | | |
| Group | Compound | [mg/kg] | [μg/kg] | Route | Schedule | Animals (n) |
| 1 | PBS (Control) | — | — | i.v. | 1x/week | 10 |
| | | | | | for 4 | |
| | | | | | weeks | |
| 2 | Brentuximab-LALA- | 6.25 | 344.7 | i.v. | 1x/week | 10 |
| | D265C-hD-c22b.8′ | | | | for 4 | |
| | | | | | weeks | |
| 3 | Brentuximab-LALA- | 12.5 | 689.4 | i.v | 1x/week | 10 |
| | D265C- hD-c22b.8′ | | | | for 4 | |
| | | | | | weeks | |
| 4 | Brentuximab-LALA- | 12.5 | 689.4 | i.v. | 1x | 10 |
| | D265C- hD-c22b.8′ | | | | | |
| 5 | Brentuximab- | 12.5 | 251.1 | i.v. | 1x/week | 10 |
| | A118C-LALA- | | | | for 4 | |
| | D265C-c22b.8′ | | | | weeks | |
| 6 | Brentuximab- | 12.5 | 251.1 | i.v. | 1x | 10 |
| | A118C-LALA- | | | | | |
| | D265C-hD-c22b.8′ | | | | | |
| 7 | DIG-LALA-D265C- | 12.5 | 697.3 | i.v. | 1x/week | 10 |
| | hD-c22b.8′ | | | | for 4 | |
| | | | | | weeks | |
| 8 | Brentuximab-LALA- | 12.5 | — | i.v. | 1x/week | 10 |
| | D256C | | | | for 4 | |
| | | | | | weeks | |

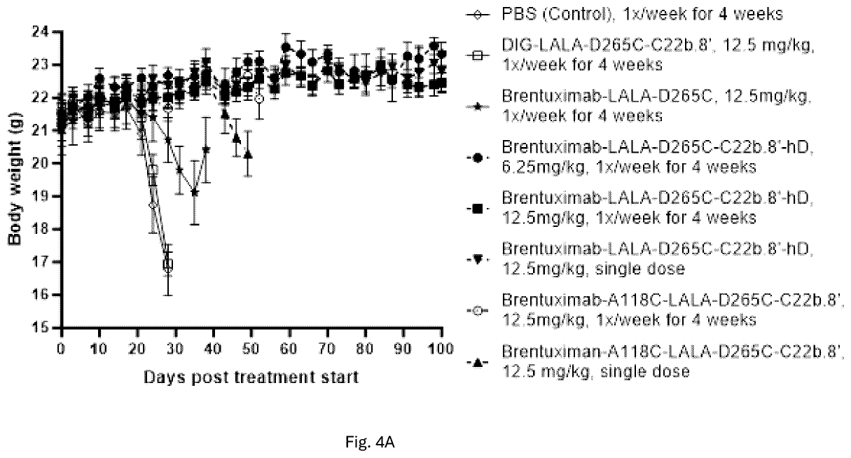

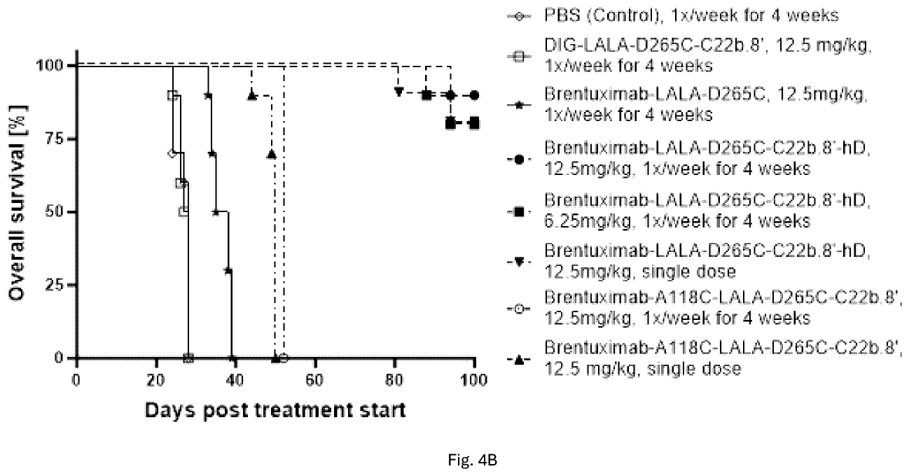

FIG. 4 shows (A) Mean absolute body weight [g] depicted from day 0 to day 100 post treatment. Mice were treated either with vehicle control, non-targeting ADC, naked antibody control, or ADC, as single or multiple doses (n=10 animals/group, mean depicted±SD). (B) Survival of animals treated with Brentuximab-LALA-D265C-hD-c22b.8′ at single or multiple doses was significantly prolonged as compared to vehicle and naked antibody control, and animals treated with Brentuximab-A118C-LALA-D265C-c22b.8′.

### IV. NAMPTi-ADC Shows Great Anti-Tumor Efficacy in NCI-N87 Subcutaneous Tumors in NMRI-Nu Mice

Female NMRI-nu mice were inoculated subcutaneously with 5×10⁶ NCI-N87 tumor cells in 200 μL RPMI medium plus 40 μL Matrigel without phenol red into their right flanks. Once a mean tumor volume of 135.94 mm³ was reached, animals were allocated to control or treatment groups according to tumor size (day 0; n=9 animals/group). On the day after (day 1), the animals of group 2 were treated once a day on weekdays for three weeks with NAMPT inhibitor FK866 intraperitoneally. The animals of group 3 and 4 were treated either with a single intravenous dose of anti-HER2 ADC (group 3) or once a week for three weeks (group 4). The tumor volume was measured twice per week by calliper and body weights were determined in parallel. Clinical signs and survival were monitored daily. The animals were sacrificed, and necropsy was performed when one or more termination criteria arose or at study termination.

TABLE 20
Experimental groups

| | | Dose | | | Animals |
|---|---|---|---|---|---|
| Group | Treatment 1 | [mg/kg] | Route | Schedule | (n) |
| 1 | PBS (Control) | — | i.p. | 1x/day for 3 | 8-10 |
| | | | | weeks | |
| 2 | NAMPTi | 5 | i.p. | 1x/day for 3 | 8-10 |
| | (FK866) | | | weeks | |
| 3 | T-LALA-D265C- | 50 | i.v. | Single dose | 8-10 |
| | hD-c42b.10′ | | | | |
| 4 | T-LALA-D265C- | 12.5 | i.v. | 1x/week for 4 | 8-10 |
| | hD-c42b.10′ | | | weeks | |

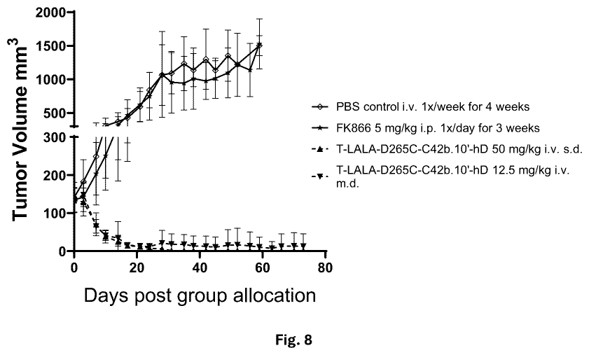

FIG. 8: Mean tumor volume [mm³] depicted from day 0 to day 73 post group allocation, measured by calliper. Mice were treated either with vehicle control, FK866, or ADC, as single (s.d.) or multiple doses (m.d.) (n=9 animals/group, mean depicted±SD). Treatment with ADC significantly inhibited tumor growth in vivo. All animals treated with ADC, survived until study end at day 73. Of these animals, all animals expect one animal were tumor-free until study end.

### Stability Regarding Lysosomal Degradation

Inhibitor 36 undergoes hydration of the cyanoguanidine moiety to guanylurea under acid pH. Additionally, the inventor demonstrated that in case of the biological lysosomal set up, hydration can be catalysed by lysosomal hydrolases at the lysosomal pH≈5.5 in contrast to the pH<2 required on the synthetic hydration (as reported for compound 113). This observation led to the next generation of NAMPT inhibitors from where the lead candidate is C42a.

The inhibitor C42a was set under identical conditions as those that triggered the hydration of 36. The compound C42a was stable for more than 2 weeks.

The cyanoguanidine group contained in NAMPT inhibitors such as 36 and C22a tend towards hydration and degradation at pH values below 2, leading to the formation of guanylurea intermediates according to the general reaction scheme described by Williams, et al. (J. Chem. Soc., Perkin Trans. 2, 1984, 1009-1013). Since the lysosomal pH is acidic and contains numerous hydrolases, NAMPT inhibitors bearing cyanoguanidines may therefore be degraded in the lysosome. Hence, the guanylurea 113 as hydration product of inhibitor 36 was chemically synthetised as a reference standard. When the acid hydration reaction involving TFA was performed at pH values above 2 (after 48 hours at 37° C.), the reaction did not occur. However, at pH values below 2, the expected guanylurea 113 was obtained in excellent yield and with minimal side products within 24 hours at 37° C. The product was confirmed by mass spectrometry and NMR analysis. The inhibitor C42a was designed as an alternative to cyanoguanidine compounds containing urea as non-hydratable group. Extensive tests at pH values below 2 at 37° C. for more than 2 weeks showed no hydration of C42a.

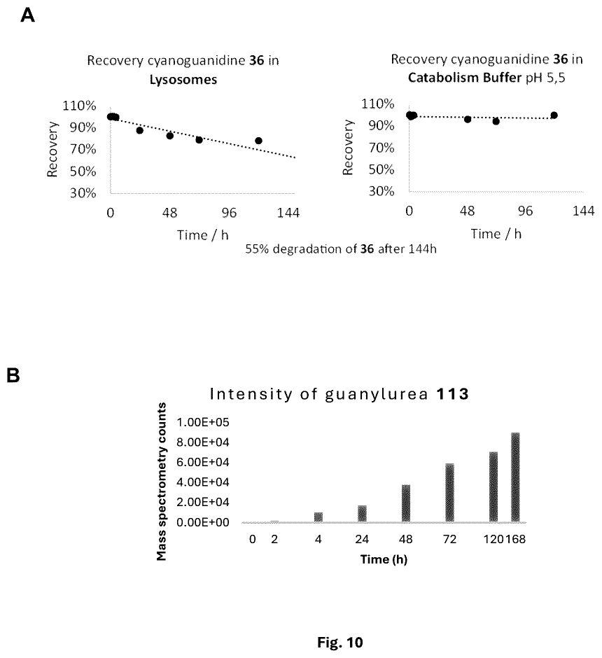

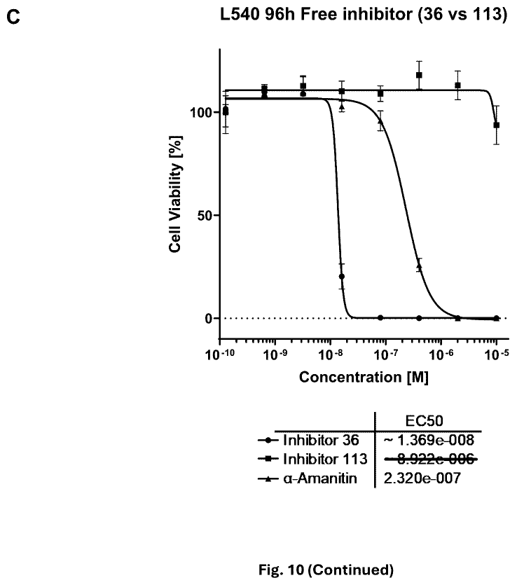

When tested in lysosomes 36 shows a fast and sustained degradation, while no degradation is observed in the absence of lysosomal enzymes. (See FIG. 10 A)

The degradation of 36 inversely correlates to the generation of the hydrated product 113. (See FIG. 10 B)

Regarding the cytotoxic activity, the hydration of the cyanoguanidine group led to compound 113, with significantly reduced toxicity in L540 cells compared to inhibitor 36 as shown in FIG. 10 C.

### Methods for Lysosomal Stability Assay:

The aim of this assay was to determine whether NAMPT inhibitors containing cyanoguanidine groups are degraded in lysosomes after release from the ADC.

The test compound was incubated at a concentration of 17.7 μM in a mixture of human liver lysosomal extract (tebu-bio, Seikisui Xenotech, Ref: H0610.L) and 1× catabolism buffer (tebu-bio, Seikisui Xenotech, Ref: K5200) for five days at 37° C. Samples were taken at 0, 2, 4, 24, 48, 72, 120, and 168 hours. Control samples without lysosomal extract were also set up under the same conditions as the test sample. All measurements and samples were taken in duplicate.

To measure the reaction, the compound was quenched by adding 380 μL of internal standard buffer (30 ng/mL of internal standard in ACN) to 20 μL of the sample. An amanitin-stable isotope-labelled analogue was used as the internal standard.

22 μL of the quenched sample was analysed using UPLC-MS-MS QTrap 5500 (Column: Waters ACQUITY UPLC BEH Amide, 1.7 m, 2.1×100 mm, product code: 186004801). This assay was related to compound 113. Compound 113 was expected to be the main degradation compound after lysosomal enzymatic digestion and was used as a reference for the detection of catabolites after digestion in the HPLC-MS analysis.

The ion fragments detection method was multiple reaction monitoring (MRM). In this method, the mass is measured by a tandem mass spectrometer where the initial molecular ion is selected as initial mass. Subsequently, the fragmentation of this molecular ion in the second stage of the tandem mass spectrometer is followed by selection of a product ion of the fragmentation reaction of the precursor ions. This method assures significantly more accurate detection and quantitation of molecules in complex matrices.

TABLE 21
Fragment detection

| | Solvent 1: H₂O + | Solvent 2: ACN + |
|---|---|---|
| Time (min) | 0.1% Formic acid | 0.1% Formic acid |
| 0 | 5 | 95 |
| 2 | 50 | 50 |
| 3 | 50 | 50 |
| 3.1 | 5 | 95 |
| 5.6 | 5 | 95 |
| 6.1 | 5 | 95 |

UPLC method was used to analyse the samples from the lysosomal stability assay.

1. A compound having the structure 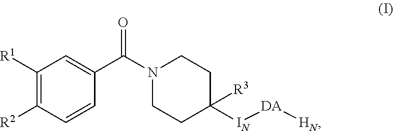
wherein R¹, R², R³, R^{L}, I_{N}, D_{A}, H_{N} are independently:
R¹ is OH, NH₂, N₃, SH, H, NHR^{L}; OR^{L}, or SR^{L};
R² is OH, NH₂, N₃, SH, H, NHR^{L}, OR^{L}, or SR^{L};
R³ is H-bond donor group, selected from the group consisting of OH, NH₂, SH, SO₃H, COOH, and CONH₂;
I_{N} is an interconnecting unit, selected from C₁₋₆ alkyl, 5 or 6-membered aromatic ring, a 5 or 6-membered heteroaromatic ring, or a combination thereof;
DA is a H-bond donor acceptor group selected from cyanoguanidine, acrylamide, urea, thiourea; and
H_{N} is heteroaromatic or heterocyclic ring selected from the group consisting of pyridyl, isoindolinyl, indolyl, isoquinolinyl, quinolinyl, and imidazopyridinyl;
R^{L} is a linker having the structure L-Z,
wherein L is a linker selected from a cleavable or non-cleavable linker; and
Z is a thiol-reactive, or amine-reactive chemical moiety, and wherein if R¹ is NHR^{L}, OR^{L}, or SR^{L}, R² is not NHR^{L}, OR^{L}, SR^{L} and if R² is NHR^{L}, OR^{L}, SR^{L}, R¹ is not NHR^{L} OR^{L}, SR^{L}.

2. The compound of claim 1, wherein H_{N} is selected from the group consisting of

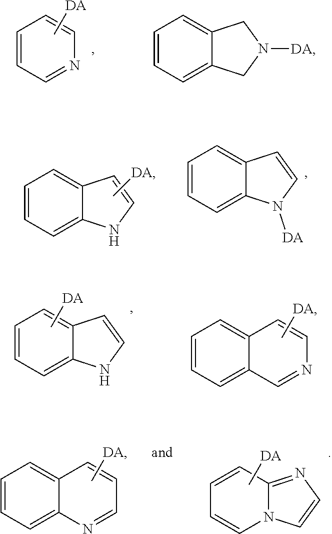

3. (canceled)

4. The compound of claim 1 having the structure selected from the group consisting of

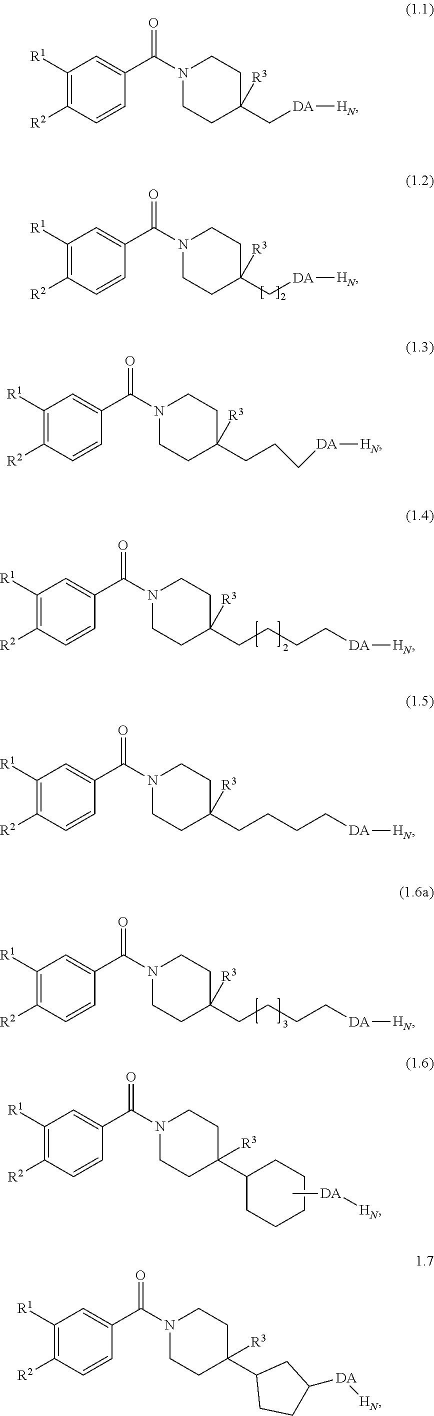

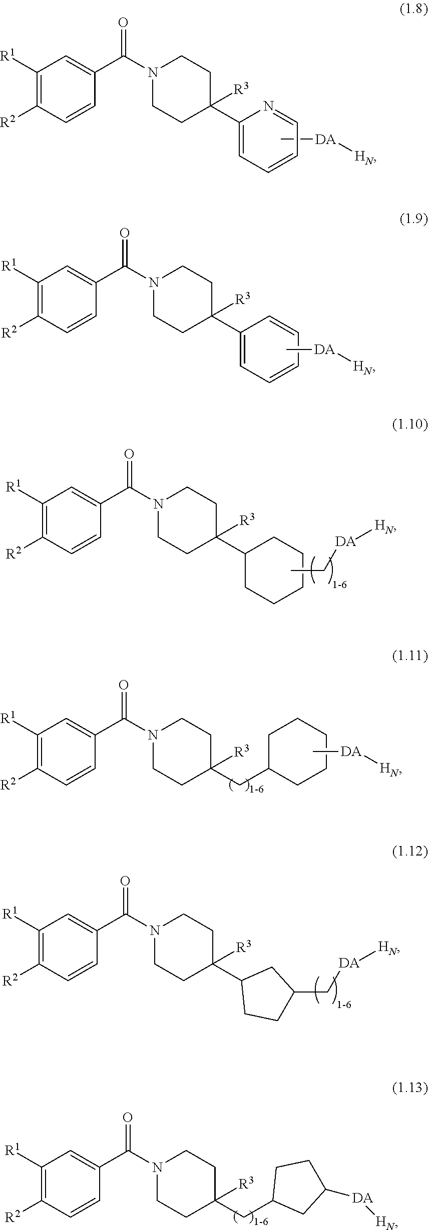

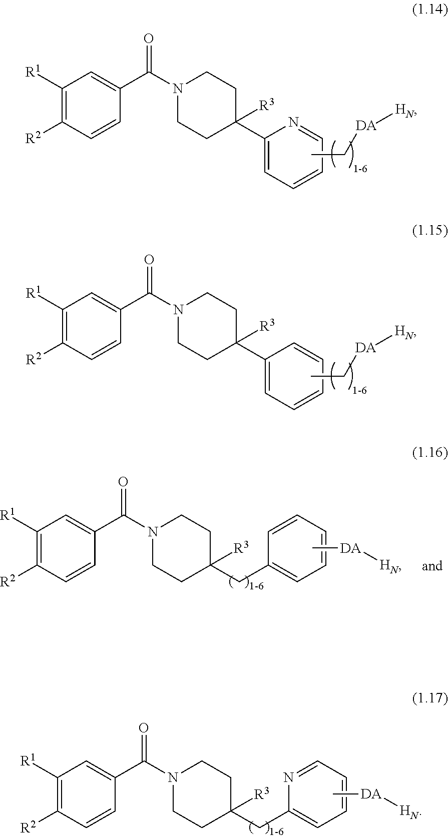

5. The compound of claim 4, wherein R³ is selected from the group consisting of —OH, —NH₂ and CONH₂; D_{A} is selected from the group consisting of cyanoguanidine, acrylamide, urea, and thiourea; and H_{N} is selected from the group consisting of pyridyl, isoindolinyl, indolyl, isoquinolinyl, quinolinyl, and imidazopyridinyl.

6-15. (canceled)

16. The compound of claim 5, selected from the group consisting of 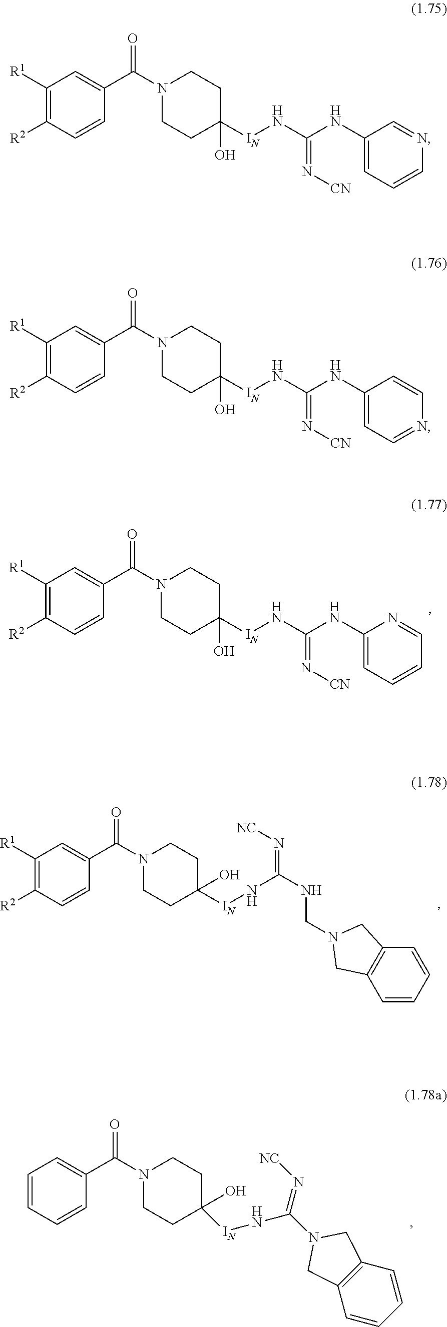 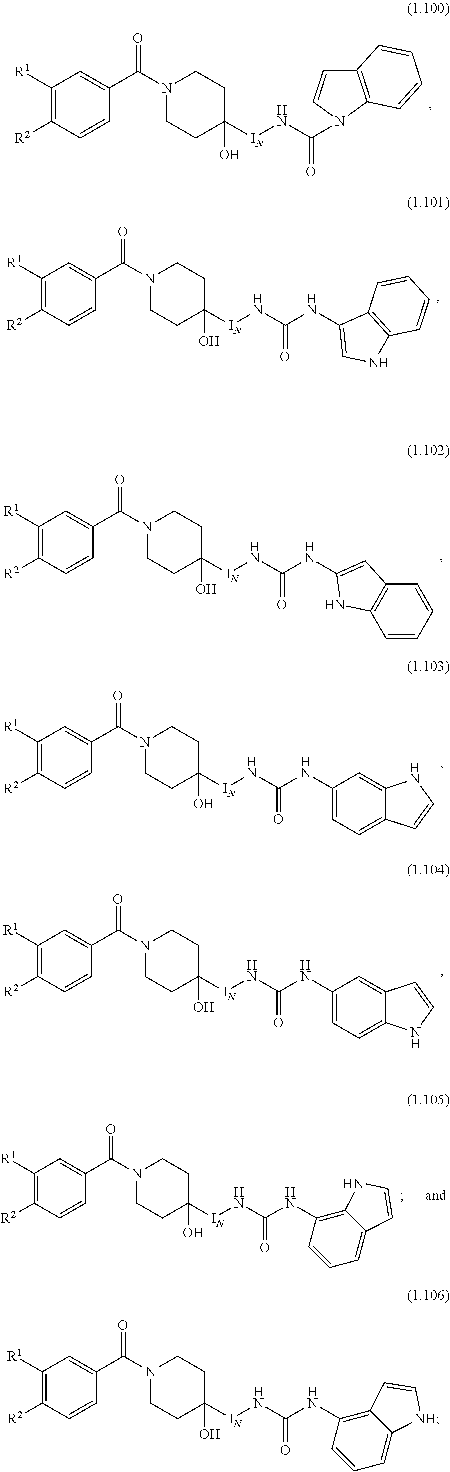
wherein
I_{N} is selected from C₁-C₆ alkyl, optionally, I_{N} is selected from C₃-C₆ alkyl
R¹ is OH, NH₂, N₃, SH, H, NHR^{L}; OR^{L}, or SR^{L}; and
R² is OH, NH₂, N₃, SH, H, NHR^{L}, OR^{L}, or SR^{L}.

17. (canceled)

18. (canceled)

19. The compound of claim 16, wherein I_{N} is C₄ and R¹ is NHR^{L}, R² is selected from the group consisting of H, OH, and SH, and R^{L} is a cleavable linker or a non-cleavable linker.

20. (canceled)

21. (canceled)

22. The compound of claim 16, wherein R¹ is selected from the group consisting of NHR^{L} OR^{L}, and SR^{L}; R² is selected from the group consisting of H, OH, NH₂, N₃, and SH; and R^{L} is a cleavable linker or a non-cleavable linker.

23-38. (canceled)

39. The compound of claim 22, wherein R^{L} is a cleavable linker, wherein the cleavable linker is selected from the group consisting of an enzymatically cleavable linker, preferably a protease-cleavable linker, and a chemically cleavable linker, optionally a linker comprising a disulfide bridge.

40. The compound of claim 39, wherein said cleavable linker is cleaved by Cathepsin A or B, matrix metalloproteinases (MMPs), elastases, glutathione (GSH), or β-glucuronidase and β-galactosidase, optionally Cathepsin B.

41. The compound of claim 40, wherein the Cathepsin B-cleavable linker comprises a di-peptide selected from the group consisting of Val-Cit, Val-Ala, or Phe-Lys, Val-Lys, Ala-Lys, Phe-Cit, Leu-Cit, Ile-Cit, Phe-Arg, Trp-Cit, cBu-Ala, cBu-Cit, Glu-Val-Ala, Glu-Val-Cit, Glu-cBu-Ala, and Glu-cBu-Cit.

42. (canceled)

43. The compound of claim 42, wherein the compound is selected from the group consisting of

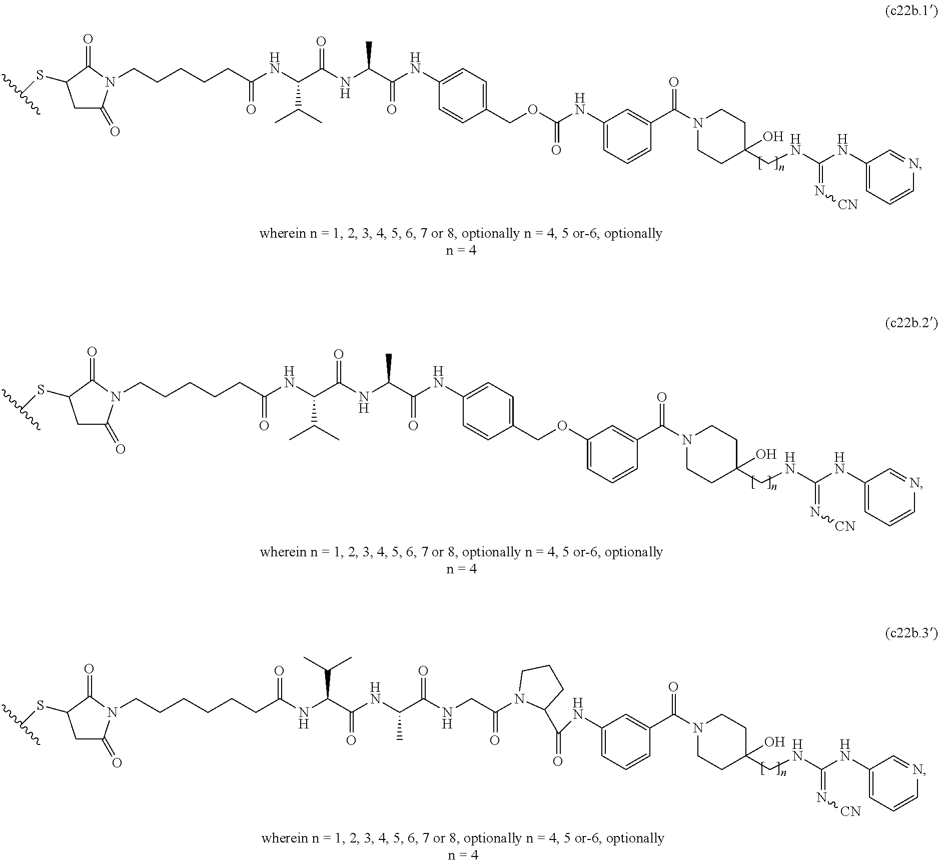

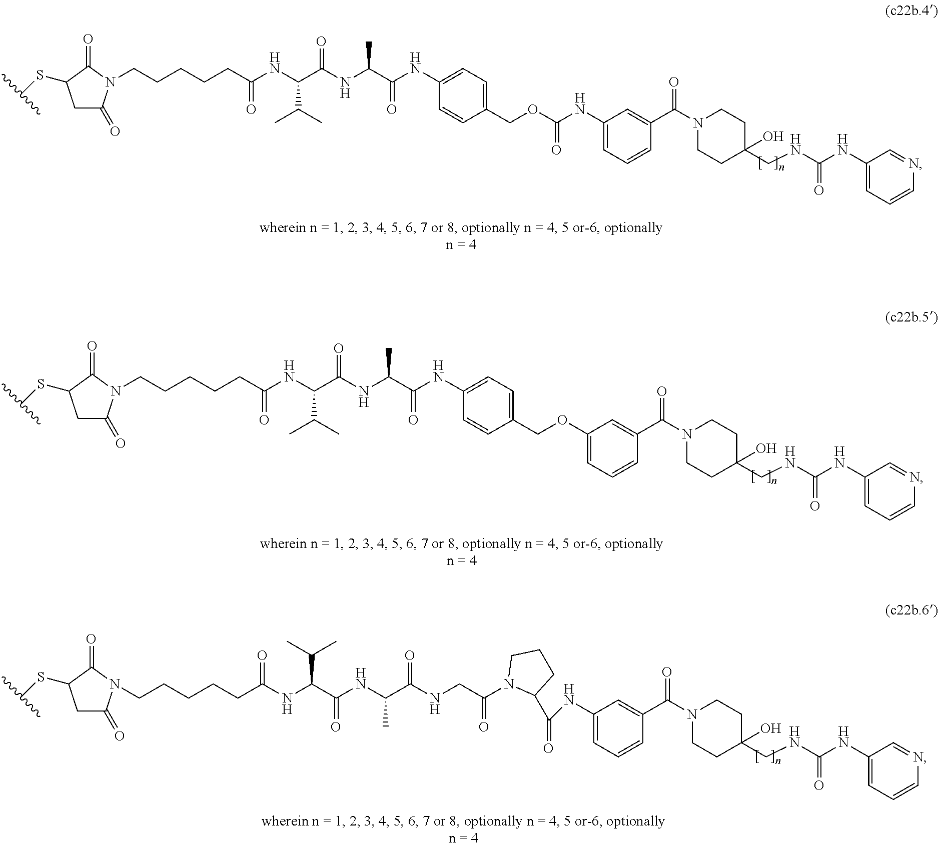

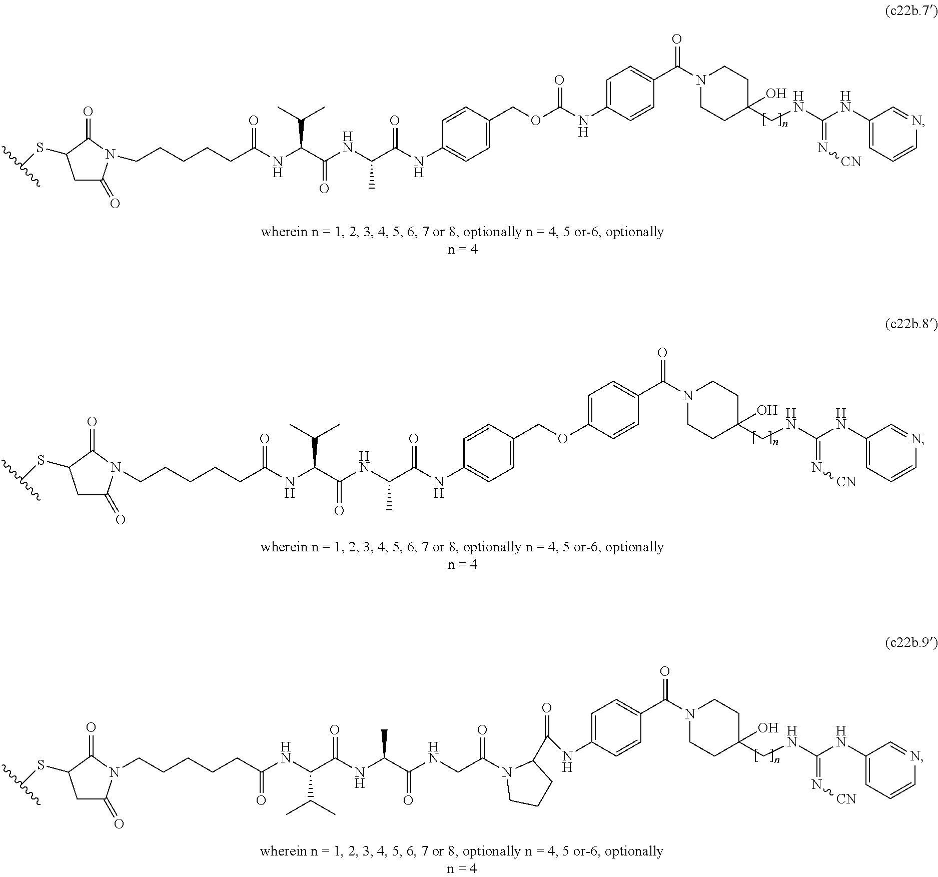

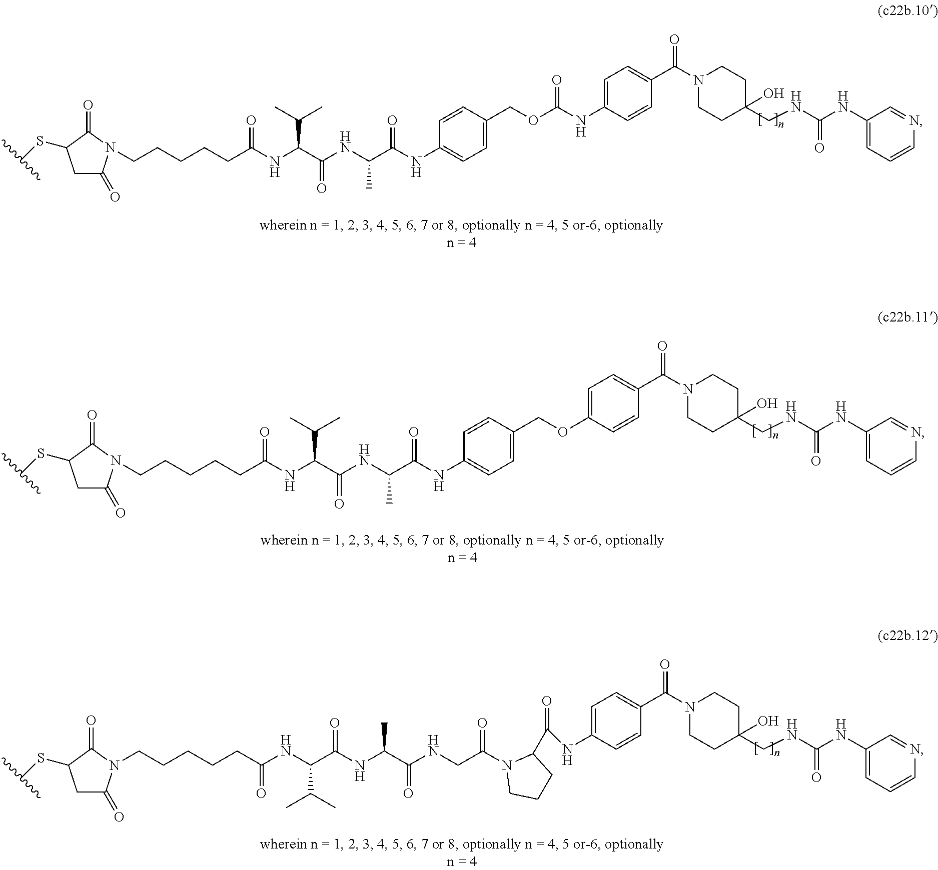

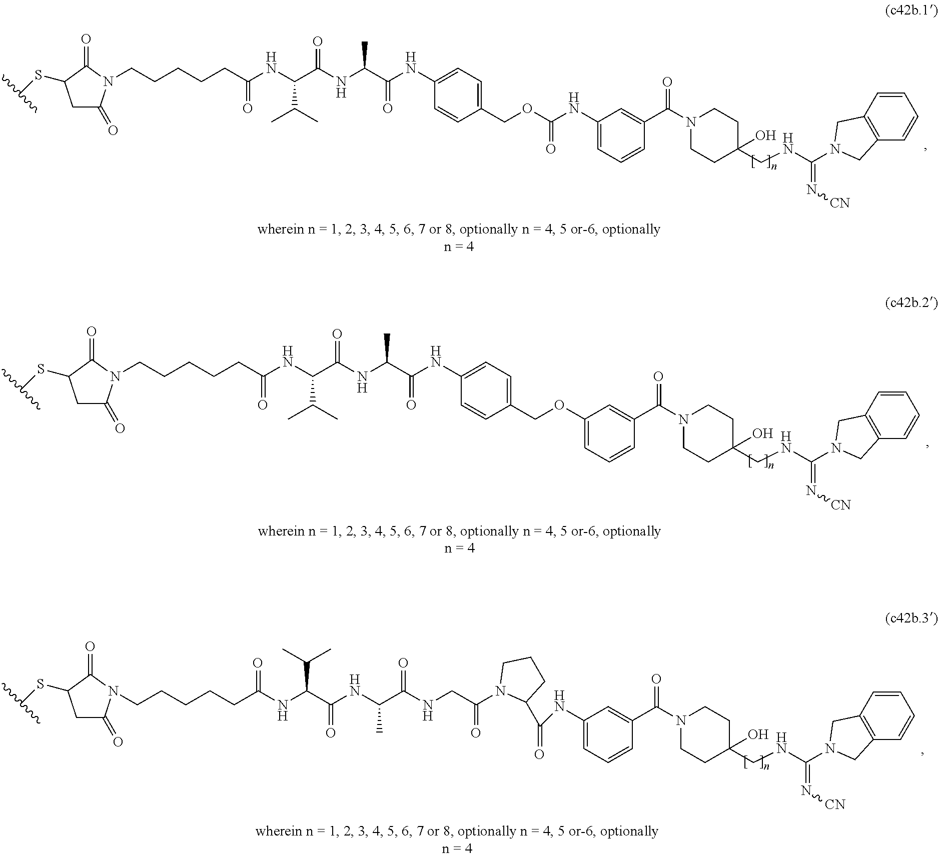

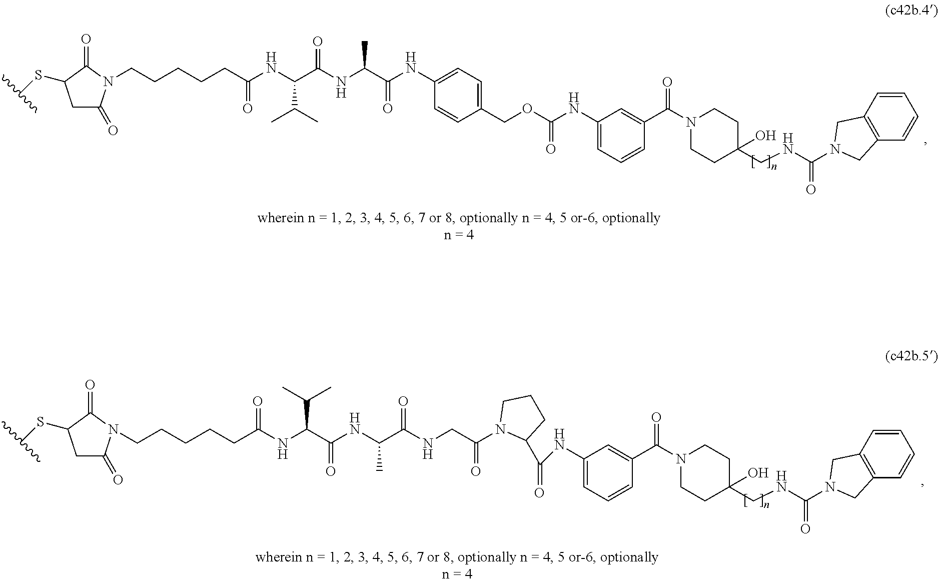

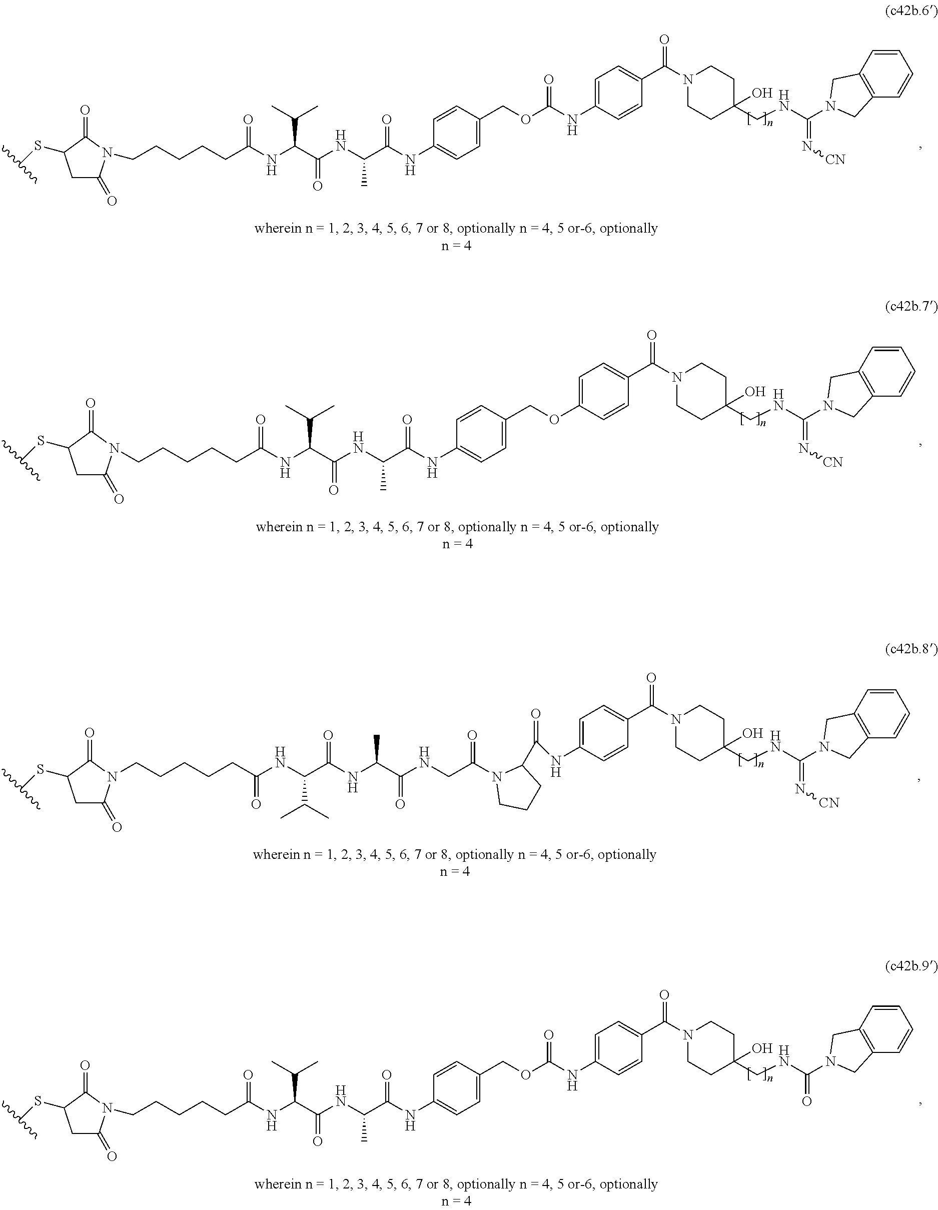

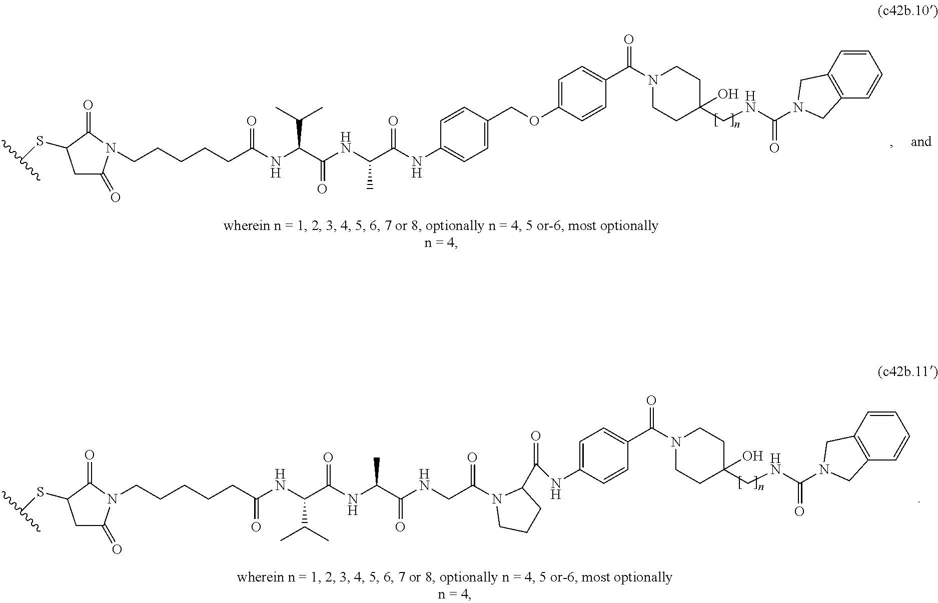

44. The compound of claim 43, wherein the compound is selected from the group consisting of

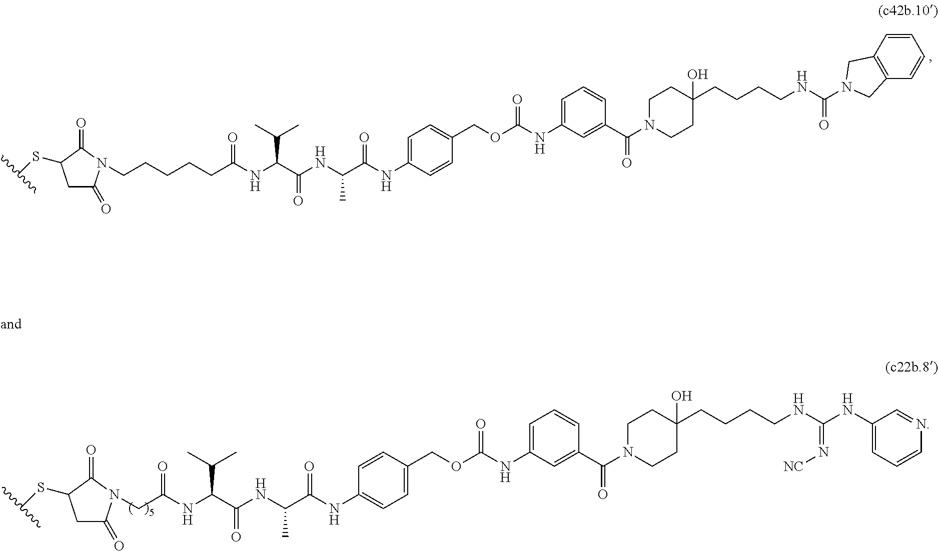

45. (canceled)

46. An antibody-drug conjugate (ADC)
Ab-(Z′-L-T)ₖ,
wherein,
Ab is an antibody or antigen-binding fragment thereof; or antibody-like protein,
Z is a chemical moiety formed from a coupling reaction between a reactive substituent present on L and a reactive substituent present within an antibody, or an antigen-binding fragment thereof;
L is a linker selected from a cleavable or non-cleavable linker;
T is a NAMPT inhibitor; and k is from about 1 to about 12.

47. The ADC of claim 46, wherein the antibody or antigen-binding fragment thereof is selected from the group consisting of monoclonal antibody or antigen-binding fragment thereof, a humanized antibody or antigen-binding fragment thereof, a human monoclonal antibody or antigen-binding fragment thereof, a bispecific antibody or antigen-binding fragment thereof, a dual-variable immunoglobulin domain, an intact antibody, a single-chain Fv molecule (scFv), a diabody, a triabody, a nanobody, an antibody-like protein scaffold, a Fv fragment, a Fab fragment, a F(ab′)₂ molecule, and a tandem di-scFv.

48. (canceled)

49. (canceled)

50. The ADC of claim 49, wherein the antibody, or an antigen-binding fragment thereof has an IgG isotype, optionally IgG1 or IgG4.

51. (canceled)

52. The ADC of claim 51, wherein the antibody comprises a heavy chain constant (Fc) region which comprises at least one amino acid substitution selected from the group consisting of L234A, L235A, A118C, S239C, and D265C (according to EU numbering system).

53. The ADC of claim 52, wherein the antibody comprises a heavy chain constant (Fc) region which comprises the amino acid substitution D265C; or the amino acid substitutions L234A, L235A and D265C; or the amino acid substitutions L234A, L235A, A118C, and D265C; or the amino acid substitutions L234A, L235A, S239C, and D265C (according to EU numbering system).

54. (canceled)

55. (canceled)

56. (canceled)

57. (canceled)

58. The ADC of claim 46, wherein the NAMPT inhibitor-linker conjugate (Z-L-T) is conjugated to at least one cysteine residue selected from heavy chain 118Cys, heavy chain 239Cys, or heavy chain 265Cys (according to EU numbering system); or at least two cysteine residues selected from heavy chain 118Cys and heavy chain 265Cys, or heavy chain 239Cys and/or heavy chain 265Cys.

59. (canceled)

60. The ADC of claim 46, wherein the NAMPT inhibitor-linker conjugate (Z-L-T) is conjugated to the antibody via naturally occurring reactive amine moieties or naturally occurring reactive cysteine residues.

61. (canceled)

62. (canceled)

63. The ADC of claim 46, wherein the ADC has a DAR of about 2, 4, 6 to about 8, 10, 12, optionally from about 4, 6, 8 to about 10, 12, or from about 10 to about 12.

64. The ADC of claim 63, wherein the NAMPT inhibitor linker conjugate (Z-L-T) is selected from the group consisting of

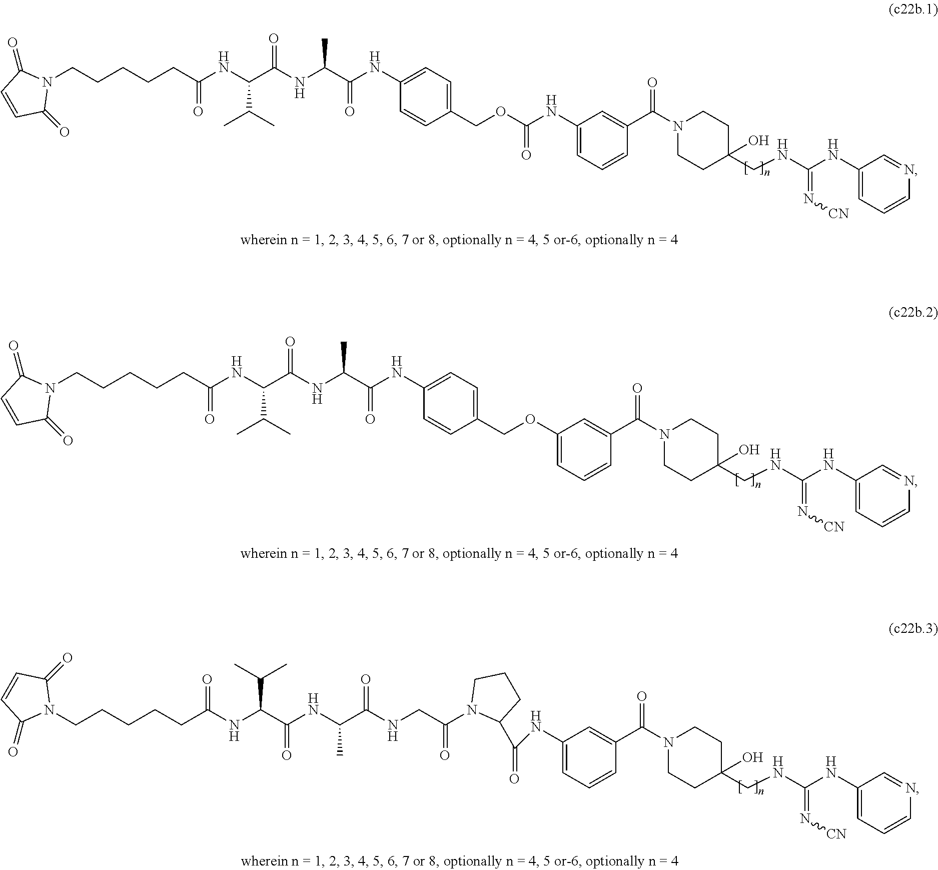

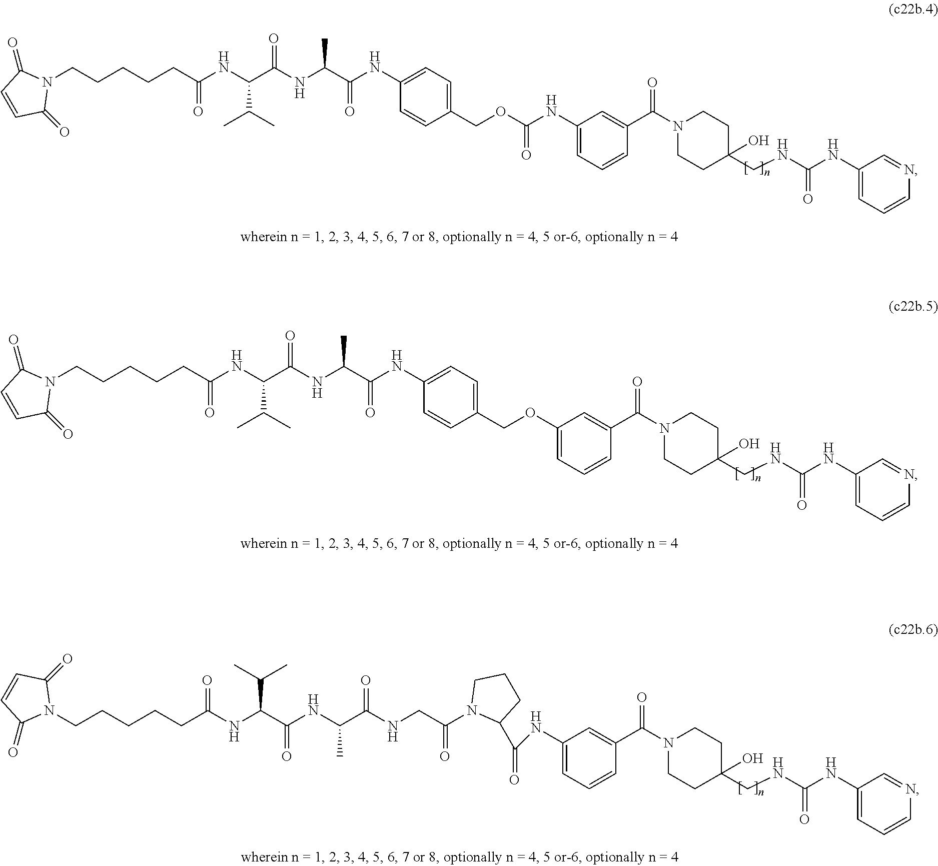

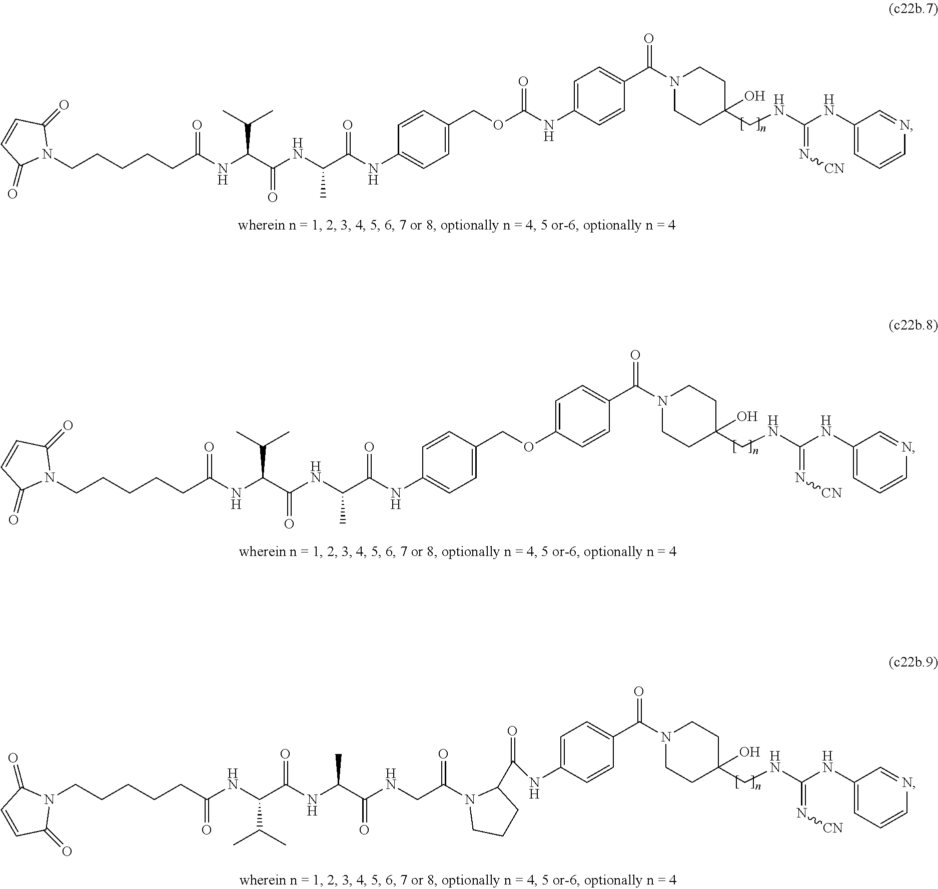

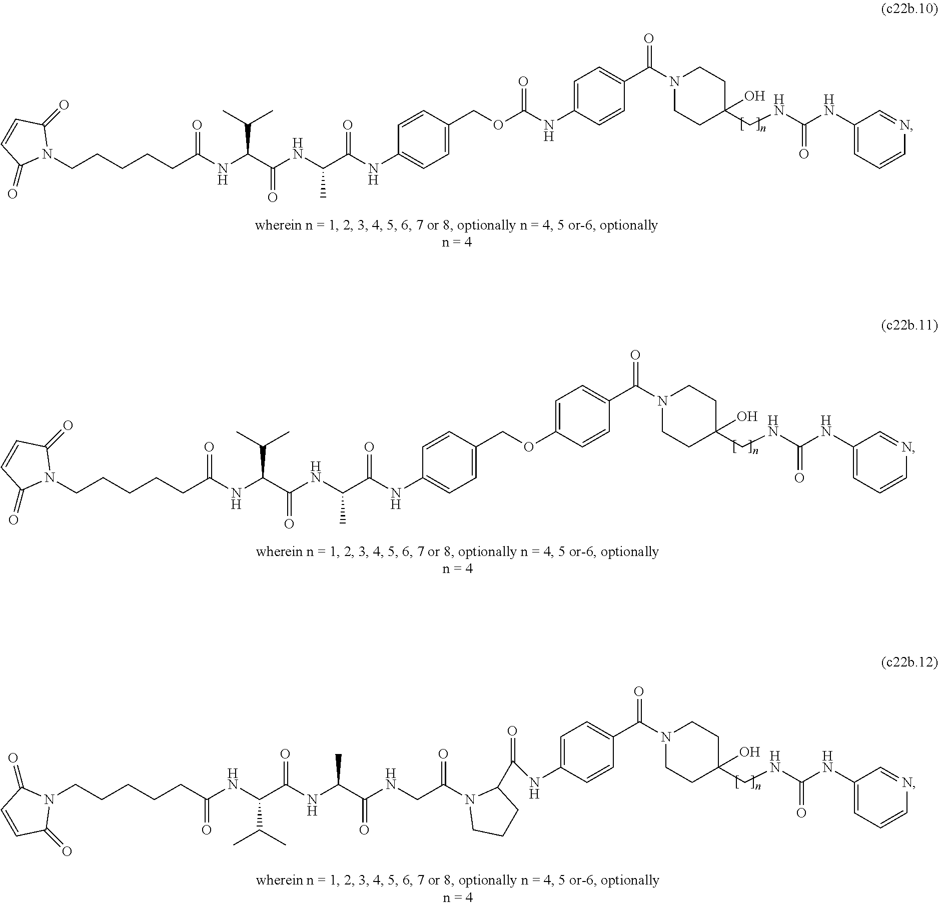

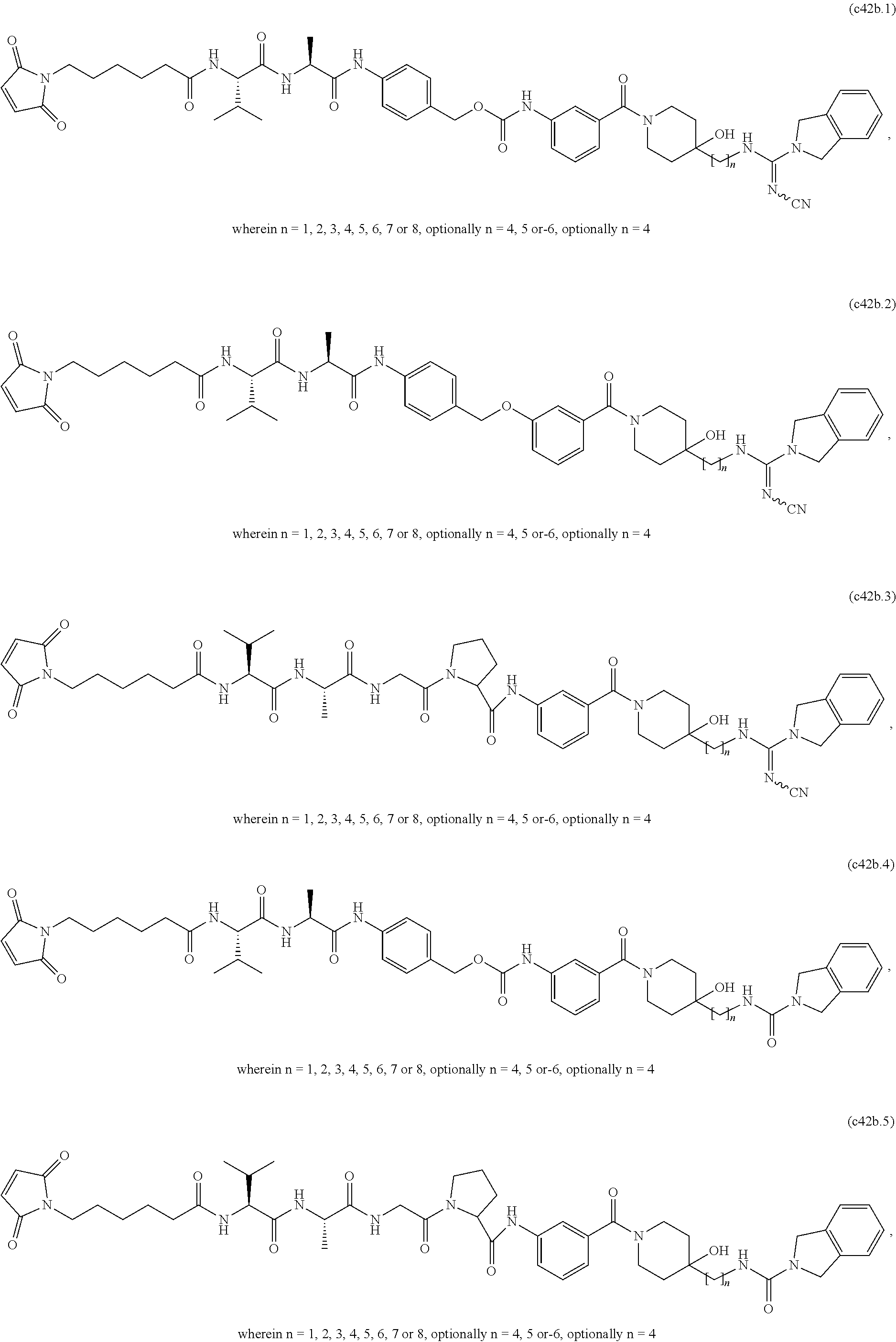

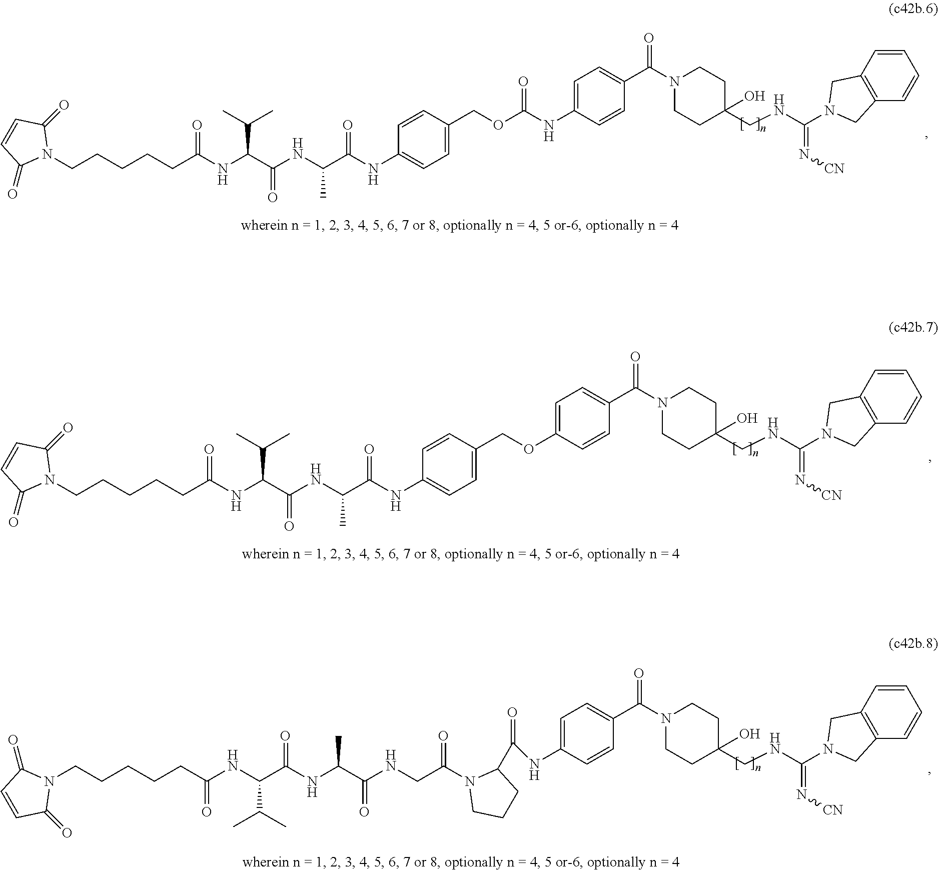

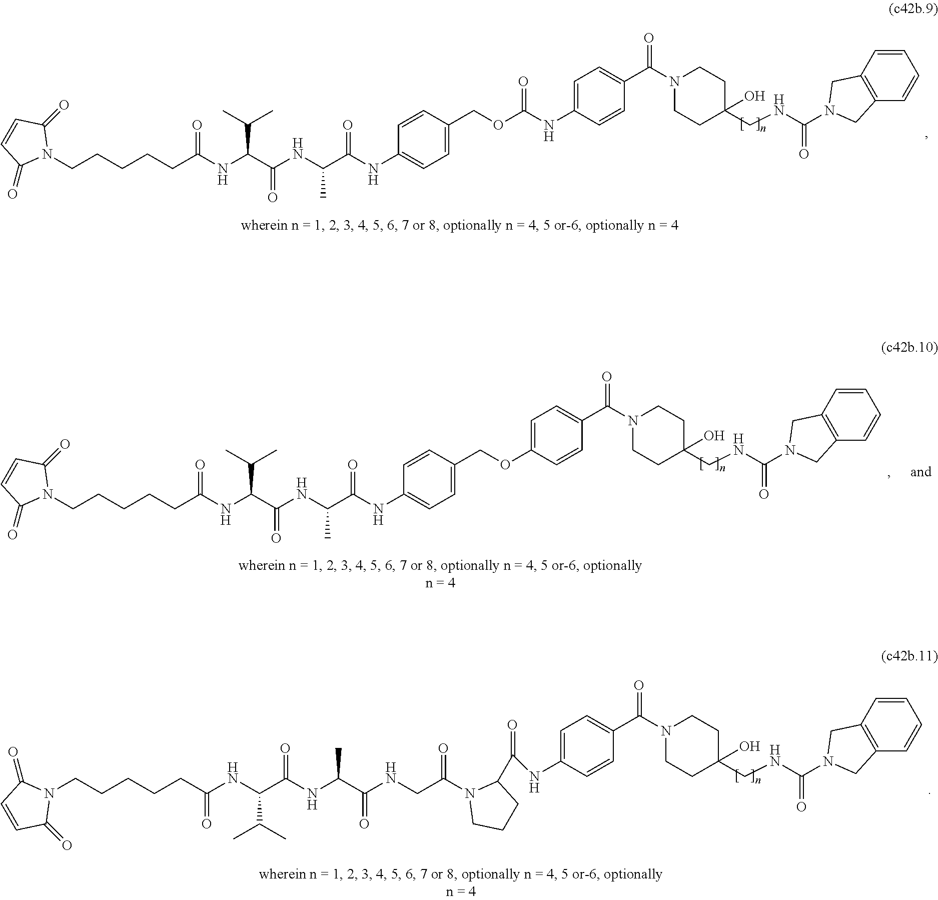

65. The ADC of claim 62, wherein the NAMPT inhibitor linker conjugate (Z-L-T) is

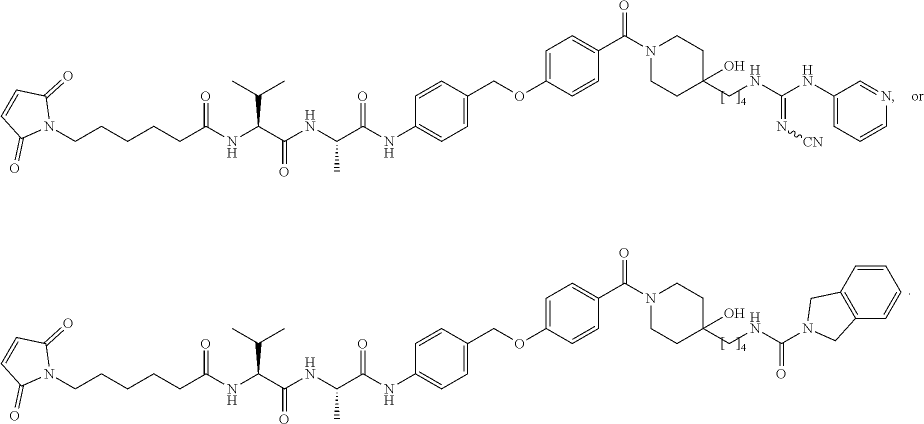

66. (canceled)

67. The ADC of claim 52, wherein the antibody or antigen-binding fragment thereof specifically binds to a tumor antigen or tumor-associated antigen.

68. The ADC of claim 64, wherein the tumor antigen is selected from the group consisting of CD2, CD5, CD19, CD20, CD30, CD37, CD45, CD117, CD123, CD137, BCMA (CD269), HER3, NY-ESO-1, tyrosinase, Melan-A/MART-1, Her-2/neu, survivin, telomerase, WT1, CEA, gp100, Pmell7, mammaglobin-A, NY—BR-1, ERBB2, OA1, PAP, R AB 38/NY-MEL-1, TRP-1/gp75, TRP-2, BAGE-1, D393-CD20n, cyclin-A1, GAGE-1, GAGE-2, GAGE-8, GnTVf, HERV-K-MEL, KK-LC-1, KM-HIN-1, LAGE-1, LY6K, MAGE-A1, MAGE-A2, MAGE-A3, MAGE-A4, MAGE-A6, MAGE-A9, MAGE-A10, MAGE-A12, MAGE-C1, MAGE-C2, mucin K, NA88-A, SAGE, spl7, SSX-2, SSX-4, TAG-1, TAG-3, TRAG-3, XAGE-lb, BCR-AB1, AIM-2, ALDH1A1, BCLX(L), BING-4, CALCA, CD274, CPSF, cyclin D1, DKK1, ENAH, EpCAM, EphA3, EZH2, FGF5, glypican-3, G250, HLA-DOB, hepsin, IDO1, IGF2B3, IL12Ralpha2, intestinal carboyxyl esterase, IGFIR, alpha-foetoprotein, kallikrein 4, KIF20A, Lengsin, M-CSF, M-CSP, mdm-2, MELOE-1, midkine, MMP-2, MMP-7, MUC1, MUC5AC, p53, PAX5, PBF, PRAME, prostate-specific membrane antigen (PSMA), RAGE-1, RGS5, RhoC, RNF43, RU2AS, SOX10, STEAP1, telomerase, TPBG, mesothelin, Axl, VEGF, EGFR, AFP, CA125, GUCY2C, TROP2, VEGFR-1, VEGFR-2, or VEGFR-3.

69. (canceled)

70. (canceled)

71. A composition comprising the compound of claim 1, optionally the composition is a pharmaceutical composition.

72. A composition comprising the ADC of claim 46, wherein the composition is a pharmaceutical composition.

73. (canceled)

74. A method of treating a patient afflicted with cancer, wherein the method comprises administering to said patient a pharmacologically effective amount of the ADC of claim 46.

75. (canceled)

76. The method of claim 74, wherein the cancer is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer (particularly HER2-positive breast cancer), ovarian cancer (particularly HER2-positive ovarian cancer), triple-negative breat cancer (TNBC), prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, pharynx squamous cell carcinoma, medulloblastoma, craniopharyngioma, ependymoma, epidermoid carcinoma of the vulva, pinealoma, hemangioblastoma, acoustic neuroma, oligodendroglioma, meningioma, skin cancer, melanoma, neuroblastoma, retinoblastoma, acute lymphoblastic leukemia “ALL”, acute lymphoblastic B-cell leukemia, acute lymphoblastic T-cell leukemia, acute myeloblastic leukemia “AML”, acute promyelocytic leukemia “APL”, acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia “CML”, chronic lymphocytic leukemia “CLL”, hairy cell leukemia, multiple myeloma, lymphoblastic, myelogenous, lymphocytic, myelocytic leukemias, Lymphomas include Hodgkin's disease, non-Hodgkin's Lymphoma, Multiple myeloma, Waldenstrom's macroglobulinemia, Heavy chain disease, Polycythemia vera.

77. The method of claim 74, wherein the cancer is deficient in the nicotinic acid pathway.

78. A method of inhibiting nicotinamide phosphoribosyltransferase (NAMPT), wherein the method comprises exposing cells to a pharmacologically effective amount of the ADC of claim 46.

79. (canceled)

80. (canceled)
